(19) United States
(12) Patent Application Publication
(10) Pub. No.: US 2009/0076075 A1
(43) Pub. Date: Mar. 19, 2009

(54) QUINOLINE DERIVATIVES

(76) Inventors: Frederic Henri Jung, Reims (FR); Remy Robert Morgentin, Reims (FR); Patrick Ple, Reims (FR)

Correspondence Address: MORGAN LEWIS & BOCKIUS LLP, 1111 PENNSYLVANIA AVENUE NW, WASHINGTON, DC, 20004, US

(21) Appl. No.: 12/280,852

(22) PCT Filed: Mar. 1, 2007
(86) PCT No.: PCT/GB2007/000713

§ 371 Date: Aug. 27, 2008

(30) Foreign Application Priority Data
Mar. 2, 2006 (EP) 06300183.8
Oct. 31, 2006 (EP) 06301103.5

(51) Int. Cl. A61K 31/4709; C07D 401/14

(52) U.S. Cl. 514/312; 546/153

(57) ABSTRACT

The invention concerns quinoline derivatives of Formula I

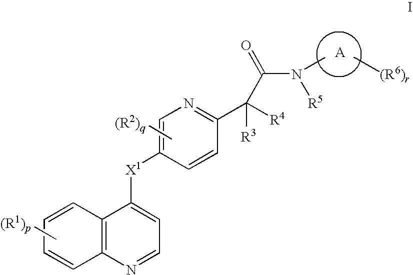

or a pharmaceutically-acceptable salt thereof, wherein each of X¹, p, R¹, q, R², R³, R⁴, R⁵Ring A, r and R⁶ has any of the meanings defined hereinbefore in the description; processes for their preparation, pharmaceutical compositions containing them and their use in the manufacture of a medicament for use in the treatment of cell proliferative disorders.

The invention concerns certain novel quinoline derivatives, or pharmaceutically-acceptable salts thereof, which possess anti-cancer activity and are accordingly useful in methods of treatment of the human or animal body. The invention also concerns processes for the manufacture of said quinoline derivatives, pharmaceutical compositions containing them and their use in therapeutic methods, for example in the manufacture of medicaments for use in the prevention or treatment of cancers in a warm-blooded animal such as man, including use in the prevention or treatment of solid tumour disease.

Many of the current treatment regimes for the abnormal cell growth found in cell proliferation diseases such as psoriasis and cancer utilise compounds which inhibit DNA synthesis. Such compounds are toxic to cells generally but their toxic effect on rapidly dividing cells such as tumour cells can be beneficial. Alternative approaches to anti-cancer agents which act by mechanisms other than the inhibition of DNA synthesis have the potential to display enhanced selectivity of action.

Eukaryotic cells are continually responding to many diverse extracellular signals that enable communication between cells within an organism. These signals regulate a wide variety of physical responses in the cell including proliferation, differentiation, apoptosis and motility. The extracellular signals take the form of a diverse variety of soluble factors including growth factors as well as paracrine, autocrine and endocrine factors. By binding to specific transmembrane receptors, growth factor ligands communicate extracellular signals to the intracellular signalling pathways, thereby causing the individual cell to respond to extracellular signals. Many of these signal transduction processes utilise the reversible process of the phosphorylation of proteins involving specific kinases and phosphatases.

As phosphorylation is such an important regulatory mechanism in the signal transduction process, it is not surprising that aberrations in the process result in abnormal cell differentiation, transformation and growth. For example, it has been discovered that a cell may become cancerous by virtue of the transformation of a portion of its DNA into an oncogene. Several such oncogenes encode proteins which are receptors for growth factors, for example tyrosine kinase enzymes. Tyrosine kinases may also be mutated to constitutively active forms that result in the transformation of a variety of human cells. Alternatively, the over-expression of normal tyrosine kinase enzymes may also result in abnormal cell proliferation.

Tyrosine kinase enzymes may be divided into two groups:—the receptor tyrosine kinases and the non-receptor tyrosine kinases. About 90 tyrosine kinase have been identified in the human genome, of which about 60 are of the receptor type and about 30 are of the non-receptor type. These can be categorized into 20 receptor tyrosine kinase sub-families according to the families of growth factors that they bind and into 10 non-receptor tyrosine kinase sub-families (Robinson et al, Oncogene, 2000, 19, 5548-5557). The classification includes the EGF family of receptor tyrosine kinases such as the EGF, TGFα, Neu and erbB receptors, the insulin family of receptor tyrosine kinases such as the insulin and IGF1 receptors and insulin-related receptor (IRR) and the Class III family of receptor tyrosine kinases such as the platelet-derived growth factor (PDGF) receptor tyrosine kinases, for example the PDGFα and PDGFβ receptors, the stem cell factor receptor tyrosine kinase (SCF RTK, commonly known as c-Kit), the fms-related tyrosine kinase 3 (Flt3) receptor tyrosine kinase and the colony-stimulating factor 1 receptor (CSF-1R) tyrosine kinase.

It has been discovered that such mutated and over-expressed forms of tyrosine kinases are present in a large proportion of common human cancers such as the leukaemias, breast cancer, prostate cancer, non-small cell lung cancer (NSCLC) including adenocarcinomas and squamous cell cancer of the lung, gastrointestinal cancer including colon, rectal and stomach cancer, bladder cancer, oesophageal cancer, ovarian cancer and pancreatic cancer. As further human tumour tissues are tested, it is expected that the widespread prevalence and relevance of tyrosine kinases will be further established. For example, it has been shown that EGFR tyrosine kinase is mutated and/or over-expressed in several human cancers including in tumours of the lung, head and neck, gastrointestinal tract, breast, oesophagus, ovary, uterus, bladder and thyroid.

Platelet-derived growth factor (PDGF) is a major mitogen for connective tissue cells and other cell types. The PDGF receptors comprising PDGFα and PDGFβ receptor isozymes display enhanced activity in blood vessel disease (for example atherosclerosis and restenosis, for example in the process of restenosis subsequent to balloon angioplasty and heart arterial by-pass surgery). Such enhanced PDGF receptor kinase activity is also observed in other cell proliferative disorders such as fibrotic diseases (for example kidney fibrosis, hepatic cirrhosis, lung fibrosis and multicystic renal dysplasia), glomerulonephritis, inflammatory diseases (for example rheumatoid arthritis and inflammatory bowel disease), multiple sclerosis, psoriasis, hypersensitivity reactions of the skin, allergic asthma, insulin-dependent diabetes, diabetic retinopathy and diabetic nephropathy.

The PDGF receptors can also contribute to cell transformation in cancers and leukaemias by autocrine stimulation of cell growth. It has been shown that PDGF receptor kinases are mutated and/or over-expressed in several human cancers including in tumours of the lung (non-small cell lung cancer and small cell lung cancer), gastrointestine (such as colon, rectal and stomach tumours), prostate, breast, kidney, liver, brain (such as glioblastoma), oesophagus, ovary, pancreas and skin (such as dermatofibrosarcoma protruberans) and in leukaemias and lymphomas such as chronic myelogenous leukaemia (CML), chronic myelomonocytic leukaemia (CMML), acute lymphocyte leukaemia (ALL) and multiple myeloma. Enhanced cell signalling by way of the PDGF receptor tyrosine kinases can contribute to a variety of cellular effects including cell proliferation, cellular mobility and invasiveness, cell permeability and cellular apoptosis.

Accordingly, antagonism of the activity of PDGF receptor kinases is expected to be beneficial in the treatment of a number of cell proliferative disorders such as cancer, especially in inhibiting tumour growth and metastasis and in inhibiting the progression of leukaemia.

In addition, PDGF is involved in angiogenesis, the process of forming new blood vessels, that is critical for continuing tumour growth. Normally, angiogenesis plays an important role in processes such as embryonic development, wound healing and several components of female reproductive function. However, undesirable or pathological angiogenesis has been associated with a number of disease states including diabetic retinopathy, psoriasis, cancer, rheumatoid arthritis, atheroma, Kaposi's sarcoma and haemangioma. Angiogenesis is stimulated via the promotion of the growth of endothelial cells. Several polypeptides with in vitro endothelial cell growth promoting activity have been identified including acidic and basic fibroblast growth factors (aFGF and bFGF) and vascular endothelial growth factor (VEGF). By virtue of the restricted expression of its receptors, the growth factor activity of VEGF, in contrast to that of aFGF and bFGF, is relatively specific towards endothelial cells. Recent evidence indicates that VEGF is an important stimulator of both normal and pathological angiogenesis and vascular permeability. This cytokine induces a vascular sprouting phenotype by inducing endothelial cell proliferation, protease expression and migration which subsequently leads to the formation of capillary tubes that promote the formation of the hyperpermeable, immature vascular network which is characteristic of pathological angiogenesis. The receptor tyrosine kinase (RTK) sub-family that binds VEGF comprises the kinase insert domain-containing receptor KDR (also referred to as Flk-1), the fins-like tyrosine kinase receptor Flt-1 and the fms-like tyrosine kinase receptor Flt-4. Two of these related RTKs, namely Flt-1 and KDR, have been shown to bind VEGF with high affinity.

Accordingly, antagonism of the activity of VEGF is expected to be beneficial in the treatment of a number of disease states that are associated with angiogenesis and/or increased vascular permeability such as cancer, especially in inhibiting the development of tumours.

It is known that several compounds with PDGF receptor kinase inhibitory activity are progressing toward clinical development. The 2-anilinopyrimidine derivative known as imatinib (STI571; Nature Reviews, 2002, 1, 493-502; Cancer Research, 1996, 56, 100-104) has been shown to inhibit PDGF receptor kinase activity although its current clinical use is for the treatment of CML based on its additional activity as an inhibitor of BCR-ABL kinase. STI571 inhibits the growth of glioblastoma tumours arising from injection into the brains of nude mice of the human glioblastoma lines U343 and U87 (Cancer Research, 2000, 60, 5143-5150). The compound also inhibits the in vivo growth of dermatofibrosarcoma protruberans cell cultures (Cancer Research, 2001, 61, 5778-5783). Based on the PDGF receptor kinase inhibitory activity of the compound, clinical trials are being carried out in glioblastoma and in prostate cancer. Several other PDGF receptor kinase inhibitors are being investigated including quinoline, quinazoline and quinoxaline derivatives (Cytokine & Growth Factor Reviews, 2004, 15, 229-235).

It is known from International Patent Application WO 92/20642 that certain aryl and heteroaryl compounds inhibit EGF and/or PDGF receptor tyrosine kinase. There is the disclosure of certain quinoline derivatives therein but no specific mention is made therein of 2-(2-pyridyl)acetamide derivatives; in particular, there is no specific mention made therein of quinolin-4-yloxy-substituted 2-(2-pyridyl)acetamide derivatives.

It is disclosed in many published patent applications such as International Patent Application WO 96/09294 that 4-anilinoquinazolines, 4-aryloxyquinazolines, 4-anilinoquinolines or 4-aryloxyquinolines possess tyrosine kinase enzyme inhibitory activity. However, there is no specific mention made therein of quinolin-4-yloxy-substituted 2-(2-pyridyl)acetamide compounds.

For example, it is known from International Patent Applications WO 02/36570 and WO 02/44166 that certain aryl and heteroaryl compounds inhibit MEK receptor tyrosine kinase. There is the disclosure therein of certain quinoline derivatives therein but no specific mention is made therein of 2-(2-pyridyl)acetamide derivatives; in particular, there is no specific mention made therein of quinolin-4-yloxy-substituted 2-(2-pyridyl)acetamide derivatives.

For example, it is known from International Patent Application WO 02/092571 that certain 3-carbamoylquinoline compounds inhibit JAK kinase. There is the disclosure therein of certain quinolin-4-ylamino-substituted 2-phenylacetamide derivatives but there is no specific mention made therein of 2-(2-pyridyl)acetamide derivatives; in particular, there is no specific mention made therein of N-aryl- or N-heteroaryl-2-(2-pyridyl)acetamide derivatives.

It is known from International Patent Application WO 2005/021554 that thienopyridine-substituted 2-phenylacetamide compounds inhibit VEGF receptor tyrosine kinases and provide an antiangiogenic effect. There is the disclosure in example 87 therein of a single quinolin-4-yloxy-substituted 2-phenylacetamide, namely of the compound N-(5-chloropyridin-2-yl)-2-[4-(7-methoxyquinolin-4-yloxy)phenyl]acetamide.

We have now found that surprisingly certain novel quinolin-4-yloxy-substituted 2-(2-pyridyl)acetamide compounds possess potent activity against cell proliferative disorders. It is believed that the compounds provide a useful treatment of cell proliferative disorders, for example to provide an anti-tumour effect, by way of a contribution from inhibition of PDGF receptor tyrosine kinases.

A further characteristic of hyperproliferative diseases such as cancer is damage to the cellular pathways that control progress through the cell cycle which, in normal eukaryotic cells, involves an ordered cascade of protein phosphorylation. As for signal transduction mechanisms, several families of protein kinases appear to play critical roles in the cell cycle cascade. The most widely studied of these cell cycle regulators is the cyclin dependent kinase family (the CDKs). Activity of specific CDKs at specific times is essential both to initiate and coordinate progress through the cell cycle. For example, the CDK4 protein appears to control entry into the cell cycle (the G0-G1-S transition) by phosphorylating the retinoblastoma gene product pRb which stimulates the release of the transcription factor E2F from pRb which, in turn, acts to increase the transcription of genes necessary for entry into S phase. The catalytic activity of CDK4 is stimulated by binding to a partner protein, Cyclin D. One of the first demonstrations of a direct link between cancer and the cell cycle was made with the observation that the Cyclin D1 gene was amplified and Cyclin D protein levels increased in many human tumours.

More recently, protein kinases that are structurally distinct from the CDK family have been identified which play critical roles in regulating the cell cycle and which also appear to be important in oncogenesis. They include the human homologues of the Drosophila aurora and S. cerevisiae Ip11 proteins. The three human homologues of these genes Aurora-A, Aurora-B and Aurora-C encode cell cycle regulated serine-threonine protein kinases that show a peak of expression and kinase activity through G2 and mitosis. Several observations implicate the involvement of human aurora proteins in cancer, especially Aurora-A and Aurora-B. Abrogation of Aurora-A expression and function by antisense oligonucleotide treatment of human tumour cell lines leads to cell cycle arrest and exerts an anti-proliferative effect. Additionally, small molecule inhibitors of Aurora-A and Aurora-B have been demonstrated to have an anti-proliferative effect in human tumour cells.

It is disclosed in International Patent Applications WO 01/21594, WO 01/21596 and WO 01/21597 that certain quinazoline derivatives that carry an anilino or phenoxy group linked to the 4-position of the quinazoline ring possess Aurora kinase inhibitory activity. There is no specific mention therein of 2-(2-pyridyl)acetamide derivatives; in particular, there is no specific mention made therein of quinazoline-substituted or quinoline-substituted 2-(2-pyridyl)acetamide derivatives.

It is disclosed in International Patent Application WO 01/55116 that certain quinoline derivatives that carry a heteroaryl group linked to the 4-position of the quinoline ring by, for example, a NH or O group possess Aurora kinase inhibitory activity. There is no specific mention therein of 2-(2-pyridyl)acetamide derivatives; in particular, there is no specific mention made therein of quinoline-substituted 2-(2-pyridyl)acetamide derivatives.

It is disclosed in International Patent Applications WO 02/00649, WO 03/055491, WO 04/058752, WO 04/058781 and WO 04/094410 that certain quinazoline derivatives that carry a 5-membered heteroaryl group linked to the 4-position of the quinazoline ring by, for example, a NH or O group possess Aurora kinase inhibitory activity. There is no mention therein of 2-(2-pyridyl)acetamide derivatives; in particular, there is no specific mention made therein of quinazoline-substituted 2-(2-pyridyl)acetamide derivatives.

As stated above, we have now found that surprisingly certain novel quinolin-4-yloxy-substituted 2-(2-pyridyl)acetamide compounds possess potent activity against cell proliferative disorders. Without wishing to imply that the compounds disclosed in the present invention possess pharmacological activity only by virtue of an effect on one or two biological processes, it is believed that the compounds provide a useful treatment of cell proliferative disorders, for example to provide an anti-tumour effect, by way of a contribution from inhibition of PDGF receptor tyrosine kinases. In particular, it is believed that the compounds of the present invention provide a useful treatment of cell proliferative disorders by way of a contribution from inhibition of the PDGFα and/or PDGFβ receptor tyrosine kinases.

Many of the compounds of the present invention possess potent inhibitory activity against the PDGF receptor family of tyrosine kinases, for example the PDGFα and/or PDGFβ receptor tyrosine kinases, whilst possessing less potent inhibitory activity against other tyrosine kinase enzymes, for example against one or more other Class III family receptor tyrosine kinases such as Flt3 receptor tyrosine kinase and the CSF-1R tyrosine kinase, against the EGF receptor tyrosine kinase, or against VEGF receptor tyrosine kinases such as KDR and Flt-1. Furthermore, certain compounds of the present invention possess substantially better potency against the PDGF receptor family of tyrosine kinases, particularly against the PDGFβ receptor tyrosine kinase than against EGF receptor tyrosine kinase or VEGF receptor tyrosine kinases such as KDR. Such compounds possess sufficient potency that they may be used in an amount sufficient to inhibit the PDGF receptor family of tyrosine kinases, particularly PDGFβ receptor tyrosine kinase whilst demonstrating little activity against EGF receptor tyrosine kinase or against VEGF receptor tyrosine kinases such as KDR.

According to one aspect of the invention there is provided a quinoline derivative of the Formula I

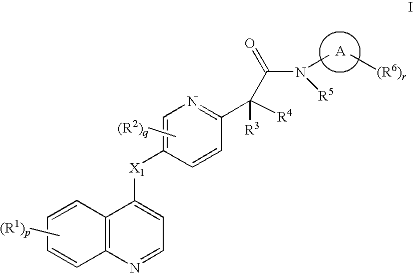

wherein X¹ is O or N(R⁷) where R⁷ is hydrogen or (1-8C)allyl;

p is 0, 1, 2 or 3;

each R¹ group, which may be the same or different, is selected from halogeno, trifluoromethyl, cyano, hydroxy, mercapto, amino, carboxy, (1-6C)alkoxycarbonyl, carbamoyl, (1-8C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulphinyl, (1-6C)alkylsulphonyl, (1-6C)alkylamino, di-[(1-6C)alkyl]amino, N-(1-6C)alkylcarbamoyl, N,N-di-[(1-6C)alkyl]carbamoyl, N-(1-6C)alkylsulphamoyl, N,N-di-[(1-6C)alkyl]sulphamoyl, (2-6C)alkanoyl, (2-6C)alkanoylamino and N-(1-6C)alkyl-(2-6C)alkanoylamino, or from a group of the formula:

Q¹-X²-

wherein X² is selected from O, S, SO, SO₂, N(R⁸), CO, CON(R⁸), N(R⁸)CO, OC(R⁸)₂ and N(R⁸)C(R⁸)₂, wherein each R⁸ is hydrogen or (1-8C)alkyl, and Q¹ is aryl, aryl-(1-6C)alkyl, (3-8C)cycloalkyl, (3-8C)cycloalkyl-(1-6C)alkyl, (3-8C)cycloalkenyl, is (3-8C)cycloalkenyl-(1-6C)alkyl, heteroaryl, heteroaryl-(1-6C)alkyl, heterocyclyl or heterocyclyl-(1-6C)alkyl,

and wherein any aryl, (3-8C)cycloalkyl, (3-8C)cycloalkenyl, heteroaryl or heterocyclyl group within a R¹ substituent optionally bears 1, 2 or 3 substituents, which may be the same or different, selected from halogeno, trifluoromethyl, cyano, nitro, hydroxy, amino, carboxy, carbamoyl, ureido, (1-8C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, (2-6C)alkanoyl, (2-6C)alkanoyloxy, N-(1-6C)alkylcarbamoyl, N,N-di-[(1-6C)alkyl]carbamoyl, (2-6C)alkanoylamino, N-(1-6C)alkyl-(2-6C)alkanoylamino, N-(1-6C)alkylureido, N′-(1-6C)alkylureido, N′,N′-di-[(1-6C)alkyl]ureido, N,N′-di-[(1-6C)alkyl]ureido, N,N′,N′-tri-[(1-6C)alkyl]ureido, N-(1-6C)alkylsulphamoyl, N,N-di-[(1-6C)alkyl]sulphamoyl, (1-6C)alkanesulphonylamino and N-(1-6C)alkyl-(1-6C)alkanesulphonylamino, or from a group of the formula:

-X³-R⁹

wherein X³ is a direct bond or is selected from O and N(R¹⁰), wherein R¹⁰ is hydrogen or (1-8C)alkyl, and R⁹ is halogeno-(1-6C)alkyl, hydroxy-(1-6C)alkyl, mercapto-(1-6C)alkyl, (1-6C)alkoxy-(1-6C)alkyl, (1-6C)alkylthio-(1-6C)alkyl, (1-6C)alkylsulphinyl-(1-6C)alkyl, (1-6C)alkylsulphonyl-(1-6C)alkyl, cyano-(1-6C)alkyl, amino-(1-6C)alkyl, (1-6C)alkylamino-(1-6C)alkyl, di-[(1-6C)alkyl]amino-(1-6C)alkyl, (2-6C)alkanoylamino-(1-6C)alkyl, N-(1-6C)alkyl-(2-6C)alkanoylamino-(1-6C)alkyl, (1-6C)alkoxycarbonylamino-(1-6C)alkyl, ureido-(1-6C)alkyl, N-(1-6C)alkylureido-(1-6C)alkyl, N′-(1-6C)alkylureido-(1-6C)alkyl, N′,N′-di-[(1-6C)alkyl]ureido-(1-6C)alkyl, N,N′-di-[(1-6C)alkyl]ureido-(1-6C)alkyl or N,N′,N′-tri-[(1-6C)alkyl]ureido-(1-6C)alkyl, or from a group of the formula:

-X⁴-Q²

wherein X⁴ is a direct bond or is selected from O, CO and N(R¹¹), wherein R¹¹ is hydrogen or (1-8C)alkyl, and Q² is aryl, aryl-(1-6C)alkyl, heteroaryl, heteroaryl-(1-6C)alkyl, heterocyclyl or heterocyclyl-(1-6C)alkyl which optionally bears 1 or 2 substituents, which may be the same or different, selected from halogeno, hydroxy, (1-8C)alkyl and (1-6C)alkoxy,

and wherein any aryl, heteroaryl or heterocyclyl group within a substituent on R¹ optionally bears a (1-3C)alkylenedioxy group,

and wherein any heterocyclyl group within a R¹ substituent optionally bears 1 or 2 oxo or thioxo substituents,

and wherein any CH, CH₂ or CH₃ group within a R¹ substituent optionally bears on each said CH, CH₂ or CH₃ group one or more halogeno or (1-8C)alkyl substituents and/or a substituent selected from hydroxy, mercapto, amino, cyano, carboxy, carbamoyl, ureido, (1-6C)alkoxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylureido, N′-(1-6C)alkylureido, N′,N′-di-[(1-6C)alkyl]ureido, N,N′-di-[(1-6C)alkyl]ureido, N,N′,N′-tri-[(1-6C)alkyl]ureido, N-(1-6C)alkylsulphamoyl, N,N-di-[(1-6C)alkyl]sulphamoyl, (1-6C)alkanesulphonylamino and N-(1-6C)alkyl-(1-6C)alkanesulphonylamino,

and wherein adjacent carbon atoms in any (2-6C)alkylene chain within a R¹ substituent are optionally separated by the insertion into the chain of a group selected from O, S, SO, SO₂, N¹²), CO, CH(OR¹²), CON(R¹²), N(R¹²)CO, N(R¹²)CON(R¹²), SO₂N(R¹²), N(R¹²) SO₂, CH═CH and C≡C wherein R¹² is hydrogen or (1-8C)alkyl, or, when the inserted group is N(R¹²), R¹² may also be (2-6C)alkanoyl;

q is 0, 1 or 2;

each R² group, which may be the same or different, is selected from halogeno, trifluoromethyl, cyano, carboxy, hydroxy, amino, carbamoyl, (1-8C)alkyl, (2-8C)alkenyl, (2-8C)alkynyl, (1-6C)alkoxy, (1-6C)alkylamino, di-[(1-6C)alkyl]amino, N-(1-6C)alkylcarbamoyl, N,N-di-[(1-6C)alkyl]carbamoyl, halogeno-(1-6C)alkyl, hydroxy-(1-6C)alkyl, (1-6C)alkoxy-(1-6C)alkyl, cyano-(1-6C)alkyl, carboxy-(1-6C)alkyl, (1-6C)alkoxycarbonyl-(1-6C)alkyl, amino-(1-6C)alkyl, (1-6C)alkylamino-(1-6C)alkyl, di-[(1-6C)alkyl]amino-(1-6C)alkyl, carbamoyl-(1-6C)alkyl, N-(1-6C)alkylcarbamoyl-(1-6C)alkyl, N,N-di-[(1-6C)alkyl]carbamoyl-(1-6C)alkyl, (2-6C)alkanoylamino-(1-6C)alkyl and N-(1-6C)alkyl-(2-6C)alkanoylamino-(1-6C)alkyl;

R³ is hydrogen, (1-8C)alkyl, (2-8C)alkenyl or (2-8C)alkynyl;

R⁴ is hydrogen, (1-8C)alkyl, (2-8C)alkenyl, (2-8C)alkynyl, halogeno-(1-6C)alkyl, hydroxy-(1-6C)alkyl, (1-6C)alkoxy-(1-6C)alkyl, cyano-(1-6C)alkyl, carboxy-(1-6C)alkyl, amino-(1-6C)alkyl, (1-6C)alkylamino-(1-6C)alkyl, di-[(1-6C)alkyl]amino-(1-6C)alkyl, carbamoyl-(1-6C)alkyl, N-(1-6C)alkylcarbamoyl-(1-6C)alkyl, N,N-di-[(1-6C)alkyl]carbamoyl-(1-6C)alkyl, (1-6C)alkoxycarbonyl-(1-6C)alkyl, (2-6C)alkanoylamino-(1-6C)alkyl or N-(1-6C)alkyl-(2-6C)alkanoylamino-(1-6C)alkyl;

or R³ and R⁴ together with the carbon atom to which they are attached form a (3-8C)cycloalkyl group;

R⁵ is hydrogen, (1-8C)alkyl, (2-8C)alkenyl or (2-8C)alkynyl or a group of the formula:

-X⁵-R¹³

wherein X⁵ is a direct bond or is selected from O and N(R¹⁴), wherein R¹⁴ is hydrogen or (1-8C)alkyl, and R¹³ is halogeno-(1-6C)alkyl, hydroxy-(1-6C)alkyl, (1-6C)alkoxy-(1-6C)alkyl or cyano-(1-6C)alkyl;

Ring A is a 6-membered monocyclic or a 10-membered bicyclic aryl ring or a 5- or 6-membered monocyclic or a 9- or 10-membered bicyclic heteroaryl ring with up to three ring heteroatoms selected from oxygen, nitrogen and sulphur;

r is 0, 1, 2 or 3; and

each R⁶ group, which may be the same or different, is selected from halogeno, trifluoromethyl, cyano, hydroxy, mercapto, amino, carboxy, carbamoyl, sulphamoyl, ureido, (1-8C)alkyl, (2-8C)alkenyl, (2-8C)alkynyl, (1-6C)alkoxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, (2-6C)alkanoyl, (2-6C)alkanoyloxy, N-(1-6C)alkylcarbamoyl, N,N-di-[(1-6C)alkyl]carbamoyl, (2-6C)alkanoylamino, N-(1-6C)alkyl-(2-6C)alkanoylamino, N′-(1-6C)alkylureido, N′,N′-di-[(1-6C)alkyl]ureido, N-(1-6C)alkylsulphamoyl, N,N-di-[(1-6C)alkyl]sulphamoyl, (1-6C)alkanesulphonylamino and N-(1-6C)alkyl-(1-6C)alkanesulphonylamino, or from a group of the formula:

-X⁶-R¹⁵

wherein X⁶ is a direct bond or is selected from O and N(R¹⁶), wherein R¹⁶ is hydrogen or (1-8C)alkyl, and R¹⁵ is halogeno-(1-6C)alkyl, hydroxy-(1-6C)alkyl, mercapto-(1-6C)alkyl, (1-6C)alkoxy-(1-6C)alkyl, (1-6C)alkylthio-(1-6C)alkyl, (1-6C)alkylsulphinyl-(1-6C)alkyl, (1-6C)alkylsulphonyl-(1-6C)alkyl, cyano-(1-6C)alkyl, amino-(1-6C)alkyl, (1-6C)alkylamino-(1-6C)alkyl, di-[(1-6C)alkyl]amino-(1-6C)alkyl, (2-6C)alkanoylamino-(1-6C)alkyl, N-(1-6C)alkyl-(2-6C)alkanoylamino-(1-6C)alkyl, carboxy-(1-6C)alkyl, (1-6C)alkoxycarbonyl-(1-6C)alkyl, carbamoyl-(1-6C)alkyl, N-(1-6C)alkylcarbamoyl-(1-6C)alkyl, N,N-di-[(1-6C)alkyl]carbamoyl-(1-6C)alkyl, sulphamoyl-(1-6C)alkyl, N-(1-6C)alkylsulphamoyl-(1-6C)alkyl, N,N-di-[(1-6C)alkyl]sulphamoyl-(1-6C)alkyl, ureido-(1-6C)alkyl, N-(1-6C)alkylureido-(1-6C)alkyl, N′-(1-6C)alkylureido-(1-6C)alkyl, N′,N′-di-[(1-6C)alkyl]ureido-(1-6C)alkyl, N,N′-di-[(1-6C)alkyl]ureido-(1-6C)alkyl, N,N′,N′-tri-[(1-6C)alkyl]ureido-(1-6C)alkyl, (1-6C)alkanesulphonylamino-(1-6C)alkyl or N-(1-6C)alkyl-(1-6C)alkanesulphonylamino-(1-6C)alkyl, or from a group of the formula:

-X⁷-Q³

wherein X⁷ is a direct bond or is selected from O, S, SO, SO₂, N(R¹⁷), CO, CH(OR¹⁷), CON(R¹⁷), N(R¹⁷)CO, N(R¹⁷)CON(R¹⁷), SO₂N(R¹⁷), N(R¹⁷)SO₂, C(R¹⁷)₂O, C(R¹⁷)₂S and C(R¹⁷)₂N(R¹⁷), wherein each R¹⁷ is hydrogen or (1-8C)alkyl, and Q³ is aryl, aryl-(1-6C)alkyl, (3-8C)cycloalkyl, (3-8C)cycloalkyl-(1-6C)alkyl, (3-8C)cycloalkenyl, (3-8C)cycloalkenyl-(1-6C)alkyl, heteroaryl, heteroaryl-(1-6C)alkyl, heterocyclyl or heterocyclyl-(1-6C)alkyl,

or two R⁶ groups together form a bivalent group that spans adjacent ring positions on Ring A selected from OC(R¹⁸)₂O, OC(R¹⁸)₂C(R¹⁸)₂O, OC(R¹⁸)₂C(R¹⁸)₂, C(R¹⁸)₂OC(R¹⁸)₂, C(R¹⁸)₂C(R¹⁸)₂C(R¹⁸)₂, C(R¹⁸)₂C(R¹⁸)₂C(R¹⁸)₂C(R¹⁸)₂, OC(R¹⁸)₂N(R¹⁹), N(R¹⁹)C(R¹⁸)₂N(R¹⁹), N(R¹⁹)C(R¹⁸)₂C(R¹⁸)₂, N(R¹⁹)C(R¹⁸)₂C(R¹⁸)₂C(R¹⁸)₂, O C(R¹⁸)₂C(R¹⁸)₂N(R¹⁹), C(R¹⁸)₂N(R¹⁹)C(R¹⁸)₂, CO.N(R¹⁸)C(R¹⁸)₂, N(R¹⁸)CO.C(R¹⁸)₂, N(R¹⁹)C(R¹⁸)₂CO, CO.N(R¹⁸)CO, N(R¹⁹)N(R¹⁸)CO, N(R¹⁸)CO.N(R¹⁸), O.CO.N(R¹⁸), O.CO.C(R¹⁸)₂ and CO.OC(R¹⁸)₂ wherein each R¹⁸ is hydrogen, (1-8C)alkyl, (2-8C)alkenyl or (2-8C)alkynyl, and wherein R¹⁹ is hydrogen, (1-8C)alkyl, (2-8C)alkenyl, (2-8C)alkynyl or (2-6C)alkanoyl,

and wherein any aryl, (3-8C)cycloalkyl, (3-8C)cycloalkenyl, heteroaryl or heterocyclyl group within an R⁶ group optionally bears 1, 2 or 3 substituents, which may be the same or different, selected from halogeno, trifluoromethyl, cyano, nitro, hydroxy, amino, carboxy, carbamoyl, ureido, (1-8C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, (2-6C)alkanoyl, (2-6C)alkanoyloxy, N-(1-6C)alkylcarbamoyl, N,N-di-[(1-6C)alkyl]carbamoyl, (2-6C)alkanoylamino, N-(1-6C)alkyl-(2-6C)alkanoylamino, N′-(1-6C)alkylureido, N′,N′-di-[(1-6C)alkyl]ureido, N-(1-6C)alkylureido, N,N′-di-[(1-6C)alkyl]ureido, N,N′,N′-tri-[(1-6C)alkyl]ureido, N-(1-6C)alkylsulphamoyl, N,N-di-[(1-6C)alkyl]sulphamoyl, (1-6C)alkanesulphonylamino and N-(1-6C)alkyl-(1-6C)alkanesulphonylamino, or from a group of the formula:

-X⁸-R²⁰

wherein X⁸ is a direct bond or is selected from O and N(R²¹), wherein R²¹ is hydrogen or (1-8C)alkyl, and R²⁰ is halogeno-(1-6C)alkyl, hydroxy-(1-6C)alkyl, mercapto-(1-6C)alkyl, (1-6C)alkoxy-(1-6C)alkyl, (1-6C)alkylthio-(1-6C)alkyl, (1-6C)alkylsulphinyl-(1-6C)alkyl, (1-6C)alkylsulphonyl-(1-6C)alkyl, cyano-(1-6C)alkyl, amino-(1-6C)alkyl, (1-6C)alkylamino-(1-6C)alkyl, di-[(1-6C)alkyl]amino-(1-6C)alkyl, (2-6C)alkanoylamino-(1-6C)alkyl or N-(1-6C)alkyl-(2-6C)alkanoylamino-(1-6C)alkyl, or from a group of the formula:

-X⁹-Q⁴

wherein X⁹ is a direct bond or is selected from O, CO and N(R²²), wherein R²² is hydrogen or (1-8C)alkyl, and Q⁴ is aryl, aryl-(1-6C)alkyl, heteroaryl, heteroaryl-(1-6C)alkyl, heterocyclyl or heterocyclyl-(1-6C)alkyl which optionally bears 1 or 2 substituents, which may be the same or different, selected from halogeno, hydroxy, (1-8C)alkyl and (1-6C)alkoxy,

and wherein any aryl, heteroaryl or heterocyclyl group within an R⁶ group optionally bears a (1-3C)alkylenedioxy group,

and wherein any heterocyclyl group within an R⁶ group optionally bears 1 or 2 oxo or thioxo substituents,

and wherein any CH, CH₂ or CH₃ group within an R⁶ group optionally bears on each said CH, CH₂ or CH₃ group one or more halogeno or (1-8C)alkyl substituents and/or a substituent selected from hydroxy, mercapto, amino, cyano, carboxy, carbamoyl, ureido, (2-8C)alkenyl, (2-8C)alkynyl, (1-6C)alkoxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N′-(1-6C)alkylureido, N′,N′-di-[(1-6C)alkyl]ureido, N-(1-6C)alkylureido, N,N′-di-[(1-6C)alkyl]ureido, N,N′,N′-tri-[(1-6C)alkyl]ureido, N-(1-6C)alkylsulphamoyl, N-(1-6C)alkylsulphamoyl, N,N-di-[(1-6C)alkyl]sulphamoyl, (1-6C)alkanesulphonylamino and N-(1-6C)alkyl-(1-6C)alkanesulphonylamino,

and wherein adjacent carbon atoms in any (2-6C)alkylene chain within an R⁶ group are optionally separated by the insertion into the chain of a group selected from O, S, SO, SO₂, N(R²³), N(R²³)CO, CON(R²³), N(R²³)CON(R²³), CO, CH(OR²³), N(R²³)SO₂, SO₂N(R²³), CH═CH and C≡C wherein R²³ is hydrogen or (1-8C)alkyl, or, when the inserted group is N(R²³), R²³ may also be (2-6C)alkanoyl;

or a pharmaceutically-acceptable salt thereof.

In this specification the generic term “(1-8C)alkyl” includes both straight-chain and branched-chain alkyl groups such as propyl, isopropyl and tert-butyl, and also (3-8C)cycloalkyl groups such as cyclopropyl, cyclobutyl, cyclopentyl, cyclohexyl and cycloheptyl, and also (3-6C)cycloalkyl-(1-2C)alkyl groups such as cyclopropylmethyl, 2-cyclopropylethyl, cyclobutylmethyl, 2-cyclobutylethyl, cyclopentylmethyl, 2-cyclopentylethyl, cyclohexylmethyl and 2-cyclohexylethyl. However references to individual alkyl groups such as “propyl” are specific for the straight-chain version only, references to individual branched-chain alkyl groups such as “isopropyl” are specific for the branched-chain version only and references to individual cycloalkyl groups such as “cyclopentyl” are specific for that 5-membered ring only. An analogous convention applies to other generic terms, for example (1-6C)alkoxy includes (3-6C)cycloalkyloxy groups and (3-5C)cycloalkyl-(1-2C)alkoxy groups, for example methoxy, ethoxy, propoxy, isopropoxy, cyclopropyloxy, cyclobutyloxy, cyclopentyloxy, cyclohexyloxy, cyclopropylmethoxy, 2-cyclopropylethoxy, cyclobutylmethoxy, 2-cyclobutylethoxy and cyclopentylmethoxy; (1-6C)alkylamino includes (3-6C)cycloalkylamino groups and (3-5C)cycloalkyl-(1-2C)alkylamino groups, for example methylamino, ethylamino, propylamino, cyclopropylamino, cyclobutylamino, cyclohexylamino, cyclopropylmethylamino, 2-cyclopropylethylamino, cyclobutylmethylamino, 2-cyclobutylethylamino and cyclopentylmethylamino; and di-[(1-6Calkyl]amino includes di-[(3-6C)cycloalkyl]amino groups and di-[(3-5C)cycloalkyl-(1-2C)alkyl]amino groups, for example dimethylamino, diethylamino, dipropylamino, N-cyclopropyl-N-methylamino, N-cyclobutyl-N-methylamino, N-cyclohexyl-N-ethylamino, N-cyclopropylmethyl-N-methylamino, N-(2-cyclopropylethyl)-N-methylamino and N-cyclopentylmethyl-N-methylamino.

It is to be understood that, insofar as certain of the compounds of Formula I defined above may exist in optically active or racemic forms by virtue of one or more asymmetric carbon atoms, the invention includes in its definition any such optically active or racemic form which possesses the above-mentioned activity. The synthesis of optically active forms may be carried out by standard techniques of organic chemistry well known in the art, for example by synthesis from optically active starting materials or by resolution of a racemic form. Similarly, the above-mentioned activity may be evaluated using the standard laboratory techniques referred to hereinafter.

It is to be understood that certain compounds of Formula I defined above may exhibit the phenomenon of tautomerism. In particular, tautomerism may affect heteroaryl rings within the definition of Ring A or heterocyclic groups within the R¹ and R⁶ groups that bear 1 or 2 oxo or thioxo substituents. It is to be understood that the present invention includes in its definition any such tautomeric form, or a mixture thereof, which possesses the above-mentioned activity and is not to be limited merely to any one tautomeric form utilised within the formulae drawings or named in the Examples. For example, Ring A may be a pyrazolyl group. When, for example, such a pyrazolyl group is linked to the N atom of the CON(R⁵) group from the 3-position, a tautomeric mixture of compounds comprising a 1H-pyrazol-3-yl group and a 1H-pyrazol-5-yl group may be present. In general, just one of any such tautomeric forms is named in the Examples that follow hereinafter or is presented in any relevant formulae drawings that follow hereinafter.

In structural Formula I, it is to be understood that there is a hydrogen atom at the 2-position on the quinoline ring. It is to be understood thereby that the R¹ substituents may only be located at the 3-, 5-, 6-, 7- or 8-positions on the quinoline ring i.e. that the 2-position remains unsubstituted. Conveniently, the 3-position on the quinoline ring also remains unsubstituted or the R¹ substituent at the 3-position on the quinoline ring is a cyano group. More conveniently, R¹ substituents may only be located at the 5-, 6- or 7-positions on the quinoline ring. Yet more conveniently, R¹ substituents may only be located at the 6- and/or 7-positions on the quinoline ring.

In structural Formula I, it is further to be understood that any R² group that may be present on the central pyridyl group may be located at any available position. Conveniently, no R² group is present (q=0). Alternatively, there is a single R² group. More conveniently, there is a single R² group that is located at the 2-position (relative to the C(R³)(R⁴) group).

In structural Formula I, it is to be understood that any R⁶ group may be located at any available position on Ring A. For example, an R⁶ group may be located at the 3- or 4-position (relative to the CON(R⁵) group) when Ring A is a 6-membered ring or, for example, it may be located at the 3-position (relative to the CON(R⁵) group) when Ring A is a 5-membered ring.

Suitable values for the generic radicals referred to above include those set out below.

A suitable value for any one of the ‘Q’ groups (Q¹ to Q⁴) within the R¹ or R⁶ groups when the ‘Q’ group is aryl or for the aryl group within any ‘Q’ group is, for example, phenyl or naphthyl, preferably phenyl.

A suitable value for any one of the ‘Q’ groups (Q¹ or Q³) within the R¹ or R⁶ groups when the ‘Q’ group is (3-8C)cycloalkyl or for the (3-8C)cycloalkyl group within any ‘Q’ group is, for example, cyclopropyl, cyclobutyl, cyclopentyl, cyclohexyl, cycloheptyl, bicyclo[2.2.1]heptyl or cyclooctyl.

A suitable value for the (3-8C)cycloalkyl group formed when R³ and R⁴ together with the carbon atom to which they are attached form a (3-8C)cycloalkyl group is, for example, cyclopropyl, cyclobutyl, cyclopentyl, cyclohexyl or cycloheptyl.

A suitable value for any one of the ‘Q’ groups (Q¹ or Q³) within the R¹ or R⁶ groups when the ‘Q’ group is (3-8C)cycloalkenyl or for the (3-8C)cycloalkenyl group within any ‘Q’ group is, for example, cyclobutenyl, cyclopentenyl, cyclohexenyl, cycloheptenyl or cyclooctenyl.

A suitable value for any one of the ‘Q’ groups (Q¹ to Q⁴) within the R¹ or R⁶ groups when the ‘Q’ group is heteroaryl or for the heteroaryl group within any ‘Q’ group is, for example, an aromatic 5- or 6-membered monocyclic ring or a 9- or 10-membered bicyclic ring with up to five ring heteroatoms selected from oxygen, nitrogen and sulphur, for example furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, benzofuranyl, indolyl, benzothienyl, benzoxazolyl, benzimidazolyl, benzothiazolyl, indazolyl, benzofurazanyl, quinolyl, isoquinolyl, quinazolinyl, quinoxalinyl, cinnolinyl or naphthyridinyl.

A suitable value for any one of the ‘Q’ groups (Q¹ to Q⁴) within the R¹ or R⁶ groups when the ‘Q’ group is heterocyclyl or for the heterocyclyl group within any ‘Q’ group is, for example, a non-aromatic saturated or partially saturated 3 to 10 membered monocyclic or bicyclic ring with up to five heteroatoms selected from oxygen, nitrogen and sulphur, for example oxiranyl, oxetanyl, tetrahydrofuranyl, tetrahydropyranyl, oxepanyl, tetrahydrothienyl, 1,1-dioxotetrahydrothienyl, tetrahydrothiopyranyl, 1,1-dioxotetrahydrothiopyranyl, aziridinyl, azetidinyl, pyrrolinyl, pyrrolidinyl, imidazolinyl, imidazolidinyl, pyrazolinyl, pyrazolidinyl, molpholinyl, tetrahydro-1,4-thiazinyl, 1,1-dioxotetrahydro-1,4-thiazinyl, piperidinyl, homopiperidinyl, piperazinyl, homopiperazinyl, 2-azabicyclo[2.2.1]heptyl, quinuclidinyl, chromanyl, isochromanyl, indolinyl, isoindolinyl, dihydropyridinyl, tetrahydropyridinyl, dihydropyrimidinyl or tetrahydropyrimidinyl, preferably tetrahydrofuranyl, tetrahydropyranyl, tetrahydrothiopyranyl, pyrrolinyl, pyrrolidinyl, morpholinyl, piperidinyl, piperazinyl, indolinyl or isoindolinyl. A suitable value for such a group which bears 1 or 2 oxo or thioxo substituents is, for example, 2-oxopyrrolidinyl, 2-thioxopyrrolidinyl, 2-oxoimidazolidinyl, 2-thioxoimidazolidinyl, 2-oxopiperidinyl, 4-oxo-1,4-dihydropyridinyl, 2,5-dioxopyrrolidinyl, 2,5-dioxoimidazolidinyl or 2,6-dioxopiperidinyl.

A suitable value for any ‘Q’ group when it is heteroaryl-(1-6C)alkyl is, for example, heteroarylmethyl, 2-heteroarylethyl and 3-heteroarylpropyl. The invention comprises corresponding suitable values for ‘Q’ groups when, for example, rather than a heteroaryl-(1-6C)alkyl group, an aryl-(1-6C)alkyl, (3-8C)cycloalkyl-(1-6C)alkyl, (3-8C)cycloalkenyl-(1-6C)alkyl or heterocyclyl-(1-6C)alkyl group is present.

A suitable value for Ring A when it is a 6-membered monocyclic or a 10-membered bicyclic aryl ring or a 5- or 6-membered monocyclic or a 9- or 10-membered bicyclic heteroaryl ring with up to three ring heteroatoms selected from oxygen, nitrogen and sulphur is, for example, phenyl, naphthyl, furyl, pyrrolyl, thienyl, oxazolyl, isoxazolyl, imidazolyl, pyrazolyl, thiazolyl, isothiazolyl, oxadiazolyl, thiadiazolyl, triazolyl, pyridyl, pyridazinyl, pyrimidinyl, pyrazinyl, 1,3,5-triazenyl, benzofuranyl, indolyl, benzothienyl, benzoxazolyl, benzimidazolyl, benzothiazolyl, indazolyl, benzofurazanyl, quinolyl, isoquinolyl, quinazolinyl, quinoxalinyl, cinnolinyl or naphthyridinyl. Conveniently, Ring A is a phenyl, furyl, pyrrolyl, thienyl, oxazolyl, isoxazolyl, imidazolyl, pyrazolyl, thiazolyl, pyridyl, pyrimidinyl, pyrazinyl or pyridazinyl ring. Conveniently, Ring A is a phenyl, pyridyl, pyrimidinyl, pyrazinyl or pyridazinyl ring. A suitable value for Ring A when it is a 5-membered monocyclic heteroaryl ring with up to three ring heteroatoms selected from oxygen, nitrogen and sulphur is, for example, furyl, pyrrolyl, thienyl, oxazolyl, isoxazolyl, imidazolyl, pyrazolyl, thiazolyl, isothiazolyl, oxadiazolyl, thiadiazolyl or triazolyl. Conveniently, Ring A is an oxazolyl, isoxazolyl, imidazolyl, pyrazolyl, thiazolyl, isothiazolyl, oxadiazolyl or thiadiazolyl ring.

Suitable values for any of the ‘R’ groups (R¹ to R²³), or for various groups within an R¹, R² or R⁶ substituent include:—

| | |
|---|---|
| for halogeno | fluoro, chloro, bromo and iodo; |
| for (1-8C)alkyl: | methyl, ethyl, propyl, isopropyl, tert-butyl, |
| | cyclobutyl, cyclohexyl, cyclohexylmethyl and |
| | 2-cyclopropylethyl; |
| for (2-8C)alkenyl: | vinyl, isopropenyl, allyl and but-2-enyl; |
| for (2-8C)alkynyl: | ethynyl, 2-propynyl and but-2-ynyl; |
| for (1-6C)alkoxy: | methoxy, ethoxy, propoxy, isopropoxy and butoxy; |
| for (2-6C)alkenyloxy: | vinyloxy and allyloxy; |
| for (2-6C)alkynyloxy: | ethynyloxy and 2-propynyloxy; |
| for (1-6C)alkylthio: | methylthio, ethylthio and propylthio; |
| for (1-6C)alkylsulphinyl: | methylsulphinyl and ethylsulphinyl; |
| for (1-6C)alkylsulphonyl: | methylsulphonyl and ethylsulphonyl; |
| for (1-6C)alkylamino: | methylamino, ethylamino, propylamino, |
| | isopropylamino and butylamino; |
| for di-[(1-6C)alkyl]amino: | dimethylamino, diethylamino, |
| | N-ethyl-N-methylamino and diisopropylamino; |
| for (1-6C)alkoxycarbonyl: | methoxycarbonyl, ethoxycarbonyl, propoxycarbonyl |
| | and tert-butoxycarbonyl; |
| for N-(1-6C)alkylcarbamoyl: | N-methylcarbamoyl, N-ethylcarbamoyl and |
| | N-propylcarbamoyl; |
| for N,N-di-[(1-6C)alkyl]carbamoyl: | N,N-dimethylcarbamoyl, N-ethyl- |
| | N-methylcarbamoyl and N,N-diethylcarbamoyl; |
| for (2-6C)alkanoyl: | acetyl, propionyl and isobutyryl; |
| for (2-6C)alkanoyloxy: | acetoxy and propionyloxy; |
| for (2-6C)alkanoylamino: | acetamido and propionamido; |
| for N-(1-6C)alkyl-(2-6C)alkanoylamino: | N-methylacetamido and N-methylpropionamido; |
| for N′-(1-6C)alkylureido: | N′-methylureido and N′-ethylureido; |
| for N′,N′-di-[(1-6C)alkyl]ureido: | N′,N′-dimethylureido and N′-methyl-N′-ethylureido; |
| for N-(1-6C)alkylureido: | N-methylureido and N-ethylureido; |
| for N,N′-di-[(1-6C)alkyl]ureido: | N,N′-dimethylureido, N-methyl-N′-ethylureido and |
| | N-ethyl-N′-methylureido; |
| for N,N′,N′-di-[(1-6C)alkyl]ureido: | N,N′,N′-trimethylureido, |
| | N-ethyl-N′,N′-dimethylureido and |
| | N-methyl-N′,N′-diethylureido; |
| for N-(1-6C)alkylsulphamoyl: | N-methylsulphamoyl and N-ethylsulphamoyl; |
| for N,N-di-[(1-6C)alkyl]sulphamoyl: | N,N-dimethylsulphamoyl; |
| for (1-6C)alkanesulphonylamino: | methanesulphonylamino and ethanesulphonylamino; |
| for N-(1-6C)alkyl-(1-6C)alkanesulphonylamino: | N-methylmethanesulphonylamino and |
| | N-methylethanesulphonylamino; |
| for halogeno-(1-6C)alkyl: | chloromethyl, 2-fluoroethyl, 2-chloroethyl, |
| | 1-chloroethyl, 2,2-difluoroethyl, 2,2,2-trifluoroethyl, |
| | 3-fluoropropyl, 3-chloropropyl, 3,3-difluoropropyl |
| | and 3,3,3-trifluoropropyl; |
| for hydroxy-(1-6C)alkyl: | hydroxymethyl, 2-hydroxyethyl, 1-hydroxyethyl and |
| | 3-hydroxypropyl; |
| for mercapto-(1-6C)alkyl: | mercaptomethyl, 2-mercaptoethyl, 1-mercaptoethyl |
| | and 3-mercaptopropyl; |
| for (1-6C)alkoxy-(1-6C)alkyl: | methoxymethyl, ethoxymethyl, 1-methoxyethyl, |
| | 2-methoxyethyl, 2-ethoxyethyl and |
| | 3-methoxypropyl; |
| for (1-6C)alkylthio-(1-6C)alkyl: | methylthiomethyl, ethylthiomethyl, |
| | 2-methylthioethyl, 1-methylthioethyl and |
| | 3-methylthiopropyl; |
| for (1-6C)alkylsulphinyl-(1-6C)alkyl: | methylsulphinylmethyl, ethylsulphinylmethyl, |
| | 2-methylsulphinylethyl, 1-methylsulphinylethyl and |
| | 3-methylsulphinylpropyl; |
| for (1-6C)alkylsulphonyl-(1-6C)alkyl: | methylsulphonylmethyl, ethylsulphonylmethyl, |
| | 2-methylsulphonylethyl, 1-methylsulphonylethyl and |
| | 3-methylsulphonylpropyl; |
| for cyano-(1-6C)alkyl: | cyanomethyl, 2-cyanoethyl, 1-cyanoethyl and |
| | 3-cyanopropyl; |
| for amino-(1-6C)alkyl: | aminomethyl, 2-aminoethyl, 1-aminoethyl, |
| | 3-aminopropyl, 1-aminopropyl and 5-aminopropyl; |
| for (1-6C)alkylamino-(1-6C)alkyl: | methylaminomethyl, ethylaminomethyl, |
| | 1-methylaminoethyl, 2-methylaminoethyl, |
| | 2-ethylaminoethyl and 3-methylaminopropyl; |
| for di-[(1-6C)alkyl]amino-(1-6C)alkyl: | dimethylaminomethyl, diethylaminomethyl, |
| | 1-dimethylaminoethyl, 2-dimethylaminoethyl and |
| | 3-dimethylaminopropyl; |
| for (2-6C)alkanoylamino-(1-6C)alkyl: | acetamidomethyl, propionamidomethyl, |
| | 2-acetamidoethyl and 1-acetamidoethyl; |
| for N-(1-6C)alkyl-(2-6C)alkanoylamino-(1-6C)alkyl: | N-methylacetamidomethyl, |
| | N-methylpropionamidomethyl, |
| | 2-(N-methylacetamido)ethyl and |
| | 1-(N-methylacetamido)ethyl; |
| for (1-6C)alkoxycarbonylamino-(1-6C)alkyl: | methoxycarbonylaminomethyl, |
| | ethoxycarbonylaminomethyl, |
| | tert-butoxycarbonylaminomethyl and |
| | 2-methoxycarbonylaminoethyl. |
| for ureido-(1-6C)alkyl: | ureidomethyl, 2-ureidoethyl and 1-ureidoethyl; |
| for N′-(1-6C)alkylureido-(1-6C)alkyl: | N′-methylureidomethyl, 2-(N′-methylureido)ethyl |
| | and 1-(N′-methylureido)ethyl; |
| for N′,N′-di-[(1-6C)alkyl]ureido-(1-6C)alkyl: | N′,N′-dimethylureidomethyl, |
| | 2-(N′,N′-dimethylureido)ethyl and |
| | 1-(N′,N′-dimethylureido)ethyl; |
| for N-(1-6C)alkylureido-(1-6C)alkyl: | N-methylureidomethyl, 2-(N-methylureido)ethyl and |
| | 1-(N-methylureido)ethyl; |
| for N,N′-di-[(1-6C)alkyl]ureido-(1-6C)alkyl: | N,N′-dimethylureidomethyl, |
| | 2-(N,N′-dimethylureido)ethyl and |
| | 1-(N,N′-dimethylureido)ethyl; |
| for N,N′,N′-tri-[(1-6C)alkyl]ureido-(1-6C)alkyl: | N,N′,N′-trimethylureidomethyl, |
| | 2-(N,N′,N′-trimethylureido)ethyl and |
| | 1-(N,N′,N′-trimethylureido)ethyl; |
| for carboxy-(1-6C)alkyl: | carboxymethyl, 1-carboxyethyl, 2-carboxyethyl, |
| | 3-carboxypropyl and 4-carboxybutyl; |
| for (1-6C)alkoxycarbonyl-(1-6C)alkyl: | methoxycarbonylmethyl, ethoxycarbonylmethyl, |
| | tert-butoxycarbonylmethyl, 1-methoxycarbonylethyl, |
| | 1-ethoxycarbonylethyl, 2-methoxycarbonylethyl, |
| | 2-ethoxycarbonylethyl, 3-methoxycarbonylpropyl |
| | and 3-ethoxycarbonylpropyl; |
| for carbamoyl-(1-6C)alkyl: | carbamoylmethyl, 1-carbamoylethyl, |
| | 2-carbamoylethyl and 3-carbamoylpropyl; |
| for N-(1-6C)alkylcarbamoyl-(1-6C)alkyl: | N-methylcarbamoylmethyl, |
| | N-ethylcarbamoylmethyl, N-propylcarbamoylmethyl, |
| | 1-(N-methylcarbamoyl)ethyl, |
| | 1-(N-ethylcarbamoyl)ethyl, |
| | 2-(N-methylcarbamoyl)ethyl, |
| | 2-(N-ethylcarbamoyl)ethyl and |
| | 3-(N-methylcarbamoyl)propyl; |
| for N,N-di-[(1-6C)alkyl]carbamoyl-(1-6C)alkyl: | N,N-dimethylcarbamoylmethyl, |
| | N-ethyl-N-methylcarbamoylmethyl, |
| | N,N-diethylcarbamoylmethyl, |
| | 1-(N,N-dimethylcarbamoyl)ethyl, |
| | 1-(N,N-diethylcarbamoyl)ethyl, |
| | 2-(N,N-dimethylcarbamoyl)ethyl, |
| | 2-(N,N-diethylcarbamoyl)ethyl, |
| | 3-(N,N-dimethylcarbamoyl)propyl and |
| | 4-(N,N-dimethylcarbamoyl)butyl; |
| for sulphamoyl-(1-6C)alkyl: | sulphamoylmethyl, 1-sulphamoylethyl, |
| | 2-sulphamoylethyl and 3-sulphamoylpropyl; |
| for N-(1-6C)alkylsulphamoyl-(1-6C)alkyl: | N-methylsulphamoylmethyl, |
| | 1-(N-methylsulphamoyl)ethyl, |
| | 2-(N-methylsulphamoyl)ethyl, and |
| | 3-(N-methylsulphamoyl)propyl; |
| for N,N-di-[(1-6C)alkyl]sulphamoyl-(1-6C)alkyl: | N,N-dimethylsulphamoylmethyl, |
| | 1-(N,N-dimethylsulphamoyl)ethyl, |
| | 2-(N,N-dimethylsulphamoyl)ethyl and |
| | 3-(N,N-dimethylsulphamoyl)propyl; |
| for (1-6C)alkanesulphonylamino-(1-6C)alkyl: | methanesulphonylaminomethyl, |
| | 2-(methanesulphonylamino)ethyl and |
| | 1-(methanesulphonylamino)ethyl; and |
| for N-(1-6C)alkyl-(1-6C)alkanesulphonylamino-(1-6C)alkyl: | N-methylmethanesulphonylaminomethyl, |
| | 2-(N-methylmethanesulphonylamino)ethyl and |
| | 1-(N-methylmethanesulphonylamino)ethyl. |

A suitable value for a (1-3C)alkylenedioxy group that may be present within a R¹ or R⁶ group is, for example, methylenedioxy, ethylidenedioxy, isopropylidenedioxy or ethylenedioxy and the oxygen atoms thereof occupy adjacent ring positions.

When, as defined hereinbefore, an R¹ group forms a group of the formula Q¹-X²- and, for example, X² is a OC(R⁸)₂ linking group, it is the carbon atom, not the oxygen atom, of the OC(R⁸)₂ linking group which is attached to the quinoline ring and the oxygen atom is attached to the Q¹ group. Similarly, when, as defined hereinbefore, an R⁶ group forms a group of the formula -X⁷-Q³ and, for example, X⁷ is a C(R¹⁷)₂O linking group, it is the oxygen atom of the C(R¹⁷)₂O linking group which is attached to the Q³ group.

A suitable (2-6C)alkylene chain within a R¹ or R⁶ group is, for example, an ethylene, trimethylene, tetramethylene or pentamethylene chain.

As defined hereinbefore, adjacent carbon atoms in any (2-6C)alkylene chain within a R¹ or R⁶ group may be optionally separated by the insertion into the chain of a group such as O, CON(R¹²) or CON(R²³) respectively, and C≡C. For example, insertion of an O atom into the alkylene chain within a 4-methoxybutoxy group gives rise to, for example, a 2-(2-methoxyethoxy)ethoxy group, for example, insertion of a C≡C group into the ethylene chain within a 2-hydroxyethoxy group gives rise to a 4-hydroxybut-2-ynyloxy group and, for example, insertion of a CONH group into the ethylene chain within a 3-methoxypropoxy group gives rise to, for example, a 2-(2-methoxyacetamido)ethoxy group.

When, as defined hereinbefore, any CH, CH₂ or CH₃ group within a R¹ or R⁶ group optionally bears on each said CH, CH₂ or CH₃ group one or more halogeno or (1-8C)alkyl substituents, there is suitably 1 halogeno or (1-8C)alkyl substituent present on each said CH group, there are suitably 1 or 2 such substituents present on each said CH₂ group and there are suitably 1, 2 or 3 such substituents present on each said CH₃ group.

When, as defined hereinbefore, any CH, CH₂ or CH₃ group within a R¹ or R⁶ group optionally bears on each said CH, CH₂ or CH₃ group a substituent as defined hereinbefore, suitable R¹ or R⁶ groups so formed include, for example, hydroxy-substituted (1-8C)alkyl groups such as hydroxymethyl, 1-hydroxyethyl and 2-hydroxyethyl, hydroxy-substituted (1-6C)alkoxy groups such as 2-hydroxypropoxy and 3-hydroxypropoxy, (1-6C)alkoxy-substituted (1-6C)alkoxy groups such as 2-methoxyethoxy and 3-ethoxypropoxy, hydroxy-substituted amino-(2-6C)alkoxy groups such as 3-amino-2-hydroxypropoxy, hydroxy-substituted (1-6C)alkylamino-(2-6C)alkoxy groups such as 2-hydroxy-3-methylaminopropoxy, hydroxy-substituted di-[(1-6C)alkyl]amino-(2-6C)alkoxy groups such as 3-dimethylamino-2-hydroxypropoxy, hydroxy-substituted amino-(2-6C)alkylamino groups such as 3-amino-2-hydroxypropylamino, hydroxy-substituted (1-6C)alkylamino-(2-6C)alkylamino groups such as 2-hydroxy-3-methylaminopropylamino and hydroxy-substituted di-[(1-6C)alkyl]amino-(2-6C)alkylamino groups such as 3-dimethylamino-2-hydroxypropylamino.

When, as defined hereinbefore, any CH, CH₂ or CH₃ group within a R¹ or R⁶ group optionally bears on each said CH, CH₂ or CH₃ group a substituent as defined hereinbefore, suitable R¹ or R⁶ groups so formed also include, for example, hydroxy-substituted (1-6C)alkylamino-(1-6C)alkyl groups such as 2-hydroxy-3-methylaminopropyl and 2-hydroxyethylaminomethyl and hydroxy-substituted di-[(1-6C)alkyl]amino-(1-6C)alkyl groups such as 3-dimethylamino-2-hydroxypropyl and di-(2-hydroxyethyl)aminomethyl.

It is further to be understood that when, as defined hereinbefore, any CH, CH₂ or CH₃ group within a R¹ or R⁶ group optionally bears on each said CH, CH₂ or CH₃ group a substituent as defined hereinbefore, such an optional substituent may be present on a CH, CH₂ or CH₃ group within the hereinbefore defined substituents that may be present on an aryl, heteroaryl or heterocyclyl group within a R¹ or R⁶ group. For example, if the R¹ or R⁶ group includes an aryl or heteroaryl group that is substituted by a (1-8C)alkyl group, the (1-8C)alkyl group may be optionally substituted on a CH, CH₂ or CH₃ group therein by one of the hereinbefore defined substituents therefor. For example, if the R¹ or R⁶ group includes a heteroaryl group that is substituted by, for example, a (1-6C)alkylamino-(1-6C)alkyl group, the terminal CH₃ group of the (1-6C)alkylamino group may be further substituted by, for example, a (1-6C)alkylsulphonyl group or a (2-6C)alkanoyl group. Further, for example, if the R¹ or R⁶ group includes a heterocyclyl group such as a piperidinyl or piperazinyl group that is substituted on a nitrogen atom thereof by, for example, a (2-6C)alkanoyl group, the terminal CH₃ group of the (2-6C)alkanoyl group may be further substituted by, for example, a di-[(1-6C)alkyl]amino group. For example, the R¹ or R⁶ group may include a N-(2-dimethylaminoacetyl)piperidin-4-yl group or a 4-(2-dimethylaminoacetyl)piperazin-1-yl group. Further, for example, if the R¹ or R⁶ group includes a heterocyclyl group such as a azetidinyl, piperidinyl or piperazinyl group that is substituted on a nitrogen atom thereof by, for example, a (2-6C)alkanoyl group, a CH₂ group of the (2-6C)alkanoyl group may be further substituted by, for example, a hydroxy group. For example, the R¹ or R⁶ group may include a N-(2-hydroxypropionyl)piperidin-4-yl group.

As defined hereinbefore, two R⁶ groups together may form a bivalent group, for example OC(R¹⁸)₂O, that spans adjacent ring positions on Ring A. When Ring A is, for example, a phenyl group, a suitable group so formed is a 2,3-methylenedioxyphenyl or a 3,4-methylenedioxyphenyl group. When a further optional R⁶ group is present, for example a halogeno group, a suitable group so formed is, for example, a 6-fluoro-2,3-methylenedioxyphenyl group. Further, when Ring A is, for example, a phenyl group and two R⁶ groups together form, for example, a OC(R¹⁸)₂C(R¹⁸)₂ group, a suitable group so formed is, for example, a 2,3-dihydrobenzofuran-5-yl group or a 2,3-dihydrobenzofuran-6-yl group. Further, when Ring A is, for example, a phenyl group and two R⁶ groups together form, for example, a N(R¹⁹)C(R¹⁸)₂C(R¹⁸)₂ group, a suitable group so formed is, for example, an indolin-5-yl group or a indolin-6-yl group. Further, when Ring A is, for example, a phenyl group and two R⁶ groups together form, for example, a N(R¹⁸)CO.C(R¹⁸)₂ group, a suitable group so formed is, for example, a 2-oxoindolin-5-yl group or a 2-oxoindolin-6-yl group.

A suitable pharmaceutically-acceptable salt of a compound of the Formula I is, for example, an acid-addition salt of a compound of the Formula I, for example an acid-addition salt with an inorganic or organic acid such as hydrochloric, hydrobromic, sulphuric, trifluoroacetic or citric acid; or, for example, a salt of a compound of the Formula I which is sufficiently acidic, for example an alkali or alkaline earth metal salt such as a calcium or magnesium salt, or an ammonium salt, or a salt with an organic base such as methylamine, dimethylamine, trimethylamine, piperidine, morpholine or tris-(2-hydroxyethyl)amine. A further suitable pharmaceutically-acceptable salt of a compound of the Formula I is, for example, a salt formed within the human or animal body after administration of a compound of the Formula I.

It is further to be understood that a suitable pharmaceutically-acceptable solvate of a compound of the Formula I also forms an aspect of the present invention. A suitable pharmaceutically-acceptable solvate is, for example, a hydrate such as a hemi-hydrate, a mono-hydrate, a di-hydrate or a tri-hydrate or an alternative quantity thereof.

It is further to be understood that a suitable pharmaceutically-acceptable pro-drug of a compound of the Formula I also forms an aspect of the present invention. Accordingly, the compounds of the invention may be administered in the form of a pro-drug, that is a compound that is broken down in the human or animal body to release a compound of the invention. A pro-drug may be used to alter the physical properties and/or the pharmacokinetic properties of a compound of the invention. A pro-drug can be formed when the compound of the invention contains a suitable group or substituent to which a property-modifying group can be attached.

Examples of pro-drugs include in vivo cleavable ester derivatives that may be formed at a carboxy group or a hydroxy group in a compound of the Formula I and in vivo cleavable amide derivatives that may be formed at a carboxy group or an amino group in a compound of the Formula I.

Accordingly, the present invention includes those compounds of the Formula I as defined hereinbefore when made available by organic synthesis and when made available within the human or animal body by way of cleavage of a pro-drug thereof. Accordingly, the present invention includes those compounds of the Formula I that are produced by organic synthetic means and also such compounds that are produced in the human or animal body by way of metabolism of a precursor compound, that is a compound of the Formula I may be a synthetically-produced compound or a metabolically-produced compound.

A suitable pharmaceutically-acceptable pro-drug of a compound of the Formula I is one that is based on reasonable medical judgement as being suitable for administration to the human or animal body without undesirable pharmacological activities and without undue toxicity.

Various forms of pro-drug have been described, for example in the following documents:—
a) Methods in Enzymology, Vol. 42, p. 309-396, edited by K. Widder, et al. (Academic Press, 1985);
b) Design of Pro-drugs, edited by H. Bundgaard, (Elsevier, 1985);
c) A Textbook of Drug Design and Development, edited by Krogsgaard-Larsen and H. Bundgaard, Chapter 5 “Design and Application of Pro-drugs”, by H. Bundgaard p. 113-191 (1991);
d) H. Bundgaard, Advanced Drug Delivery Reviews, 8, 1-38 (1992);
e) H. Bundgaard, et al., Journal of Pharmaceutical Sciences, 77, 285 (1988);
f) N. Kakeya, et al., Chem. Pharm. Bull., 32, 692 (1984);
g) T. Higuchi and V. Stella, “Pro-Drugs as Novel Delivery Systems”, A.C.S. Symposium Series, Volume 14; and
h) E. Roche (editor), “Bioreversible Carriers in Drug Design”, Pergamon Press, 1987.

A suitable pharmaceutically-acceptable pro-drug of a compound of the Formula I that possesses a carboxy group is, for example, an in vivo cleavable ester thereof. An in vivo cleavable ester of a compound of the Formula I containing a carboxy group is, for example, a pharmaceutically-acceptable ester which is cleaved in the human or animal body to produce the parent acid. Suitable pharmaceutically-acceptable esters for carboxy include (1-6C)alkyl esters such as methyl, ethyl and tert-butyl, (1-6C)alkoxymethyl esters such as methoxymethyl esters, (1-6C)alkanoyloxymethyl esters such as pivaloyloxymethyl esters, 3-phthalidyl esters, (3-8C)cycloalkylcarbonyloxy-(1-6C)alkyl esters such as cyclopentylcarbonyloxymethyl and 1-cyclohexylcarbonyloxyethyl esters, 2-oxo-1,3-dioxolenylmethyl esters such as 5-methyl-2-oxo-1,3-dioxolen-4-ylmethyl esters and (1-6C)alkoxycarbonyloxy-(1-6C)alkyl esters such as methoxycarbonyloxymethyl and 1-methoxycarbonyloxyethyl esters.

A suitable pharmaceutically-acceptable pro-drug of a compound of the Formula I that possesses a hydroxy group is, for example, an in vivo cleavable ester or ether thereof. An in vivo cleavable ester or ether of a compound of the Formula I containing a hydroxy group is, for example, a pharmaceutically-acceptable ester or ether which is cleaved in the human or animal body to produce the parent hydroxy compound. Suitable pharmaceutically-acceptable ester forming groups for a hydroxy group include inorganic esters such as phosphate esters (including phosphoramidic cyclic esters). Further suitable pharmaceutically-acceptable ester forming groups for a hydroxy group include (1-10C)alkanoyl groups such as acetyl, benzoyl, phenylacetyl and substituted benzoyl and phenylacetyl groups, (1-10C)alkoxycarbonyl groups such as ethoxycarbonyl, N,N-[di-(1-4C)alkyl]carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(1-4C)alkylpiperazin-1-ylmethyl. Suitable pharmaceutically-acceptable ether forming groups for a hydroxy group include α-acyloxyalkyl groups such as acetoxymethyl and pivaloyloxymethyl groups.

A suitable pharmaceutically-acceptable pro-drug of a compound of the Formula I that possesses a carboxy group is, for example, an in vivo cleavable amide thereof, for example an amide formed with an amine such as ammonia, a (1-4C)alkylamine such as methylamine, a di-(1-4C)alkylamine such as dimethylamine, N-ethyl-N-methylamine or diethylamine, a (1-4C)alkoxy-(2-4C)alkylamine such as 2-methoxyethylamine, a phenyl-(1-4C)alkylamine such as benzylamine and amino acids such as glycine or an ester thereof.

A suitable pharmaceutically-acceptable pro-drug of a compound of the Formula I that possesses an amino group is, for example, an in vivo cleavable amide derivative thereof. Suitable pharmaceutically-acceptable amides from an amino group include, for example an amide formed with (1-10C)alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(1-4C)alkylpiperazin-1-ylmethyl.

The in vivo effects of a compound of the Formula I may be exerted in part by one or more metabolites that are formed within the human or animal body after administration of a compound of the Formula I. As stated hereinbefore, the in vivo effects of a compound of the Formula I may also be exerted by way of metabolism of a precursor compound (a pro-drug).

Particular novel compounds of the invention include, for example, quinoline derivatives of the Formula I, or pharmaceutically-acceptable salts thereof, wherein, unless otherwise stated, each of X¹, p, R¹, q, R², R³, R⁴, R⁵, Ring A, r and R⁶ has any of the meanings defined hereinbefore or in paragraphs (a) to (iii) hereinafter:—

### (a) X¹ is O or NH;

### (b) X¹ is O;

### (c) X¹ is NH;

(d) p is 0, 1, 2 or 3, and each R¹ group that is present is selected from halogeno, trifluoromethyl, cyano, hydroxy, amino, carboxy, (1-6C)alkoxycarbonyl, carbamoyl, (1-8C)alkyl, (2-8C)alkenyl, (2-8C)alkynyl, (1-6C)alkoxy, (2-6C)alkenyloxy, (2-6C)alkynyloxy, (1-6C)alkylamino, di-[(1-6C)alkyl]amino, N-(1-6C)alkylcarbamoyl and N,N-di-[(1-6C)alkyl]carbamoyl, or from a group of the formula:

Q¹-X²-

wherein X² is selected from O, N(R⁸), CO, CON(R⁸), N(R⁸)CO and OC(R⁸)₂ wherein R⁸ is hydrogen or (1-8C)alkyl, and Q¹ is aryl, aryl-(1-6C)alkyl, (3-8C)cycloalkyl-(1-6C)alkyl, heteroaryl, heteroaryl-(1-6C)alkyl, heterocyclyl or heterocyclyl-(1-6C)alkyl,

and wherein any aryl, (3-8C)cycloalkyl, heteroaryl or heterocyclyl group within a substituent on R¹ optionally bears 1, 2 or 3 substituents, which may be the same or different, selected from halogeno, trifluoromethyl, hydroxy, amino, carbamoyl, (1-8C)alkyl, (2-8C)alkenyl, (2-8C)alkynyl, (1-6C)alkoxy, (1-6C)alkylsulphonyl, (1-6C)alkylamino, di-[(1-6C)alkyl]amino, (2-6C)alkanoyl, N-(1-6C)alkylcarbamoyl, N,N-di-[(1-6C)alkyl]carbamoyl, (2-6C)alkanoylamino and N-(1-6C)alkyl-(2-6C)alkanoylamino, or from a group of the formula:

-X³-R⁹

wherein X³ is a direct bond or is selected from O and N(R¹⁰), wherein R¹⁰ is hydrogen or (1-8C)alkyl, and R⁹ is halogeno-(1-6C)alkyl, hydroxy-(1-6C)alkyl, (1-6C)alkoxy-(1-6C)alkyl, (1-6C)alkylsulphonyl-(1-6C)alkyl, cyano-(1-6C)alkyl, amino-(1-6C)alkyl, (1-6C)alkylamino-(1-6C)alkyl, di-[(1-6C)alkyl]amino-(1-6C)alkyl, (2-6C)alkanoylamino-(1-6C)alkyl or N-(1-6C)alkyl-(2-6C)alkanoylamino-(1-6C)alkyl, or from a group of the formula:

-X⁴-Q²

wherein X⁴ is a direct bond or is selected from O, CO and N(R¹¹), wherein R¹¹ is hydrogen or (1-8C)alkyl, and Q² is heterocyclyl or heterocyclyl-(1-6C)alkyl which optionally bears 1 or 2 substituents, which may be the same or different, selected from halogeno, (1-8C)alkyl and (1-6C)alkoxy,

and wherein any heterocyclyl group within a substituent on R¹ optionally bears a (1-3C)alkylenedioxy group,

and wherein any heterocyclyl group within a substituent on R¹ optionally bears 1 or 2 oxo substituents,

and wherein any CH, CH₂ or CH₃ group within a R¹ substituent optionally bears on each said CH, CH₂ or CH₃ group one or more halogeno or (1-8C)alkyl groups and/or a substituent selected from hydroxy, amino, cyano, carboxy, carbamoyl, ureido, (1-6C)alkoxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoylamino, N-(1-6C)alkyl-(2-6C)alkanoylamino, N-(1-6C)alkylsulphamoyl, N,N-di-[(1-6C)alkyl]sulphamoyl, (1-6C)alkanesulphonylamino and N-(1-6C)alkyl-(1-6C)alkanesulphonylamino,

and wherein adjacent carbon atoms in any (2-6C)alkylene chain within a R¹ substituent are optionally separated by the insertion into the chain of a group selected from O, N(R¹²), CON(R¹²), N(R¹²)CO, CH═CH and C≡C wherein R¹² is hydrogen or (1-8C)alkyl, or, when the inserted group is N(R¹²), R¹² may also be (2-6C)alkanoyl;

(e) p is 1 or 2 and the R¹ groups are located at the 6- and/or 7-positions and the R¹ group at the 6-position is selected from halogeno, trifluoromethyl, cyano, hydroxy, amino, carboxy, (1-6C)alkoxycarbonyl, carbamoyl, (1-8C)alkyl, (2-8C)alkenyl, (2-8C)alkynyl, (1-6C)alkoxy, (2-6C)alkenyloxy, (2-6C)alkynyloxy, (1-6C)alkylamino, di-[(1-6C)alkyl]amino, N-(1-6C)alkylcarbamoyl and N,N-di-[(1-6C)alkyl]carbamoyl, and the R¹ group at the 7-position is selected from halogeno, trifluoromethyl, cyano, hydroxy, amino, carboxy, (1-6C)alkoxycarbonyl, carbamoyl, (1-8C)alkyl, (2-8C)alkenyl, (2-8C)alkynyl, (1-6C)alkoxy, (2-6C)alkenyloxy, (2-6C)alkynyloxy, (1-6C)alkylamino, di-[(1-6C)alkyl]amino, N-(1-6C)alkylcarbamoyl and N,N-di-[(1-6C)alkyl]carbamoyl, or from a group of the formula:

Q¹-X²-

wherein X² is selected from O, N(R⁸), CO, CON(R⁸), N(R⁸)CO and OC(R⁸)₂ wherein R⁸ is hydrogen or (1-8C)alkyl, and Q¹ is aryl, aryl-(1-6C)alkyl, (3-8C)cycloalkyl-(1-6C)alkyl, heteroaryl, heteroaryl-(1-6C)alkyl, heterocyclyl or heterocyclyl-(1-6C)alkyl,

and wherein any aryl, (3-8C)cycloalkyl, heteroaryl or heterocyclyl group within a substituent on R¹ optionally bears 1, 2 or 3 substituents, which may be the same or different, selected from halogeno, trifluoromethyl, hydroxy, amino, carbamoyl, (1-8C)alkyl, (2-8C)alkenyl, (2-8C)alkynyl, (1-6C)alkoxy, (1-6C)alkylsulphonyl, (1-6C)alkylamino, di-[(1-6C)alkyl]amino, (2-6C)alkanoyl, N-(1-6C)alkylcarbamoyl, N,N-di-[(1-6C)alkyl]carbamoyl, (2-6C)alkanoylamino and N-(1-6C)alkyl-(2-6C)alkanoylamino, or from a group of the formula:

-X³-R⁹

wherein X³ is a direct bond or is selected from O and N(R¹⁰), wherein R¹⁰ is hydrogen or (1-8C)alkyl, and R⁹ is halogeno-(1-6C)alkyl, hydroxy-(1-6C)alkyl, (1-6C)alkoxy-(1-6C)alkyl, (1-6C)alkylsulphonyl-(1-6C)alkyl, cyano-(1-6C)alkyl, amino-(1-6C)alkyl, (1-6C)alkylamino-(1-6C)alkyl, di-[(1-6C)alkyl]amino-(1-6C)alkyl, (2-6C)alkanoylamino-(1-6C)alkyl or N-(1-6C)alkyl-(2-6C)alkanoylamino-(1-6C)alkyl, or from a group of the formula:

-X⁴-Q²

wherein X⁴ is a direct bond or is selected from O, CO and N(R¹¹), wherein R¹¹ is hydrogen or (1-8C)alkyl, and Q² is heterocyclyl or heterocyclyl-(1-6C)alkyl which optionally bears 1 or 2 substituents, which may be the same or different, selected from halogeno, (1-8C)alkyl and (1-6C)alkoxy,

and wherein any heterocyclyl group within a substituent on R¹ optionally bears a (1-3C)alkylenedioxy group,

and wherein any heterocyclyl group within a substituent on R¹ optionally bears 1 or 2 oxo substituents,

and wherein any CH, CH₂ or CH₃ group within a R¹ substituent optionally bears on each said CH, CH₂ or CH₃ group one or more halogeno or (1-8C)alkyl groups and/or a substituent selected from hydroxy, amino, cyano, carboxy, carbamoyl, ureido, (1-6C)alkoxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoylamino, N-(1-6C)alkyl-(2-6C)alkanoylamino, N-(1-6C)alkylsulphamoyl, N,N-di-[(1-6C)alkyl]sulphamoyl, (1-6C)alkanesulphonylamino and N-(1-6C)alkyl-(1-6C)alkanesulphonylamino,

and wherein adjacent carbon atoms in any (2-6C)alkylene chain within a R¹ substituent are optionally separated by the insertion into the chain of a group selected from O, N(R¹²), CON(R¹²), N(R¹²)CO, CH═CH and C≡C wherein R¹² is hydrogen or (1-8C)alkyl, or, when the inserted group is N(R¹²), R¹² may also be (2-6C)alkanoyl;

(f) p is 1, 2 or 3 and one R¹ group is a 3-cyano group and any other R¹ groups may be located at the 5-, 6- or 7-position or at the 5- and 7-positions or at the 6- and 7-positions and each other R¹ group is selected from fluoro, chloro, trifluoromethyl, cyano, hydroxy, amino, carboxy, methoxycarbonyl, ethoxycarbonyl, carbamoyl, methyl, ethyl, propyl, butyl, vinyl, allyl, but-3-enyl, ethynyl, 2-propynyl, but-3-ynyl, methoxy, ethoxy, propoxy, isopropoxy, butoxy, allyloxy, but-3-enyloxy, ethynyloxy, 2-propynyloxy, but-3-ynyloxy, methylamino, ethylamino, propylamino, dimethylamino, diethylamino, dipropylamino, N-methylcarbamoyl, N-ethylcarbamoyl, N,N-dimethylcarbamoyl and N,N-diethylcarbamoyl, or from a group of the formula:

Q¹-X²-

wherein X² is selected from O, NH, CO, CONH, NHCO and OCH₂ and Q¹ is phenyl, benzyl, cyclopropylmethyl, 2-thienyl, 1-imidazolyl, 1,2,3-triazol-1-yl, 1,2,4-triazol-1-yl, 2-, 3- or 4-pyridyl, 2-imidazol-1-ylethyl, 3-imidazol-1-ylpropyl, 2-(1,2,3-triazolyl)ethyl, 3-(1,2,3-triazolyl)propyl, 2-(1,2,4-triazolyl)ethyl, 3-(1,2,4-triazolyl)propyl, 2-, 3- or 4-pyridylmethyl, 2-(2-, 3- or 4-pyridyl)ethyl, 3-(2-, 3- or 4-pyridyl)propyl, tetrahydrofuran-3-yl, 3- or 4-tetrahydropyranyl, 1-, 2- or 3-pyrrolidinyl, morpholino, 1,1-dioxotetrahydro-4H-1,4-thiazin-4-yl, piperidino, piperidin-3-yl, piperidin-4-yl, 1-, 3- or 4-homopiperidinyl, piperazin-1-yl, homopiperazin-1-yl, 1-, 2- or 3-pyrrolidinylmethyl, morpholinomethyl, piperidinomethyl, 3- or 4-piperidinylmethyl, 1-, 3- or 4-homopiperidinylmethyl, 2-pyrrolidin-1-ylethyl, 3-pyrrolidin-2-ylpropyl, pyrrolidin-2-ylmethyl, 2-pyrrolidin-2-ylethyl, 3-pyrrolidin-1-ylpropyl, 4-pyrrolidin-1-ylbutyl, 2-morpholinoethyl, 3-morpholinopropyl, 4-morpholinobutyl, 2-(1,1-dioxotetrahydro-4H-1,4-thiazin-4-yl)ethyl, 3-(1,1-dioxotetrahydro-4H-1,4-thiazin-4-yl)propyl, 2-piperidinoethyl, 3-piperidinopropyl, 4-piperidinobutyl, 2-piperidin-3-ylethyl, 3-piperidin-3-ylpropyl, 2-piperidin-4-ylethyl, 3-piperidin-4-ylpropyl, 2-homopiperidin-1-ylethyl, 3-homopiperidin-1-ylpropyl, 2-(1,2,3,6-tetrahydropyridin-1-yl)ethyl, 3-(1,2,3,6-tetrahydropyridin-1-yl)propyl, 4-(1,2,3,6-tetrahydropyridin-1-yl)butyl, 2-piperazin-1-ylethyl, 3-piperazin-1-ylpropyl, 4-piperazin-1-ylbutyl, 2-homopiperazin-1-ylethyl or 3-homopiperazin-1-ylpropyl,

and wherein any aryl, (3-8C)cycloalkyl, heteroaryl or heterocyclyl group within a substituent on R¹ optionally bears 1, 2 or 3 substituents, which may be the same or different, selected from fluoro, chloro, trifluoromethyl, hydroxy, amino, carbamoyl, methyl, ethyl, allyl, 2-propynyl, methoxy, methylsulphonyl, methylamino, dimethylamino, acetyl, propionyl, isobutyryl, N-methylcarbamoyl, N,N-dimethylcarbamoyl, methylenedioxy, ethylidendioxy and isopropylidenedioxy, or optionally bears 1 substituent selected from a group of the formula:

-X³-R⁹

wherein X³ is a direct bond or is selected from O and NH and R⁹ is 2-fluoroethyl, 2,2-difluoroethyl, 2,2,2-trifluoroethyl, 3-fluoropropyl, 3,3-difluoropropyl, 3,3,3-trifluoropropyl, 2-hydroxyethyl, 3-hydroxypropyl, 2-methoxyethyl, 3-methoxypropyl, cyanomethyl, aminomethyl, 2-aminoethyl, 3-aminopropyl, methylaminomethyl, 2-methylaminoethyl, 3-methylaminopropyl, 2-ethylaminoethyl, 3-ethylaminopropyl, dimethylaminomethyl, 2-dimethylaminoethyl, 3-dimethylaminopropyl, acetamidomethyl or N-methylacetamidomethyl, and from a group of the formula:

-X⁴-Q²

wherein X⁴ is a direct bond or is selected from O, CO and NH and Q² is pyrrolidin-1-ylmethyl, 2-pyrrolidin-1-ylethyl, 3-pyrrolidin-1-ylpropyl, morpholinomethyl, 2-morpholinoethyl, 3-morpholinopropyl, piperidinomethyl, 2-piperidinoethyl, 3-piperidinopropyl, piperazin-1-ylmethyl, 2-piperazin-1-ylethyl or 3-piperazin-1-ylpropyl, each of which optionally bears 1 or 2 substituents, which may be the same or different, selected from fluoro, chloro, methyl and methoxy,

and wherein any heterocyclyl group within a substituent on R¹ optionally bears 1 or 2 oxo substituents,

and wherein any CH, CH₂ or CH₃ group within a R¹ substituent optionally bears on each said CH, CH₂ or CH₃ group one or more fluoro, chloro or methyl groups or a substituent selected from hydroxy, amino, cyano, methoxy, methylsulphonyl, methylamino, dimethylamino, diisopropylamino, N-ethyl-N-methylamino, N-isopropyl-N-methylamino, acetyl, acetamido and N-methylacetamido,

and wherein adjacent carbon atoms in any (2-6C)alkylene chain within a R¹ substituent are optionally separated by the insertion into the chain of a group selected from O, NH, N(Me), N(COMe), CONH, NHCO, CH═CH and C≡C;

(g) p is 2 and the R¹ groups are located at the 5- and 7-positions or at the 6- and 7-positions and the R¹ groups, which may be the same or different, are selected from cyano, hydroxy, amino, carboxy, methoxycarbonyl, ethoxycarbonyl, carbamoyl, methyl, ethyl, propyl, butyl, vinyl, ethynyl, methoxy, ethoxy, propoxy, isopropoxy, butoxy, but-3-enyloxy, methylamino, ethylamino, dimethylamino, diethylamino, N-methylcarbamoyl, N-ethylcarbamoyl, N,N-dimethylcarbamoyl, N,N-diethylcarbamoyl, cyclopentyloxy, cyclohexyloxy, phenoxy, benzyloxy, tetrahydrofuran-3-yloxy, tetrahydropyran-3-yloxy, tetrahydropyran-4-yloxy, cyclopropylmethoxy, 2-imidazol-1-ylethoxy, 3-imidazol-1-ylpropoxy, 2-(1,2,3-triazol-1-yl)ethoxy, 3-(1,2,3-triazol-1-yl)propoxy, 2-(1,2,4-triazol-1-yl)ethoxy, 3-(1,2,4-triazol-1-yl)propoxy, pyrid-2-ylmethoxy, pyrid-3-ylmethoxy, pyrid-4-ylmethoxy, 2-pyrid-2-ylethoxy, 2-pyrid-3-ylethoxy, 2-pyrid-4-ylethoxy, 3-pyrid-2-ylpropoxy, 3-pyrid-3-ylpropoxy, 3-pyrid-4-ylpropoxy, pyrrolidin-1-yl, morpholino, piperidino, piperazin-1-yl, pyrrolidin-1-ylcarbonyl, morpholinocarbonyl, piperidinocarbonyl, piperazin-1-ylcarbonyl, 2-pyrrolidin-1-ylethoxy, 3-pyrrolidin-1-ylpropoxy, 4-pyrrolidin-1-ylbutoxy, pyrrolidin-3-yloxy, pyrrolidin-2-ylmethoxy, 2-pyrrolidin-2-ylethoxy, 3-pyrrolidin-2-ylpropoxy, 2-morpholinoethoxy, 3-morpholinopropoxy, 4-morpholinobutoxy, 2-(1,1-dioxotetrahydro-4H-1,4-thiazin-4-yl)ethoxy, 3-(1,1-dioxotetrahydro-4H-1,4-thiazin-4-yl)propoxy, 2-piperidinoethoxy, 3-piperidinopropoxy, 4-piperidinobutoxy, piperidin-3-yloxy, piperidin-4-yloxy, piperidin-3-ylmethoxy, piperidin-4-ylmethoxy, 2-piperidin-3-ylethoxy, 3-piperidin-3-ylpropoxy, 2-piperidin-4-ylethoxy, 3-piperidin-4-ylpropoxy, 2-homopiperidin-1-ylethoxy, 3-homopiperidin-1-ylpropoxy, 2-(1,2,3,6-tetrahydropyridin-1-yl)ethoxy 3-(1,2,3,6-tetrahydropyridin-1-yl)propoxy, 4-(1,2,3,6-tetrahydropyridin-1-yl)butoxy, 2-piperazin-1-ylethoxy, 3-piperazin-1-ylpropoxy, 4-piperazin-1-ylbutoxy, 2-homopiperazin-1-ylethoxy, 3-homopiperazin-1-ylpropoxy, 2-pyrrolidin-1-ylethylamino, 3-pyrrolidin-1-ylpropylamino, 4-pyrrolidin-1-ylbutylamino, pyrrolidin-3-ylamino, pyrrolidin-2-ylmethylamino, 2-pyrrolidin-2-ylethylamino, 3-pyrrolidin-2-ylpropylamino, 2-morpholinoethylamino, 3-morpholinopropylamino, 4-morpholinobutylamino, 2-(1,1-dioxotetrahydro-4H-1,4-thiazin-4-yl)ethylamino, 3-(1,1-dioxotetrahydro-4H-1,4-thiazin-4-yl)propylamino, 2-piperidinoethylamino, 3-piperidinopropylamino, 4-piperidinobutylamino, piperidin-3-ylamino, piperidin-4-ylamino, piperidin-3-ylmethylamino, 2-piperidin-3-ylethylamino, piperidin-4-ylmethylamino, 2-piperidin-4-ylethylamino, 2-homopiperidin-1-ylethylamino, 3-homopiperidin-1-ylpropylamino, 2-piperazin-1-ylethylamino, 3-piperazin-1-ylpropylamino, 4-piperazin-1-ylbutylamino, 2-homopiperazin-1-ylethylamino or 3-homopiperazin-1-ylpropylamino,

and wherein any phenyl, imidazolyl, triazolyl, pyridyl or heterocyclyl group within a substituent on R¹ optionally bears 1 or 2 substituents, which may be the same or different, selected from fluoro, chloro, trifluoromethyl, hydroxy, amino, carbamoyl, methyl, ethyl, methoxy, ethoxy, N-methylcarbamoyl, N,N-dimethylcarbamoyl, methylenedioxy, ethylidendioxy and isopropylidenedioxy, and a pyrrolidin-2-yl, piperidin-3-yl, piperidin-4-yl, piperazin-1-yl or homopiperazin-1-yl group within a R¹ substituent is optionally N-substituted is with allyl, 2-propynyl, methylsulphonyl, ethylsulphonyl, acetyl, propionyl, isobutyryl, 2-fluoroethyl, 2,2-difluoroethyl, 2,2,2-trifluoroethyl, 3-fluoropropyl, 3,3-difluoropropyl, 3,3,3-trifluoropropyl, 2-methoxyethyl, 3-methoxypropyl, cyanomethyl, 2-aminoethyl, 3-aminopropyl, 2-methylaminoethyl, 3-methylaminopropyl, 2-dimethylaminoethyl, 3-dimethylaminopropyl, 2-pyrrolidin-1-ylethyl, 3-pyrrolidin-1-ylpropyl, 2-morpholinoethyl, 3-morpholinopropyl, 2-piperidinoethyl, 3-piperidinopropyl, 2-piperazin-1-ylethyl or 3-piperazin-1-ylpropyl, the last 8 of which substituents each optionally bears 1 or 2 substituents, which may be the same or different, selected from fluoro, chloro, methyl and methoxy,

and wherein any heterocyclyl group within a substituent on R¹ optionally bears 1 or 2 oxo substituents,

and wherein any CH, CH₂ or CH₃ group within a R¹ substituent optionally bears on each said CH, CH₂ or CH₃ group one or more fluoro, chloro or methyl groups or a substituent selected from hydroxy, amino, methoxy, methylsulphonyl, methylamino, dimethylamino, diisopropylamino, N-ethyl-N-methylamino, N-isopropyl-N-methylamino, N-methyl-N-propylamino, acetamido and N-methylacetamido,

and wherein adjacent carbon atoms in any (2-6C)alkylene chain within a R¹ substituent are optionally separated by the insertion into the chain of a group selected from O, NH, N(Me), CH═CH and C≡C;

(h) p is 2 and the R¹ groups are located at the 6- and 7-positions and the R¹ groups, which may be the same or different, are selected from cyano, hydroxy, amino, methoxycarbonyl, ethoxycarbonyl, carbamoyl, methyl, ethyl, methoxy, ethoxy, propoxy, isopropoxy, butoxy, methylamino, ethylamino, dimethylamino, diethylamino, N-methylcarbamoyl, N-ethylcarbamoyl, N,N-dimethylcarbamoyl, N,N-diethylcarbamoyl, pyrrolidin-1-ylcarbonyl, morpholinocarbonyl, piperidinocarbonyl, piperazin-1-ylcarbonyl, 2-pyrrolidin-1-ylethoxy, 3-pyrrolidin-1-ylpropoxy, 4-pyrrolidin-1-ylbutoxy, pyrrolidin-3-yloxy, pyrrolidin-2-ylmethoxy, 2-pyrrolidin-2-ylethoxy, 3-pyrrolidin-2-ylpropoxy, 2-morpholinoethoxy, 3-morpholinopropoxy, 4-morpholinobutoxy, 2-(1,1-dioxotetrahydro-4H-1,4-thiazin-4-yl)ethoxy, 3-(1,1-dioxotetrahydro-4H-1,4-thiazin-4-yl)propoxy, 2-piperidinoethoxy, 3-piperidinopropoxy, 4-piperidinobutoxy, piperidin-3-yloxy, piperidin-4-yloxy, piperidin-3-ylmethoxy, 2-piperidin-3-ylethoxy, piperidin-4-ylmethoxy, 2-piperidin-4-ylethoxy, 2-homopiperidin-1-ylethoxy, 3-homopiperidin-1-ylpropoxy, 3-(1,2,3,6-tetrahydropyridin-1-yl)propoxy, 2-piperazin-1-ylethoxy, 3-piperazin-1-ylpropoxy, 2-homopiperazin-1-ylethoxy and 3-homopiperazin-1-ylpropoxy,

and wherein any heterocyclyl group within a substituent on R¹ optionally bears 1 or 2 substituents, which may be the same or different, selected from fluoro, chloro, trifluoromethyl, hydroxy, amino, methyl, ethyl, methoxy, methylenedioxy, ethylidendioxy and isopropylidenedioxy, and a pyrrolidin-2-yl, pyrrolidin-3-yl, piperidin-3-yl, piperidin-4-yl, piperazin-1-yl or homopiperazin-1-yl group within a R¹ substituent is optionally N-substituted with methyl, ethyl, propyl, allyl, 2-propynyl, methylsulphonyl, acetyl, propionyl, isobutyryl, 2-fluoroethyl, 2,2-difluoroethyl, 2,2,2-trifluoroethyl or cyanomethyl,

and wherein any heterocyclyl group within a substituent on R¹ optionally bears 1 or 2 oxo substituents,

and wherein any CH, CH₂ or CH₃ group within a R¹ substituent optionally bears on each said CH, CH₂ or CH₃ group one or more chloro groups or a substituent selected from hydroxy, amino, methoxy, methylsulphonyl, methylamino, dimethylamino, diisopropylamino, N-ethyl-N-methylamino and N-isopropyl-N-methylamino,

and wherein adjacent carbon atoms in any (2-6C)alkylene chain within a R¹ substituent are optionally separated by the insertion into the chain of a group selected from O, NH, CH═CH and C≡C;

(i) p is 2 and the R¹ groups are located at the 6- and 7-positions and the R¹ groups, which may be the same or different, are selected from hydroxy, amino, methoxycarbonyl, ethoxycarbonyl, carbamoyl, methyl, ethyl, methoxy, ethoxy, propoxy, isopropoxy, butoxy, methylamino, ethylamino, dimethylamino, diethylamino, N-methylcarbamoyl, N-ethylcarbamoyl, N,N-dimethylcarbamoyl, N,N-diethylcarbamoyl, pyrrolidin-1-ylcarbonyl, morpholinocarbonyl, piperidinocarbonyl, piperazin-1-ylcarbonyl, 2-pyrrolidin-1-ylethoxy, 3-pyrrolidin-1-ylpropoxy, 4-pyrrolidin-1-ylbutoxy, pyrrolidin-3-yloxy, pyrrolidin-2-ylmethoxy, 2-pyrrolidin-2-ylethoxy, 3-pyrrolidin-2-ylpropoxy, 2-morpholinoethoxy, 3-morpholinopropoxy, 4-morpholinobutoxy, 2-(1,1-dioxotetrahydro-4H-1,4-thiazin-4-yl)ethoxy, 3-(1,1-dioxotetrahydro-4H-1,4-thiazin-4-yl)propoxy, 2-piperidinoethoxy, 3-piperidinopropoxy, 4-piperidinobutoxy, 3-piperidinyloxy, 4-piperidinyloxy, piperidin-3-ylmethoxy, piperidin-4-ylmethoxy, 2-piperidin-3-ylethoxy, 2-piperidin-4-ylethoxy, 2-homopiperidin-1-ylethoxy, 3-homopiperidin-1-ylpropoxy, 3-(1,2,3,6-tetrahydropyridin-1-yl)propoxy, 2-piperazin-1-ylethoxy, 3-piperazin-1-ylpropoxy, 2-homopiperazin-1-ylethoxy and 3-homopiperazin-1-ylpropoxy,

and wherein any heterocyclyl group within a substituent on R¹ optionally bears 1 or 2 substituents, which may be the same or different, selected from fluoro, chloro, trifluoromethyl, hydroxy, amino, methyl, ethyl, methoxy, methylenedioxy, ethylidendioxy and isopropylidenedioxy, and a pyrrolidin-2-yl, pyrrolidin-3-yl, piperidin-3-yl, piperidin-4-yl, piperazin-1-yl or homopiperazin-1-yl group within a R¹ substituent is optionally N-substituted with methyl, ethyl, propyl, allyl, 2-propynyl, methylsulphonyl, acetyl, propionyl, isobutyryl, 2-fluoroethyl, 2,2-difluoroethyl, 2,2,2-trifluoroethyl or cyanomethyl,

and wherein any heterocyclyl group within a substituent on R¹ optionally bears 1 or 2 oxo substituents,

and wherein any CH, CH₂ or CH₃ group within a R¹ substituent optionally bears on each said CH, CH₂ or CH₃ group one or more chloro groups or a substituent selected from hydroxy, amino, methoxy, methylsulphonyl, methylamino, dimethylamino, diisopropylamino, N-ethyl-N-methylamino and N-isopropyl-N-methylamino,

and wherein adjacent carbon atoms in any (2-6C)allylene chain within a R¹ substituent are optionally separated by the insertion into the chain of a group selected from O, NH, CH═CH and C≡C;

(j) p is 2 and the R¹ groups are located at the 6- and 7-positions and the R¹ group at the 6-position is selected from cyano, hydroxy, methoxycarbonyl, ethoxycarbonyl, carbamoyl, methoxy, ethoxy, propoxy, N-methylcarbamoyl, N-ethylcarbamoyl, N,N-dimethylcarbamoyl, N,N-diethylcarbamoyl, pyrrolidin-1-ylcarbonyl, morpholinocarbonyl, piperidinocarbonyl and piperazin-1-ylcarbonyl, and the R¹ group at the 7-position is selected from methoxy, ethoxy, propoxy, 2-pyrrolidin-1-ylethoxy, 3-pyrrolidin-1-ylpropoxy, 4-pyrrolidin-1-ylbutoxy, pyrrolidin-3-yloxy, pyrrolidin-2-ylmethoxy, 2-pyrrolidin-2-ylethoxy, 3-pyrrolidin-2-ylpropoxy, 2-morpholinoethoxy, 3-morpholinopropoxy, 4-morpholinobutoxy, 2-(1,1-dioxotetrahydro-4H-1,4-thiazin-4-yl)ethoxy, 3-(1,1-dioxotetrahydro-4H-1,4-thiazin-4-yl)propoxy, 2-piperidinoethoxy, 3-piperidinopropoxy, 4-piperidinobutoxy, piperidin-3-yloxy, piperidin-4-yloxy, piperidin-3-ylmethoxy, 2-piperidin-3-ylethoxy, piperidin-4-ylmethoxy, 2-piperidin-4-ylethoxy, 2-homopiperidin-1-ylethoxy, 3-homopiperidin-1-ylpropoxy, 3-(1,2,3,6-tetrahydropyridin-1-yl)propoxy, 2-piperazin-1-ylethoxy, 3-piperazin-1-ylpropoxy, 2-homopiperazin-1-ylethoxy and 3-homopiperazin-1-ylpropoxy,

and wherein any heterocyclyl group within a substituent on R¹ optionally bears 1 or 2 substituents, which may be the same or different, selected from fluoro, chloro, trifluoromethyl, hydroxy, amino, methyl, ethyl, methoxy, methylenedioxy, ethylidendioxy and isopropylidenedioxy, and a pyrrolidin-2-yl, pyrrolidin-3-yl, piperidin-3-yl, piperidin-4-yl, piperazin-1-yl or homopiperazin-1-yl group within a R¹ substituent is optionally N-substituted with methyl, ethyl, propyl, allyl, 2-propynyl, methylsulphonyl, acetyl, propionyl, isobutyryl, 2-fluoroethyl, 2,2-difluoroethyl, 2,2,2-trifluoroethyl or cyanomethyl,

and wherein any heterocyclyl group within a substituent on R¹ optionally bears 1 or 2 oxo substituents,

and wherein any CH, CH₂ or CH₃ group within a R¹ substituent optionally bears on each said CH, CH₂ or CH₃ group one or more chloro groups or a substituent selected from hydroxy, amino, methoxy, methylsulphonyl, methylamino, dimethylamino, diisopropylamino, N-ethyl-N-methylamino and N-isopropyl-N-methylamino;

(k) q is 0;
(l) q is 1 or 2 and each R² group, which may be the same or different, is selected from halogeno, trifluoromethyl, cyano, carbamoyl, hydroxy, amino, (1-8C)alkyl, (2-8C)alkenyl, (2-8C)alkynyl, (1-6C)alkoxy, (1-6C)alkylamino, di-[(1-6C)alkyl]amino, N-(1-6C)alkylcarbamoyl and N,N-di-[(1-6C)alkyl]carbamoyl;
(m) q is 1 or 2 and each R² group, which may be the same or different, is selected from halogeno, trifluoromethyl, cyano, hydroxy, amino, (1-8C)alkyl, (2-8C)alkenyl, (2-8C)alkynyl, (1-6C)alkoxy, (1-6C)alkylamino and di-[(1-6C)alkyl]amino;
(n) q is 1 or 2 and each R² group, which may be the same or different, is selected from fluoro, chloro, trifluoromethyl, cyano, hydroxy, amino, methyl, methoxy, methylamino and dimethylamino;
(o) q is 1 and the R² group which is located at the 2-position (relative to the C(R³)(R⁴) group) is a (1-6C)alkoxy group;
(p) q is 1 and the R² group which is located at the 2-position (relative to the C(R³)(R⁴) group) is selected from fluoro, chloro, trifluoromethyl, cyano, carbamoyl, hydroxy, amino, methyl, methoxy, methylamino, dimethylamino, N-methylcarbamoyl and N,N-dimethylcarbamoyl;
(q) q is 0 or q is 1 and the R² group which is located at the 2-position (relative to the C(R³)(R⁴) group) is selected from fluoro, chloro, trifluoromethyl, cyano, hydroxy, amino, methyl, methoxy, methylamino and dimethylamino;
(r) q is 1 and the R² group which is located at the 2-position (relative to the C(R³)(R⁴) group) is selected from fluoro, chloro, cyano, methyl and methoxy;
(s) q is 1 and the R² group which is located at the 2-position (relative to the C(R³)(R⁴) group) is a methoxy group;
(t) R³ is hydrogen, methyl or ethyl;
(u) R³ is hydrogen;
(v) R⁴ is hydrogen, methyl, ethyl, propyl, 2-fluoroethyl, 2,2-difluoroethyl, 2,2,2-trifluoroethyl, 3-fluoropropyl, 3,3-difluoropropyl, 3,3,3-trifluoropropyl, 2-hydroxyethyl, 3-hydroxypropyl, 2-methoxyethyl, 3-methoxypropyl, cyanomethyl, 2-cyanoethyl, aminomethyl, 2-aminoethyl, 3-aminopropyl, methylaminomethyl, 2-methylaminoethyl, 3-methylaminopropyl, 2-ethylaminoethyl, 3-ethylaminopropyl, dimethylaminomethyl, 2-dimethylaminoethyl, 3-dimethylaminopropyl, acetamidomethyl or N-methylacetamidomethyl;
(w) R⁴ is hydrogen, methyl or ethyl;
(x) R⁴ is hydrogen;
(y) R³ and R⁴ together with the carbon atom to which they are attached form a cyclopropyl, cyclobutyl, cyclopentyl or cyclohexyl group;
(z) R⁵ is hydrogen, methyl, ethyl, propyl, allyl, 2-propynyl, 2-fluoroethyl, 2,2-difluoroethyl, 2,2,2-trifluoroethyl, 3-fluoropropyl, 3,3-difluoropropyl, 3,3,3-trifluoropropyl, 2-hydroxyethyl, 3-hydroxypropyl, 2-methoxyethyl, 3-methoxypropyl, cyanomethyl, 2-cyanoethyl or 3-cyanopropyl;
(aa) R⁵ is methyl or ethyl;
(bb) R⁵ is hydrogen;
(cc) Ring A is a 6-membered monocyclic aryl ring or a 5- or 6-membered monocyclic heteroaryl ring with up to three ring heteroatoms selected from oxygen, nitrogen and sulphur;
(dd) Ring A is a phenyl ring;
(ee) Ring A is a 6-membered monocyclic heteroaryl ring with up to three nitrogen heteroatoms;
(ff) Ring A is a 5-membered monocyclic heteroaryl ring with up to three ring heteroatoms selected from oxygen, nitrogen and sulphur;
(gg) Ring A is a phenyl, furyl, pyrrolyl, thienyl, oxazolyl, isoxazolyl, imidazolyl, pyrazolyl, thiazolyl, isothiazolyl, oxadiazolyl, thiadiazolyl, pyridyl, pyrimidinyl, pyrazinyl or pyridazinyl ring;
(hh) Ring A is a phenyl, pyridyl, pyrimidinyl, pyrazinyl or pyridazinyl ring;
(ii) Ring A is a furyl, pyrrolyl, thienyl, oxazolyl, isoxazolyl, imidazolyl, pyrazolyl, thiazolyl, isothiazolyl, oxadiazolyl or thiadiazolyl ring;
(jj) when Ring A is a 6-membered ring, and one or two R⁶ groups are present, one R⁶ group is located at the 3- or 4-position (relative to the CON(R⁵) group);
(kk) when Ring A is a 5-membered ring, and one or two R⁶ groups are present, one R⁶ group is located at the 3-position (relative to the CON(R⁵) group);
(ll) Ring A is a phenyl, pyridyl, pyrimidinyl, pyrazinyl or pyridazinyl ring that bears one or two R⁶ groups and one R⁶ group is located at the 3- or 4-position (relative to the CON(R⁵) group);
(mm) Ring A is a furyl, pyrrolyl, thienyl, oxazolyl, isoxazolyl, imidazolyl, pyrazolyl, thiazolyl, isothiazolyl, oxadiazolyl or thiadiazolyl ring that bears one or two R⁶ groups and one R⁶ group is located at the 3-position (relative to the CON(R⁵) group);
(nn) Ring A is a 9- or 10-membered bicyclic heteroaryl ring with up to three ring heteroatoms selected from oxygen, nitrogen and sulphur;
(oo) Ring A is a benzofuranyl, indolyl, benzothienyl, benzoxazolyl, benzimidazolyl, benzothiazolyl, indazolyl, benzotriazolyl, 1H-pyrrolo[3,2-b]pyridinyl, quinolyl, isoquinolyl, quinazolinyl, quinoxalinyl or naphthyridinyl ring;
(pp) r is 0, 1, 2 or 3 and each R⁶ group that is present, which may be the same or different, is selected from halogeno, trifluoromethyl, cyano, hydroxy, amino, (1-8C)alkyl, (2-8C)alkenyl, (2-8C)alkynyl, (1-6C)alkoxy, (1-6C)alkylamino, di-[(1-6C)alkyl]amino, (2-6C)alkanoylamino and N-(1-6C)alkyl-(2-6C)alkanoylamino;
(qq) r is 1 or 2 and each R⁶ group, which may be the same or different, is selected from fluoro, chloro, trifluoromethyl, cyano, hydroxy, amino, methyl, ethyl, propyl, isopropyl, butyl, sec-butyl, isobutyl, tert-butyl, methoxy, ethoxy, methylamino, ethylamino, dimethylamino and diethylamino;
(rr) r is 1 and the R⁶ group is selected from fluoro, chloro, trifluoromethyl, hydroxy, amino, methyl, ethyl, propyl, isopropyl, butyl, sec-butyl, isobutyl, tert-butyl, methoxy, ethoxy, methylamino, ethylamino, dimethylamino and diethylamino;
(ss) r is 1, 2 or 3 and one R⁶ group is a group of the formula:

-X⁶-R¹⁵

wherein X⁶ is a direct bond or is selected from O and N(R¹⁶), wherein R¹⁶ is hydrogen or (1-8C)alkyl, and R¹⁵ is halogeno-(1-6C)alkyl, hydroxy-(1-6C)alkyl, mercapto-(1-6C)alkyl, (1-6C)alkoxy-(1-6C)alkyl, (1-6C)alkylthio-(1-6C)alkyl, (1-6C)alkylsulphinyl-(1-6C)alkyl, (1-6C)alkylsulphonyl-(1-6C)alkyl, cyano-(1-6C)alkyl, amino-(1-6C)alkyl, (1-6C)alkylamino-(1-6C)alkyl, di-[(1-6C)alkyl]amino-(1-6C)alkyl, (2-6C)alkanoylamino-(1-6C)alkyl, N-(1-6C)alkyl-(2-6C)alkanoylamino-(1-6C)alkyl, carboxy-(1-6C)alkyl, (1-6C)alkoxycarbonyl-(1-6C)alkyl, carbamoyl-(1-6C)alkyl, N-(1-6C)alkylcarbamoyl-(1-6C)alkyl or N,N-di-[(1-6C)alkyl]carbamoyl-(1-6C)alkyl provided that, when X⁶ is O or N(R¹⁶), there are at least two carbon atoms between X⁶ and any heteroatom in the R¹⁵ group,

and any other R⁶ group that is present is selected from halogeno, trifluoromethyl, cyano, hydroxy, amino, (1-8C)alkyl, (2-8C)alkenyl, (2-8C)alkynyl, (1-6C)alkoxy, (1-6C)alkylamino, di-[(1-6C)alkyl]amino, (2-6C)alkanoylamino and N-(1-6C)alkyl-(2-6C)alkanoylamino,

and wherein any CH, CH₂ or CH₃ group within an R⁶ group optionally bears on each said CH, CH₂ or CH₃ group one or more halogeno or (1-8C)alkyl substituents and/or a substituent selected from hydroxy, amino, cyano, carboxy, carbamoyl, ureido, (1-6C)alkoxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoylamino and N-(1-6C)alkyl-(2-6C)alkanoylamino;

(tt) r is 1, 2 or 3 and one R⁶ group is a group of the formula:

-X⁷-Q³

wherein X⁷ is a direct bond or is selected from O, N(R¹⁷), CON(R¹⁷), N(R¹⁷)CO and C(R¹⁷)₂O, wherein each R¹⁷ is hydrogen or (1-8C)alkyl, and Q³ is aryl, aryl-(1-6C)alkyl, (3-8C)cycloalkyl, (3-8C)cycloalkyl-(1-6C)alkyl, heteroaryl, heteroaryl-(1-6C)alkyl, heterocyclyl or heterocyclyl-(1-6C)alkyl provided that, when X⁷ is selected from O, N(R¹⁷), CON(R¹⁷) or C(R¹⁷)₂O, there are at least two carbon atoms between X⁷ and any heteroatom in Q³ that is not in a heteroaryl ring,

and any other R⁶ group that is present is selected from halogeno, trifluoromethyl, cyano, hydroxy, amino, (1-8C)alkyl, (2-8C)alkenyl, (2-8C)alkynyl, (1-6C)alkoxy, (1-6C)alkylamino, di-[(1-6C)alkyl]amino, (2-6C)alkanoylamino and N-(1-6C)alkyl-(2-6C)alkanoylamino,

and wherein any aryl, (3-8C)cycloalkyl, heteroaryl or heterocyclyl group within an R⁶ group optionally bears 1, 2 or 3 substituents, which may be the same or different, selected from halogeno, trifluoromethyl, cyano, hydroxy, amino, carboxy, carbamoyl, ureido, (1-8C)alkyl, (2-8C)alkenyl, (2-8C)alkynyl, (1-6C)alkoxy, (1-6C)alkylamino and di-[(1-6C)alkyl]amino, or from a group of the formula:

-X⁸-R²⁰

wherein X⁸ is a direct bond or is selected from O and N(R²¹), wherein R²¹ is hydrogen or (1-8C)alkyl, and R²⁰ is halogeno-(1-6C)alkyl, hydroxy-(1-6C)alkyl, (1-6C)alkoxy-(1-6C)alkyl, cyano-(1-6C)alkyl, amino-(1-6C)alkyl, (1-6C)alkylamino-(1-6C)alkyl or di-[(1-6C)alkyl]amino-(1-6C)alkyl,

and wherein any heterocyclyl group within an R⁶ group optionally bears 1 or 2 oxo or thioxo substituents,

and wherein any CH, CH₂ or CH₃ group within an R⁶ group optionally bears on each said CH, CH₂ or CH₃ group one or more halogeno or (1-8C)alkyl substituents and/or a substituent selected from hydroxy, amino, cyano, carboxy, carbamoyl, ureido, (1-6C)alkoxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoylamino and N-(1-6C)alkyl-(2-6C)alkanoylamino;

(uu) r is 1, 2 or 3 and one R⁶ group is a group of the formula:

-X⁶-R¹⁵

wherein X⁶ is a direct bond or is selected from O and N(R¹⁶), wherein R¹⁶ is hydrogen or (1-8C)alkyl, and R¹⁵ is hydroxy-(1-6C)alkyl, (1-6C)alkoxy-(1-6C)alkyl, (1-6C)alkylthio-(1-6C)alkyl, (1-6C)alkylsulphinyl-(1-6C)alkyl, (1-6C)alkylsulphonyl-(1-6C)alkyl, cyano-(1-6C)alkyl, amino-(1-6C)alkyl, (1-6C)alkylamino-(1-6C)alkyl, di-[(1-6C)alkyl]amino-(1-6C)alkyl, (2-6C)alkanoylamino-(1-6C)alkyl, N-(1-6C)alkyl-(2-6C)alkanoylamino-(1-6C)alkyl, aryl, aryl-(1-6C)alkyl, (3-8C)cycloalkyl, (3-8C)cycloalkyl-(1-6C)alkyl, heteroaryl, heteroaryl-(1-6C)alkyl, heterocyclyl or heterocyclyl-(1-6C)alkyl, provided that, when X⁶ is O or N(R¹⁶), there are at least two carbon atoms between X⁶ and any heteroatom in the R¹⁶ group,

and any other R⁶ group that is present is selected from halogeno, trifluoromethyl, cyano, hydroxy, amino, (1-8C)alkyl, (2-8C)alkenyl, (2-8C)alkynyl, (1-6C)alkoxy, (1-6C)alkylamino, di-[(1-6C)alkyl]amino, (2-6C)alkanoylamino and N-(1-6C)alkyl-(2-6C)alkanoylamino,

and wherein any aryl, (3-8C)cycloalkyl, heteroaryl or heterocyclyl group within the R⁶ group optionally bears 1 or 2 substituents, which may be the same or different, selected from halogeno, trifluoromethyl, cyano, hydroxy, amino, (1-8C)alkyl, (1-6C)alkoxy, (1-6C)alkylamino and di-[(1-6C)alkyl]amino, or from a group of the formula:

-X⁸-R²⁰

wherein X⁸ is a direct bond and R²⁰ is halogeno-(1-6C)alkyl, hydroxy-(1-6C)alkyl, (1-6C)alkoxy-(1-6C)alkyl, cyano-(1-6C)alkyl, amino-(1-6C)alkyl, (1-6C)alkylamino-(1-6C)alkyl or di-[(1-6C)alkyl]amino-(1-6C)alkyl,

and wherein any CH, CH₂ or CH₃ group within the R⁶ group optionally bears on each said CH, CH₂ or CH₃ group 1, 2 or 3 halogeno or (1-8C)alkyl substituents and/or a substituent selected from hydroxy, amino, cyano, (3-8C)alkenyl, (3-8C)alkynyl, (1-6C)alkoxy, (1-6C)alkylsulphonyl, (1-6C)alkylamino, di-[(1-6C)alkyl]amino, (2-6C)alkanoylamino and N-(1-6C)alkyl-(2-6C)alkanoylamino;

(vv) r is 1, 2 or 3 and one R⁶ group is a group of the formula:

-X⁶-R¹⁵

wherein X⁶ is a direct bond or is selected from O and N(R¹⁶), wherein R¹⁶ is hydrogen or (1-8C)alkyl, and R¹⁵ is hydroxy-(1-6C)alkyl, (1-6C)alkoxy-(1-6C)alkyl, amino-(1-6C)alkyl, (1-6C)alkylamino-(1-6C)alkyl, di-[(1-6C)alkyl]amino-(1-6C)alkyl, aryl, aryl-(1-6C)alkyl, (3-8C)cycloalkyl, (3-8C)cycloalkyl-(1-6C)alkyl, heteroaryl, heteroaryl-(1-6C)alkyl, heterocyclyl or heterocyclyl-(1-6C)alkyl, provided that, when X⁶ is O or N(R¹⁶), there are at least two carbon atoms between X⁶ and any heteroatom in the R¹⁵ group,

and any other R⁶ group that is present is selected from halogeno, trifluoromethyl, cyano, hydroxy, amino, (1-8C)alkyl, (1-6C)alkoxy, (1-6C)alkylamino, di-[(1-6C)alkyl]amino, (2-6C)alkanoylamino and N-(1-6C)alkyl-(2-6C)alkanoylamino,

and wherein any aryl, (3-8C)cycloalkyl, heteroaryl or heterocyclyl group within the R⁶ group optionally bears 1 or 2 substituents, which may be the same or different, selected from halogeno, trifluoromethyl, hydroxy, amino, (1-8C)alkyl, (1-6C)alkoxy, (1-6C)alkylamino, di-[(1-6C)alkyl]amino, hydroxy-(1-6C)alkyl, amino-(1-6C)alkyl, (1-6C)alkylamino-(1-6C)alkyl and di-[(1-6C)alkyl]amino-(1-6C)alkyl;

(ww) r is 1 or 2 and one R⁶ group is a group of the formula:

-X⁶-R¹⁵

wherein X⁶ is a direct bond or is selected from O, NH and N(Me), and R¹⁵ is hydroxymethyl, 1-hydroxyethyl, 2-hydroxyethyl, 1-hydroxy-1-methylethyl, 3-hydroxypropyl, methoxymethyl, 1-methoxyethyl, 2-methoxyethyl, 1-methoxy-1-methylethyl, 3-methoxypropyl, cyanomethyl, 1-cyanoethyl, 2-cyanoethyl, 1-cyano-1-methylethyl, 3-cyanopropyl, aminomethyl, 1-aminoethyl, 2-aminoethyl, 1-amino-1-methylethyl, 3-aminopropyl, methylaminomethyl, 1-methylaminoethyl, 2-methylaminoethyl, 1-methylamino-1-methylethyl, 3-methylaminopropyl, ethylaminomethyl, 1-ethylaminoethyl, 2-ethylaminoethyl, 1-ethylamino-1-methylethyl, 3-ethylaminopropyl, isopropylaminomethyl, 1-isopropylaminoethyl, dimethylaminomethyl, 1-dimethylaminoethyl, 2-dimethylaminoethyl, 1-dimethylamino-1-methylethyl, 3-dimethylaminopropyl, phenyl, benzyl, cyclopropyl, cyclobutyl, cyclopentyl, cyclohexyl, furyl, thienyl, oxazolyl, imidazolyl, thiazolyl, pyridyl, pyrimidinyl, tetrahydrofuranyl, tetrahydropyranyl, tetrahydrothiopyranyl, pyrrolinyl, pyrrolidinyl, imidazolidinyl, pyrazolidinyl, morpholinyl, tetrahydro-1,4-thiazinyl, piperidinyl, homopiperidinyl, piperazinyl, homopiperazinyl, indolinyl, isoindolinyl, pyrrolinylmethyl, pyrrolidinylmethyl, 2-pyrrolidinylethyl, 3-pyrrolidinylpropyl, imidazolidinylmethyl, pyrazolidinylmethyl, morpholinylmethyl, 2-(morpholinyl)ethyl, 3-(morpholinyl)propyl, tetrahydro-1,4-thiazinylmethyl, 2-(tetrahydro-1,4-thiazinyl)ethyl, 3-(tetrahydro-1,4-thiazinyl)propyl, piperidinylmethyl, 2-(piperidinyl)ethyl, 3-(piperidinyl)propyl, homopiperidinylmethyl, piperazinylmethyl, 2-(piperazinyl)ethyl, 3-(piperazinyl)propyl or homopiperazinylmethyl, provided that, when X⁶ is O, NH or N(Me), there are at least two carbon atoms between X⁶ and any heteroatom in the R¹⁵ group,

and wherein any aryl, (3-8C)cycloalkyl, heteroaryl or heterocyclyl group within the R⁶ group optionally bears 1 or 2 substituents, which may be the same or different, selected from fluoro, chloro, trifluoromethyl, hydroxy, amino, methyl, ethyl, methoxy, ethoxy, methylamino, dimethylamine, hydroxymethyl, 2-hydroxyethyl, 3-hydroxypropyl, aminomethyl, 2-aminoethyl, 3-aminopropyl, methylaminomethyl, 2-methylaminoethyl, 3-methylaminopropyl, dimethylaminomethyl, 2-dimethylaminoethyl and 3-dimethylaminopropyl,

and any other R⁶ group that is present is selected from fluoro, chloro, trifluoromethyl, cyano, hydroxy, amino, methyl, methoxy, methylamino and dimethylamino;

(xx) r is 1 or 2 and the first R⁶ group is a group of the formula:

-X⁶-R¹⁵

wherein X⁶ is a direct bond or O and R¹⁵ is hydroxymethyl, 1-hydroxyethyl, 2-hydroxyethyl, 3-hydroxypropyl, methoxymethyl, 1-methoxyethyl, 2-methoxyethyl, 1-methoxy-1-methylethyl, 3-methoxypropyl, cyanomethyl, 1-cyanoethyl, 2-cyanoethyl, 3-cyanopropyl, aminomethyl, 1-aminoethyl, 2-aminoethyl, 3-aminopropyl, methylaminomethyl, 1-methylaminoethyl, 2-methylaminoethyl, 3-methylaminopropyl, ethylaminomethyl, 1-ethylaminoethyl, 2-ethylaminoethyl, 1-ethylamino-1-methylethyl, 3-ethylaminopropyl, isopropylaminomethyl, 1-isopropylaminoethyl, dimethylaminomethyl, 1-dimethylaminoethyl, 2-dimethylaminoethyl, 3-dimethylaminopropyl, phenyl, benzyl, cyclopropyl, cyclopentyl, cyclohexyl, thienyl, imidazolyl, thiazolyl, thiadiazolyl, pyrrolidinyl, morpholinyl, tetrahydro-1,4-thiazinyl, piperidinyl, homopiperidinyl, piperazinyl, homopiperazinyl, pyrrolidinylmethyl, 2-(pyrrolidinyl)ethyl, 3-(pyrrolidinyl)propyl, morpholinylmethyl, 2-(morpholinyl)ethyl, 3-(morpholinyl)propyl, piperidinylmethyl, 2-(piperidinyl)ethyl, 3-(piperidinyl)propyl, homopiperidinylmethyl, piperazinylmethyl, 2-(piperazinyl)ethyl, 3-(piperazinyl)propyl or homopiperazinylmethyl, provided that, when X⁶ is O, there are at least two carbon atoms between X⁶ and any heteroatom in the R¹⁵ group,

and wherein any aryl, (3-8C)cycloalkyl, heteroaryl or heterocyclyl group within the R⁶ group optionally bears a substituent selected from fluoro, chloro, trifluoromethyl, hydroxy, amino, methyl, methoxy, methylamino and dimethylamino and any such aryl, (3-8C)cycloalkyl, heteroaryl or heterocyclyl group within the R⁶ group optionally bears a further substituent selected from hydroxymethyl, cyanomethyl, aminomethyl, methylaminomethyl and dimethylaminomethyl,

and any second R⁶ group that is present is selected from fluoro, chloro, trifluoromethyl, cyano, hydroxy, amino, methyl, methoxy, methylamino and dimethylamino;

(yy) r is 1 or 2 and the first R⁶ group is selected from hydroxymethyl, 1-hydroxyethyl, 2-hydroxyethyl, methoxymethyl, 1-methoxyethyl, 2-methoxyethyl, cyanomethyl, 1-cyanoethyl, 2-cyanoethyl, aminomethyl, 1-aminoethyl, 2-aminoethyl, methylaminomethyl, 1-methylaminoethyl, 2-methylaminoethyl, ethylaminomethyl, 1-ethylaminoethyl, 2-ethylaminoethyl, isopropylaminomethyl, 1-isopropylaminoethyl, 2-isopropylaminoethyl, dimethylaminomethyl, 1-dimethylaminoethyl, 2-dimethylaminoethyl, phenyl, benzyl, cyclopropyl, cyclopentyl, cyclohexyl, thienyl, imidazolyl, thiazolyl, thiadiazolyl, pyrrolidinyl, morpholinyl, tetrahydro-1,4-thiazinyl, piperidinyl, homopiperidinyl, piperazinyl, homopiperazinyl, pyrrolidinylmethyl, 2-(pyrrolidinyl)ethyl, morpholinylmethyl, 2-(morpholinyl)ethyl, piperidinylmethyl, 2-(piperidinyl)ethyl, homopiperidinylmethyl, piperazinylmethyl, 2-(piperazinyl)ethyl and homopiperazinylmethyl,

and wherein any aryl, (3-8C)cycloalkyl, heteroaryl or heterocyclyl group within the R⁶ group optionally bears a substituent selected from fluoro, chloro, trifluoromethyl, hydroxy, amino, methyl, methoxy, methylamino and dimethylamino and any such aryl, (3-8C)cycloalkyl, heteroaryl or heterocyclyl group within the R⁶ group optionally bears a further substituent selected from hydroxymethyl, cyanomethyl, aminomethyl, methylaminomethyl and dimethylaminomethyl,

and any second R⁶ group that is present is selected from fluoro, chloro, trifluoromethyl, cyano, hydroxy, amino, methyl, methoxy, methylamino and dimethylamino;

(zz) r is 1 or 2 and the first R⁶ group is selected from fluoro, chloro, cyano, hydroxy, amino, methyl, ethyl, propyl, isopropyl, butyl, sec-butyl, isobutyl, tert-butyl, cyclopropyl, cyclobutyl, cyclopentyl, methoxy, ethoxy, methylamino, ethylamino, propylamino, isopropylamino, cyclopropylamino, 2-hydroxyethylamino, 2-methoxyethylamino, dimethylamino, N-cyclopropyl-N-methylamino, acetyl, hydroxymethyl, 1-hydroxyethyl, aminomethyl, methylaminomethyl, ethylaminomethyl, propylaminomethyl, isopropylaminomethyl, cyclopropylaminomethyl, 2-hydroxyethylaminomethyl, dimethylaminomethyl, diethylaminomethyl, N-ethyl-N-methylaminomethyl, cyclopropylaminomethyl, N-cyclopropyl-N-methylaminomethyl, furylmethylaminomethyl, pyrrolylmethylaminomethyl, pyridylmethylaminomethyl, phenyl, furyl, thienyl, imidazolyl, oxazolyl, thiazolyl, pyrrolidinyl, morpholinyl, piperidinyl, homopiperidinyl, piperazinyl, homopiperazinyl, azetidinylmethyl, pyrrolidinylmethyl, morpholinylmethyl, piperidinylmethyl, homopiperidinylmethyl, piperazinylmethyl and homopiperazinylmethyl,

and wherein any aryl, (3-8C)cycloalkyl, heteroaryl or heterocyclyl group within the R⁶ group optionally bears a substituent selected from fluoro, chloro, trifluoromethyl, hydroxy, amino, methyl, methoxy, methylamino, dimethylamino, hydroxymethyl, cyanomethyl, aminomethyl, methylaminomethyl and dimethylaminomethyl,

and any second R⁶ group that is present is selected from fluoro, chloro, trifluoromethyl, cyano, hydroxy, amino, methyl, methoxy, methylamino and dimethylamino;

(aaa) r is 1 and the R⁶ group is selected from fluoro, chloro, trifluoromethyl, hydroxy, amino, methyl, ethyl, propyl, isopropyl, butyl, sec-butyl, isobutyl, tert-butyl, cyclopropyl, cyclobutyl, cyclopentyl, hydroxymethyl, 2-hydroxyethyl, methoxymethyl, 2-methoxyethyl, methylaminomethyl, ethylaminomethyl, isopropylaminomethyl, cyclopropylaminomethyl, dimethylaminomethyl, methoxy, ethoxy, methylamino, ethylamino, dimethylamino and diethylamino;
(bbb) two R⁶ groups together form a bivalent group that spans adjacent ring positions on Ring A selected from OC(R¹⁸)₂O, OC(R¹⁸)₂C(R¹⁸)₂O, OC(R¹⁸)₂C(R¹⁸)₂, C(R¹⁸)₂OC(R¹⁸)₂, C(R¹⁸)₂C(R¹⁸)₂C(R¹⁸)₂, C(R¹⁸)₂C(R¹⁸)₂C(R¹⁸)₂C(R¹⁸)₂, OC(R¹⁸)₂N(R¹⁹), N(R¹⁹)C(R¹⁸)₂N(R¹⁹), N(R¹⁹)C(R¹⁸)₂C(R¹⁸)₂, N(R¹⁹)C(R¹⁸)₂C(R¹⁸)₂C(R¹⁸)₂ and C(R¹⁸)₂N(R¹⁹)C(R¹⁸)₂, wherein each of R¹⁸ and R¹⁹ is hydrogen, (1-8C)alkyl, (2-8C)alkenyl or (2-8C)alkynyl;
(ccc) two R⁶ groups together form a bivalent group that spans adjacent ring positions on Ring A selected from OC(R¹⁸)₂O, OC(R¹⁸)₂C(R¹⁸)₂O, C(R¹⁸)₂OC(R¹⁸)₂, OC(R¹⁸)₂N(R¹⁹), N(R¹⁹)C(R¹⁸)₂N(R¹⁹), N(R¹⁹)C(R¹⁸)₂C(R¹⁸)₂, N(R¹⁹)C(R¹⁸)₂C(R¹⁸)₂C(R¹⁸)₂ and C(R¹⁸)₂N(R¹⁹)C(R¹⁸)₂, wherein each of R¹⁸ and R¹⁹ is hydrogen, methyl, ethyl or propyl;
(ddd) two R⁶ groups together form a bivalent group that spans adjacent ring positions on Ring A selected from OCH₂O, OCH₂CH₂O, OCH₂NH, NHCH₂CH₂ and NHCH₂CH₂CH₂;
(eee) two R⁶ groups together form a bivalent group that spans adjacent ring positions on Ring A selected from OCH₂O and OCH₂CH₂O;
(fff) p is 0 or p is 1 or 2 and the R¹ groups are located at the 6- and/or 7-positions and are selected from halogeno, trifluoromethyl, cyano, hydroxy, amino, carbamoyl, (1-6C)alkoxycarbonyl, (1-8C)alkyl, (2-8C)alkenyl, (2-8C)alkynyl, (1-6C)alkoxy, (2-6C)alkenyloxy, (2-6C)alkynyloxy, (1-6C)alkylamino, di-[(1-6C)alkyl]amino, N-(1-6C)alkylcarbamoyl and N,N-di-[(1-6C)alkyl]carbamoyl,
and q is 1 and the R² group is located at the 2-position (relative to the C(R³)(R⁴) group) and is selected from halogeno, trifluoromethyl, cyano, carbamoyl, hydroxy, amino, (1-8C)alkyl, (2-8C)alkenyl, (2-8C)alkynyl, (1-6C)alkoxy, (1-6C)alkylamino, di-[(1-6C)alkyl]amino, N-(1-6C)alkylcarbamoyl and N,N-di-[(1-6C)alkyl]carbamoyl;
(ggg) p is 0 or p is 1 or 2 and the R¹ groups are located at the 6- and/or 7-positions and are selected from fluoro, chloro, trifluoromethyl, cyano, hydroxy, amino, carbamoyl, methoxycarbonyl, ethoxycarbonyl, methyl, ethyl, methoxy, ethoxy, methylamino, dimethylamino, N-methylcarbamoyl and N,N-dimethylcarbamoyl,
and q is 1 and the R² group which is located at the 2-position (relative to the C(R³)(R⁴) group) is selected from fluoro, chloro, trifluoromethyl, cyano, carbamoyl, hydroxy, amino, methyl, ethyl, methoxy, ethoxy, methylamino, dimethylamino, N-methylcarbamoyl and N,N-dimethylcarbamoyl;
(hhh) p is 0 or p is 1 or 2 and the R¹ groups are located at the 6- and/or 7-positions and are selected from fluoro, chloro, cyano, carbamoyl, methoxycarbonyl, methoxy, ethoxy, N-methylcarbamoyl and N,N-dimethylcarbamoyl, and q is 1 and the R² group which is located at the 2-position (relative to the C(R³)(R⁴) group) is selected from carbamoyl, methoxy, ethoxy, N-methylcarbamoyl and N,N-dimethylcarbamoyl; and
(iii) p is 0 or p is 1 or 2 and the R¹ groups are located at the 6- and/or 7-positions and are selected from fluoro, cyano, carbamoyl, methoxycarbonyl, methoxy, ethoxy, N-methylcarbamoyl and N,N-dimethylcarbamoyl, and q is 1 and the R² group which is located at the 2-position (relative to the C(R³)(R⁴) group) is selected from methoxy and ethoxy.

A particular compound of the invention is a quinoline derivative of the Formula I wherein:—

X¹ is O;

p is 2 and the R¹ groups are located at the 6- and 7-positions and the R¹ group at the 6-position is selected from cyano, hydroxy, methoxycarbonyl, ethoxycarbonyl, carbamoyl, methoxy, ethoxy, propoxy, N-methylcarbamoyl, N-ethylcarbamoyl, N,N-dimethylcarbamoyl, N,N-diethylcarbamoyl, pyrrolidin-1-ylcarbonyl, morpholinocarbonyl, piperidinocarbonyl and piperazin-1-ylcarbonyl, and the R¹ group at the 7-position is selected from methoxy, ethoxy, propoxy, 2-pyrrolidin-1-ylethoxy, 3-pyrrolidin-1-ylpropoxy, 4-pyrrolidin-1-ylbutoxy, pyrrolidin-3-yloxy, pyrrolidin-2-ylmethoxy, 2-pyrrolidin-2-ylethoxy, 3-pyrrolidin-2-ylpropoxy, 2-morpholinoethoxy, 3-morpholinopropoxy, 4-morpholinobutoxy, 2-(1,1-dioxotetrahydro-4H-1,4-thiazin-4-yl)ethoxy, 3-(1,1-dioxotetrahydro-4H-1,4-thiazin-4-yl)propoxy, 2-piperidinoethoxy, 3-piperidinopropoxy, 4-piperidinobutoxy, piperidin-3-yloxy, piperidin-4-yloxy, piperidin-3-ylmethoxy, 2-piperidin-3-ylethoxy, piperidin-4-ylmethoxy, 2-piperidin-4-ylethoxy, 2-homopiperidin-1-ylethoxy, 3-homopiperidin-1-ylpropoxy, 3-(1,2,3,6-tetrahydropyridin-1-yl)propoxy, 2-piperazin-1-ylethoxy, 3-piperazin-1-ylpropoxy, 2-homopiperazin-1-ylethoxy and 3-homopiperazin-1-ylpropoxy,

and wherein any heterocyclyl group within a substituent on R¹ optionally bears 1 or 2 substituents, which may be the same or different, selected from fluoro, chloro, trifluoromethyl, hydroxy, amino, methyl, ethyl, methoxy, methylenedioxy, ethylidendioxy and isopropylidenedioxy, and a pyrrolidin-2-yl, pyrrolidin-3-yl, piperidin-3-yl, piperidin-4-yl, piperazin-1-yl or homopiperazin-1-yl group within a R¹ substituent is optionally N-substituted with methyl, ethyl, propyl, allyl, 2-propynyl, methylsulphonyl, acetyl, propionyl, isobutyryl, 2-fluoroethyl, 2,2-difluoroethyl, 2,2,2-trifluoroethyl or cyanomethyl,

and wherein any heterocyclyl group within a substituent on R¹ optionally bears 1 or 2 oxo substituents,

and wherein any CH, CH₂ or CH₃ group within a R¹ substituent optionally bears on each said CH, CH₂ or CH₃ group one or more chloro groups or a substituent selected from hydroxy, amino, methoxy, methylsulphonyl, methylamino, dimethylamino, diisopropylamino, N-ethyl-N-methylamino and N-isopropyl-N-methylamino;

q is 0 or q is 1 and the R² group which is located at the 2-position (relative to the C(R³)(R⁴) group) is selected from fluoro, chloro, trifluoromethyl, cyano, hydroxy, amino, methyl, methoxy, methylamino and dimethylamino;

each of R³ and R⁴ is hydrogen;

R⁵ is hydrogen, methyl or ethyl;

Ring A is a phenyl, pyridyl, pyrimidinyl, pyrazinyl or pyridazinyl ring; and

r is 0 or r is 1 or 2 and one R⁶ group is located at the 3- or 4-position (relative to the CON(R⁵)group), and each R⁶ group, which may be the same or different, is selected from fluoro, chloro, trifluoromethyl, cyano, hydroxy, amino, methyl, methoxy, methylamino and dimethylamino,

or r is 1 or 2 and one R⁶ group is located at the 3- or 4-position (relative to the CON(R⁵) group) and is a group of the formula:

-X⁶-R¹⁵

wherein X⁶ is a direct bond or O and R¹⁵ is hydroxymethyl, 1-hydroxyethyl, 2-hydroxyethyl, 3-hydroxypropyl, methoxymethyl, 1-methoxyethyl, 2-methoxyethyl, 1-methoxy-1-methylethyl, 3-methoxypropyl, cyanomethyl, 1-cyanoethyl, 2-cyanoethyl, 3-cyanopropyl, aminomethyl, 1-aminoethyl, 2-aminoethyl, 3-aminopropyl, methylaminomethyl, 1-methylaminoethyl, 2-methylaminoethyl, 3-methylaminopropyl, ethylaminomethyl, 1-ethylaminoethyl, 2-ethylaminoethyl, 1-ethylamino-1-methylethyl, 3-ethylaminopropyl, isopropylaminomethyl, 1-isopropylaminoethyl, dimethylaminomethyl, 1-dimethylaminoethyl, 2-dimethylaminoethyl, 3-dimethylaminopropyl, phenyl, benzyl, cyclopropyl, cyclopentyl, cyclohexyl, thienyl, imidazolyl, thiazolyl, thiadiazolyl, pyrrolidinyl, morpholinyl, tetrahydro-1,4-thiazinyl, piperidinyl, homopiperidinyl, piperazinyl, homopiperazinyl, pyrrolidinylmethyl, 2-(pyrrolidinyl)ethyl, 3-(pyrrolidinyl)propyl, molpholinylmethyl, 2-(morpholinyl)ethyl, 3-(molpholinyl)propyl, piperidinylmethyl, 2-(piperidinyl)ethyl, 3-(piperidinyl)propyl, homopiperidinylmethyl, piperazinylmethyl, 2-(piperazinyl)ethyl, 3-(piperazinyl)propyl or homopiperazinylmethyl, provided that, when X⁶ is O, there are at least two carbon atoms between X⁶ and any heteroatom in the R¹⁵ group,

and wherein any aryl, (3-8C)cycloalkyl, heteroaryl or heterocyclyl group within the R⁶ group optionally bears a substituent selected from fluoro, chloro, trifluoromethyl, hydroxy, amino, methyl, methoxy, methylamino and dimethylamino and any such aryl, (3-8C)cycloalkyl, heteroaryl or heterocyclyl group within the R⁶ group optionally bears a further substituent selected from hydroxymethyl, cyanomethyl, aminomethyl, methylaminomethyl and dimethylaminomethyl,

and any second R⁶ group that is present is selected from fluoro, chloro, trifluoromethyl, cyano, hydroxy, amino, methyl, methoxy, methylamino and dimethylamino;

or a pharmaceutically-acceptable salt thereof.

A further particular compound of the invention is a quinoline derivative of the Formula I wherein:—

X¹ is O;

p is 2 and the first R¹ group is located at the 6-position and is selected from cyano, carbamoyl, methoxy, N-methylcarbamoyl and N,N-dimethylcarbamoyl, and the second R¹ group is located at the 7-position and is selected from methoxy, ethoxy, 2-methoxyethoxy, 3-methoxypropoxy, 2-methylsulphonylethoxy, 3-methylsulphonylpropoxy, 2-(2-methoxyethoxy)ethoxy, 2-pyrrolidin-1-ylethoxy, 3-pyrrolidin-1-ylpropoxy, 2-[(3RS,4SR)-3,4-methylenedioxypyrrolidin-1-yl]ethoxy, 3-[(3RS,4SR)-3,4-methylenedioxypyrrolidin-1-yl]propoxy, 2-morpholinoethoxy, 3-morpholinopropoxy, 2-(1,1-dioxotetrahydro-4H-1,4-thiazin-4-yl)ethoxy, 3-(1,1-dioxotetrahydro-4H-1,4-thiazin-4-yl)propoxy, 2-piperidinoethoxy, 3-piperidinopropoxy, 2-piperidin-3-ylethoxy, 2-(N-methylpiperidin-3-yl)ethoxy, 3-piperidin-3-ylpropoxy, 3-(N-methylpiperidin-3-yl)propoxy, 2-piperidin-4-ylethoxy, 2-(N-methylpiperidin-4-yl)ethoxy, 3-piperidin-4-ylpropoxy, 3-(N-methylpiperidin-4-yl)propoxy, 2-(1,2,3,6-tetrahydropyridin-1-yl)ethoxy, 3-(1,2,3,6-tetrahydropyridin-1-yl)propoxy, 2-(4-hydroxypiperidin-1-yl)ethoxy, 3-(4-hydroxypiperidin-1-yl)propoxy, 2-piperazin-1-ylethoxy, 3-piperazin-1-ylpropoxy, 4-piperazin-1-ylbutoxy, 2-(4-methylpiperazin-1-yl)ethoxy, 3-(4-methylpiperazin-1-yl)propoxy, 4-(4-methylpiperazin-1-yl)butoxy, 2-(4-allylpiperazin-1-yl)ethoxy, 3-(4-allylpiperazin-1-yl)propoxy, 2-(4-prop-2-ynylpiperazin-1-yl)ethoxy, 3-(4-prop-2-ynylpiperazin-1-yl)propoxy, 2-(4-methylsulphonylpiperazin-1-yl)ethoxy, 3-(4-methylsulphonylpiperazin-1-yl)propoxy, 2-(4-acetylpiperazin-1-yl)ethoxy, 3-(4-acetylpiperazin-1-yl)propoxy, 4-(4-acetylpiperazin-1-yl)butoxy, 2-(4-isobutyrylpiperazin-1-yl)ethoxy, 3-(4-isobutyrylpiperazin-1-yl)propoxy, 4-(4-isobutyrylpiperazin-1-yl)butoxy, 2-[4-(2-fluoroethyl)piperazin-1-yl]ethoxy, 3-[4-(2-fluoroethyl)piperazin-1-yl]propoxy, 2-[4-(2,2,2-trifluoroethyl)piperazin-1-yl]ethoxy, 3-[4-(2,2,2-trifluoroethyl)piperazin-1-yl]propoxy, 2-(4-cyanomethylpiperazin-1-yl)ethoxy, 3-(4-cyanomethylpiperazin-1-yl)propoxy, 2-[2-(4-methylpiperazin-1-yl)ethoxy]ethoxy, 2-(4-pyridyloxy)ethoxy, 3-pyridylmethoxy and 2-cyanopyrid-4-ylmethoxy;

q is 0 or q is 1 and the R¹ group which is located at the 2-position (relative to the C(R³)(R⁴) group) is selected from fluoro, chloro, cyano, methyl and methoxy;

each of R³ and R⁴ is hydrogen;

R⁵ is hydrogen or methyl;

Ring A is a phenyl, pyridyl, pyrimidinyl, pyrazinyl or pyridazinyl ring; and

r is 0 or r is 1 or 2 and one R⁶ group is located at the 3- or 4-position (relative to the CON(R⁵) group), and each R⁶ group, which may be the same or different, is selected from fluoro, chloro, trifluoromethyl, hydroxy, amino, methyl, methoxy, methylamino and dimethylamino,

or r is 1 or 2 and one R⁶ group is located at the 3- or 4-position (relative to the CON(R⁵) group) and is selected from hydroxymethyl, 1-hydroxyethyl, 2-hydroxyethyl, methoxymethyl, 1-methoxyethyl, 2-methoxyethyl, cyanomethyl, 1-cyanoethyl, 2-cyanoethyl, aminomethyl, 1-aminoethyl, 2-aminoethyl, methylaminomethyl, 1-methylaminoethyl, 2-methylaminoethyl, ethylaminomethyl, 1-ethylaminoethyl, 2-ethylaminoethyl, isopropylaminomethyl, 1-isopropylaminoethyl, 2-isopropylaminoethyl, dimethylaminomethyl, 1-dimethylaminoethyl, 2-dimethylaminoethyl, pyrrolidinylmethyl, morpholinylmethyl, piperidinylmethyl and piperazinylmethyl,

and wherein any heterocyclyl group within the R⁶ group optionally bears a substituent selected from fluoro, chloro, trifluoromethyl, hydroxy, amino, methyl, methoxy, methylamino and dimethylamino,

and any second R⁶ group that is present is selected from fluoro, chloro, trifluoromethyl, cyano, hydroxy, amino, methyl, methoxy, methylamino and dimethylamino;

or a pharmaceutically-acceptable salt thereof.

A further particular compound of the invention is a quinoline derivative of the Formula I wherein:—

X¹ is O;

p is 2 and the R¹ groups, which may be the same or different, are located at the 6- and 7-positions and are selected from cyano, methoxy, ethoxy, propoxy, 2-hydroxyethoxy, 3-hydroxypropoxy, 2-methoxyethoxy, 3-methoxypropoxy, 2-methylsulphonylethoxy, 3-methylsulphonylpropoxy and 2-(2-methoxyethoxy)ethoxy;

q is 0 or q is 1 and the R² group which is located at the 2- or 3-position (relative to the C(R³)(R⁴) group) is fluoro, chloro, methyl or methoxy;

each of R³ and R⁴ is hydrogen;

R⁵ is hydrogen, methyl or ethyl;

Ring A is phenyl; and

r is 1 or 2 and the first R⁶ group is located at the 3-position (relative to the CON(R⁵) group) and is selected from fluoro, chloro, methoxy, ethoxy, methylamino, ethylamino, dimethylamino, cyclopropylamino, N-cyclopropyl-N-methylamino, hydroxymethyl, aminomethyl, methylaminomethyl, ethylaminomethyl, isopropylaminomethyl, cyclopropylaminomethyl, dimethylaminomethyl, diethylaminomethyl, N-ethyl-N-methylaminomethyl, N-cyclopropyl-N-methylaminomethyl, azetidinylmethyl, pyrrolidinylmethyl, morpholinylmethyl, piperidinylmethyl, homopiperidinylmethyl, piperazinylmethyl and homopiperazinylmethyl,

and any second R⁶ group that is present is selected from fluoro, chloro, methyl, ethyl, methoxy and ethoxy,

and wherein any heterocyclyl group within the R⁶ group optionally bears a methyl, ethyl or hydroxymethyl substituent;

or a pharmaceutically-acceptable salt thereof.

A further particular compound of the invention is a quinoline derivative of the Formula I wherein:—

X¹ is O;

p is 2 and the first R¹ group is a 6-cyano or 6-methoxy group and the second R¹ group is located at the 7-position and is selected from methoxy, ethoxy, 2-hydroxyethoxy and 2-methoxyethoxy;

q is 0 or q is 1 and the R² group is fluoro;

each of R³ and R⁴ is hydrogen;

R⁵ is hydrogen, methyl or ethyl;

Ring A is phenyl; and

r is 1 or 2 and the first R⁶ group is located at the 3-position (relative to the CON(R⁵) group) and is selected from fluoro, chloro, methoxy, methylamino, ethylamino, dimethylamino, cyclopropylamino, hydroxymethyl, aminomethyl, methylaminomethyl, ethylaminomethyl, propylaminomethyl, isopropylaminomethyl, cyclopropylaminomethyl, dimethylaminomethyl, diethylaminomethyl, N-ethyl-N-methylaminomethyl, N-cyclopropyl-N-methylaminomethyl, azetidin-1-ylmethyl, pyrrolidin-1-ylmethyl, morpholinomethyl, piperidinomethyl and piperazin-1-ylmethyl,

and any second R⁶ group that is present is selected from fluoro, chloro, methyl, ethyl, methoxy and ethoxy,

and wherein any heterocyclyl group within the R⁶ group optionally bears a methyl, ethyl or hydroxymethyl substituent;

or a pharmaceutically-acceptable salt, solvate or pro-drug thereof.

A further particular compound of the invention is a quinoline derivative of the Formula I wherein:—

X¹ is O;

p is 2 and the R¹ groups, which may be the same or different, are located at the 6- and 7-positions and are selected from cyano, methoxy, ethoxy, propoxy, 2-hydroxyethoxy, 3-hydroxypropoxy, 2-methoxyethoxy, 3-methoxypropoxy, 2-methylsulphonylethoxy, 3-methylsulphonylpropoxy and 2-(2-methoxyethoxy)ethoxy;

q is 0 or q is 1 and the R² group is fluoro, chloro, methyl or methoxy;

each of R³ and R⁴ is hydrogen;

R⁵ is hydrogen, methyl or ethyl;

Ring A is pyridyl, pyrimidinyl, pyrazinyl or pyridazinyl; and

r is 0, 1 or 2 and each R⁶ group that is present is selected from fluoro, chloro, trifluoromethyl, cyano, methyl, ethyl, propyl, isopropyl, tert-butyl, cyclopropyl, cyclobutyl, cyclopentyl, methoxy, ethoxy, methylamino, ethylamino, propylamino, isopropylamino, cyclopropylamino, 2-hydroxyethylamino, 2-methoxyethylamino, dimethylamino, N-cyclopropyl-N-methylamino, acetyl, hydroxymethyl, aminomethyl, methylaminomethyl, ethylaminomethyl, propylaminomethyl, isopropylaminomethyl, cyclopropylaminomethyl, dimethylaminomethyl, diethylaminomethyl, N-ethyl-N-methylaminomethyl, N-cyclopropyl-N-methylaminomethyl, pyrrolidin-1-yl, piperidino, morpholino, piperazin-1-yl, pyrrolidin-1-ylmethyl, morpholinomethyl, piperidinomethyl and piperazin-1-ylmethyl,

and wherein any heterocyclyl group within the R⁶ group optionally bears a methyl or ethyl substituent;

or a pharmaceutically-acceptable salt thereof.

A further particular compound of the invention is a quinoline derivative of the Formula I wherein:—

X¹ is O;

p is 2 and the first R¹ group is a 6-cyano or 6-methoxy group and the second R¹ group is located at the 7-position and is selected from methoxy, ethoxy, 2-hydroxyethoxy and 2-methoxyethoxy;

q is 0 or q is 1 and the R² group is fluoro, chloro, methyl or methoxy;

each of R³ and R⁴ is hydrogen;

R⁵ is hydrogen, methyl or ethyl;

Ring A is 2-pyridyl, 3-pyridyl, 4-pyridyl, 2-pyrimidinyl, 4-pyrimidinyl, 5-pyrimidinyl, 2-pyrazinyl, 3-pyridazinyl or 4-pyridazinyl; and

r is 0 or r is 1 or 2 and any first R⁶ group that is present is selected from methylamino, ethylamino, propylamino, isopropylamino, cyclopropylamino, 2-hydroxyethylamino, 2-methoxyethylamino, dimethylamino, N-cyclopropyl-N-methylamino, pyrrolidin-1-yl, piperidino, morpholino and piperazin-1-yl, and any second R⁶ group that is present is selected from fluoro, chloro, methyl, ethyl, methoxy and ethoxy,

and wherein any heterocyclyl group within the R⁶ group optionally bears a methyl or ethyl substituent;

or a pharmaceutically-acceptable salt thereof.

A particular compound of the invention is a quinoline derivative of the Formula I wherein:—

X¹ is O;

p is 2 and the R¹ groups are located at the 6- and 7-positions and the R¹ group at the 6-position is selected from cyano, hydroxy, methoxycarbonyl, ethoxycarbonyl, carbamoyl, methoxy, ethoxy, propoxy, N-methylcarbamoyl, N-ethylcarbamoyl, N,N-dimethylcarbamoyl, N,N-diethylcarbamoyl, pyrrolidin-1-ylcarbonyl, morpholinocarbonyl, piperidinocarbonyl and piperazin-1-ylcarbonyl, and the R¹ group at the 7-position is selected from methoxy, ethoxy, propoxy, 2-pyrrolidin-1-ylethoxy, 3-pyrrolidin-1-ylpropoxy, 4-pyrrolidin-1-ylbutoxy, pyrrolidin-3-yloxy, pyrrolidin-2-ylmethoxy, 2-pyrrolidin-2-ylethoxy, 3-pyrrolidin-2-ylpropoxy, 2-morpholinoethoxy, 3-morpholinopropoxy, 4-morpholinobutoxy, 2-(1,1-dioxotetahydro-4H-1,4-thiazin-4-yl)ethoxy, 3-(1,1-dioxotetrahydro-4H-1,4-thiazin-4-yl)propoxy, 2-piperidinoethoxy, 3-piperidinopropoxy, 4-piperidinobutoxy, piperidin-3-yloxy, piperidin-4-yloxy, piperidin-3-ylmethoxy, 2-piperidin-3-ylethoxy, piperidin-4-ylmethoxy, 2-piperidin-4-ylethoxy, 2-homopiperidin-1-ylethoxy, 3-homopiperidin-1-ylpropoxy, 3-(1,2,3,6-tetrahydropyridin-1-yl)propoxy, 2-piperazin-1-ylethoxy, 3-piperazin-1-ylpropoxy, 2-homopiperazin-1-ylethoxy and 3-homopiperazin-1-ylpropoxy,

and wherein any heterocyclyl group within a substituent on R¹ optionally bears 1 or 2 substituents, which may be the same or different, selected from fluoro, chloro, trifluoromethyl, hydroxy, amino, methyl, ethyl, methoxy, methylenedioxy, ethylidendioxy and isopropylidenedioxy, and a pyrrolidin-2-yl, pyrrolidin-3-yl, piperidin-3-yl, piperidin-4-yl, piperazin-1-yl or homopiperazin-1-yl group within a R¹ substituent is optionally N-substituted with methyl, ethyl, propyl, allyl, 2-propynyl, methylsulphonyl, acetyl, propionyl, isobutyryl, 2-fluoroethyl, 2,2-difluoroethyl, 2,2,2-trifluoroethyl or cyanomethyl,

and wherein any heterocyclyl group within a substituent on R¹ optionally bears 1 or 2 oxo substituents,

and wherein any CH, CH₂ or CH₃ group within a R¹ substituent optionally bears on each said CH, CH₂ or CH₃ group one or more chloro groups or a substituent selected from hydroxy, amino, methoxy, methylsulphonyl, methylamino, dimethylamino, diisopropylamino, N-ethyl-N-methylamino and N-isopropyl-N-methylamino;

q is 0 or q is 1 and the R² group is selected from fluoro, chloro, trifluoromethyl, cyano, hydroxy, amino, methyl, methoxy, methylamino and dimethylamino;

each of R³ and R⁴ is hydrogen;

R⁵ is hydrogen, methyl or ethyl;

Ring A is a furyl, pyrrolyl, thienyl, oxazolyl, isoxazolyl, imidazolyl, pyrazolyl, thiazolyl, isothiazolyl, oxadiazolyl or thiadiazolyl ring; and

r is 0 or r is 1 or 2 and one R⁶ group is located at the 3-position (relative to the CON(R⁵) group), and each R⁶ group, which may be the same or different, is selected from fluoro, chloro, trifluoromethyl, cyano, hydroxy, amino, methyl, ethyl, propyl, isopropyl, butyl, sec-butyl, isobutyl, tert-butyl, methoxy, ethoxy, methylamino, ethylamino, dimethylamino and diethylamino,

or r is 1 or 2 and one R⁶ group is located at the 3-position (relative to the CON(R⁵) group) and is a group of the formula:

-X⁶-R¹⁵

wherein X⁶ is a direct bond or O and R¹⁵ is hydroxymethyl, 1-hydroxyethyl, 2-hydroxyethyl, 3-hydroxypropyl, methoxymethyl, 1-methoxyethyl, 2-methoxyethyl, 1-methoxy-1-methylethyl, 3-methoxypropyl, cyanomethyl, 1-cyanoethyl, 2-cyanoethyl, 3-cyanopropyl, aminomethyl, 1-aminoethyl, 2-aminoethyl, 3-aminopropyl, methylaminomethyl, 1-methylaminoethyl, 2-methylaminoethyl, 3-methylaminopropyl, ethylaminomethyl, 1-ethylaminoethyl, 2-ethylaminoethyl, 1-ethylamino-1-methylethyl, 3-ethylaminopropyl, isopropylaminomethyl, 1-isopropylaminoethyl, dimethylaminomethyl, 1-dimethylaminoethyl, 2-dimethylaminoethyl, 3-dimethylaminopropyl, phenyl, benzyl, cyclopropyl, cyclopentyl, cyclohexyl, thienyl, imidazolyl, thiazolyl, thiadiazolyl, pyrrolidinyl, morpholinyl, tetrahydro-1,4-thiazinyl, piperidinyl, homopiperidinyl, piperazinyl, homopiperazinyl, pyrrolidinylmethyl, 2-(pyrrolidinyl)ethyl, 3-(pyrrolidinyl)propyl, morpholinylmethyl, 2-(morpholinyl)ethyl, 3-(morpholinyl)propyl, piperidinylmethyl, 2-(piperidinyl)ethyl, 3-(piperidinyl)propyl, homopiperidinylmethyl, piperazinylmethyl, 2-(piperazinyl)ethyl, 3-(piperazinyl)propyl or homopiperazinylmethyl, provided that, when X⁶ is O, there are at least two carbon atoms between X⁶ and any heteroatom in the R¹⁵ group,

and wherein any aryl, (3-8C)cycloalkyl, heteroaryl or heterocyclyl group within the R⁶ group optionally bears a substituent selected from fluoro, chloro, trifluoromethyl, hydroxy, amino, methyl, methoxy, methylamino and dimethylamino and any such aryl, (3-8C)cycloalkyl, heteroaryl or heterocyclyl group within the R⁶ group optionally bears a further substituent selected from hydroxymethyl, cyanomethyl, aminomethyl, methylaminomethyl and dimethylaminomethyl,

and any second R⁶ group that is present is selected from fluoro, chloro, trifluoromethyl, cyano, hydroxy, amino, methyl, methoxy, methylamino and dimethylamino;

or a pharmaceutically-acceptable salt thereof.

A further particular compound of the invention is a quinoline derivative of the Formula I wherein:—

X¹ is O;

p is 2 and the first R¹ group is located at the 6-position and is selected from cyano, carbamoyl, methoxy, N-methylcarbamoyl and N,N-dimethylcarbamoyl, and the second R¹ group is located at the 7-position and is selected from methoxy, ethoxy, 2-methoxyethoxy, 3-methoxypropoxy, 2-methylsulphonylethoxy, 3-methylsulphonylpropoxy, 2-(2-methoxyethoxy)ethoxy, 2-pyrrolidin-1-ylethoxy, 3-pyrrolidin-1-ylpropoxy, 2-[(3RS,4SR)-3,4-methylenedioxypyrrolidin-1-yl]ethoxy, 3-[(3RS,4SR)-3,4-methylenedioxypyrrolidin-1-yl]propoxy, 2-morpholinoethoxy, 3-morpholinopropoxy, 2-(1,1-dioxotetrahydro-4H-1,4-thiazin-4-yl)ethoxy, 3-(1,1-dioxotetrahydro-4H-1,4-thiazin-4-yl)propoxy, 2-piperidinoethoxy, 3-piperidinopropoxy, 2-piperidin-3-ylethoxy, 2-(N-methylpiperidin-3-yl)ethoxy, 3-piperidin-3-ylpropoxy, 3-(N-methylpiperidin-3-yl)propoxy, 2-piperidin-4-ylethoxy, 2-(N-methylpiperidin-4-yl)ethoxy, 3-piperidin-4-ylpropoxy, 3-(N-methylpiperidin-4-yl)propoxy, 2-(1,2,3,6-tetrahydropyridin-1-yl)ethoxy, 3-(1,2,3,6-tetrahydropyridin-1-yl)propoxy, 2-(4-hydroxypiperidin-1-yl)ethoxy, 3-(4-hydroxypiperidin-1-yl)propoxy, 2-piperazin-1-ylethoxy, 3-piperazin-1-ylpropoxy, 4-piperazin-1-ylbutoxy, 2-(4-methylpiperazin-1-yl)ethoxy, 3-(4-methylpiperazin-1-yl)propoxy, 4-(4-methylpiperazin-1-yl)butoxy, 2-(4-allylpiperazin-1-yl)ethoxy, 3-(4-allylpiperazin-1-yl)propoxy, 2-(4-prop-2-ynylpiperazin-1-yl)ethoxy, 3-(4-prop-2-ynylpiperazin-1-yl)propoxy, 2-(4-methylsulphonylpiperazin-1-yl)ethoxy, 3-(4-methylsulphonylpiperazin-1-yl)propoxy, 2-(4-acetylpiperazin-1-yl)ethoxy, 3-(4-acetylpiperazin-1-yl)propoxy, 4-(4-acetylpiperazin-1-yl)butoxy, 2-(4-isobutyrylpiperazin-1-yl)ethoxy, 3-(4-isobutyrylpiperazin-1-yl)propoxy, 4-(4-isobutyrylpiperazin-1-yl)butoxy, 2-[4-(2-fluoroethyl)piperazin-1-yl]ethoxy, 3-[4-(2-fluoroethyl)piperazin-1-yl]propoxy, 2-[4-(2,2,2-trifluoroethyl)piperazin-1-yl]ethoxy, 3-[4-(2,2,2-trifluoroethyl)piperazin-1-yl]propoxy, 2-(4-cyanomethylpiperazin-1-yl)ethoxy, 3-(4-cyanomethylpiperazin-1-yl)propoxy, 2-[2-(4-methylpiperazin-1-yl)ethoxy]ethoxy, 2-(4-pyridyloxy)ethoxy, 3-pyridylmethoxy and 2-cyanopyrid-4-ylmethoxy;

q is 0 or q is 1 and the R¹ group which is located at the 2-position (relative to the C(R³)(R⁴) group) is selected from fluoro, chloro, cyano, methyl and methoxy;

each of R³ and R⁴ is hydrogen;

R⁵ is hydrogen or methyl;

Ring A is an oxazolyl, isoxazolyl, imidazolyl, pyrazolyl, thiazolyl, isothiazolyl, oxadiazolyl or thiadiazolyl ring; and

r is 0 or r is 1 or 2 and one R⁶ group is located at the 3-position (relative to the CON(R⁵) group), and each R⁶ group, which may be the same or different, is selected from fluoro, chloro, trifluoromethyl, hydroxy, amino, methyl, ethyl, propyl, isopropyl, butyl, sec-butyl, isobutyl, tert-butyl, cyclopropyl, cyclobutyl, cyclopentyl, methoxy, ethoxy, methylamino, ethylamino, dimethylamino and diethylamino,

or r is 1 or 2 and one R⁶ group is located at the 3-position (relative to the CON(R⁵) group) and is selected from hydroxymethyl, 1-hydroxyethyl, 2-hydroxyethyl, methoxymethyl, 1-methoxyethyl, 2-methoxyethyl, cyanomethyl, 1-cyanoethyl, 2-cyanoethyl, aminomethyl, 1-aminoethyl, 2-aminoethyl, methylaminomethyl, 1-methylaminoethyl, 2-methylaminoethyl, ethylaminomethyl, 1-ethylaminoethyl, 2-ethylaminoethyl, isopropylaminomethyl, 1-isopropylaminoethyl, 2-isopropylaminoethyl, dimethylaminomethyl, 1-dimethylaminoethyl, 2-dimethylaminoethyl, pyrrolidinylmethyl, morpholinylmethyl, piperidinylmethyl and piperazinylmethyl,

and wherein any heterocyclyl group within the R⁶ group optionally bears a substituent selected from fluoro, chloro, trifluoromethyl, hydroxy, amino, methyl, methoxy, methylamino and dimethylamino,

and any second R⁶ group that is present is selected from fluoro, chloro, trifluoromethyl, cyano, hydroxy, amino, methyl, methoxy, methylamino and dimethylamino;

or a pharmaceutically-acceptable salt thereof.

A further particular compound of the invention is a quinoline derivative of the Formula I wherein

X¹ is O;

p is 2 and the first R¹ group is located at the 6-position and is selected from cyano, carbamoyl, methoxy, N-methylcarbamoyl and N,N-dimethylcarbamoyl, and the second R¹ group is located at the 7-position and is selected from methoxy, ethoxy, propoxy, 2-hydroxyethoxy, 3-hydroxypropoxy, 2-methoxyethoxy, 3-methoxypropoxy, 2-methylsulphonylethoxy, 3-methylsulphonylpropoxy and 2-(2-methoxyethoxy)ethoxy;

q is 0 or q is 1 and the R² group which is located at the 2-position (relative to the C(R³)(R⁴) group) is selected from fluoro, chloro, cyano, methyl and methoxy;

each of R³ and R⁴ is hydrogen;

R⁵ is hydrogen or methyl;

Ring A is selected from oxazolyl, isoxazolyl, imidazolyl, pyrazolyl, thiazolyl, isothiazolyl, oxadiazolyl and thiadiazolyl; and

r is 0, 1 or 2 and each R⁶ group that is present is selected from fluoro, chloro, trifluoromethyl, hydroxy, amino, methyl, ethyl, propyl, isopropyl, butyl, sec-butyl, isobutyl, tert-butyl, cyclopropyl, cyclobutyl, cyclopentyl, hydroxymethyl, 2-hydroxyethyl, methoxymethyl, 2-methoxyethyl, methylaminomethyl, ethylaminomethyl, isopropylaminomethyl, cyclopropylaminomethyl, dimethylaminomethyl, methoxy, ethoxy, methylamino, ethylamino, dimethylamino and diethylamino;

or a pharmaceutically-acceptable salt thereof.

A further particular compound of the invention is a quinoline derivative of the Formula I wherein:—

X¹ is O;

p is 2 and the first R¹ group is located at the 6-position and is selected from cyano, carbamoyl, methoxy, N-methylcarbamoyl and N,N-dimethylcarbamoyl, and the second R¹ group is located at the 7-position and is selected from methoxy, ethoxy, 2-hydroxyethoxy and 2-methoxyethoxy;

q is 0 or q is 1 and the R² group which is located at the 2-position (relative to the C(R³)(R⁴) group) is selected from fluoro, chloro, cyano, methyl and methoxy;

each of R³ and R⁴ is hydrogen;

R⁵ is hydrogen or methyl;

Ring A is 2-oxazolyl, 3-isoxazolyl, 5-isoxazolyl, 2-imidazolyl, 3-pyrazolyl, 4-pyrazolyl, 2-thiazolyl, 3-isothiazolyl, 5-isothiazolyl, 1,2,4-oxadiazol-5-yl and 1,3,4-oxadiazol-5-yl; and

r is 1 or 2 and each R⁶ group that is present is selected from methyl, ethyl, propyl, isopropyl, tert-butyl, cyclopropyl, hydroxymethyl, 2-hydroxyethyl, methoxymethyl, 2-methoxyethyl, methylaminomethyl, ethylaminomethyl, isopropylaminomethyl, cyclopropylaminomethyl, dimethylaminomethyl, amino, methylamino, ethylamino, dimethylamino and diethylamino;

or a pharmaceutically-acceptable salt thereof.

In general, compounds falling within the following compound definitions of the present invention possess substantially better potency against the PDGF receptor family of tyrosine kinases, particularly against the PDGFβ receptor tyrosine kinase than against VEGF receptor tyrosine kinases such as KDR.

A particular novel compound of this aspect of the invention is a quinoline derivative of the Formula I, or a pharmaceutically-acceptable salt thereof, wherein:—

p is 0 or p is 1 or 2 and the R¹ groups are located at the 6- and/or 7-positions and are selected from halogeno, trifluoromethyl, cyano, hydroxy, amino, carbamoyl, (1-6C)alkoxycarbonyl, (1-8C)alkyl, (2-8C)alkenyl, (2-8C)alkynyl, (1-6C)alkoxy, (2-6C)alkenyloxy, (2-6C)alkynyloxy, (1-6C)alkylamino, di-[(1-6C)alkyl]amino, N-(1-6C)alkylcarbamoyl and N,N-di-[(1-6C)alkyl]carbamoyl, and

q is 1 and the R² group is located at the 2-position (relative to the C(R³)(R⁴) group) and is selected from halogeno, trifluoromethyl, cyano, carbamoyl, hydroxy, amino, (1-8C)alkyl, (2-8C)alkenyl, (2-8C)alkynyl, (1-6C)alkoxy, (1-6C)alkylamino, di-[(1-6C)alkyl]amino, N-(1-6C)alkylcarbamoyl and N,N-di-[(1-6C)alkyl]carbamoyl;

and each of X¹, R³, R⁴, R⁵, Ring A, r and R⁶ has any of the meanings defined hereinbefore.

A further particular novel compound of this aspect of the invention is a quinoline derivative of the Formula I, or a pharmaceutically-acceptable salt thereof, wherein:—

p is 0 or p is 1 or 2 and the R¹ groups are located at the 6- and/or 7-positions and are selected from fluoro, chloro, trifluoromethyl, cyano, hydroxy, amino, carbamoyl, methoxycarbonyl, ethoxycarbonyl, methyl, ethyl, methoxy, ethoxy, methylamino, dimethylamino, N-methylcarbamoyl and N,N-dimethylcarbamoyl, and

q is 1 and the R² group which is located at the 2-position (relative to the C(R³)(4) group) is selected from fluoro, chloro, trifluoromethyl, cyano, carbamoyl, hydroxy, amino, methyl, ethyl, methoxy, ethoxy, methylamino, dimethylamino, N-methylcarbamoyl and N,N-dimethylcarbamoyl;

and each of X¹, R³, R⁴, R⁵, Ring A, r and R⁶ has any of the meanings defined hereinbefore.

A further particular novel compound of this aspect of the invention is a quinoline derivative of the Formula I, or a pharmaceutically-acceptable salt thereof, wherein:—

p is 0 or p is 1 or 2 and the R¹ groups are located at the 6- and/or 7-positions and are selected from fluoro, chloro, cyano, carbamoyl, methoxycarbonyl, methoxy, ethoxy, N-methylcarbamoyl and N,N-dimethylcarbamoyl, and

q is 1 and the R² group which is located at the 2-position (relative to the C(R³)(R⁴) group) is selected from carbamoyl, methoxy, ethoxy, N-methylcarbamoyl and N,N-dimethylcarbamoyl;

and each of X¹, R³, R⁴, R⁵, Ring A, r and R⁶ has any of the meanings defined hereinbefore.

A further particular novel compound of this aspect of the invention is a quinoline derivative of the Formula I, or a pharmaceutically-acceptable salt thereof, wherein:—

p is 0 or p is 1 or 2 and the R¹ groups are located at the 6- and/or 7-positions and are selected from fluoro, cyano, carbamoyl, methoxycarbonyl, methoxy, ethoxy, N-methylcarbamoyl and N,N-dimethylcarbamoyl, and

q is 1 and the R² group which is located at the 2-position (relative to the C(R³)(R⁴) group) is selected from methoxy and ethoxy;

and each of X¹, R³, R⁴, R⁵, Ring A, r and R⁶ has any of the meanings defined hereinbefore.

A further particular compound of this aspect of the invention is a quinoline derivative of the Formula I wherein:—

X¹ is O;

p is 0 or p is 1 or 2 and the R¹ groups are located at the 6- and/or 7-positions and are selected from halogeno, trifluoromethyl, cyano, hydroxy, amino, carbamoyl, (1-6C)alkoxycarbonyl, (1-8C)alkyl, (2-8C)alkenyl, (2-8C)alkynyl, (1-6C)alkoxy, (2-6C)alkenyloxy, (2-6C)alkynyloxy, (1-6C)alkylamino, di-[(1-6C)alkyl]amino, N-(1-6C)alkylcarbamoyl and N,N-di-[(1-6C)alkyl]carbamoyl,

q is 1 and the R² group is located at the 2-position (relative to the C(R³)(R⁴) group) and is selected from halogeno, trifluoromethyl, cyano, carbamoyl, hydroxy, amino, (1-8C)alkyl, (2-8C)alkenyl, (2-8C)alkynyl, (1-6C)alkoxy, (1-6C)alkylamino, di-[(1-6C)alkyl]amino, N-(1-6C)alkylcarbamoyl and N,N-di-[(1-6C)alkyl]carbamoyl;

each of R³ and R⁴ is hydrogen;

R⁵ is hydrogen;

Ring A is a 5-membered monocyclic heteroaryl ring with up to three ring heteroatoms selected from oxygen, nitrogen and sulphur; and

r is 0, 1, 2 or 3 and each R⁶ group that is present, which may be the same or different, is selected from halogeno, trifluoromethyl, cyano, hydroxy, amino, (1-8C)alkyl, (2-8C)alkenyl, (2-8C)alkynyl, (1-6C)alkoxy, (1-6C)alkylamino, di-[(1-6C)alkyl]amino, (2-6C)alkanoylamino and N-(1-6C)alkyl-(2-6C)alkanoylamino;

or a pharmaceutically-acceptable salt thereof.

A further particular compound of this aspect of the invention is a quinoline derivative of the Formula I wherein:—

X¹ is O;

p is 0 or p is 1 or 2 and the R¹ groups are located at the 6- and/or 7-positions and are selected from fluoro, chloro, trifluoromethyl, cyano, hydroxy, amino, carbamoyl, methoxycarbonyl, ethoxycarbonyl, methyl, ethyl, methoxy, ethoxy, methylamino, dimethylamino, N-methylcarbamoyl and N,N-dimethylcarbamoyl,

q is 1 and the R² group which is located at the 2-position (relative to the C(R³)(R⁴) group) is selected from fluoro, chloro, trifluoromethyl, cyano, carbamoyl, hydroxy, amino, methyl, ethyl, methoxy, ethoxy, methylamino, dimethylamino, N-methylcarbamoyl and N,N-dimethylcarbamoyl;

each of R³ and R⁴ is hydrogen;

R⁵ is hydrogen;

Ring A is a furyl, pyrrolyl, thienyl, oxazolyl, isoxazolyl, imidazolyl, pyrazolyl, thiazolyl, isothiazolyl, oxadiazolyl or thiadiazolyl ring; and

r is 1 or 2 and each R⁶ group, which may be the same or different, is selected from fluoro, chloro, trifluoromethyl, cyano, hydroxy, amino, methyl, ethyl, propyl, isopropyl, butyl, sec-butyl, isobutyl, tert-butyl, methoxy, ethoxy, methylamino, ethylamino, dimethylamino and diethylamino;

or a pharmaceutically-acceptable salt thereof.

A further particular compound of this aspect of the invention is a quinoline derivative of the Formula I wherein

X¹ is O;

p is 0 or p is 1 or 2 and the R¹ groups are located at the 6- and/or 7-positions and are selected from fluoro, chloro, cyano, carbamoyl, methoxycarbonyl, methoxy, ethoxy, N-methylcarbamoyl and N,N-dimethylcarbamoyl,

q is 1 and the R² group which is located at the 2-position (relative to the C(R³)(R⁴) group) is selected from carbamoyl, methoxy, ethoxy, N-methylcarbamoyl and N,N-dimethylcarbamoyl;

each of R³ and R⁴ is hydrogen;

R⁵ is hydrogen;

Ring A is a furyl, pyrrolyl, thienyl, oxazolyl, isoxazolyl, imidazolyl, pyrazolyl, thiazolyl, isothiazolyl, oxadiazolyl or thiadiazolyl ring that bears one or two R⁶ groups and one R⁶ group is located at the 3-position (relative to the CON(R⁵) group); and

r is 1 or 2 and each R⁶ group, which may be the same or different, is selected from fluoro, chloro, trifluoromethyl, cyano, hydroxy, amino, methyl, ethyl, propyl, isopropyl, butyl, sec-butyl, isobutyl, tert-butyl, methoxy, ethoxy, methylamino, ethylamino, dimethylamino and diethylamino;

or a pharmaceutically-acceptable salt thereof.

A further particular compound of this aspect of the invention is a quinoline derivative of the Formula I wherein

X¹ is O;

p is 0 or p is 1 or 2 and the R¹ groups are located at the 6- and/or 7-positions and are selected from fluoro, cyano, carbamoyl, methoxycarbonyl, methoxy, ethoxy, N-methylcarbamoyl and N,N-dimethylcarbamoyl,

q is 1 and the R² group which is located at the 2-position (relative to the C(R³)(R⁴) group) is selected from methoxy and ethoxy;

each of R³ and R⁴ is hydrogen;

R⁵ is hydrogen;

Ring A is a furyl, pyrrolyl, thienyl, oxazolyl, isoxazolyl, imidazolyl, pyrazolyl, thiazolyl, isothiazolyl, oxadiazolyl or thiadiazolyl ring that bears one or two R⁶ groups and one R⁶ group is located at the 3-position (relative to the CON(R⁵) group); and

r is 1 or 2 and each R⁶ group, which may be the same or different, is selected from fluoro, chloro, trifluoromethyl, cyano, hydroxy, amino, methyl, ethyl, propyl, isopropyl, butyl, sec-butyl, isobutyl, tert-butyl, methoxy, ethoxy, methylamino, ethylamino, dimethylamino and diethylamino;

or a pharmaceutically-acceptable salt thereof.

A particular compound of this aspect of the invention is a quinoline derivative of the Formula I wherein:—

X¹ is O;

p is 0 or p is 1 or 2 and the R¹ groups are located at the 6- and/or 7-positions and are selected from fluoro, cyano, carbamoyl, methoxycarbonyl, methoxy, ethoxy, N-methylcarbamoyl and N,N-dimethylcarbamoyl,

q is 1 and the R² group which is located at the 2-position (relative to the C(R³)(R⁴) group) is selected from methoxy and ethoxy;

each of R³ and R⁴ is hydrogen;

R⁵ is hydrogen;

Ring A is 2-oxazolyl, 3-isoxazolyl, 5-isoxazolyl, 2-imidazolyl, 3-pyrazolyl, 4-pyrazolyl, 2-thiazolyl, 3-isothiazolyl, 5-isothiazolyl, 1,2,4-oxadiazol-5-yl and 1,3,4-oxadiazol-5-yl; and

r is 1 or 2 and each R⁶ group that is present is selected from methyl, ethyl, propyl, isopropyl, tert-butyl, cyclopropyl, hydroxymethyl, 2-hydroxyethyl, methoxymethyl, 2-methoxyethyl, methylaminomethyl, ethylaminomethyl, isopropylaminomethyl, cyclopropylaminomethyl, dimethylaminomethyl, amino, methylamino, ethylamino, dimethylamino and diethylamino;

or a pharmaceutically-acceptable salt thereof.

A further particular compound of this aspect of the invention is a quinoline derivative of the Formula I wherein:—

X¹ is O;

p is 0 or p is 1 or 2 and the R¹ groups are located at the 6- and/or 7-positions and are selected from fluoro, cyano, carbamoyl, methoxycarbonyl, methoxy, ethoxy, N-methylcarbamoyl and N,N-dimethylcarbamoyl,

q is 1 and the R² group which is located at the 2-position (relative to the C(R³)(4) group) is a methoxy group;

each of R³ and R⁴ is hydrogen;

R⁵ is hydrogen;

Ring A is 2-oxazolyl, 3-isoxazolyl, 5-isoxazolyl, 3-pyrazolyl, 4-pyrazolyl, 2-thiazolyl, 1,2,4-oxadiazol-5-yl and 1,3,4-oxadiazol-5-yl; and

r is 1 or 2 and each R⁶ group that is present is selected from methyl, ethyl, propyl and isopropyl;

or a pharmaceutically-acceptable salt thereof.

A further particular compound of this aspect of the invention is a quinoline derivative of the Formula I wherein:—

X¹ is O;

p is 0 or p is 1 or 2 and the R¹ groups are located at the 6- and/or 7-positions and are selected from fluoro, cyano, carbamoyl, methoxycarbonyl, methoxy, ethoxy, N-methylcarbamoyl and N,N-dimethylcarbamoyl,

q is 1 and the R² group which is located at the 2-position (relative to the C(R³)(R⁴) group) is a methoxy group;

each of R³ and R⁴ is hydrogen;

R⁵ is hydrogen;

Ring A is 2-oxazolyl, 3-isoxazolyl, 3-pyrazolyl, 4-pyrazolyl and 2-thiazolyl; and

r is 1 or 2 and each R⁶ group that is present is selected from methyl, ethyl, propyl and isopropyl;

or a pharmaceutically-acceptable salt thereof.

Particular compounds of the invention are, for example, the quinoline derivatives of the Formula I that are disclosed within the Examples that are set out hereinafter.

For example, a particular compound of the invention is the quinoline derivative of the Formula I:—
N-(1-ethyl-1H-pyrazol-4-yl)-2-[5-(6,7-dimethoxyquinolin-4-yloxy)pyridin-2-yl]acetamide;
   or a pharmaceutically-acceptable salt thereof.

A further particular compound of the invention is a quinoline derivative of the Formula I selected from:—
N-(1-ethyl-1H-pyrazol-4-yl)-2-[5-(6-cyano-7-methoxyquinolin-4-yloxy)pyridin-2-yl]acetamide,
N-(1-ethyl-1H-pyrazol-4-yl)-2-{5-[6-(N,N-dimethylcarbamoyl)-7-methoxyquinolin-4-yloxy]pyridin-2-yl}acetamide,
N-[1-(2-methoxyethyl)pyrazol-4-yl]-2-[5-(6,7-dimethoxyquinolin-4-yloxy)pyridin-2-yl]acetamide,
N-[1-(2-methoxyethyl)pyrazol-4-yl]-2-[5-(6-cyano-7-methoxyquinolin-4-yloxy)pyridin-2-yl]acetamide,
N-(5-ethyl-1H-pyrazol-3-yl)-2-{5-[6-(N,N-dimethylcarbamoyl)-7-methoxyquinolin-4-yloxy]pyridin-2-yl}acetamide,
N-(4,5-dimethylisoxazol-3-yl)-2-[5-(6,7-dimethoxyquinolin-4-yloxy)pyridin-2-yl]acetamide and N-(4,5-dimethylisoxazol-3-yl)-2-[5-(6-cyano-7-methoxyquinolin-4-yloxy)pyridin-2-ylacetamide;
   or a pharmaceutically-acceptable salt thereof.

A further particular compound of the invention is a quinoline derivative of the Formula I selected from:—
N-(1-ethyl-1H-pyrazol-4-yl)-2-[3-methoxy-5-(7-methoxyquinolin-4-yloxy)pyridin-2-yl]acetamide,
N-(1,3-dimethyl-1H-pyrazol-4-yl)-2-[3-methoxy-5-(7-methoxyquinolin-4-yloxy)pyridin-2-yl]acetamide,
N-(1-ethyl-1H-pyrazol-4-yl)-2-[5-(6-fluoroquinolin-4-yloxy)-3-methoxypyridin-2-yl]acetamide,
N-(4,5-dimethyl-1H-pyrazol-3-yl)-2-[5-(6,7-dimethoxyquinolin-4-yloxy)-3-methoxypyridin-2-yl]acetamide,
N-(4,5-dimethylisoxazol-3-yl)-2-[5-(6,7-dimethoxyquinolin-4-yloxy)-3-methoxypyridin-2-yl]acetamide,
N-(4,5-dimethylisoxazol-3-yl)-2-[3-methoxy-5-(7-methoxyquinolin-4-yloxy)pyridin-2-yl]acetamide,
N-(5-ethylisoxazol-3-yl)-2-[5-(6-fluoroquinolin-4-yloxy)-3-methoxypyridin-2-yl]acetamide and
N-(4,5-dimethylisoxazol-3-yl)-2-[5-(6-fluoroquinolin-4-yloxy)-3-methoxypyridin-2-yl]acetamide;
   or a pharmaceutically-acceptable salt thereof.

A quinoline derivative of the Formula I, or a pharmaceutically-acceptable salt thereof, may be prepared by any process known to be applicable to the preparation of chemically-related compounds. Such processes, when used to prepare a quinoline derivative of the Formula I are provided as a further feature of the invention and are illustrated by the following representative process variants in which, unless otherwise stated, each of X¹, p, R¹, q, R², R³, R⁴, R⁵, Ring A, r and R⁶ has any of the meanings defined hereinbefore. Necessary starting materials may be obtained by standard procedures of organic chemistry. The preparation of such starting materials is described in conjunction with the following representative process variants and within the accompanying Examples. Alternatively, necessary starting materials are obtainable by analogous procedures to those illustrated which are within the ordinary skill of an organic chemist.

(a) The reaction of a quinoline of the Formula II

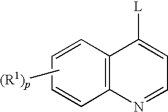

wherein L is a displaceable group and p and R¹ have any of the meanings defined hereinbefore except that any functional group is protected if necessary, with a 2-(2-pyridyl)acetamide of the Formula III

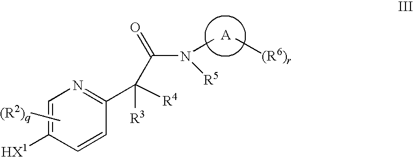

wherein X¹, q, R², R³, R⁴, R⁵, Ring A, r and R⁶ have any of the meanings defined hereinbefore except that any functional group is protected if necessary, whereafter any protecting group that is present is removed.

The reaction may conveniently be carried out in the presence of a suitable acid or in the presence of a suitable base. A suitable acid is, for example, an inorganic acid such as, for example, hydrogen chloride or hydrogen bromide. A suitable base is, for example, an organic amine base such as, for example, pyridine, 2,6-lutidine, collidine, 4-dimethylaminopyridine, triethylamine, morpholine, N-methylmorpholine or diazabicyclo[5.4.0]undec-7-ene, or, for example, an alkali or alkaline earth metal carbonate or hydroxide, for example sodium carbonate, potassium carbonate, calcium carbonate, sodium hydroxide or potassium hydroxide, or, for example, an alkali metal amide, for example sodium hexamethyldisilazane, or, for example, an alkali metal hydride, for example sodium hydride.

A suitable displaceable group L is, for example, a halogeno, alkoxy, aryloxy or sulphonyloxy group, for example a chloro, bromo, methoxy, phenoxy, pentafluorophenoxy, methanesulphonyloxy or toluene-4-sulphonyloxy group. The reaction is conveniently carried out in the presence of a suitable inert solvent or diluent, for example an alcohol or ester such as methanol, ethanol, isopropanol or ethyl acetate, a halogenated solvent such as methylene chloride, chloroform or carbon tetrachloride, an ether such as tetrahydrofuran or 1,4-dioxane, an aromatic solvent such as toluene, or a dipolar aprotic solvent such as N,N-dimethylformamide, N,N-dimethylacetamide, N-methylpyrrolidin-2-one or dimethylsulphoxide. The reaction is conveniently carried out at a temperature in the range, for example, 0 to 250° C., preferably in the range 0 to 120° C.

Typically, the quinoline of the Formula II may be reacted with a compound of the Formula III in the presence of an aprotic solvent such as N,N-dimethylformamide, conveniently in the presence of a base, for example potassium carbonate or sodium hexamethyldisilazane, and at a temperature in the range, for example, 0 to 150° C., preferably in the range, for example, 0 to 70° C.

The quinoline derivative of the Formula I may be obtained from this process in the form of the free base or alternatively it may be obtained in the form of a salt with the acid of the formula H-L wherein L has the meaning defined hereinbefore. When it is desired to obtain the free base from the salt, the salt may be treated with a suitable base, for example, an organic amine base such as, for example, pyridine, 2,6-lutidine, collidine, 4-dimethylaminopyridine, triethylamine, morpholine, N-methylmorpholine or diazabicyclo[5.4.0]undec-7-ene, or, for example, an alkali or alkaline earth metal carbonate or hydroxide, for example sodium carbonate, potassium carbonate, calcium carbonate, sodium hydroxide or potassium hydroxide.

Protecting groups may in general be chosen from any of the groups described in the literature or known to the skilled chemist as appropriate for the protection of the group in question and may be introduced by conventional methods. Protecting groups may be removed by any convenient method as described in the literature or known to the skilled chemist as appropriate for the removal of the protecting group in question, such methods being chosen so as to effect removal of the protecting group with minimum disturbance of groups elsewhere in the molecule.

Specific examples of protecting groups are given below for the sake of convenience, in which “lower”, as in, for example, lower alkyl, signifies that the group to which it is applied preferably has 1-4 carbon atoms. It will be understood that these examples are not exhaustive. Where specific examples of methods for the removal of protecting groups are given below these are similarly not exhaustive. The use of protecting groups and methods of deprotection not specifically mentioned are, of course, within the scope of the invention.

A carboxy protecting group may be the residue of an ester-forming aliphatic or arylaliphatic alcohol or of an ester-forming silanol (the said alcohol or silanol preferably containing 1-20 carbon atoms). Examples of carboxy protecting groups include straight or branched chain (1-12C)alkyl groups (for example isopropyl, and tert-butyl); lower alkoxy-lower alkyl groups (for example methoxymethyl, ethoxymethyl and isobutoxymethyl); lower acyloxy-lower alkyl groups, (for example acetoxymethyl, propionyloxymethyl, butyryloxymethyl and pivaloyloxymethyl); lower alkoxycarbonyloxy-lower alkyl groups (for example 1-methoxycarbonyloxyethyl and 1-ethoxycarbonyloxyethyl); aryl-lower alkyl groups (for example benzyl, 4-methoxybenzyl, 2-nitrobenzyl, 4-nitrobenzyl, benzhydryl and phthalidyl); tri(lower alkyl)silyl groups (for example trimethylsilyl and tert-butyldimethylsilyl); tri(lower alkyl)silyl-lower alkyl groups (for example trimethylsilylethyl); and (2-6C)alkenyl groups (for example allyl). Methods particularly appropriate for the removal of carboxyl protecting groups include for example acid-, base-, metal- or enzymically-catalysed cleavage.

Examples of hydroxy protecting groups include lower alkyl groups (for example tert-butyl), lower alkenyl groups (for example allyl); lower alkanoyl groups (for example acetyl); lower alkoxycarbonyl groups (for example tert-butoxycarbonyl); lower alkenyloxycarbonyl groups (for example allyloxycarbonyl); aryl-lower alkoxycarbonyl groups (for example benzyloxycarbonyl, 4-methoxybenzyloxycarbonyl, 2-nitrobenzyloxycarbonyl and 4-nitrobenzyloxycarbonyl); tri(lower alkyl)silyl (for example trimethylsilyl and tert-butyldimethylsilyl) and aryl-lower alkyl (for example benzyl) groups.

Examples of amino protecting groups include formyl, aryl-lower alkyl groups (for example benzyl and substituted benzyl, 4-methoxybenzyl, 2-nitrobenzyl and 2,4-dimethoxybenzyl, and triphenylmethyl); di-4-anisylmethyl and furylmethyl groups; lower alkoxycarbonyl (for example tert-butoxycarbonyl); lower alkenyloxycarbonyl (for example allyloxycarbonyl); aryl-lower alkoxycarbonyl groups (for example benzyloxycarbonyl, 4-methoxybenzyloxycarbonyl, 2-nitrobenzyloxycarbonyl and 4-nitrobenzyloxycarbonyl); trialkylsilyl (for example trimethylsilyl and tert-butyldimethylsilyl); alkylidene (for example methylidene) and benzylidene and substituted benzylidene groups.

Methods appropriate for removal of hydroxy and amino protecting groups include, for example, acid-, base-, metal- or enzymically-catalysed hydrolysis for groups such as 2-nitrobenzyloxycarbonyl, hydrogenation for groups such as benzyl and photolytically for groups such as 2-nitrobenzyloxycarbonyl.

The reader is referred to Advanced Organic Chemistry, 4th Edition, by J. March, published by John Wiley & Sons 1992, for general guidance on reaction conditions and reagents and to Protective Groups in Organic Synthesis, 2^{nd} Edition, by T. Green et al., also published by John Wiley & Son, for general guidance on protecting groups.

Quinoline starting materials of the Formula II may be obtained by conventional procedures such as those disclosed in International Patent Applications WO 98/13350 and WO 02/12226. For example, a 1,4-dihydroquinolin-4-one of the Formula IV

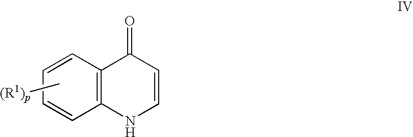

wherein p and R¹ have any of the meanings defined hereinbefore except that any functional group is protected if necessary, may be reacted with a halogenating agent such as thionyl chloride, phosphoryl chloride or a mixture of carbon tetrachloride and triphenylphosphine whereafter any protecting group that is present is removed.

The 4-chloroquinoline so obtained may be converted, if required, into a 4-pentafluorophenoxyquinoline by reaction with pentafluorophenol in the presence of a suitable base such as potassium carbonate and in the presence of a suitable solvent such as N,N-dimethylformamide.

2-(2-Pyridyl)acetamide starting materials of the Formula III may be obtained by conventional procedures. For example, an acetic acid of the Formula V

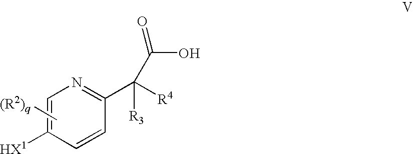

or a reactive derivative thereof, wherein X¹, q, R², R³ and R⁴ have any of the meanings defined hereinbefore except that any functional group is protected if necessary, may be reacted with an amine of the Formula VI

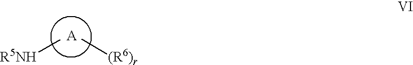

wherein R⁵, Ring A, r and R⁶ have any of the meanings defined hereinbefore except that any functional group is protected if necessary, whereafter any protecting group that is present is removed.

A suitable reactive derivative of an acetic acid of the Formula V is, for example, an acyl halide, for example an acyl chloride formed by the reaction of the acid with an inorganic acid chloride, for example thionyl chloride; a mixed anhydride, for example an anhydride formed by the reaction of the acid with a chloroformate such as isobutyl chloroformate; an active ester, for example an ester formed by the reaction of the acid with a phenol such as pentafluorophenol, with an ester such as pentafluorophenyl trifluoroacetate or with an alcohol such as methanol, ethanol, isopropanol, butanol or N-hydroxybenzotriazole; an acyl azide, for example an azide formed by the reaction of the acid with an azide such as diphenylphosphoryl azide; an acyl cyanide, for example a cyanide formed by the reaction of an acid with a cyanide such as diethylphosphoryl cyanide; or the product of the reaction of the acid with a carbodiimide such as dicyclohexylcarbodiimide or 1-(3-dimethylaminopropyl)-3-ethylcarbodiimide or with a uronium compound such as 2-(7-azabenzotriazol-1-yl)-1,1,3,3-tetramethyluronium hexafluorophosphate(V) or 2-(benzotriazol-1-yl)-1,1,3,3-tetramethyluronium tetrafluoroborate.

The reaction is conveniently carried out in the presence of a suitable inert solvent or diluent, for example an alcohol or ester such as methanol, ethanol, isopropanol or ethyl acetate, a halogenated solvent such as methylene chloride, chloroform or carbon tetrachloride, an ether such as tetrahydrofuran or 1,4-dioxane, an aromatic solvent such as toluene. Conveniently, the reaction is conveniently carried out in the presence of a dipolar aprotic solvent such as N,N-dimethylformamide, N,N-dimethylacetamide, N-methylpyrrolidin-2-one or dimethylsulphoxide. The reaction is conveniently carried out at a temperature in the range, for example, 0 to 120° C., preferably at or near ambient temperature.

Acetic acid derivatives of the Formula V and amines of the Formula VI may be obtained by conventional procedures such as those disclosed in the Examples that are set out hereinafter.

(b) The coupling, conveniently in the presence of a suitable base, of a quinoline of the Formula VII

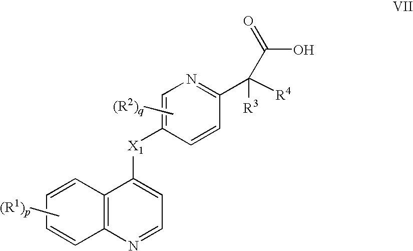

or a reactive derivative thereof as defined hereinbefore, wherein p, R¹, X¹, q, R², R³ and R⁴ have any of the meanings defined hereinbefore except that any functional group is protected if necessary, with an amine of the Formula VI

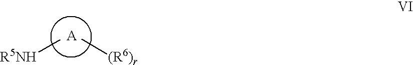

wherein R⁵, Ring A, r and R⁶ have any of the meanings defined hereinbefore except that any functional group is protected if necessary, whereafter any protecting group that is present is removed.

A suitable base is, for example, an organic amine base such as, for example, pyridine, 2,6-lutidine, collidine, 4-dimethylaminopyridine, triethylamine, morpholine, N-methylmorpholine or diazabicyclo[5.4.0]undec-7-ene, or, for example, an alkali or alkaline earth metal carbonate or hydroxide, for example sodium carbonate, potassium carbonate, calcium carbonate, sodium hydroxide or potassium hydroxide, or, for example, an alkali metal amide, for example sodium hexamethyldisilazane, or, for example, an alkali metal hydride, for example sodium hydride.

The reaction is conveniently carried out in the presence of a suitable inert solvent or diluent, for example an alcohol or ester such as methanol, ethanol, isopropanol or ethyl acetate, a halogenated solvent such as methylene chloride, chloroform or carbon tetrachloride, an ether such as tetrahydrofuran or 1,4-dioxane, an aromatic solvent such as toluene. Conveniently, the reaction is conveniently carried out in the presence of a dipolar aprotic solvent such as N,N-dimethylformamide, N,N-dimethylacetamide, N-methylpyrrolidin-2-one or dimethylsulphoxide. The reaction is conveniently carried out at a temperature in the range, for example, 0 to 120° C., preferably at or near ambient temperature.

Quinoline derivatives of the Formula VII and amines of the Formula VI may be obtained by conventional procedures such as those disclosed in the Examples that are set out hereinafter.

(c) For the production of those compounds of the Formula I wherein at least one R¹ group is a group of the formula

Q¹-X²-

wherein Q¹ is an aryl-(1-6C)alkyl, (3-7C)cycloalkyl-(1-6C)alkyl, (3-7C)cycloalkenyl-(1-6C)alkyl, heteroaryl-(1-6C)alkyl or heterocyclyl-(1-6C)alkyl group or an optionally substituted alkyl group and X² is an oxygen atom, the coupling, conveniently in the presence of a suitable dehydrating agent, of a quinoline of the Formula VIII

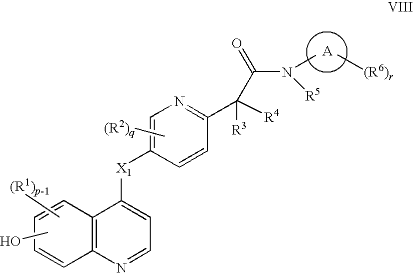

wherein each of p, R¹, X¹, q, R², R³, R⁴, R⁵, Ring A, r and R⁶ has any of the meanings defined hereinbefore except that any functional group is protected if necessary, with an appropriate alcohol wherein any functional group is protected if necessary, whereafter any protecting group that is present is removed.

A suitable dehydrating agent is, for example, a carbodiimide reagent such as dicyclohexylcarbodiimide or 1-(3-dimethylaminopropyl)-3-ethylcarbodiimide or a mixture of an azo compound such as diethyl or di-tert-butyl azodicarboxylate and a phosphine such as triphenylphosphine. The reaction is conveniently carried out in the presence of a suitable inert solvent or diluent, for example a halogenated solvent such as methylene chloride, chloroform or carbon tetrachloride and at a temperature in the range, for example, 10 to 150° C., preferably at or near ambient temperature.

Quinoline derivatives of the Formula VIII may be obtained by conventional procedures.

(d) For the production of those compounds of the Formula I wherein a R⁶ group is a group of the formula -X⁶-R¹⁵ wherein X⁶ has any of the meanings defined hereinbefore and R¹⁵ is an amino-substituted (1-6C)alkyl group (such as a dimethylaminomethyl, 2-dimethylaminoethyl or 4-methylpiperazin-1-ylmethyl group), the reaction, conveniently in the presence of a suitable base as defined hereinbefore, of a compound of the Formula I wherein a R⁶ group is a group of the formula -X⁶-R¹⁵ wherein R¹⁵ is a halogeno-substituted (1-6C)alkyl group with an appropriate amine or with a nitrogen-containing heterocyclyl compound.

The reaction is conveniently carried out in the presence of a suitable inert solvent or diluent as defined hereinbefore and at a temperature in the range, for example, 10 to 180° C., conveniently in the range 20 to 120° C., more conveniently at or near ambient temperature.

Compounds of the Formula I wherein a R⁶ group is a group of the formula -X⁶-R¹⁵ wherein R¹⁵ is a halogeno-substituted (1-6C)alkyl group may be obtained by any of the representative process variants (a), (b) or (c) that are described hereinbefore.

(e) For the production of those compounds of the Formula I wherein a R⁶ group is a group of the formula -X⁶-R¹⁵ wherein X⁶ has any of the meanings defined hereinbefore and R¹⁵ is an amino-substituted (1-6C)alkyl group (such as a methylaminomethyl, 2-methylaminoethyl or 2-hydroxyethylaminomethyl group), the reductive amination of a compound of the Formula I wherein a R⁶ group is a group of the formula -X⁶-R¹⁵ wherein R¹⁵ is a formyl or (2-6C)alkanoyl group.

A suitable reducing agent for the reductive amination reaction is, for example, a hydride reducting agent, for example an alkali metal aluminium hydride such as lithium aluminium hydride or, preferably, an alkali metal borohydride such as sodium borohydride, sodium cyanoborohydride, sodium triethylborohydride, sodium trimethoxyborohydride and sodium triacetoxyborohydride. The reaction is conveniently performed in a suitable inert solvent or diluent, for example tetrahydrofuran and diethyl ether for the more powerful reducing agents such as lithium aluminium hydride, and, for example, methylene chloride or a protic solvent such as methanol and ethanol for the less powerful reducing agents such as sodium triacetoxyborohydride and sodium cyanoborohydride. The reaction is performed at a temperature in the range, for example, 10 to 80° C., conveniently at or near ambient temperature.

Compounds of the Formula I wherein a R⁶ group is a group of the formula -X⁶-R¹⁵ wherein R¹⁵ is a formyl or (2-6C)alkanoyl group may be obtained by a conventional adaptation of any of the representative process variants (a), (b) or (c) that are described hereinbefore.

(f) For the production of those compounds of the Formula I wherein R⁵ is a (1-8C)alkyl group, the alkylation, conveniently in the presence of a suitable base as defined hereinbefore, of a compound of the Formula I wherein R⁵ is hydrogen with a suitable alkylating agent.

The reaction is conveniently carried out in the presence of a suitable inert solvent or diluent as defined hereinbefore and at a temperature in the range, for example, −10° C. to 180° C., conveniently in the range 0 to 100° C., more conveniently at or near ambient temperature.

A suitable alkylating agent is, for example, a compound wherein a (1-8C)alkyl group is attached to a suitable leaving group, for example a chloro, bromo, iodo, methoxy, phenoxy, pentafluorophenoxy, methoxysulphonyloxy, methanesulphonyloxy or toluene-4-sulphonyloxy group.

(g) For the production of those compounds of the Formula I wherein R¹ is a carboxy group, the cleavage, conveniently in the presence of a suitable base as defined hereinbefore, of a compound of the Formula I wherein R¹ is a (1-6C)alkoxycarbonyl group.

Methods appropriate for the cleavage of a (1-6C)alkoxycarbonyl group include, for example, acid-, base-, metal- or enzymically-catalysed hydrolysis. The reaction is conveniently carried out in the presence of a suitable inert solvent or diluent as defined hereinbefore and at a temperature in the range, for example, −10° C. to 100° C., conveniently at or near ambient temperature. For example, base-catalysed cleavage may be effected at ambient temperature using an alkali metal hydroxide such as lithium hydroxide in an alcohol such as methanol.

When a pharmaceutically-acceptable salt of a quinoline derivative of the Formula I is required, for example an acid-addition salt, it may be obtained by, for example, reaction of said quinoline derivative with a suitable acid.

When a pharmaceutically-acceptable pro-drug of a quinoline derivative of the Formula I is required, it may be obtained using a conventional procedure. For example, an in vivo cleavable ester of a quinoline derivative of the Formula I may be obtained by, for example, reaction of a compound of the Formula I containing a carboxy group with a pharmaceutically-acceptable alcohol or by reaction of a compound of the Formula I containing a hydroxy group with a pharmaceutically-acceptable carboxylic acid. For example, an in vivo cleavable amide of a quinoline derivative of the Formula I may be obtained by, for example, reaction of a compound of the Formula I containing a carboxy group with a pharmaceutically-acceptable amine or by reaction of a compound of the Formula I containing an amino group with a pharmaceutically-acceptable carboxylic acid.

Many of the intermediates defined herein are novel and these are provided as a fiuther feature of the invention. For example, many compounds of the Formulae III, VI and VII are novel compounds.

### Biological Assays

The following assays can be used to measure the effects of the compounds of the present invention as inhibitors of PDGFRcα, PDGFRβ and KDR tyrosine kinase enzymes, as inhibitors in vitro of the phosphorylation of PDGFR expressed in MG63 osteosarcoma cells, as inhibitors in vitro of the phosphorylation of KDR expressed in human umbilical vein endothelial cells (HUVECs), as inhibitors in vitro of the proliferation of MG63 osteosarcoma cells, as inhibitors in vitro of the proliferation of HUVECs, and as inhibitors in vivo of the growth in nude mice of xenografts of human tumour tissue such as CaLu-6 and Colo205.

### (a) In Vitro Enzyme Assays

The ability of test compounds to inhibit the phosphorylation of a tyrosine containing polypeptide substrate by the tyrosine kinase enzymes PDGFRα, PDGFRβ and KDR was assessed using conventional ELISA assays.

DNA encoding the PDGFRα, PDGFRβ or KDR receptor cytoplasmic domains may be obtained by total gene synthesis (International Biotechnology Lab., 1987, 5(3), 19-25) or by cloning. The DNA fragments may be expressed in a suitable expression system to obtain polypeptide with tyrosine kinase activity. For example, PDGFRα, PDGFRβ and KDR receptor cytoplasmic domains, obtained by expression of recombinant protein in insect cells, can be shown to display intrinsic tyrosine kinase activity. In the case of the VEGF receptor KDR (Genbank Accession No. L04947), a DNA fragment encoding most of the cytoplasmic domain, commencing with methionine 806 and including the termination codon may be cloned into a baculovirus transplacement vector [for example pAcYM1 (see The Baculovirus Expression System: A Laboratory Guide, L. A. King and R. D. Possee, Chapman and Hall, 1992) or pAc360 or pBlueBacHis (available from Invitrogen Corporation)]. This recombinant construct may be co-transfected into insect cells [for example Spodoptera frugiperda 21(Sf21) or Spodoptera frugiperda 9(Sf9)] with viral DNA (for example Pharmingen BaculoGold) to prepare recombinant baculovirus. Details of the methods for the assembly of recombinant DNA molecules and the preparation and use of recombinant baculovirus can be found in standard texts, for example Sambrook et al., 1989, Molecular cloning—A Laboratory Manual, 2nd edition, Cold Spring Harbour Laboratory Press and O'Reilly et al., 1992, Baculovirus Expression Vectors—A Laboratory Manual, W.H. Freeman and Co, New York).

For expression, Sf9 cells were infected with plaque-pure KDR recombinant virus and harvested 48 hours later. Harvested cells were washed with ice cold phosphate buffered saline solution (PBS) containing 10 mM sodium phosphate pH7.4 buffer, 138 mM sodium chloride and 2.7 mM potassium chloride) and resuspended in ice cold cell diluent comprising 20 mM Hepes pH7.5 buffer, 150 mM sodium chloride, 10% v/v glycerol, 1% v/v Triton X100, 1.5 mM magnesium chloride, 1 mM ethylene glycol-bis(βaminoethyl ether) N,N,N′,N′-tetraacetic acid (EGTA) and 1 mM PMSF (phenylmethylsulphonyl fluoride) [the PMSF is added just before use from a freshly-prepared 100 mM solution in methanol] using 1 ml cell diluent per 10 million cells. The suspension was centrifuged for 10 minutes at 13,000 rpm at 4° C. The supernatant (stock enzyme solution) was removed and stored in aliquots at −70° C.

A substrate solution [100 μl of a 2 μg/1 ml solution of the poly-amino acid Poly(Glu, Ala, Tyr) 6:3:1 (Sigma-Aldrich Company Ltd., Poole, Dorset; Catalogue No. P3899) in phosphate buffered saline (PBS)] was added to each well of a number of Nunc 96-well MaxiSorp immunoplates (Nunc, Roskilde, Denmark; Catalogue No. 439454) and the plates were sealed and stored at 4° C. for 16 hours. The excess of substrate solution was discarded and the wells were washed in turn with PBS containing 0.05% v/v Tween 20 (PBST; 300 μl/well) and twice with Hepes pH7.4 buffer (50 mM, 300 μl/well) before being blotted dry.

Each test compound was dissolved in DMSO and diluted with a 10% solution of DMSO in distilled water to give a series of dilutions (from 40 μM to 0.0012 μM). Aliquots (25 μl) of each dilution of test compound were transferred to wells in the washed assay plates. “Maximum” control wells contained diluted DMSO instead of compound. Aliquots (25 μl) of an aqueous manganese chloride solution (40 mM) containing adenosine-5′-triphosphate (ATP) was added to all test wells except the “blank” control wells which contained magnesium chloride without ATP. For PDGFRα enzyme, an ATP concentration of 14 μM was used; for PDGFRβ enzyme, an ATP concentration of 2.8 μM was used and for KDR enzyme, an ATP concentration of 8 μM was used.

Active human PDGFRα and PDGFRβ recombinant enzyme that had been expressed in Sf9 insect cells was obtained from Upstate Biotechnology Inc., Milton Keynes, UK (product 14-467 for PDGFRα, product 14-463 for PDGFRβ). Active human KDR recombinant enzyme was expressed in Sf9 insect cells as described above.

Each kinase enzyme was diluted immediately prior to use with an enzyme diluent comprising 100 mM Hepes pH7.4 buffer, 0.1 mM sodium orthovanadate, 0.1% Triton X-100 and 0.2 mM dithiothreitol. Aliquots (50 μl) of freshly diluted enzyme were added to each well and the plates were agitated at ambient temperature for 20 minutes. The solution in each well was discarded and the wells were washed twice with PBST. Mouse IgG anti-phosphotyrosine antibody (Upstate Biotechnology Inc.; product 05-321; 100 μl) was diluted by a factor of 1:3667 with PBST containing 0.5% w/v bovine serum albumin (BSA) and aliquots were added to each well. The plates were agitated at ambient temperature for 1.5 hours. The supernatant liquid was discarded and each well was washed with PBST (×2). Horse radish peroxidase (HRP)-linked sheep anti-mouse Ig antibody (Amersham Pharmacia Biotech, Chalfont St Giles, Buckinghamshire, UK; Catalogue No. NXA 931; 100 μl) was diluted by a factor of 1:550 with PBST containing 0.5% w/v BSA and added to each well. The plates were agitated at ambient temperature for 1.5 hours. The supernatant liquid was discarded and the wells were washed with PBST (×2). A sodium perborate (PCSB) capsule (Sigma-Aldrich Company Ltd., Poole, Dorset, UK; Catalogue No. P4922) was dissolved in distilled water (100 ml) to provide phosphate-citrate pH5 buffer (50 mM) containing 0.03% sodium perborate. An aliquot (50 ml) of this buffer was mixed with a 50 mg tablet of 2,2′-azinobis(3-ethylbenzothiazoline-6-sulphonic acid) (ABTS; Roche Diagnostics Ltd., Lewes, East Sussex, UK; Catalogue No. 1204 521). An aliquot (100 μl) of the resultant solution was added to each well. The plates were agitated at ambient temperature for about 20 minutes until the optical density value of the “maximum” control wells, as measured at 405 nm using a plate reading spectrophotometer, was approximately 1.0. “Blank” (no ATP) and “maximum” (no compound) control values were used to determine the dilution range of test compound that gave 50% inhibition of enzyme activity.

(b) In Vitro phospho-Tyr751 PDGFRβ ELISA Assay

This assay uses a conventional ELISA method to determine the ability of test compounds to inhibit phosphorylation of tyrosine in PDGFRβ.

An MG63 osteosarcoma cell line [American Type Culture Collection (ATCC) CCL 1427] was routinely maintained at 37° C. with 7.5% CO₂ in Dulbecco's modified Eagle's growth medium (DMEM; Sigma-Aldrich; Catalogue No. D6546) containing 10% foetal calf serum (FCS; Sigma-Aldrich; Catalogue No. F7524) and 2 mM L-glutamine (Invitrogen Ltd., Paisley, UK; Catalogue No. 25030-024).

For the assay, the cells were detached from the culture flask using a trypsin/ethylenediaminetetraacetic acid (EDTA) mixture (Invitrogen Ltd.; Catalogue No. 15400-054) and resuspended in a test medium comprising DMEM without phenol red (Sigma-Aldrich; Catalogue No. D5921) containing 1% charcoal-stripped foetal calf serum (FCS) (Sigma-Aldrich; Catalogue No. F7524, stripped by incubation with dextran-coated activated charcoal at 55° C. for 30 minutes with continuous stirring followed by removal of the charcoal by centrifugation and filter sterilisation) and 2 mM L-glutamine (Invitrogen Ltd., Catalogue No. 25030-024) to give 6×10⁴ cells per ml. Aliquots (100 μl) were seeded into each of the wells of columns 2-12 (excluding column 1) and rows B-G (excluding rows A and H) of a clear 96 well tissue culture plate (Corning Life Sciences, Koolhovenlaan, The Netherlands; Catalogue No. 3595) to give a density of about 6000 cells per well. Aliquots (100 μl) of culture media were placed in the outer wells to minimise edge effects. The cells were incubated overnight at 37° C. with 7.5% CO₂ to allow the cells to adhere to the wells.

Test compounds were prepared as 10 mM stock solutions in DMSO and serially diluted as required with DMSO to give a range of concentrations. Aliquots (3 μl) of each compound concentration were added to test medium (300 μl) to create a second dilution range. Aliquots (16 μl) of each resultant compound concentration were added to the cells in each well. “Maximum” control cells received a dilution of DMSO plus test medium only. “Minimum” control cells received a reference PDGFR inhibitor (16 μl). The cells were incubated for 90 minutes at 37° C. with 7.5% CO₂.

The resultant cells were stimulated with PDGF_{BB} using the following procedure. A lyophilised powder of PDGF_{BB}, (Sigma-Aldrich; Catalogue No. P4306) was mixed with sterile water to provide a stock solution of 10 μg/ml of PDGF_{BB}. A dilution of this stock solution into test medium provided a 182 ng/ml PDGF_{BB} solution. Aliquots thereof (44 μl) were added to compound treated cells and to the “Maximum” control cells. The “Minimum” control cells received medium only. The cells were incubated at 37° C. with 7.5% CO₂ for 5 minutes. The solution from the wells was removed and the cells were lysed by the addition of 120 μl/well of RIPA buffer comprising 60 mM tris(hydroxymethyl)aminomethane hydrochloride (Tris-HCl), 150 mM sodium chloride, 1 mM EDTA, 1% v/v Igepal CA-630, 0.25% sodium deoxycholate, 1% v/v phosphatase inhibitor cocktail 1 P2850, 1% phosphatase inhibitor cocktail 2 P5726 and 0.5% v/v protease inhibitor cocktail P8340 (all chemicals and inhibitor cocktails were obtainable from the Sigma-Aldrich Company Ltd.). The resultant tissue culture plates were shaken for 5 minutes at ambient temperature to ensure full lysis and then frozen at −20° C. until required.

MaxiSorp ELISA plates (Nunc; Catalogue No. 439454) were coated with PDGFβ antibody (R&D Systems, Abingdon, Oxfordshire, UK; Catalogue No. AF385 comprising lyophilised antibody made up with 100 μl PBS to a final concentration of 100 μl/ml). The antibody was diluted at 1:40 into carbonate-bicarbonate buffer (Sigma-Aldrich; Catalogue No. C3041; one capsule dissolved in 100 ml of distilled water) to give a 2.5 μg/ml solution. Aliquots (50 μl) were added to each well and the plates were placed at 4° C. for 16 hours. The wells were washed 5 times (1 minute soak each time) with 300 μl per well of PBST. The wells were treated with 50 μl of 3% BSA in PBST at ambient temperature for 1 hour and subsequently washed twice with 300 μl per well of PBST.

The tissue culture plates with frozen cell lysate were allowed to warm to 0° C. Aliquots (50 μl) of the MG63 cell lysate were added to the ELISA plates. Each sample was duplicated on separate plates. The ELISA plates were agitated at ambient temperature for 2 hours. The wells were washed twice with 300 μl per well of PBST. A 1:1000 dilution of phospho PDGFRβ antibody (Cell Signaling Technology, Beverley, Mass., USA; Catalogue No. 3161) was made into 1% BSA in PBST. Aliquots (50 μl) of the antibody solutions were added to each of the wells. The plates were agitated at ambient temperature for 1 hour. The plates were washed twice with 300 μl per well of PBST. A 1:2000 dilution of anti-rabbit horseradish peroxidase conjugated secondary antibody (Cell Signaling Technology; Catalogue No. 7074) was made into 1% BSA in PBST. Aliquots (50 μl) of the resultant dilution were added to each well and the plates were agitated at ambient temperature for 1 hour. The plates were washed 5 times with 300 μl per well of PBST. Chemiluminescent substrate was made up according to manufacturers instructions (Pierce Biotechnology Inc., Rockford Ill., USA; Catalogue No. 34080). Aliquots (50 μl) of chemiluminescent substrate solution were added to each of the wells, the plates were agitated for 2 minutes and luminescence was read on a SpectraFluor Plus plate reader (Tecan UK Ltd., Reading, Berkshire, UK). Analysis for each of the compounds was completed by determining a ratio of the ‘phospho antibody’ plate reading to the ‘total antibody’ plate reading for each test sample and these ratios were plotted to determine the IC₅₀ value of each test compound.

### (c) In Vitro Phospho-KDR ELISA Assay

This assay uses a conventional ELISA method to determine the ability of test compounds to inhibit phosphorylation of tyrosine in KDR (VEGFR2).

Human umbilical vein endothelial cells (HUVECs; PromoCell) were routinely incubated at 37° C. with 7.5% CO₂ in ‘growth medium’ comprising MCDB 131 (Gibco Catalogue No. 10372-019; 500 ml) containing L-glutamine (Sigma Catalogue No. G3126; 0.848 g), 1% Penicillin Streptomycin (Gibco Catalogue No. 15140-122) and Fetal Bovine Serum (PAA Laboratories Catalogue No. A15-043; 50 ml).

For the assay, the cells were detached from the culture flask using a trypsin/ethylenediaminetetraacetic acid (EDTA) mixture (Invitrogen Ltd.; Catalogue No. 15400-054) and resuspended in ‘test medium’ comprising MCDB 131 (500 ml) containing L-glutamine (0.848 g), 1% Penicillin Streptomycin and Fetal Bovine Serum (10 ml). Aliquots (1 ml) were seeded into each well of a 24 well tissue culture plate (Corning Life Sciences; Catalogue No. 3527) to give a density of approximately 3.5×10⁴ cells per well. The cells were incubated overnight at 37° C. with 7.5% CO₂ to allow adherence to the well surface. The following morning the assay medium was decanted and an aliquot (0.5 ml) of ‘serum free medium’ comprising MCDB 131 (500 ml) containing L-glutamine (0.848 g) and 1% Penicillin Streptomycin was added to each well. The plates were incubated at 37° C. for 2.5 hours.

Test compounds were prepared as 10 mM stock solutions in DMSO and serially diluted with DMSO as required. Aliquots (3 μl) of each concentration of test compound were diluted with ‘serum free medium’ (300 μl). Aliquots (50 μl) of each resultant compound concentration were added to the cells in each well. “Maximum” control cells received only a dilution of DMSO whereas the “minimum” controls received a reference KDR inhibitor to give a final concentration of 1 μM. The cells were incubated for 90 minutes at 37° C. with 7.5% CO₂.

The resultant cells were stimulated with VEGF using the following procedure. A lyophilised powder of VEGF (Sigma-Aldrich; Catalogue No. V7259) was mixed with PBS containing 0.1% filter-sterilised BSA (0.1% BSA/PBS) to provide a stock solution of 10 μg/ml of VEGF. A dilution of this stock solution into ‘serum free medium’ provided a 1000 ng/ml VEGF solution. Aliquots thereof (50 μl) were added to all wells. The cells were incubated at 37° C. with 7.5% CO₂ for 5 minutes. The solution from the wells was removed and the cells were lysed by the addition of 100 μl/well of RIPA buffer comprising 60 mM Tris-HCl, 150 mM sodium chloride, 1 mM EDTA, 1% v/v Igepal CA-630, 0.25% sodium deoxycholate, 1% v/v phosphatase inhibitor cocktail 1 P2850, 1% phosphatase inhibitor cocktail 2 P5726 and 0.5% v/v protease inhibitor cocktail P8340. The resultant tissue culture plates were shaken for 5 minutes at ambient temperature to ensure full lysis before being frozen on dry-ice and stored at −20° C. until required.

MaxiSorp ELISA plates (Nunc; Catalogue No. 439454) were coated with Phospho-VEGFR2Capture antibody (R&D Systems, Abingdon, Oxfordshire, UK; Human Phospho-VEGFR2ELISA, Catalogue No. DYC1766). The antibody was diluted in PBS to a concentration of 8 μg/ml, aliquots (100 μl) were added to each well and the plates were stored at ambient temperature for 16 hours. The wells were washed 3 times (1 minute soak each time) with 300 μl per well of PBST. The wells were treated with PBS containing 1% filter-sterilised BSA (1% BSA/PBS; 200 μl) at ambient temperature for 1 hour and subsequently washed 3 times with 300 μl per well of PBST.

The tissue culture plates with frozen cell lysate were allowed to warm to 0° C. Aliquots (100 μl) of the HUVEC cell lysate were added and the ELISA plates were agitated at ambient temperature for 3 hours. The wells were washed 3 times with 300 μl per well of PBST. A dilution of Anti-Phospho-Tyrosine-HRP Detection antibody (R&D Systems; Human Phospho-VEGFR2ELISA, Catalogue No. DYC1766) was diluted with 0.1% BSA in Tris-buffered saline solution containing 0.05% v/v Tween 20 (TBST) to make a working concentration of 600 ng/ml. Aliquots (100 μl) of the resultant dilution were added to each well and the plates were agitated at ambient temperature for 2 hours. The plates were washed 4 times with 300 μl per well of PBST. Chemiluminescent substrate was made up according to manufacturers instructions (Pierce Biotechnology Inc., Rockford Ill., USA; Catalogue No. 34080). Aliquots (50 μl) of chemiluminescent substrate solution were added to each of the wells, the plates were agitated for 2 minutes and luminescence was read on a SpectraFluor Plus plate reader (Tecan UK Ltd.). The resultant data were analysed to determine the IC₅₀ value of each test compound.

### (d) In Vitro MG63 Osteosarcoma Proliferation Assay

This assay determined the ability of a test compound to inhibit the proliferation of MG63 osteosarcoma cells (ATCC CCL 1427).

MG63 cells were seeded at 1.5×10³ cells per well into 96-well clear tissue culture-treated assay plates (Corning Life Sciences; Catalogue No. 3595) to which had been added 60 μl per well of test medium comprising DMEM without phenol red, 1% charcoal-stripped FCS and 2 mM glutamine and the cells were incubated overnight at 37° C. with 7.5% CO₂.

Test compounds were solubilised in DMSO to provide a 10 mM stock solution. Aliquots of the stock solution were diluted with the test medium described above and 20 μl aliquots of each dilution were added to appropriate wells. Serial dilutions were made to give a range of test concentrations. Control wells to which DMSO solution only was added were included on each plate. Each plate was duplicated. A lyophilised powder of PDGF_{BB} was mixed with 4 mM aqueous hydrochloric acid containing 0.1% filter-sterilised BSA to provide a stock solution of 10 μg/ml of PDGF_{BB}. A dilution of this stock solution into test medium provided a 250 ng/ml PDGF_{BB} solution. Aliquots (20 μl) thereof were added to one set of control wells to give the “maximum” control. Aliquots (20 μl) thereof were added to one set of the duplicate compound-treated plates and these were denoted as the “PDGF_{BB} stimulated” plates. The second set of duplicate compound-treated plates received media only and these were denoted as the “basal” plates. The “minimum” controls received media only. The plates were incubated at 37° C. with 7.5% CO₂ for 72 hours.

BrdU labelling reagent (Roche Diagnostics Ltd., Lewes, East Sussex, UK; Catalogue No. 647 229) was diluted by a factor of 1:100 in DMEM medium containing 1% charcoal stripped FCS and aliquots (10 μl) were added to each well to give a final concentration of 10 μM. The plates were incubated at 37° C. for 2 hours. The medium was decanted. A denaturating solution (FixDenat solution, Roche Diagnostics Ltd.; Catalogue No. 647 229; 200 μl) was added to each well and the plates were agitated at ambient temperature for 30 minutes. The supernatant was decanted and the wells were washed with PBS (200 μl per well). Anti-BrdU-Peroxidase solution (Roche Diagnostics Ltd.; Catalogue No. 647 229) was diluted by a factor of 1:100 in antibody diluent (Roche Diagnostics Ltd., Catalogue No. 647 229) and 100 μl of the resultant solution was added to each well. The plates were agitated at ambient temperature for 90 minutes. The wells were washed with PBS (×3; 300 μl) to ensure removal of non-bound antibody conjugate. The plates were blotted dry and tetramethylbenzidine substrate solution (Roche Diagnostics Ltd.; Catalogue No. 647 229; 100 μl) was added to each well. The plates were gently agitated on a plate shaker while the colour developed during a 10 to 20 minute period. Aqueous sulphuric acid (1M; 50 μl) was added to the appropriate wells to stop any further reaction and the absorbance of the wells was measured at 450 nm. The extent of inhibition of cellular proliferation at a range of concentrations of each test compound was determined and an anti-proliferative IC₅₀ value was derived.

### (e) In Vitro HUVEC Proliferation Assay

This assay determines the ability of a test compound to inhibit the growth factor-stimulated proliferation of human umbilical vein endothelial cells (HUVECs).

HUVECs were isolated in MCDB 131 (Gibco BRL) and 7.5% v/v foetal calf serum (FCS) and were plated out (at passage 2 to 8) in a mixture of MCDB 131, 2% v/v FCS, 3 μg/ml heparin and 1 μg/ml hydrocortisone, at a concentration of 1000 cells/well in 96 well plates. After a minimum of 4 hours, the cells were dosed with the appropriate growth factor (for example VEGF) and with the test compound. The cultures were incubated for 4 days at 37° C. under 7.5% CO₂. On day 4, the cell cultures were pulsed with 1 μCi/well of tritiated-thymidine (Amersham product TRA 61) and incubated for 4 hours. The cells were harvested using a 96-well plate harvester (Tomtek) and assayed for incorporation of tritium with a Beta plate counter. Incorporation of radioactivity into cells, expressed as counts per minute (cpm), was used to measure inhibition of growth factor-stimulated cell proliferation by each test compound.

### (f) In Vivo Solid Tumour Disease Model

This test measures the capacity of compounds to inhibit solid tumour growth.

CaLu-6 tumour xenografts were established in the flank of female athymic Swiss nu/nu mice, by subcutaneous injection of 1×10⁶ CaLu-6 cells/mouse in 100 μl of a 50% (v/v) solution of Matrigel in serum free culture medium. Ten days after cellular implant, mice were allocated to groups of 8-10 animals having comparable group mean tumour volumes. Tumours were measured using vernier calipers and volumes were calculated using the formula

(l×w)×√(l×w)×(π/6)

where l is the longest diameter and w the diameter perpendicular to the longest. Test compounds were administered orally once daily for a minimum of 21 days, and control animals received compound diluent only. Tumours were measured twice weekly. The level of growth inhibition was calculated by comparison of the mean tumour volume of the control group versus the treatment group using a Student's T test and/or a Mann-Whitney Rank Sum Test.

Although the pharmacological properties of the compounds of the Formula I vary with structural change as expected, in general activity possessed by compounds of the Formula I, may be demonstrated at the following concentrations or doses in one or more of the above tests (a), (b), (c), (d), (e) and (f):—

| | |
|---|---|
| Test (a):- | IC₅₀ versus PDGFRα tyrosine |
| | kinase in the range, for example, |
| | 0.1 nM-5 μM; |
| | IC₅₀ versus PDGFRβ tyrosine kinase |
| | in the range, for example, |
| | 0.1 nM-5 μM; |
| Test (b):- | IC₅₀ versus phospho-Tyr751 formation |
| | in PDGFRβ in the range, for |
| | example, 0.1 nM-1 μM; |
| Test (c):- | IC₅₀ versus phospho-tyrosine |
| | formation in KDR in the range, for |
| | example, 0.1 nM-5 μM; |
| | whilst those compounds having a more selective |
| | inhibitory activity against the PDGF receptor |
| | family of tyrosine kinases have an |
| | IC₅₀ versus phospho-tyrosine |
| | formation in KDR in the range, for |
| | example, 100 nM to greater than 5 μM; |
| Test (d):- | IC₅₀ versus MG63 osteosarcoma |
| | proliferation in the range, for example, 1 nM-5 μM; |
| Test (e):- | IC₅₀ versus HUVEC proliferation |
| | in the range, for example, 1 nM-5 μM; |
| Test (f):- | xenograft activity in the range, for |
| | example, 1-200 mg/kg/day. |

For example, the quinoline compound disclosed as Example 1 possesses activity in Test (b) with an IC₅₀ versus phospho-Tyr751 formation in PDGFRβ of approximately 2 nM; and activity in Test (c) with an IC₅₀ versus phospho-tyrosine formation in KDR of approximately 0.76 μM.

For example, the quinoline compound disclosed as the first Compound listed in Table I within Example 2 possesses activity in Test (b) with an IC₅₀ versus phospho-Tyr751 formation in PDGFRβ of approximately 1 μM.

For example, the quinoline compound disclosed as Example 3 possesses activity in Test (b) with an IC₅₀ versus phospho-Tyr751 formation in PDGFRβ of approximately 30 nM.

For example, the quinoline compound disclosed as the fifth Compound listed in Table I within Example 2 possesses activity in Test (b) with an IC₅₀ versus phospho-Tyr751 formation in PDGFRβ of approximately 2 nM; and activity in Test (c) with an IC₅₀ versus phospho-tyrosine formation in KDR of greater than 2 μM.

For example, the quinoline compound disclosed as the twelfth Compound listed in Table I within Example 2 possesses activity in Test (b) with an IC₅₀ versus phospho-Tyr751 formation in PDGFRβ of approximately 5 nM; and activity in Test (c) with an IC₅₀ versus phospho-tyrosine formation in KDR of greater than 2 μM.

For example, the quinoline compound disclosed as the fourteenth Compound listed in Table I within Example 2 possesses activity in Test (b) with an IC₅₀ versus phospho-Tyr751 formation in PDGFRβ of approximately 5 nM; and activity in Test (c) with an IC₅₀ versus phospho-tyrosine formation in KDR of greater than 2 μM.

For example, the quinoline compound disclosed as the fifthteenth Compound listed in Table I within Example 2 possesses activity in Test (b) with an IC₅₀ versus phospho-Tyr751 formation in PDGFRβ of approximately 5 nM; and activity in Test (c) with an IC₅₀ versus phospho-tyrosine formation in KDR of greater than 2 μM.

For example, the quinoline compound disclosed as the eighteenth Compound listed in Table I within Example 2 possesses activity in Test (b) with an IC₅₀ versus phospho-Tyr751 formation in PDGFRβ of approximately 5 nM; and activity in Test (c) with an IC₅₀ versus phospho-tyrosine formation in KDR of greater than 2 μM.

No untoward toxicological effects are expected when a compound of Formula I, or a pharmaceutically-acceptable salt thereof, as defined hereinbefore is administered at the dosage ranges defined hereinafter.

According to a further aspect of the invention there is provided a pharmaceutical composition which comprises a quinoline derivative of the Formula I, or a pharmaceutically-acceptable salt thereof, as defined hereinbefore in association with a pharmaceutically-acceptable diluent or carrier.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intraperitoneal or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

The amount of active ingredient that is combined with one or more excipients to produce a single dosage form will necessarily vary depending upon the host treated and the particular route of administration. For example, a formulation intended for oral administration to humans will generally contain, for example, from 1 mg to 1 g of active agent (more suitably from 1 to 250 mg, for example from 1 to 100 mg) compounded with an appropriate and convenient amount of excipients which may vary from about 5 to about 98 percent by weight of the total composition.

The size of the dose for therapeutic or prophylactic purposes of a compound of the Formula I will naturally vary according to the nature and severity of the disease state, the age and sex of the animal or patient and the route of administration, according to well known principles of medicine.

In using a compound of the Formula I for therapeutic or prophylactic purposes it will generally be administered so that a daily dose in the range, for example, 1 mg/kg to 100 mg/kg body weight is received, given if required in divided doses. In general, lower doses will be administered when a parenteral route is employed. Thus, for example, for intravenous administration, a dose in the range, for example, 1 mg/kg to 25 mg/kg body weight will generally be used. Similarly, for administration by inhalation, a dose in the range, for example, 1 mg/kg to 25 mg/kg body weight will be used. Oral administration is however preferred, particularly in tablet form. More potent compounds will generally be administered so that a daily oral dose in the range, for example, 1 mg/kg to 25 mg/kg body weight is received. The most potent compounds will generally be administered so that a daily oral dose in the range, for example, 1 mg/kg to 15 mg/kg body weight is received. Typically, unit dosage forms will contain about 10 mg to 0.5 g of a compound of this invention.

As stated above, antagonism of the activity of PDGF receptor kinases, particularly inhibition of the PDGFα and/or PDGFβ receptor tyrosine kinases, is expected to be beneficial in the treatment of a number of cell proliferative disorders such as cancer, especially in inhibiting tumour growth and metastasis and in inhibiting the progression of leukaemia.

We have now found that the novel quinoline derivatives described herein possess potent activity against cell proliferative disorders. It is believed that the compounds provide a useful treatment of cell proliferative disorders, for example to provide an anti-tumour effect, by way of a contribution from inhibition of PDGF receptor tyrosine kinases. In addition, as stated hereinbefore, PDGF is involved in angiogenesis, the process of forming new blood vessels that is critical for continuing tumour growth. It is therefore believed that the compounds of the present invention are expected to be beneficial in the treatment of a number of disease states that are associated with angiogenesis and/or increased vascular permeability such as cancer, especially in inhibiting the development of tumours.

According to this further aspect of the invention there is provided a quinoline derivative of the Formula I, or a pharmaceutically-acceptable salt thereof, as defined hereinbefore for use as a medicament in a warm-blooded animal such as man.

According to a further aspect of the invention, there is provided a quinoline derivative of the Formula I, or a pharmaceutically-acceptable salt thereof, as defined hereinbefore for use in the treatment (or prophylaxis) of cell proliferative disorders or in the treatment (or prophylaxis) of disease states associated with angiogenesis and/or vascular permeability.

According to a further aspect of the invention, there is provided the use of a quinoline derivative of the Formula I, or a pharmaceutically-acceptable salt thereof, as defined hereinbefore in the manufacture of a medicament for use in the treatment (or prophylaxis) of cell proliferative disorders or in the treatment (or prophylaxis) of disease states associated with angiogenesis and/or vascular permeability.

According to this aspect of the invention there is also provided a method for the treatment (or prophylaxis) of cell proliferative disorders in a warm-blooded animal in need of such treatment (or prophylaxis) or for the treatment (or prophylaxis) of disease states associated with angiogenesis and/or vascular permeability in a warm-blooded animal in need of such treatment (or prophylaxis) which comprises administering to said animal an effective amount of a quinoline derivative of the Formula I, or a pharmaceutically-acceptable salt thereof, as defined hereinbefore.

Suitable cell proliferative disorders include neoplastic disorders, for example, cancers of the lung (non-small cell lung cancer, small cell lung cancer and bronchioalveolar cancer), gastrointestine (such as colon, rectal and stomach tumours), prostate, breast, kidney, liver, brain (such as glioblastoma), bile duct, bone, bladder, head and neck, oesophagus, ovary, pancreas, testes, thyroid, cervix and vulva and skin (such as dermatofibrosarcoma protruberans) and in leukaemias and lymphomas such as chronic myelogenous leukaemia (CML), chronic myelomonocytic leukaemia (CMML), acute lymphocytic leukaemia (ALL), chronic neutrophilic leukaemia (CNL), acute myelogenous leukaemia (AML) and multiple myeloma.

According to this aspect of the invention there is also provided a method for treating cell proliferative disorders (such as solid tumour disease) in a warm-blooded animal in need of such treatment which comprises administering to said animal an effective amount of a quinoline derivative of the Formula I, or a pharmaceutically-acceptable salt thereof, as defined hereinbefore.

Other suitable cell proliferative disorders include non-malignant disorders such as blood vessel disease (for example atherosclerosis and restenosis, for example in the process of restenosis subsequent to balloon angioplasty and heart arterial by-pass surgery), fibrotic diseases (for example kidney fibrosis, hepatic cirrhosis, lung fibrosis and multicystic renal dysplasia), glomerulonephritis, benign prostatic hypertrophy, inflammatory diseases (for example rheumatoid arthritis and inflammatory bowel disease), multiple sclerosis, psoriasis, hypersensitivity reactions of the skin, allergic asthma, insulin-dependent diabetes, diabetic retinopathy and diabetic nephropathy.

Suitable disease states associated with angiogenesis and/or vascular permeability include, for example, the undesirable or pathological angiogenesis seen in diabetic retinopathy, psoriasis, cancer, rheumatoid arthritis, atheroma, Kaposi's sarcoma and haemangioma.

According to a further aspect of the invention there is provided a quinoline derivative of the Formula I, or a pharmaceutically-acceptable salt thereof, as defined hereinbefore for use in the treatment (or prevention) of those tumours which are sensitive to inhibition of PDGF receptor enzymes (such as PDGFα and/or PDGFβ receptor tyrosine kinase) that are involved in the signal transduction steps which lead to the proliferation, survival, invasiveness and migratory ability of tumour cells.

According to a further feature of this aspect of the invention there is provided the use of a quinoline derivative of the Formula I, or a pharmaceutically-acceptable salt thereof, as defined hereinbefore in the manufacture of a medicament for use in the treatment (or prevention) of those tumours which are sensitive to inhibition of PDGF receptor enzymes (such as PDGFα and/or PDGFβ receptor tyrosine kinase) that are involved in the signal transduction steps which lead to the proliferation, survival, invasiveness and migratory ability of tumour cells.

According to a further feature of this aspect of the invention there is provided a method for the treatment (or prevention) of a warm-blooded animal having tumours which are sensitive to inhibition of PDGF receptor enzymes (such as PDGFα and/or PDGFβ receptor tyrosine kinase) that are involved in the signal transduction steps which lead to the proliferation, survival, invasiveness and migratory ability of tumour cells which comprises administering to said animal an effective amount of a quinoline derivative of the Formula I, or a pharmaceutically-acceptable salt thereof, as defined hereinbefore.

According to a further aspect of the invention there is provided a quinoline derivative of the Formula I, or a pharmaceutically-acceptable salt thereof, as defined hereinbefore for use in providing a PDGF receptor enzyme inhibitory effect (such as a PDGFα and/or PDGFβ receptor tyrosine kinase inhibitory effect).

According to a further feature of this aspect of the invention there is provided the use of a quinoline derivative of the Formula I, or a pharmaceutically-acceptable salt thereof, as defined hereinbefore in the manufacture of a medicament for use in providing a PDGF receptor enzyme inhibitory effect (such as a PDGFα and/or PDGFβ receptor tyrosine kinase inhibitory effect).

According to a further aspect of the invention there is also provided a method for inhibiting a PDGF receptor enzyme (such as the PDGFα and/or PDGFβ receptor tyrosine kinase) which comprises administering an effective amount of a quinoline derivative of the Formula I, or a pharmaceutically-acceptable salt thereof, as defined hereinbefore.

The anti-cancer treatment defined hereinbefore may be applied as a sole therapy or may involve, in addition to the quinoline derivative of the invention, conventional surgery or radiotherapy or chemotherapy. Such chemotherapy may include one or more of the following categories of anti-tumour agents:—

(i) other antiproliferative/antineoplastic drugs and combinations thereof, as used in medical oncology, such as alkylating agents (for example cis-platin, oxaliplatin, carboplatin, cyclophosphamide, nitrogen mustard, melphalan, chlorambucil, busulphan, temozolamide and nitrosoureas); antimetabolites (for example antifolates such as fluoropyrimidines like 5-fluorouracil and tegafur, raltitrexed, methotrexate, cytosine arabinoside, hydroxyurea and gemcitabine); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine, taxoids like taxol and taxotere, and polo kinase inhibitors); and topoisomerase inhibitors (for example epipodophyllotoxins like etoposide and teniposide, amsacrine, topotecan and camptothecin);
(ii) cytostatic agents such as antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
(iii) anti-invasion agents [for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline (AZDO530; International Patent Application WO 01/94341) and bosutinib (SKI-606), and metalloproteinase inhibitors like marimastat and inhibitors of urokinase plasminogen activator receptor function];
(iv) inhibitors of growth factor function: for example such inhibitors include growth factor antibodies and growth factor receptor antibodies [for example the anti-erbB2 antibody trastuzumab and the anti-erbB1 antibodies cetuximab (C225) and panitumumab]; such inhibitors also include, for example, tyrosine kinase inhibitors [for example inhibitors of the epidermal growth factor family (for example EGFR family tyrosine kinase inhibitors such as gefitinib (ZD1839), erlotinib (OSI-774) and CI 1033, and erbB2 tyrosine kinase inhibitors such as lapatinib), inhibitors of the hepatocyte growth factor family, inhibitors of the insulin growth factor receptor, other inhibitors of the platelet-derived growth factor family and/or bcr/abl kinase such as imatinib, dasatinib (BMS-354825) and nilotinib (AMN107), inhibitors of cell signalling through MEK, AKT, PI3, c-kit, Flt3, CSF-1R and/or aurora kinases]; such inhibitors also include cyclin dependent kinase inhibitors including CDK2 and CDK4 inhibitors; and such inhibitors also include, for example, inhibitors of serine/threonine kinases (for example Ras/Raf signalling inhibitors such as farnesyl transferase inhibitors, for example sorafenib (BAY 43-9006), tipifarnib (R115777) and lonafarnib (SCH66336);
(v) antiangiogenic agents such as those which inhibit the effects of vascular endothelial growth factor, [for example an anti-vascular endothelial cell growth factor antibody such as bevacizumab (Avastin™) or, for example, a VEGF receptor tyrosine kinase inhibitor such as vandetanib (ZD6474), vatalanib (PTK787), sunitinib (SU11248), axitinib (AG-013736), pazopanib (GW 786034) and 4-(4-fluoro-2-methylindol-5-yloxy)-6-methoxy-7-(3-pyrrolidin-1-ylpropoxy)quinazoline (AZD2171; Example 240 within WO 00/47212), or, for example, a compound that works by another mechanism (for example linomide, inhibitors of integrin αvβ3 function and angiostatin)];
(vi) vascular damaging agents such as Combretastatin A4 and compounds disclosed in International Patent Applications WO 99/02166, WO 00/40529, WO 00/41669, WO 01/92224, WO 02/04434 and WO 02/08213;
(vii) antisense therapies, for example those which are directed to the targets listed above, such as ISIS 2503, an anti-ras antisense;
(viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
(ix) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

Such conjoint treatment may be achieved by way of the simultaneous, sequential or separate dosing of the individual components of the treatment. Such combination products employ the compounds of this invention within the dosage range described hereinbefore and the other pharmaceutically-active agent within its approved dosage range.

According to this aspect of the invention there is provided a combination suitable for use in the treatment of cell proliferative disorders (such as solid tumour disease) comprising a quinoline derivative of the formula I as defined hereinbefore and an additional anti-tumour agent as defined hereinbefore.

According to this aspect of the invention there is provided a pharmaceutical product comprising a quinoline derivative of the formula I as defined hereinbefore and an additional anti-tumour agent as defined hereinbefore for the conjoint treatment of cancer.

In particular, the anti-cancer treatment defined hereinbefore may involve the quinoline derivative of the invention in combination with an antiangiogenic agent, for example, an anti-vascular endothelial cell growth factor antibody such as bevacizumab and/or a VEGF receptor tyrosine kinase inhibitor such as vandetanib, vatalanib, sunitinib or AZD2171.

According to this aspect of the invention there is provided a combination suitable for use in the treatment of cell proliferative disorders (such as solid tumour disease) comprising a quinoline derivative of the formula I as defined hereinbefore and an antiangiogenic agent as defined hereinbefore.

According to this aspect of the invention there is also provided a pharmaceutical product comprising a quinoline derivative of the formula I as defined hereinbefore and an antiangiogenic agent as defined hereinbefore for the conjoint treatment of cancer.

The anti-cancer treatment defined hereinbefore may also involve the quinoline derivative of the invention in combination with an anti-invasion agent, for example, a c-Src kinase family inhibitor such as AZD0530 or bosutinib.

According to this aspect of the invention there is provided a combination suitable for use in the treatment of cell proliferative disorders (such as solid tumour disease) comprising a quinoline derivative of the formula I as defined hereinbefore and an anti-invasion agent as defined hereinbefore.

According to this aspect of the invention there is also provided a pharmaceutical product comprising a quinoline derivative of the formula I as defined hereinbefore and an anti-invasion agent as defined hereinbefore for the conjoint treatment of cancer.

The anti-cancer treatment defined hereinbefore may also involve the quinoline derivative of the invention in combination with both an antiangiogenic agent, for example, ane anti-vascular endothelial cell growth factor antibody such as bevacizumab and/or a VEGF receptor tyrosine kinase inhibitor such as vandetanib, vatalanib, sunitinib or AZD2171, and an anti-invasion agent, for example, a c-Src kinase family inhibitor such as AZD0530 or bosutinib.

According to this aspect of the invention there is provided a combination suitable for use in the treatment of cell proliferative disorders (such as solid tumour disease) comprising a quinoline derivative of the formula I as defined hereinbefore, an antiangiogenic agent as defined hereinbefore and an anti-invasion agent as defined hereinbefore.

According to this aspect of the invention there is also provided a pharmaceutical product comprising a quinoline derivative of the formula I as defined hereinbefore, an antiangiogenic agent as defined hereinbefore and an anti-invasion agent as defined hereinbefore for the conjoint treatment of cancer.

In any of the conjoint treatments of cancer described hereinbefore, a bisphosphonate compound may optionally also be present.

Bisphosphonate compounds are diphosphonic acid derivatives that are capable of regulating metal cation (especially calcium) processing within warm-blooded animals such as humans. Accordingly, bisphosphonates are useful in the prevention or treatment of diseases such as osteoporosis and osteolytic bone disease, for example the osteolytic lesions that may occur with metastatic cancers such as renal, thyroid and lung cancers, in particular with breast and prostate cancers. Suitable bisphosphonates include tiludronic acid, ibandronic acid, incadronic acid, risedronic acid, zoledronic acid, clodronic acid, neridronic acid, pamidronic acid and alendronic acid.

Although the compounds of the Formula I are primarily of value as therapeutic agents for use in warm-blooded animals (including man), they are also useful whenever it is required to inhibit the effects of PDGF receptor tyrosine kinase enzymes. Thus, they are useful as pharmacological standards for use in the development of new biological tests and in the search for new pharmacological agents.

The invention will now be illustrated in the following Examples in which, generally:

(i) operations were carried out at ambient temperature, i.e. in the range 17 to 25° C., and under an atmosphere of an inert gas such as nitrogen or argon unless otherwise stated;

(ii) reactions conducted under microwave radiation were performed using an instrument such as a ‘Smith Synthesiser’ (300 KWatts) on either the normal or high setting, which instrument makes use of a temperature probe to adjust the microwave power ouput automatically in order to maintain the required temperature; alternatively an ‘Emrys Optimizer’ microwave instrument may be used;

(iii) in general, the course of reactions was followed by thin layer chromatography (TLC) and/or analytical high pressure liquid chromatography (HPLC); the reaction times that are given are not necessarily the minimum attainable;

(iv) when necessary, organic solutions were dried over anhydrous magnesium sulphate, work-up procedures were carried out after removal of residual solids by filtration, evaporations were carried out by rotary evaporation in vacuo;

(v) yields, where present, are not necessarily the maximum attainable, and, when necessary, reactions were repeated if a larger amount of the reaction product was required;

(vi) in general, the structures of the end-products of the Formula I were confirmed by nuclear magnetic resonance (NMR) and/or mass spectral techniques; electrospray mass spectral data were obtained, for example using a Waters ZMD or Waters ZQ LC/mass spectrometer acquiring both positive and negative ion data, generally, only ions relating to the parent structure are reported; proton NMR (¹H NMR) chemical shift values were measured on the delta scale, for example using a Bruker Spectrospin DPX300 spectrometer operating at a field strength of 300 MHz; the following abbreviations have been used: s, singlet; d, doublet; t, triplet; q, quartet; m, multiplet; br, broad;

(vii) unless stated otherwise compounds containing an asymmetric carbon and/or sulphur atom were not resolved;

(viii) intermediates were not necessarily fully purified but their structures and purity were assessed by TLC, analytical HPLC, infra-red (IR) and/or NMR analysis;

(ix) column chromatography (by the flash procedure) and medium pressure liquid chromatography (MPLC) were performed on silica gel, for example using Merck Kieselgel silica (Art. 9385) or using columns from Armen Instrument (56890-Saint Ave, France);

(x) preparative HPLC was performed on C18 reversed-phase silica, for example on a Waters ‘Xterra’ preparative reversed-phase column (5 microns silica, 19 mm diameter, 100 mm length) or on a Novasep SAS ‘Prochrom DAC’ preparative reversed-phase column using decreasingly polar solvent mixtures as eluent, for example decreasingly polar mixtures of 1% aqueous acetic acid or 1% aqueous ammonium hydroxide (d=0.88) solution and acetonitrile;

(xi) where certain compounds were obtained as an acid-addition salt, for example a mono-hydrochloride salt or a di-hydrochloride salt, the stoichiometry of the salt was based on the number and nature of the basic groups in the compound; generally, elemental analysis data were not obtained to determine the exact stoichiometry of the salt;

(xii) the following abbreviations have been used:—

| | |
|---|---|
| DMF | N,N-dimethylformamide |
| DMA | N,N-dimethylacetamide |
| DMSO | dimethyl sulphoxide |
| THF | tetrahydrofuran |
| NMP | N-methylpyrrolidin-2-one |

### EXAMPLE 1

### N-(1-ethylpyrazol-4-yl)-2-[5-(6,7-dimethoxyquinolin-4-yloxy)pyridin-2-yl]acetamide

Diisopropylethylamine (0.154 ml), 4-amino-1-ethyl-1H-pyrazole (0.098 g) and 2-(7-azabenzotriazol-1-yl)-1,1,3,3-tetramethyluronium hexafluorophosphate(V) (0.335 g) were added in turn to a stirred mixture of 2-[5-(6,7-dimethoxyquinolin-4-yloxy)pyridin-2-yl]acetic acid (0.25 g) and DMF (3 ml) and the resultant mixture was stirred at ambient temperature for 16 hours. The reaction mixture was purified by preparative HPLC using a Waters ‘βBasic Hypersil’ reversed-phase column (5 microns silica, 30 mm diameter, 250 mm length) and decreasingly polar mixtures of water (containing 0.2% ammonium carbonate) and acetonitrile as eluent. The product so obtained was triturated under a 19:1 mixture of diethyl ether and ethanol. The resultant solid was dried under vacuum. There was thus obtained the title compound (0.076 g); ¹H NMR: (DMSOd₆) 1.3 (t, 3H), 3.85 (s, 2H), 3.9 (s, 3H), 3.95 (s, 3H), 4.1 (q, 2H), 6.5 (d, 1H), 7.4 (m, 2H), 7.5 (m, 2H), 7.75 (m, 1H), 7.9 (s, 1H), 8.5 (m, 2H); Mass Spectrum: M+H⁺ 434.

The 2-[5-(6,7-dimethoxyquinolin-4-yloxy)pyridin-2-yl]acetic acid used as a starting material was prepared as follows:—

A mixture of 5-hydroxy-2-methylpyridine (100 g), benzyl chloride (116 ml), potassium carbonate (177.2 g) and DMA (1.5 litres) was stirred and heated to 90° C. for 5 hours. The mixture was filtered and the filtrate was evaporated under vacuum. The residue was purified by column chromatography on silica using an increasingly polar solvent gradient of mixtures of petroleum ether and ethyl acetate (95:5 up to 1:1) as eluent. There was thus obtained 5-benzyloxy-2-methylpyridine (135.6 g); ¹H NMR: (CDCl₃) 2.5 (s, 3H), 5.1 (s, 2H), 7.05 (d, 1H), 7.15 (m, 1H), 7.4 (m, 5H), 8.25 (d, 1H).

A mixture of the material so obtained, 3-choloroperbenzoic acid (184.3 g) and methylene chloride (2 litres) was stirred at ambient temperature for 1 hour. The mixture was washed in turn with a 5% aqueous sodium carbonate solution and with water, dried over magnesium sulphate and evaporated. There was thus obtained 5-benzyloxy-2-methylpyridine N-oxide (135 g); ¹H NMR: (CDCl₃) 2.45 (s, 3H), 5.05 (s, 2H), 6.9 (d, 1H), 7.1 (d, 1H), 7.4 (m, 5H), 8.1 (d, 1H).

The material so obtained was added portionwise to stirred acetic anhydride (296 ml) that was heated to 135° C. (bath temperature) and the resultant mixture was stirred at this temperature for an additional 30 minutes. The mixture was poured into a mixture of ice and water and stirred for 1.5 hours. Sodium chloride was added to saturate the aqueous phase and the mixture was extracted with ethyl acetate. The organic phase was dried over magnesium sulphate and evaporated. The crude product was purified by column chromatography on silica using increasingly polar solvent mixtures of methylene chloride and diethyl ether (100:0 up to 11:9) as eluent. There was thus obtained 2-acetoxymethyl-5-benzyloxypyridine (119 g); Mass Spectrum: M+H⁺ 258.

The material so obtained was added portionwise to a stirred solution of potassium hydroxide (32.9 g) in methanol (330 ml). The resultant mixture was heated to reflux for 1 hour. The bulk of the solvent was removed by evaporation and the residue was partitioned between diethyl ether and water. The organic phase was dried over magnesium sulphate and evaporated. There was thus obtained 5-benzyloxy-2-hydroxymethylpyridine (93.7 g); Mass Spectrum: M+H⁺ 216.

The material so obtained was dissolved in methylene chloride (700 ml) and the solution was cooled to 0° C. Thionyl chloride (34.8 ml) was added dropwise whilst the mixture was cooled to 0° C. The resultant mixture was allowed to warm to ambient temperature and the mixture was stirred for 1 hour. The solvent was evaporated and the residual solid was triturated under diethyl ether. The resultant solid was isolated filtration. There was thus obtained 5-benzyloxy-2-chloromethylpyridine hydrochloride (115.6 g); Mass Spectrum: M+H⁺ 234.

The material so obtained was suspended in ethanol (450 ml) and potassium iodide (1.5 g), water (150 ml) and potassium cyanide (55.6 g) were added in turn. The reaction mixture was stirred and heated to reflux for 4 hours. The resultant mixture was cooled to ambient temperature. A saturated aqueous sodium bicarbonate solution was added and the mixture was extracted with methylene chloride. The organic phase was washed with water and brine, dried over magnesium sulphate and evaporated. The solid so obtained was triturated under diethyl ether. The resultant solid was collected by filtration to give 2-(5-benzyloxypyridin-2-yl)acetonitrile (85.1 g); ¹H NMR: (CDCl₃) 3.9 (s, 2H), 5.1 (s, 2H), 7.4 (m, 7H), 8.3 (d, 1H).

The material so obtained was dissolved in methanol. A 25% sodium hydroxide solution in water (170 ml) was added and the mixture was heated to reflux for 18 hours. The mixture was cooled to ambient temperature and the solvent was evaporated. The residue was dissolved in water and filtered. The filtrate was cooled to 0° C. and acidified to pH4.7 by the addition of 6N aqueous hydrochloric acid. The resultant precipitate was isolated, washed with water and triturated under diethyl ether. The resultant solid was dried under vacuum. There was thus obtained 2-(5-benzyloxypyridin-2-yl)acetic acid (84.1 g); Mass Spectrum: M+H⁺ 244.

2-tert-Butyl-1,3-diisopropylisourea [629 g; obtained as a liquid by the reaction of diisopropylcarbodiimide (496 ml), tert-butanol (303 ml) and cuprous chloride (4.71 g) at ambient temperature under argon for 48 hours and filtration of the mixture] was added to a stirred suspension of 2-(5-benzyloxypyridin-2-yl)acetic acid (84.1 g) in methylene chloride (1.4 litres) and the mixture was stirred at ambient temperature for 48 hours. The resultant precipitate was removed by filtration and the filtrate was concentrated under vacuum. The residue was purified by column chromatography on silica using increasingly polar solvent mixtures of methylene chloride and ethyl acetate (100:0 up to 19:1) as eluent. There was thus obtained tert-butyl 2-(5-benzyloxypyridin-2-yl)acetate (63.7 g) as an oil which crystallised upon standing; ¹H NMR: (DMSOd₆) 1.4 (s, 9H), 3.65 (s, 2H), 5.2 (s, 2H), 7.25 (d, 1H), 7.4 (m, 6H), 8.25 (d, 1H).

Palladium hydroxide on carbon catalyst (10 g; about 20% palladium and about 50% water) was added to a stirred mixture of tert-butyl 2-(5-benzyloxypyridin-2-yl)acetate (58.7 g), 1,4-cyclohexadiene (200 ml) and ethanol (1 litre). The resultant mixture was heated to reflux for 2 hours. The mixture was cooled to ambient temperature and the catalyst was removed by filtration. The filtrate was evaporated. The residue was triturated under diethyl ether. The resultant solid was isolated. There was thus obtained tert-butyl 2-(5-hydroxypyridin-2-yl)acetate as a white solid (39.6 g); ¹H NMR: (DMSOd₆) 1.4 (s, 9H), 3.6 (s, 2H), 7.1 (m, 2H), 8.05 (d, 1H), 9.8 (br s, 1H).

A mixture of 4-chloro-6,7-dimethoxyquinoline (3 g; International Patent Application WO 98/13350, Example 2 thereof), tert-butyl (5-hydroxypyridin-2-yl)acetate (3.22 g), 4-dimethylaminopyridine (4.9 g) and chlorobenzene (30 ml) was stirred and heated to 130° C. for 8 hours. The resultant mixture was cooled to ambient temperature and diluted with diethyl ether. The mixture was filtered and the filtrate was washed with water and extracted with 1N aqueous hydrochloric acid (60 ml, twice). The acidic solution was washed with diethyl ether and cautiously basified by the addition of solid sodium bicarbonate. The resultant aqueous mixture was extracted with ethyl acetate. The organic phase was washed with water and with brine, dried over magnesium sulphate and evaporated. There was thus obtained tert-butyl 2-[5-(6,7-dimethoxyquinolin-4-yloxy)pyridin-2-yl]acetate as an oil (4.38 g) which was used without further purification; Mass Spectrum: M+H⁺ 397.

Trifluoroacetic acid (20 ml) was added to a stirred mixture of tert-butyl 2-[5-(6,7-dimethoxyquinolin-4-yloxy)pyridin-2-yl]acetate (4.38 g) and methylene chloride (60 ml) that had been cooled to 0° C. The resultant mixture was stirred at ambient temperature for 16 hours. The solvent was evaporated. Toluene was added and the mixture was evaporated to remove traces of trifluoroacetic acid. The residue was triturated under a mixture of diethyl ether and ethyl acetate. The resultant solid was suspended in methylene chloride and methanol was added until solubilisation. Diisopropylethylamine (5 ml) was added and the mixture was stirred at ambient temperature for 30 minutes. The resultant mixture was evaporated and the residual oil which triturated under a mixture of diethyl ether and ethyl acetate. The resultant solid was isolated and dried under vacuum. There was thus obtained 2-[5-(6,7-dimethoxyquinolin-4-yloxy)pyridin-2-yl]acetic acid (2.43 g); Mass Spectrum: M+H⁺ 341.

The 4-amino-1-ethyl-1H-pyrazole used as a starting material was prepared as follows:—

4-Nitropyrazole is available commercially from the N.D. Zelinsky Institute, Organic Chemistry, Leninsky prospect 47, 117913 Moscow B-334, Russia. The compound may also be prepared as follows:—

Fuming nitric acid (9.5 ml) was added dropwise to a stirred solution of pyrazole (13.6 g) in glacial acetic acid (51 ml) that had been cooled to −10° C. using an ice-salt bath. A voluminous precipitate was formed. Acetic anhydride (27 ml) was added dropwise and the resultant mixture was stirred at ambient temperature for 2.5 hours. The mixture was poured onto ice and the acidity of the mixture was reduced to pH5 by the addition of potassium carbonate. The precipitate was isolated by filtration. The resultant solid was dissolved in water and the aqueous solution was extracted with diethyl ether. The organic solution was dried over magnesium sulphate and filtered. Petroleum ether (b.p. 60-80° C., 50 ml) was added to the filtrate which was concentrated by evaporation to a volume of about 50 ml. A precipitate formed which was isolated by filtration. This solid was believed to be 1-nitropyrazole (20.6 g); ¹H NMR: (DMSOd₆) 6.71 (s, 1H), 7.88 (s, 1H), 8.81 (s, 1H). The compound may be explosive and should be handled cautiously.

Concentrated sulphuric acid (80 ml) was added dropwise to a stirred sample of 1-nitropyrazole (20.3 g) that was cooled in an ice-bath. The resultant mixture was stirred for 16 hours and allowed to warm to ambient temperature. The mixture was poured onto ice and stirred for 20 minutes. The resultant solid was isolated and washed with water. The filtrate was neutralised by the addition of potassium carbonate and extracted with diethyl ether. The recovered solid was added to the diethyl ether solution and the resultant solution was washed with a saturated aqueous sodium chloride solution, dried over magnesium sulphate and filtered. Petroleum ether (b.p. 60-80° C.) was added to the filtrate which was concentrated by evaporation to a volume of about 50 ml. A precipitate formed which was isolated by filtration. There was thus obtained 4-nitropyrazole (16 g); ¹H NMR: (DMSOd₆+CF₃CO₂H) 8.57 (s, 2H).

Diethyl sulphate (5.23 ml) was slowly added to a stirred solution of 4-nitropyrazole (2.26 g) in 1N aqueous sodium hydroxide solution (22 ml) that had been warmed to 30° C. and the resultant mixture was stirred at that temperature for 48 hours. The mixture was cooled to ambient temperature and the precipitate was isolated, washed with cold water and dried under vacuum. There was thus obtained 1-ethyl-4-nitro-1H-pyrazole (1.71 g); ¹H NMR: (DMSOd₆) 1.4 (t, 3H), 4.2 (q, 2H), 8.25 (s, 1H), 8.9 (s, 1H).

A mixture of a portion (0.8 g) of the material so obtained, platinum oxide (0.1 g), ethyl acetate (10 ml) and ethanol (30 ml) was stirred under 3 atmospheres pressure of hydrogen for 2 hours. The catalyst was removed by filtration and the filtrate was evaporated. There was thus obtained the required starting material in 89% yield; ¹H NMR: (DMSOd₆) 1.27 (t, 3H), 3.77 (br s, 2H), 3.92 (q, 2H), 6.87 (s, 1H), 7.01 (s, 1H).

### EXAMPLE 2

Using an analogous procedure to that described in Example 1, the appropriate 2-pyridin-2-ylacetic acid was reacted with the appropriate amine to give the compounds described in Table I. Unless otherwise stated, each amine was a commercially available material.

TABLE I

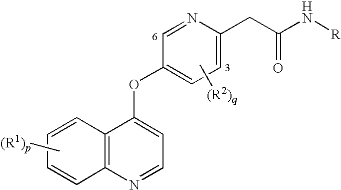

| No. & | | | |
|---|---|---|---|
| Note | (R¹)ₚ | (R²)_{q} | R |
| [1] | 6,7-dimethoxy | H | 5-ethylpyrazol-3-yl |
| [2] | 6,7-dimethoxy | H | 5-ethylisoxazol-3-yl |
| [3] | 6,7-dimethoxy | H | 4-methylthiazol-2-yl |
| [4] | 6,7-dimethoxy | H | 5-methylthiazol-2-yl |
| [5] | 7-methoxy | 3-methoxy | 1-ethylpyrazol-4-yl |
| [6] | 7-methoxy | 3-methoxy | 1,3-dimethylpyrazol-4-yl |
| [7] | 7-methoxy | 3-methoxy | 1,5-dimethylpyrazol-4-yl |
| [8] | 6-fluoro | 3-methoxy | 1-ethylpyrazol-4-yl |
| [9] | 7-(N-methyl- | 3-methoxy | 1-ethylpyrazol-4-yl |
| | carbamoyl)- | | |
| | 6-methoxy | | |
| [10] | 7-methoxy | 3-methoxy | 5-ethylpyrazol-3-yl |
| [11] | 6-fluoro | 3-methoxy | 5-ethylpyrazol-3-yl |
| [12] | 6,7-dimethoxy | 3-methoxy | 4,5-dimethylpyrazol-3-yl |
| [13] | 7-methoxy | 3-methoxy | 5-ethylisoxazol-3-yl |
| [14] | 7-methoxy | 3-methoxy | 4,5-dimethylisoxazol-3-yl |
| [15] | 6-fluoro | 3-methoxy | 5-ethylisoxazol-3-yl |
| [16] | 6-fluoro | 3-methoxy | 4,5-dimethylisoxazol-3-yl |
| [17] | 7-(N-methyl- | 3-methoxy | 4,5-dimethylisoxazol-3-yl |
| | carbamoyl)- | | |
| | 6-methoxy | | |
| [18] | 6,7-dimethoxy | 3-methoxy | 4,5-dimethylisoxazol-3-yl |
| [19] | 7-methoxy | 3-methoxy | 4-methylthiazol-2-yl |
| [20] | 7-methoxy | 3-methoxy | 3-dimethylaminomethyl- |
| | | | 5-methylphenyl |
| [21] | 6-fluoro | 3-methoxy | 3-dimethylaminomethyl- |
| | | | 5-methylphenyl |
| [22] | 7-(N-methyl- | 3-methoxy | 3-dimethylaminomethyl- |
| | carbamoyl)- | | 5-methylphenyl |
| | 6-methoxy | | |
| [23] | 5,7-dimethoxy | 3-methoxy | 1-ethylpyrazol-4-yl |
| [24] | 5,7-dimethoxy | 3-methoxy | 1,3-dimethylpyrazol-4-yl |
| [25] | 6-fluoro | 3-methoxy | 1-isopropylpyrazol-4-yl |
| [26] | 6,7-dimethoxy | 3-methoxy | 1-methylpyrazol-4-yl |
| [27] | 6,7-dimethoxy | 3-methoxy | 1,3-dimethylpyrazol-4-yl |
| [28] | 6,7-dimethoxy | 3-methoxy | 1-ethyl-3-methylpyrazol-4-yl |
| [29] | 6-methoxy | 3-methoxy | 1-ethylpyrazol-4-yl |
| [30] | 6-methoxy | 3-methoxy | 1-isopropylpyrazol-4-yl |
| [31] | 6-methoxy | 3-methoxy | 1,3-dimethylpyrazol-4-yl |
| [32] | 7-ethoxy-6-methoxy | 3-methoxy | 1-ethylpyrazol-4-yl |
| [33] | 7-ethoxy-6-methoxy | 3-methoxy | 1,3-dimethylpyrazol-4-yl |
| [34] | 7-ethoxy-6-methoxy | 3-methoxy | 1-ethyl-3-methylpyrazol-4-yl |
| [35] | 6,7-dimethoxy | 3-methoxy | 1,3-dimethylpyrazol-5-yl |
| [36] | 6,7-dimethoxy | 3-methoxy | 4-methylisoxazol-3-yl |
| [37] | 6-methoxy | 3-methoxy | 3-dimethylaminomethyl- |
| | | | 5-methylphenyl |

Notes Characterising data for each product are shown below.
[1] An additional portion of 2-(7-azabenzotriazol-1-yl)-1,1,3,3-tetramethyluronium hexafluorophosphate was added and the reaction mixture was heated to 50° C. for 16 hours. The product gave the following characterising data:- ¹H NMR: (DMSOd₆) 1.2(t, 3 H), 2.55(q, 2 H), 3.9(s, 2 H), 3.95(s, 3 H), 4.0(s, 3 H), 6.3(br s, 1 H), 6.55(d, 1 H), 7.4(s, 1 H), 7.5(m, 2 H), 7.75(m, 1 H), 8.55(m, 2 H); Mass Spectrum: M + H⁺ 434.

The 5-amino-3-ethyl-1H-pyrazole used as a starting material was prepared as follows:—

Acetonitrile (1.17 ml) was added dropwise to a stirred solution of n-butyllithium (1.6M in hexane, 14.06 ml) that had been cooled to −78° C. and the mixture was stirred at that temperature for 1 hour. Ethyl propionate (1.5 ml) was added dropwise and the reaction medium was allowed to warm to −45° C. and stirred at that temperature for 2 hours. The resultant mixture was acidifed to pH2 by the addition of 2N aqueous hydrochloric acid and concentrated by evaporation. The residue was extracted with methylene chloride and the organic extract was dried over magnesium sulphate and evaporated. There was thus obtained 3-oxopentanenitrile in 80% yield; ¹H NMR: (CDCl₃) 1.14 (t, 3H), 2.66 (q, 2H), 3.46 (s, 2H).

A mixture of a portion (0.6 g) of the material so obtained, hydrazine hydrate (0.28 ml) and ethanol (45 ml) was heated at 70° C. for 12 hours. The solvent was evaporated and the residue was purified by column chromatography on silica using a 19:1 mixture of methylene chloride and methanol as eluent. There was thus obtained the required starting material in 51% yield; ¹H NMR: (DMSOd₆) 1.04 (t, 3H), 2.41 (q, 2H), 4.4 (br s, 2H).

[2] ¹H NMR: (DMSOd₆) 1.2 (t, 3H), 2.75 (q, 2H), 3.9 (s, 2H), 3.95 (s, 3H), 4.0 (s, 3H), 6.5 (d, 1H), 6.65 (s, 1H), 7.45 (s, 1H), 7.55 (m, 2H), 7.75 (m, 1H), 8.55 (m, 2H); Mass Spectrum: M+H⁺ 435.

The 3-amino-5-ethylisoxazole used as a starting material is described in International Patent Application WO 2005/026113 (pages 33 and 34 thereof).

[3] ¹H NMR: (DMSOd₆) 2.25 (s, 3H), 3.9 (s, 3H), 3.95 (s, 3H), 4.05 (s, 2H), 6.55 (d, 1H), 6.75 (s, 1H), 7.4 (s, 1H), 7.5 (s, 1H), 7.55 (d, 1H), 7.75 (m, 1H), 8.5 (m, 2H); Mass Spectrum: M+H⁺ 437.

[4] An additional portion of 2-(7-azabenzotriazol-1-yl)-1,1,3,3-tetramethyluronium hexafluorophosphate was added after 4 hours and the reaction mixture was stirred for a further 16 hours. The product gave the following characterising data:—¹H NMR: (DMSOd₆) 2.35 (s, 3H), 3.9 (s, 3H), 3.95 (s, 3H), 4.0 (s, 2H), 6.55 (d, 1H), 7.15 (s, 1H), 7.4 (s, 1H), 7.5 (s, 1H), 7.55 (d, 1H), 7.75 (m, 1H), 8.55 (m, 2H); Mass Spectrum: M+H⁺ 437.

[5] ¹H NMR: (DMSOd₆) 1.32 (t, 3H), 3.79 (s, 2H), 3.81 (s, 3H), 3.94 (s, 3H), 4.07 (q, 2H), 6.55 (d, 1H), 7.32 (m, 1H), 7.41 (s, 1H), 7.43 (d, 1H), 7.51 (d, 1H), 7.87 (s, 1H), 8.09 (d, 1H), 8.22 (d, 1H), 8.65 (d, 1H), 10.09 (s, 1H); Mass Spectrum: M+H⁺ 434.

The 2-[3-methoxy-5-(7-methoxyquinolin-4-yloxy)pyridin-2-yl]acetic acid used as a starting material was prepared as follows:—

Under an atmosphere of argon, a 1M solution of lithium hexamethyldisilazane in THF (80 ml) was added dropwise to a stirred mixture of 5-bromo-2-chloro-3-methoxypyridine (International Patent Application WO 01/81347, within Example 10 thereof; 8 g), acetonitrile (4.1 ml) and THF (80 ml) that had been cooled to 18° C. Additional acetonitrile (4.1 ml) and 1M lithium hexamethyldisilazane solution (80 ml) were added in turn. The reaction mixture was allowed to warm to ambient temperature and poured into a stirred mixture of water (300 ml) and diethyl ether (300 ml). The aqueous phase was extracted with diethyl ether. The organic phases were combined, dried over magnesium sulphate and evaporated. The residue was purified by column chromatography on silica using a solvent gradient of 4:1 to 1:1 petroleum ether and diethyl ether as eluent. There was thus obtained 2-(5-bromo-3-methoxypyridin-2-yl)acetonitrile (4.8 g); ¹H NMR: (CDCl₃) 3.85 (s, 2H), 3.91 (s, 3H), 7.35 (d, 1H), 8.25 (d, 1H); Mass Spectrum: M+H⁺ 227 and 229.

Trimethylsilyl chloride (21.55 ml) was added to a stirred mixture of 2-(5-bromo-3-methoxypyridin-2-yl)acetonitrile (6.6 g) and methanol (70 ml). The resultant mixture was heated to 50° C. for 12 hours. The mixture was concentrated by evaporation and the residue was dissolved in diethyl ether. A saturated aqueous sodium bicarbonate solution was added until gas evolution ceased. The resultant aqueous phase was separated and extracted with diethyl ether. The organic phases were combined, dried over magnesium sulphate and evaporated. The residue was passed through silica (70 g) using a 1:1 mixture of petroleum ether and diethyl ether as eluent. There was thus obtained methyl 2-(5-bromo-3-methoxypyridin-2-yl)acetate (7.1 g); ¹H NMR: (CDCl₃) 3.71 (s, 3H), 3.83 (s, 2H), 3.85 (s, 3H), 7.3 (d, 1H), 8.21 (d, 1H); Mass Spectrum: M+H⁺ 260 and 262.

Under an atmosphere of argon, bis(pinacolato)diboron (8.23 g), a [1,1′-bis(diphenylphosphino)ferrocene]dichloropalladium(II) 1:1 complex with methylene chloride (0.661 g) and potassium carbonate (8.21 g) were added in turn to a stirred mixture of methyl 2-(5-bromo-3-methoxypyridin-2-yl)acetate (7.02 g) and 1,4-dioxane (300 ml). The resultant mixture was stirred and heated to 90° C. for 6 hours. The mixture was cooled to ambient temperature and partially concentrated by evaporation. The residue was diluted with methylene chloride and the mixture was washed in turn with water and brine. The organic phase was recovered, dried over magnesium sulphate and evaporated. There was thus obtained methyl 2-[3-methoxy-5-(4,4,5,5-tetramethyl-1,3,2-dioxaborolan-2-yl)pyridin-2-yl]acetate as a dark oil (16.8 g) that contained some 1,4-dioxane; ¹H NMR: (CDCl₃) 1.35 (s, 6H), 3.69 (s, 3H), 3.87 (s, 3H), 3.92 (s, 2H), 7.5 (d, 1H), 8.49 (d, 1H); Mass Spectrum: M+H⁺ 308.

The material so obtained was dissolved in methylene chloride (300 ml). An aqueous hydrogen peroxide solution (30%, 15 ml) was added and the resultant mixture was stirred vigorously at ambient temperature for 2 hours. The two phases were separated. The aqueous phase was extracted with methylene chloride. The organic phases were combined, washed with brine, dried over magnesium sulphate and evaporated. The residue was purified by column chromatography on silica using increasingly polar mixtures of diethyl ether and ethyl acetate as eluent. There was thus obtained methyl 2-(5-hydroxy-3-methoxypyridin-2-yl)acetate (3.2 g); ¹H NMR: (DMSOd₆) 3.58 (s, 3H), 3.63 (s, 2H), 3.74 (s, 3H), 6.8 (d, 1H), 7.63 (d, 1H); Mass Spectrum; M+H⁺ 198.

Under an atmosphere of argon, a mixture of methyl 2-(5-hydroxy-3-methoxypyridin-2-yl)acetate (0.749 g), 4-chloro-7-methoxyquinoline (J. Med. Chem., 1998, 41, 4918-4926; 0.772 g), 4-dimethylaminopyridine (1.49 g) and chlorobenzene (10 ml) was stirred and heated to reflux for 5 hours. The resultant mixture was cooled to ambient temperature and partitioned between water and ethyl acetate. The organic phase was washed with dilute aqueous hydrochloric acid, dried over magnesium sulphate and evaporated. The residue was stirred under diethyl ether. The resultant solid was isolated. There was thus obtained methyl 2-[3-methoxy-5-(7-methoxyquinolin-4-yloxy)pyridin-2-yl]acetate (0.99 g); ¹H NMR: (DMSOd₆) 3.64 (s, 3H), 3.82 (s, 3H), 3.83 (s, 2H), 3.95 (s, 3H), 6.57 (d, 1H), 7.31 (m, 1H), 7.43 (d, 1H), 7.54 (d, 1H), 8.09 (s, 1H), 8.21 (d, 1H), 8.65 (d, 1H); Mass Spectrum: M+H⁺ 355.

A mixture of a portion (0.18 g) of the material so obtained, a 2N aqueous sodium hydroxide solution (0.765 ml) and methanol (2 ml) was stirred at ambient temperature for 2 hours. A 2N aqueous hydrochloric acid solution of (0.765 ml) was added. The resultant solid was isolated, washed with water and dried at 50° C. under vacuum. There was thus obtained 2-[3-methoxy-5-(7-methoxyquinolin-4-yloxy)pyridin-2-yl]acetic acid (0.15 g); ¹H NMR: (DMSOd₆) 3.74 (s, 2H), 3.82 (s, 3H), 3.95 (s, 3H), 6.57 (d, 1H), 7.33 (m, 1H), 7.44 (d, 1H), 7.52 (d, 1H), 8.08 (s, 1H), 8.23 (d, 1H), 8.66 (d, 1H); Mass Spectrum: M+H⁺ 341.

[6] ¹H NMR: (DMSOd₆) 2.14 (s, 3H), 3.71 (s, 3H), 3.84 (s, 3H), 3.87 (s, 2H), 3.96 (s, 3H), 6.57 (d, 1H), 7.33 (m, 1H), 7.45 (d, 1H), 7.52 (d, 1H), 7.82 (s, 1H), 8.1 (d, 1H), 8.24 (d, 1H), 8.67 (d, 1H), 9.53 (s, 1H); Mass Spectrum: M+H⁺ 434.

The 4-amino-1,3-dimethyl-1H-pyrazole used as a starting material was obtainable commercially from Sigma-Aldrich, Gillingham, SP8 4XT, UK). The compound may also be prepared according to the procedure disclosed in Chemical Abstracts volume 94, Abstract No. 103228 (Zhurnal Obshchei Khimii, 1980, 50, 2106-9).

[7] ¹H NMR: (DMSOd₆) 2.18 (s, 3H), 3.69 (s, 3H), 3.82 (br s, 5H), 3.95 (s, 3H), 6.56 (d, 1H), 7.32 (m, 1H), 7.43 (d, 1H), 7.45 (s, 1H), 7.5 (d, 1H), 8.09 (d, 1H), 8.23 (d, 1H), 8.65 (d, 1H), 9.4 (s, 1H); Mass Spectrum: M+H⁺ 434.

The 4-amino-1,5-dimethyl-1H-pyrazole used as a starting material was prepared as follows:—

Under an atmosphere of argon, diisopropylethylamine (3.49 ml) and diphenylphosphoryl azide (2.37 ml) were added in turn to a stirred mixture of 1,5-dimethyl-1H-pyrazole-4-carboxylic acid (1.4 g), tert-butanol (4 ml) and 1,4-dioxane (40 ml) and the reaction mixture was stirred at ambient temperature for 10 minutes. The resultant mixture was heated to 110° C. for 3 hours. The solvent was evaporated and the reaction product was purified by column chromatography on silica using ethyl acetate as the eluent. There was thus obtained 4-(tert-butoxycarbonylamino)-1,5-dimethyl-1H-pyrazole (0.225 g); ¹H NMR: (DMSOd₆) 1.4 (s, 9H), 2.2 (s, 3H), 3.55 (s, 1H), 6.0 (br s, 1H), 9.3 (br s, 1H).

A mixture of the material so obtained, a 4M solution of hydrogen chloride in 1,4-dioxane (0.96 ml) and methylene chloride (5 ml) was stirred at ambient temperature for 3 days. The resultant solid was collected by filtration, washed with diethyl ether and dried under vacuum. There was thus obtained 4-amino-1,5-dimethyl-1H-pyrazole (0.078 g), as a hydrochloride salt, ¹H NMR: (DMSOd₆) 2.25 (s, 3H), 3.65 (s, 3H), 5.85 (s, 1H).

The 1,5-dimethyl-1H-pyrazole-4-carboxylic acid used as a starting material was obtainable commercially. The compound may also be prepared according to the procedure disclosed in Australian Journal of Chemistry, 1983, 36, 135-147.

[8] ¹H NMR: (DMSOd₆) 1.33 (t, 3H), 3.8 (s, 2H), 3.82 (s, 3H), 4.07 (q, 2H), 6.76 (d, 1H), 7.61 (s, 1H), 7.54 (d, 1H), 7.74-7.82 (m, 1H), 7.87 (s, 1H), 8.01 (m, 1H), 8.10-8.18 (m, 2H), 8.73 (d, 1H), 10.1 (s, 1H); Mass Spectrum: M+H⁺ 422.

The 2-[5-(6-fluoroquinolin-4-yloxy)-3-methoxypyridin-2-yl]acetic acid used as a starting material was prepared as follows:—

Under an atmosphere of argon, a mixture of 4-chloro-6-fluoroquinoline (U.S. Pat. No. 4,560,692, within example 12 thereof; 0.7 g), methyl 2-(5-hydroxy-3-methoxypyridin-2-yl)acetate (0.759 g), 4-dimethylaminopyridine (1.42 g) and chlorobenzene (9 ml) was stirred and heated to 135° C. for 12 hours. The resultant mixture was cooled to ambient temperature. Ethyl acetate was added and the mixture was filtered. The filtrate was evaporated and the residue was purified by column chromatography on silica using a solvent gradient of 3:2 to 0:10 of methylene chloride and ethyl acetate as eluent. There was thus obtained methyl 2-[5-(6-fluoroquinolin-4-yloxy)-3-methoxypyridin-2-yl]acetate (1.03 g); ¹H NMR: (DMSOd₆) 3.64 (s, 3H), 3.83 (s, 2H), 3.84 (s, 3H), 6.78 (d, 1H), 7.57 (d, 1H), 7.77 (m, 1H), 8.01 (m, 1H), 8.13 (m, 2H), 8.72 (d, 1H); Mass Spectrum: M+H⁺ 343.

A mixture of the material so obtained, methanol (5 ml) and a solution of sodium hydroxide (0.241 g) in water (4 ml) was stirred at ambient temperature for 4 hours. The methanol was evaporated and the aqueous phase was extracted with ethyl acetate. The aqueous phase was acidified to pH4.5 by the dropwise addition of 2N aqueous hydrochloric acid. The resultant precipitate was isolated, washed in turn with water and diethyl ether and dried under vacuum. There was thus obtained the required starting material (0.815 g); ¹H NMR: (DMSOd₆) 3.74 (s, 2H), 3.83 (s, 3H), 6.76 (d, 1H), 7.54 (d, 1H), 7.77 (m, 1H), 8.0 (m, 1H), 8.13 (m, 2H), 8.72 (d, 1H); Mass Spectrum: M+H⁺ 329.

[9] ¹H NMR: (DMSOd₆) 1.31 (t, 3H), 2.85 (d, 3H), 3.8 (s, 2H), 3.82 (s, 3H), 4.01 (s, 3H), 4.07 (q, 2H), 6.72 (d, 1H), 7.41 (s, 1H), 7.53 (d, 1H), 7.67 (s, 1H), 7.97 (s, 1H), 8.12 (d, 1H), 8.23 (s, 1H), 8.4 (q, 1H), 8.63 (s, 1H), 10.1 (s, 1H); Mass Spectrum: M+H⁺ 491.

The 2-{3-methoxy-5-[6-methoxy-7-(N-methylcarbamoyl)quinolin-4-yloxy]pyridin-2-yl}acetic acid used as a starting material was prepared as follows:—

A mixture of methyl 2-methoxy-5-nitrobenzoate (20.3 g), 5% platinum-on-carbon catalyst (1.5 g) and ethyl acetate (300 ml) was stirred under 1.4 atmospheres pressure of hydrogen for 5 hours. The catalyst was removed by filtration and the filtrate was evaporated. There was thus obtained methyl 5-amino-2-methoxybenzoate (17 g); ¹H NMR: (CDCl₃) 3.84 (s, 3H), 3.89 (s, 3H), 6.86 (m, 2H), 7.19 (m, 1H); Mass Spectrum: M+H⁺ 182.

A mixture of methyl 5-amino-2-methoxybenzoate (17 g; see also Canadian Journal of Chemistry, 1973, 51, 162-170), 5-methoxymethylene-2,2-dimethyl-1,3-dioxane-4,6-dione (17.5 g) and isopropanol (200 ml) was stirred at ambient temperature for 10 minutes. A yellow precipitate formed which was isolated by filtration, washed in turn with isopropanol and diethyl ether and dried under vacuum. There was thus obtained 5-(4-methoxy-3-methoxycarbonylanilinomethylene)-2,2-dimethyl-1,3-dioxane-4,6-dione (28.9 g); ¹H NMR: (CDCl₃) 1.76 (s, 6H), 3.93 (s, 3H), 3.95 (s, 3H), 7.05 (d, 1H), 7.35 (m, 1H), 7.74 (d, 1H), 8.56 (d, 1H); Mass Spectrum: M+H⁺ 336.

5-(4-Methoxy-3-methoxycarbonylanilinomethylene)-2,2-dimethyl-1,3-dioxane-4,6-dione (28.9 g) was added portionwise to a mixture (200 ml) of biphenyl and diphenyl ether (‘Dowtherm A’) that had been warmed to 260° C. The solution was stirred at that temperature for 5 minutes. The resultant mixture was cooled to ambient temperature and added to a mixture of petroleum ether (250 ml) and diethyl ether (250 ml). The precipitate was collected by filtration and washed with petroleum ether. The material so obtained was purified by column chromatography on silica using increasingly polar solvent mixtures of methylene chloride and methanol (from 10:0 to 17:3) as eluent. There was thus obtained a 7:3 mixture (11.7 g) of 6-methoxy-7-methoxycarbonyl-1,4-dihydroquinolin-4-one and 6-methoxy-5-methoxycarbonyl-1,4-dihydroquinolin-4-one; ¹H NMR: (DMSOd₆) 3.85 (s, 3H), 3.88 (s, 3H), 6.05 (d, 1H), 7.61 (s, 1H), 7.87 (s, 1H), 7.94 (d, 1H) and 3.75 (s, 3H), 3.82 (s, 3H), 5.92 (d, 1H), 7.55 (d, 1H), 7.63 (d, 1H), 7.88 (m, 1H); Mass Spectrum: M+H⁺ 234.

A portion (9.41 g) of the mixture of quinolin-4-ones so obtained was dissolved in methanol (100 ml). Lithium hydroxide (5.04 g) was added and the mixture was stirred at ambient temperature for 16 hours. The solvent was evaporated and water (100 ml) was added to the residue. The mixture was neutralised to pH7 by the addition of 6N aqueous hydrochloric acid. The aqueous solution was extracted with methylene chloride and with ethyl acetate. The aqueous solution was acidified to pH2 by the addition of 6N aqueous hydrochloric acid. The resultant precipitate was isolated, washed with water and with diethyl ether and dried under vacuum. There was thus obtained 7-carboxy-6-methoxy-1,4-dihydroquinolin-4-one (6.1 g); ¹H NMR: (DMSOd₆) 3.88 (s, 3H), 6.04 (d, 1H), 7.59 (s, 1H), 7.83 (s, 1H), 7.93 (d, 1H), 11.85 (br s, 1H), 13.16 (br s, 1H); Mass Spectrum: M+H⁺ 220.

A mixture of a portion (2 g) of the material so obtained and phosphorous oxychloride (4.17 ml) was heated to 105° C. for 1 hour. The resultant mixture was cooled to ambient temperature and diluted with methylene chloride (50 ml). The solution so obtained was slowly poured into a 2M solution of methylamine in THF (100 ml) that was cooled in an ice-bath. The mixture was stirred and allowed to warm to ambient temperature. The reaction mixture was washed in turn with a saturated aqueous sodium bicarbonate solution and brine, dried over magnesium sulphate and evaporated. The residue was triturated under diethyl ether. The resultant solid was isolated, washed with diethyl ether and dried under vacuum. There was thus obtained 4-chloro-6-methoxy-7-(N-methylcarbamoyl)quinoline (1.54 g); ¹H NMR: (DMSOd₆) 2.84 (d, 3H), 4.03 (s, 3H), 7.51 (s, 1H), 7.78 (d, 1H), 8.23 (s, 1H), 8.4 (br d, 1H), 8.74 (d, 1H); Mass Spectrum: M+H⁺ 252 and 254.

Under an atmosphere of argon, a mixture of 4-chloro-6-methoxy-7-(N-methylcarbamoyl)quinoline (1.19 g), methyl 2-(5-hydroxy-3-methoxypyridin-2-yl)acetate (1.12 g), caesium carbonate (1.85 g) and DMF (10 ml) was stirred and heated to 120° C. for 6 hours. The mixture was cooled to ambient temperature, diluted with water and extracted with ethyl acetate. The organic phase was evaporated and the residue was purified by column chromatography on silica using a solvent gradient of 1:1:0 to 9:9:1 of methylene chloride ethyl acetate and methanol as eluent. There was thus obtained methyl 2-{3-methoxy-5-[6-methoxy-7-(N-methylcarbamoyl)quinolin-4-yloxy]pyridin-2-yl}acetate (1.05 g); ¹H NMR: (CDCl₃) 3.09 (d, 3H), 3.76 (s, 3H), 3.85 (s, 3H), 3.94 (s, 2H), 4.12 (s, 3H), 6.64 (d, 1H), 7.04 (d, 1H), 7.67 (s, 1H), 7.81 (br d, 1H), 8.13 (d, 1H), 8.66 (d, 1H), 8.97 (s, 1H); Mass Spectrum: M+H⁺ 412.

A mixture of the material so obtained, methanol (15 ml) and a 2N aqueous solution of sodium hydroxide (3.65 ml) was stirred at ambient temperature for 1 hour. The mixture was acidified to pH3 by the addition of 1N aqueous hydrochloric acid. The resultant precipitate was isolated, washed in turn with water, ethyl acetate and diethyl ether and dried under vacuum. There was thus obtained 2-{3-methoxy-5-[6-methoxy-7-(N-methylcarbamoyl)quinolin-4-yloxy]pyridin-2-yl}acetic acid (0.78 g); ¹H NMR: (DMSOd₆) 2.84 (d, 3H), 3.74 (s, 2H), 3.83 (s, 3H), 4.01 (s, 3H), 6.73 (d, 1H), 7.53 (d, 1H), 7.66 (s, 1H), 8.11 (d, 1H), 8.23 (s, 1H), 8.39 (br d, 1H), 8.63 (d, 1H); Mass Spectrum: M+H⁺ 398.

[10] The reaction mixture was heated to 60° C. for 24 hours. The product gave the following characterising data:—¹H NMR: (DMSOd₆) 1.16 (s, 3H), 2.56 (q, 2H), 3.81 (s, 3H), 3.83 (s, 2H), 3.95 (s, 3H), 6.27 (s, 1H), 6.47 (d, 1H), 7.32 (m, 1H), 7.43 (d, 1H), 7.5 (d, 1H), 8.08 (d, 1H), 8.23 (d, 1H), 8.65 (d, 1H), 10.4 (s, 1H), 11.98 (s, 1H); Mass Spectrum: M+H⁺ 434.

[11] The reaction mixture was heated to 70° C. for 12 hours. The product gave the following characterising data:—¹H NMR: (DMSOd₆) 1.16 (t, 3H), 2.56 (q, 2H), 3.81 (s, 3H) 3.84 (s, 2H), 6.26 (s, 1H), 6.76 (d, 1H), 7.53 (d, 1H), 7.74-7.82 (m, 1H), 8.11 (d, 1H), 8.14 (m, 1H), 8.73 (d, 1H), 10.41 (s, 1H), 11.98 (br s, 1H); Mass Spectrum: M+H⁺ 422.

[12] ¹H NMR: (DMSOd₆) 1.77 (s, 3H), 2.1 (s, 3H), 3.82 (s, 2H), 3.83 (s, 3H), 3.94 (s, 3H), 3.96 (s, 3H), 6.59 (d, 1H), 7.42 (s, 1H), 7.48 (d, 1H), 7.53 (s, 1H), 8.08 (d, 1H), 8.52 (d, 1H), 9.63 (br s, 1H), 12.0 (br s, 1H); Mass Spectrum: M+H⁺ 464.

The 2-[5-(6,7-dimethoxyquinolin-4-yloxy)-3-methoxypyridin-2-yl]acetic acid used as a starting material was prepared as follows:—

Using analogous procedures to those described in the last two paragraphs of the portion of Note [5] hereinbefore that is concerned with the preparation of starting materials, 4-chloro-6,7-dimethoxyquinoline was reacted with methyl 2-(5-hydroxy-3-methoxypyridin-2-yl)acetate to give methyl 2-[5-(6,7-dimethoxyquinolin-4-yloxy)-3-methoxypyridin-2-yl]acetate, which compound was hydrolysed with 2N aqueous sodium hydroxide solution to give the required starting material.

The 3-amino-4,5-dimethyl-1H-pyrazole used as a starting material is described in UK Patent Specification No. 788,140 (within Example 1 thereof).

[13] ¹H NMR: (DMSOd₆) 1.2 (t, 3H), 2.72 (q, 2H), 3.81 (s, 3H), 3.89 (s, 2H), 3.95 (s, 3H), 6.55 (d, 1H), 6.62 (s, 1H), 7.32 (m, 1H), 7.43 (d, 1H), 7.52 (d, 1H), 8.09 (d, 1H), 8.22 (d, 1H), 8.65 (d, 1H), 11.12 (s, 1H); Mass Spectrum: M+H⁺ 435.

[14] ¹H NMR: (DMSOd₆) 1.82 (s, 3H), 2.3 (s, 3H), 3.83 (s, 3H), 3.89 (s, 2H), 3.95 (s, 3H), 6.58 (d, 1H), 7.32 (m, 1H), 7.43 (d, 1H), 7.52 (d, 1H), 8.09 (d, 1H), 8.22 (d, 1H), 8.65 (d, 1H), 10.4 (br s, 1H); Mass Spectrum: M+H⁺ 435.

The 3-amino-4,5-dimethylisoxazole used as a starting material is described in Tetrahedron Letters, 1996, 37, 3339-3342.

[15] ¹H NMR: (DMSOd₆) 1.2 (t, 3H), 2.73 (q, 2H), 3.82 (s, 3H), 3.9 (s, 2H), 6.62 (s, 1H), 6.76 (d, 1H), 7.55 (d, 1H), 7.75-7.81 (m, 1H), 8.01 (m, 1H), 8.12 (d, 1H), 8.14 (m, 1H), 8.73 (d, 1H), 11.12 (s, 1H); Mass Spectrum: M+H⁺ 423.

[16] The product was recovered from the reaction mixture by filtration. The material so obtained was suspended in a dilute aqueous sodium bicarbonate solution and stirred for 30 minutes. The solid was isolated by filtration, washed with water and dried under vacuum. The product so obtained gave the following characterising data:—¹H NMR: (DMSOd₆) 1.82 (s, 3H), 2.3 (s, 3H), 3.84 (s, 3H), 3.9 (s, 2H), 6.78 (s, 1H), 7.55 (d, 1H), 7.73-7.82 (m, 1H), 8.01 (m, 1H), 8.1-8.18 (m, 2H), 8.73 (d, 1H), 10.35 (br s, 1H); Mass Spectrum: M+H⁺ 423.

[17] ¹H NMR: (DMSOd₆) 1.81 (s, 3H), 2.3 (s, 3H), 2.85 (d, 3H), 3.84 (s, 3H), 3.9 (s, 2H), 4.01 (s, 3H), 6.75 (d, 1H), 7.54 (d, 1H), 7.67 (s, 1H), 8.12 (d, 1H), 8.33 (s, 1H), 8.4 (q, 1H), 8.64 (s, 1H), 10.35 (s, 1H); Mass Spectrum: M+H⁺ 492.

[18] DMA was used in place of DMF as the reaction solvent. The reaction mixture was stirred at ambient temperature for 14 hours and subsequently heated to 70° C. for 2 hours. The product gave the following characterising data:—¹H NMR: (DMSOd₆) 1.82 (s, 3H), 2.3 (s, 3H), 3.83 (s, 3H), 3.89 (s, 2H), 3.94 (s, 3H), 3.96 (s, 3H), 6.58 (d, 1H), 7.42 (s, 1H), 7.5 (d, 1H), 7.52 (s, 1H), 8.09 (d, 1H), 8.52 (d, 1H), 10.34 (br s, 1H); Mass Spectrum: M+H⁺ 465.

[19] The product was recovered from the reaction mixture by filtration, washed in turn with DMF, ethyl acetate and diethyl ether and dried under vacuum. The product so obtained gave the following characterising data:—¹H NMR: (DMSOd₆) 2.27 (d, 3H), 3.82 (s, 3H), 3.94 (s, 3H), 3.95 (s, 2H), 6.57 (d, 1H), 6.74 (q, 1H), 7.32 (m, 1H), 7.43 (d, 1H), 7.53 (d, 1H), 8.09 (d, 1H), 8.22 (d, 1H), 8.65 (s, 1H), 12.20 (br s, 1H); Mass Spectrum: M+H⁺ 437.

[20] ¹H NMR: (DMSOd₆) 2.13 (s, 6H), 2.25 (s, 3H), 3.29 (s, 2H), 3.82 (s, 3H), 3.85 (s, 2H), 3.95 (s, 3H), 6.55 (d, 1H), 6.78 (s, 1H), 7.32 (m, 1H), 7.34 (s, 1H), 7.37 (s, 1H), 7.44 (d, 1H), 7.52 (d, 1H), 8.09 (d, 1H), 8.23 (d, 1H), 8.65 (d, 1H), 10.05 (s, 1H); Mass Spectrum: M+H⁺ 487.

The 3-dimethylaminomethyl-5-methylaniline used as a starting material was prepared as follows:—

A mixture of 1,3-dimethyl-5-nitrobenzene (15.15 g), N-bromosuccinimide (2 g), benzoyl peroxide (0.484 g) and carbon tetrachloride (250 ml) was stirred and heated to reflux. Further portions of N-bromosuccinimide (totaling 21 g) were added portionwise during 4 hours to the heated reaction mixture. The mixture was cooled to ambient temperature. Petroleum ether (b.p. 60-80° C.) was added. The mixture was filtered and the filtrate was evaporated to give an oil (25 g) which was shown by NMR analysis to be a mixture of 3-methyl-5-nitrobenzyl bromide (76%), unreacted starting material (˜19%) and 3-bromomethyl-5-nitrobenzyl bromide (˜15%). This mixture was used in the next step.

A portion (2.3 g) of the oil so obtained was dissolved in ethanol (5 ml) and dimethylamine (6 equivalents) was added portionwise in order to prevent a significant exotherm. The resultant reaction mixture was stirred at ambient temperature for 12 hours. The mixture was evaporated and the residue was purified by column chromatography on silica using increasingly polar mixtures of methylene chloride and diethyl ether as eluent. There was thus obtained N,N-dimethyl-N-(3-methyl-5-nitrobenzyl)amine (0.98 g); ¹H NMR: (DMSOd₆) 2.17 (s, 6H), 2.43 (s, 3H), 3.48 (s, 2H), 7.58 (s, 1H), 7.94 (m, 2H); Mass Spectrum: M+H⁺ 195.

A mixture of N,N-dimethyl-N-(3-methyl-5-nitrobenzyl)amine (2.2 g), platinum oxide (0.11 g) and ethyl acetate (40 ml) was stirred under 1.8 atmospheres pressure of hydrogen for 30 minutes. The catalyst was removed by filtration and the filtrate was evaporated. The material so obtained was dried under vacuum at ambient temperature for 2 hours. There was thus obtained 3-dimethylaminomethyl-5-methylaniline as a solid (1.7 g); ¹H NMR: (DMSOd₆) 2.09 (s, 6H), 2.12 (s, 3H), 3.16 (s, 2H), 4.87 (s, 2H), 6.24 (s, 2H), 6.31 (s, 1H).

[21] ¹H NMR: (DMSOd₆) 2.13 (s, 6H), 2.26 (s, 3H), 3.29 (s, 2H), 3.83 (s, 3H), 3.86 (s, 2H), 6.76 (d, 1H), 6.78 (s, 1H), 7.35 (s, 1H), 7.37 (s, 1H), 7.55 (d, 1H), 7.75-7.80 (m, 1H), 8.02 (m, 1H), 8.13 (d, 1H), 8.14 (m, 1H), 8.73 (d, 1H), 10.05 (s, 1H); Mass Spectrum: M+H⁺ 475.

[22] ¹H NMR: (DMSOd₆) 2.13 (s, 6H), 2.26 (s, 3H), 2.85 (d, 3H), 3.22 (s, 2H), 3.83 (s, 3H), 3.86 (s, 2H), 4.01 (s, 3H), 6.71 (d, 1H), 6.78 (s, 1H), 7.35 (s, 1H), 7.37 (s, 1H), 7.54 (d, 1H), 7.67 (s, 1H), 8.13 (s, 1H), 8.23 (s, 1H), 8.4 (q, 1H), 8.64 (s, 1H), 10.06 (s, 1H); Mass Spectrum: M+H⁺ 544.

[23] The reaction mixture was heated to 45° C. for 4 hours. The product was purified by column chromatography on silica using a solvent gradient from methylene chloride to a 23:2 mixture of methylene chloride and methanol as eluent. The product gave the following characterising data:—¹H NMR: (DMSOd₆) 1.32 (t, 3H), 3.75 (s, 2H), 3.79 (s, 3H), 3.83 (s, 3H), 3.91 (s, 3H), 4.06 (q, 2H), 6.58 (d, 1H), 6.67 (d, 1H), 7.02 (d, 1H), 7.32 (d, 1H), 7.4 (s, 1H), 7.85 (s, 1H), 7.86 (d, 1H), 8.58 (d, 1H), 10.05 (s, 1H); Mass Spectrum: M+H⁺ 464.

The 2-[5-(5,7-dimethoxyquinolin-4-yloxy)-3-methoxypyridin-2-yl]acetic acid used as a starting material was prepared as follows:—

Diisopropylethylamine (2.63 ml) was added to a stirred mixture of 5,7-dimethoxy-1,4-dihydroquinolin-4-one (Med. Chem. Letters, 1997, 789-792 and Arch. Pharm. Res., 1998, 445-451; 2.05 g) and chlorobenzene (25 ml). Under an atmosphere of nitrogen, the mixture was cooled to 15° C. and a solution of phosphorous oxychloride (1.29 ml) in chlorobenzene (5 ml) was added dropwise. The resultant solution was stirred and heated to 65° C. for 3 hours. The mixture was cooled in an ice bath and an aqueous solution of sodium bicarbonate (5 g) was added. The resultant mixture was extracted with diethyl ether. The organic phase was washed with brine, dried over magnesium sulphate and evaporated. There was thus obtained 4-chloro-5,7-dimethoxyquinoline (2.15 g); ¹H NMR: (DMSOd₆) 3.91 (s, 3H), 3.92 (s, 3H), 6.76 (d, 1H), 7.05 (d, 1H), 7.39 (d, 1H), 8.61 (d, 1H); Mass Spectrum: M+H⁺224.

A mixture of 4-chloro-5,7-dimethoxyquinoline (1.2 g), methyl 2-(5-hydroxy-3-methoxypyridin-2-yl)acetate (1.1 g), pyridine (2.16 ml) and chlorobenzene (35 ml) was stirred and heated to reflux for 2 hours. The mixture was cooled and concentrated by evaporation. Water was added to the residue and sodium bicarbonate was added until gas evolution ceased. The mixture was extracted with a 1:1 mixture of diethyl ether and ethyl acetate and the organic phase was dried over magnesium sulphate and evaporated. The residue was purified by column chromatography on silica using a gradient from methylene chloride to a 24:1 of methylene chloride and methanol as eluent. There was thus obtained methyl-2-[5-(5,7-dimethoxyquinolin-4-yloxy)-3-methoxypyridin-2-yl]acetate (1.8 g); ¹H NMR: (DMSOd₆) 3.62 (s, 3H), 3.79 (s, 3H), 3.8 (s, 2H), 3.81 (s, 3H), 3.91 (s, 3H), 6.62 (d, 1H), 6.67 (d, 1H), 7.03 (d, 1H), 7.33 (d, 1H), 7.85 (d, 1H), 8.59 (d, 1H); Mass Spectrum: M+H⁺ 385.

A mixture of the material so obtained, lithium hydroxide monohydrate (0.287 g), water (17.5 ml) and THF (17.5 ml) was stirred at ambient temperature for 1 hour. The solution was acidified to pH5 by the addition of an aqueous citric acid solution. The precipitate was isolated, washed successively with water, ethanol and diethyl ether and dried under vacuum. There was thus obtained 2-[5-(5,7-dimethoxyquinolin-4-yloxy)-3-methoxypyridin-2-yl]acetic acid (1.63 g); ¹H NMR: (DMSOd₆) 3.69 (s, 2H), 3.8 (s, 3H), 3.82 (s, 3H), 3.91 (s, 3H), 6.6 (d, 1H), 6.67 (s, 1H), 7.03 (s, 1H), 7.32 (s, 1H), 7.85 (s, 1H), 8.59 (d, 1H); Mass Spectrum: M+H⁺ 371.

[24] The reaction mixture was heated to 45° C. for 4 hours. The product was purified by column chromatography on silica using a solvent gradient from methylene chloride to a 23:2 mixture of methylene chloride and methanol as eluent. The product gave the following characterising data:—¹H NMR: (DMSOd₆) 2.12 (s, 3H), 3.69 (s, 3H), 3.79 (s, 3H), 3.81 (s, 2H), 3.83 (s, 3H), 3.91 (s, 3H), 6.58 (d, 1H), 6.67 (d, 1H), 7.02 (d, 1H), 7.31 (d, 1H), 7.79 (s, 1H), 7.86 (d, 1H), 8.58 (d, 1H), 9.48 (br s, 1H); Mass Spectrum: M+H⁺ 464.

[25] DMA was used in place of DMF as the reaction solvent. The product gave the following characterising data:—¹H NMR: (DMSOd₆) 1.37 (d, 6H), 3.8 (s, 2H), 3.82 (s, 3H), 4.44 (m, 1H), 6.75 (d, 1H), 7.41 (s, 1H), 7.54 (s, 1H), 7.76 (m, 1H), 7.87 (s, 1H), 8.02 (m, 1H), 8.13 (m, 2H), 8.72 (d, 1H), 10.1 (s, 1H); Mass Spectrum: M+H⁺ 436.

The 4-amino-1-isopropyl-1H-pyrazole used as a starting material was prepared as follows:—

A mixture of 4-nitropyrazole (1.13 g), isopropyl iodide (1 ml), potassium carbonate (1.38 g) and DMF (30 ml) was stirred and heated to 70° C. for 2 hours. The resultant mixture was poured into water and the precipitate was isolated, washed with water and dried under vacuum. There was thus obtained 1-isopropyl-4-nitro-1H-pyrazole (0.845 g); ¹H NMR: (DMSOd₆) 1.44 (d, 6H), 4.59 (m, 1H), 8.26 (s, 1H), 8.93 (s, 1H).

A mixture of a portion (0.8 g) of the material so obtained, platinum oxide (0.1 g), ethyl acetate (10 ml) and ethanol (30 ml) was, stirred under 3 atmospheres pressure of hydrogen for 2 hours. The catalyst was removed by filtration and the filtrate was evaporated. There was thus obtained the required starting material as a colourless oil (0.607 g); ¹H NMR: (DMSOd₆) 1.31 (d, 6H), 3.76 (br s, 2H), 4.27 (m, 1H), 6.88 (s, 1H), 7.03 (s, 1H).

[26] DMA was used in place of DMF as the reaction solvent. The product gave the following characterising data:—¹H NMR: (DMSOd₆) 3.78 (s, 3H), 3.79 (s, 2H), 3.81 (s, 3H), 3.94 (s, 3H), 3.96 (s, 3H), 6.56 (d, 1H), 7.4 (s, 1H), 7.42 (s, 1H), 7.49 (d, 1H), 7.53 (s, 1H), 7.83 (s, 1H), 8.08 (d, 1H), 8.52 (d, 1H), 10.09 (s, 1H), 12.0 (br s, 1H); Mass Spectrum: M+H⁺ 450.

The 4-amino-1-methyl-1H-pyrazole used as a starting material was prepared as follows:—

Dimethyl sulphate (5 ml) was slowly added to a stirred solution of 4-nitropyrazole (2 g) in 1N aqueous sodium hydroxide solution (20 ml) that had been warmed to 30° C. and the resultant mixture was stirred at that temperature for 48 hours. The mixture was cooled to ambient temperature and the precipitate was isolated, washed with cold water and dried under vacuum. There was thus obtained 1-methyl-4-nitro-1H-pyrazole (1.5 g); ¹H NMR: (DMSOd₆) 3.91 (s, 1H), 8.24 (s, 1H), 8.85 (s, 1H).

A mixture of a portion (0.7 g) of the material so obtained, platinum oxide (0.05 g), ethyl acetate (5 ml) and ethanol (15 ml) was stirred under 3 atmospheres pressure of hydrogen for 2 hours. The catalyst was removed by filtration and the filtrate was evaporated. There was thus obtained the required starting material (0.6 g); ¹H NMR: (DMSOd₆) 3.64 (s, 3H), 6.86 (s, 1H), 6.97 (s, 1H).

[27] The product was purified by column chromatography on silica using increasingly polar mixtures of methylene chloride and methanol as eluent. The product gave the following characterising data:—¹H NMR: (DMSOd₆) 2.13 (s, 3H), 3.69 (s, 3H), 3.82 (s, 3H), 3.85 (s, 2H), 3.94 (s, 3H), 3.96 (s, 3H), 6.56 (d, 1H), 7.42 (s, 1H), 7.48 (d, 1H), 7.53 (s, 1H), 7.81 (s, 1H), 8.08 (d, 1H), 8.52 (d, 1H), 9.51 (s, 1H); Mass Spectrum: M+H⁺ 464.

[28] DMA was used in place of DMF as the reaction solvent. The product gave the following characterising data:—¹H NMR: (DMSOd₆) 1.09 (t, 3H), 2.14 (s, 3H), 3.82 (s, 3H), 3.85 (s, 2H), 3.94 (s, 3H), 3.96 (s, 3H), 3.99 (q, 2H), 6.54 (d, 1H), 7.42 (s, 1H), 7.48 (d, 1H), 7.52 (s, 1H), 7.84 (s, 1H), 8.08 (d, 1H), 8.51 (d, 1H), 9.51 (s, 1H); Mass Spectrum: M+H⁺ 478.

[29] DMA was used in place of DMF as the reaction solvent. The product gave the following characterising data:—¹H NMR: (DMSOd₆) 1.33 (t, 3H), 3.8 (s, 2H), 3.82 (s, 3H), 3.94 (s, 3H), 4.07 (q, 2H), 6.67 (d, 1H), 7.41 (s, 1H), 7.48 (m, 1H), 7.51 (s, 1H), 7.87 (s, 1H), 7.97 (d, 1H), 8.1 (d, 1H), 8.58 (d, 1H), 10.09 (s, 1H); Mass Spectrum: M+H⁺ 434.

The 2-[3-methoxy-5-(6-methoxyquinolin-4-yloxy)pyridin-2-yl]acetic acid used as a starting material was prepared as follows:—

Under an atmosphere of argon, a mixture of 4-chloro-6-methoxyquinoline (International Application WO 2006/021448, within Example 48 thereof; 0.5 g), methyl 2-(5-hydroxy-3-methoxypyridin-2-yl)acetate (0.509 g), 4-dimethylaminopyridine (0.947 g) and chlorobenzene (5 ml) was stirred and heated to 140° C. for 16 hours. The mixture was cooled to ambient temperature and concentrated by evaporation. The residue was purified by column chromatography on silica using ethyl acetate as eluent. There was thus obtained methyl 2-[3-methoxy-5-(6-methoxyquinolin-4-yloxy)pyridin-2-yl]acetate (0.47 g); ¹H NMR: (DMSOd₆) 3.75 (s, 3H), 3.84 (s, 3H), 3.93 (s, 2H), 3.97 (s, 3H), 6.62 (d, 1H), 7.03 (d, 1H), 7.43 (m, 1H), 7.55 (d, 1H), 8.03 (d, 1H), 8.12 (d, 1H), 8.58 (d, 1H); Mass Spectrum: M+H⁺ 355.

A mixture of the material so obtained, sodium hydroxide (0.159 g), water (3 ml), THF (5 ml) and methanol (8 ml) was stirred at ambient temperature for 14 hours. The resultant mixture was concentrated by evaporation and partitioned between water and ethyl acetate. The aqueous phase was acidified to pH5 by the addition of 6N aqueous hydrochloric acid. The resultant precipitate was isolated and dried under vacuum. There was thus obtained 2-[3-methoxy-5-(6-methoxyquinolin-4-yloxy)pyridin-2-yl]acetic acid (0.363 g); ¹H NMR: (DMSOd₆) 3.74 (s, 2H), 3.83 (s, 3H), 3.94 (s, 3H), 6.68 (d, 1H), 7.48 (m, 1H), 7.58 (d, 1H), 7.97 (d, 1H), 8.09 (d, 1H), 8.58 (d, 1H); Mass Spectrum: M+H⁺ 341.

[30] ¹H NMR: (DMSOd₆) 1.37 (d, 6H), 3.8 (s, 2H), 3.82 (s, 3H), 3.94 (s, 3H), 4.43 (m, 1H), 6.67 (d, 1H), 7.41 (s, 1H), 7.48 (m, 1H), 7.51 (d, 1H), 7.58 (d, 1H), 7.87 (s, 1H), 7.97 (d, 1H), 8.1 (d, 1H), 8.58 (d, 1H), 10.09 (s, 1H); Mass Spectrum: M+H⁺ 448.

[31] DMA was used in place of DMF as the reaction solvent. The product gave the following characterising data:—¹H NMR: (DMSOd₆) 2.13 (s, 3H), 3.7 (s, 3H), 3.82 (s, 3H), 3.85 (s, 2H), 3.94 (s, 3H), 6.67 (d, 1H), 7.47 (m, 1H), 7.51 (s, 1H), 7.59 (s, 1H), 7.81 (d, 1H), 7.97 (d, 1H), 8.1 (s, 1H), 8.58 (d, 1H), 9.51 (br s, 1H); Mass Spectrum: M+H⁺ 434.

[32] DMA was used in place of DMF as the reaction solvent. The product gave the following characterising data:—¹H NMR: (DMSOd₆) 1.33 (t, 3H), 1.43 (t, 3H), 3.79 (s, 2H), 3.82 (s, 3H), 3.94 (s, 3H), 4.07 (q, 2H), 4.21 (q, 2H), 6.54 (d, 1H), 7.4 (d, 1H), 7.48 (d, 1H), 7.52 (s, 1H), 7.87 (s, 1H), 8.08 (d, 1H), 8.51 (d, 1H), 10.09 (s, 1H); Mass Spectrum: M+H⁺478.

The 2-[5-(7-ethoxy-6-methoxyquinolin-4-yloxy)-3-methoxypyridin-2-yl]acetic acid used as a starting material was prepared as follows:—

1,1′-(Azodicarbonyl)dipiperidine (2.24 g) was added portionwise to a stirred suspension of 4-chloro-7-hydroxy-6-methoxyquinoline (International Application WO 98/13350, within example 3 thereof; 1.6 g), ethanol (0.868 ml), tributylphosphine (2.9 ml) and methylene chloride (50 ml) and the resultant mixture was stirred at ambient temperature for 16 hours. The mixture was filtered and the filtrate was concentrated by evaporation. The residue was purified by column chromatography on silica using a solvent gradient from methylene chloride to a 1:1 mixture of methylene chloride and ethyl acetate as eluent. There was thus obtained 4-chloro-7-ethoxy-6-methoxyquinoline (1.2 g); ¹H NMR: (CDCl₃) 1.57 (t, 3H), 4.06 (s, 3H), 4.28 (q, 2H), 7.35 (d, 1H), 7.41 (d, 1H), 8.57 (d, 1H); Mass Spectrum: M+H⁺ 238.

Under an atmosphere of argon, a mixture of 4-chloro-7-ethoxy-6-methoxyquinoline (0.5 g), methyl 2-(5-hydroxy-3-methoxypyridin-2-yl)acetate (0.456 g), 4-dimethylaminopyridine (0.771 g) and chlorobenzene (8 ml) was stirred and heated to 145° C. for 16 hours. The mixture was cooled to ambient temperature and concentrated by evaporation. The residue was purified by column chromatography on silica using a solvent gradient from ethyl acetate to a 19:1 mixture of ethyl acetate and methanol as eluent. There was thus obtained methyl 2-[5-(7-ethoxy-6-methoxyquinolin-4-yloxy)-3-methoxypyridin-2-yl]acetate (0.626 g); 1H NMR: (DMSOd₆) 1.43 (t, 3H), 3.64 (s, 3H), 3.82 (s, 3H), 3.83 (s, 2H), 3.94 (s, 3H), 4.21 (q, 2H), 6.56 (d, 1H), 7.3 (d, 1H), 7.52 (m, 2H), 8.08 (s, 1H), 8.5 (d, 1H); Mass Spectrum: M+H⁺ 399.

A mixture of the material so obtained, sodium hydroxide (0.249 g), water (3 ml), THF (2 ml) and methanol (8 ml) was stirred at ambient temperature for 14 hours. The resultant mixture was concentrated by evaporation and partitioned between water and ethyl acetate. The aqueous phase was acidified to pH4.5 by the addition of 2N aqueous hydrochloric acid. The resultant precipitate was isolated and dried under vacuum. There was thus obtained 2-[5-(7-ethoxy-6-methoxyquinolin-4-yloxy)-3-methoxypyridin-2-yl]acetic acid (0.528 g); ¹H NMR: (DMSOd₆) 1.43 (t, 3H), 3.73 (s, 2H), 3.82 (s, 3H), 3.94 (s, 3H), 4.21 (q, 2H), 6.54 (d, 1H), 7.4 (s, 1H), 7.49 (d, 1H), 7.52 (s, 1H), 8.07 (d, 1H), 8.5 (d, 1H); Mass Spectrum: M+H⁺ 385.

[33] DMA was used in place of DMF as the reaction solvent. The product gave the following characterising data:—¹H NMR: (DMSOd₆) 1.43 (t, 3H), 2.13 (s, 3H), 3.7 (s, 3H), 3.82 (s, 3H), 3.85 (s, 2H), 3.94 (s, 3H), 4.21 (q, 2H), 6.54 (d, 1H), 7.4 (d, 1H), 7.48 (d, 1H), 7.52 (s, 1H), 7.81 (s, 1H), 8.08 (d, 1H), 8.51 (d, 1H), 9.51 (s, 1H); Mass Spectrum: M+H⁺ 478.

[34] ¹H NMR: (DMSOd₆) 1.3 (t, 3H), 1.43 (t, 3H), 2.14 (s, 3H), 3.82 (s, 3H), 3.85 (s, 2H), 3.94 (s, 3H), 3.97 (q, 2H), 4.21 (q, 2H), 6.53 (d, 1H), 7.4 (s, 1H), 7.48 (d, 1H), 7.52 (s, 1H), 7.84 (s, 1H), 8.08 (d, 1H), 8.5 (d, 1H), 9.51 (s, 1H); Mass Spectrum: M+H⁺ 492.

[35] DMA was used in place of DMF as the reaction solvent. The reaction mixture was stirred at ambient temperature for 14 hours and subsequently heated to 55° C. for 25 hours. The product gave the following characterising data:—¹H NMR: (DMSOd₆) 2.08 (s, 3H), 3.6 (s, 3H), 3.84 (s, 3H), 3.88 (s, 2H), 3.94 (s, 3H), 3.96 (s, 3H), 5.97 (s, 1H) 6.57 (d, 1H), 7.42 (s, 1H), 7.5 (d, 1H), 7.52 (s, 1H), 8.09 (d, 1H), 8.52 (d, 1H), 10.04 (s, 1H); Mass Spectrum: M+H⁺ 464.

[36] DMA was used in place of DMF as the reaction solvent. The reaction mixture was stirred at ambient temperature for 14 hours and subsequently heated to 70° C. for 2 hours. The product gave the following characterising data:—¹H NMR: (DMSOd₆) 1.9 (d, 3H), 3.83 (s, 3H), 3.85 (s, 2H), 3.91 (s, 3H), 3.96 (s, 3H), 6.58 (d, 1H), 7.42 (s, 1H), 7.5 (d, 1H), 7.52 (s, 1H), 8.09 (d, 1H), 8.52 (d, 1H), 8.6 (q, 1H), 10.44 (br s, 1H); Mass Spectrum: M+H⁺ 451.

The 3-amino-4-methylisoxazole used as starting material was prepared as follows:—

Bromine (1.9 ml) was added to a solution of methacrylonitrile (3.65 ml) in methanol (6 ml) that had been cooled to 0° C. The resultant mixture was stirred and heated to 35° C. for 2 hours. The mixture was cooled to 0° C. Hydroxyurea (4.3 g) was added followed by the dropwise addition of a solution of sodium hydroxide (4.72 g) in water (5 ml). The resultant mixture was heated to reflux for 2.5 hours. The mixture was cooled to ambient temperature and partitioned between ethyl acetate and water. The organic solution was dried over magnesium sulphate and evaporated. The residue was purified by column chromatography on silica using a solvent gradient of 1:1 to 0:100 of methylene chloride and ethyl acetate as eluent. There was thus obtained the required starting material (1.11 g); ¹H NMR: (DMSOd₆) 1.81 (d, 3H), 5.43 (br s, 2H), 8.09 (d, 1H); Mass Spectrum: M+H⁺ 99.

[37] ¹H NMR: (DMSOd₆) 2.13 (s, 6H), 2.26 (s, 3H), 3.29 (s, 2H), 3.83 (s, 3H), 3.86 (s, 2H), 3.94 (s, 3H), 6.67 (d, 1H), 6.78 (s, 1H), 7.36 (d, 1H), 7.49 (m, 1H), 7.52 (d, 1H), 7.59 (d, 1H), 7.97 (d, 1H), 8.11 (d, 1H), 8.59 (d, 1H), 10.05 (s, 1H); Mass Spectrum: M+H⁺ 487.

### EXAMPLE 3

### N-(5-ethylpyrazol-3-yl)-2-[5-(6-cyano-7-methoxyquinolin-4-yloxy)pyridin-2-yl]acetamide

1-(3-Dimethylaminopropyl)-3-ethylcarbodiimide hydrochloride (0.395 g) was added to a stirred mixture of 2-[5-(6-cyano-7-methoxyquinolin-4-yloxy)pyridin-2-yl]acetic acid (0.25 g), 5-amino-3-ethyl-1H-pyrazole (0.13 g), diisopropylethylamine (0.36 ml) and DMF (4 ml) at ambient temperature. The resultant mixture was stirred at ambient temperature for 16 hours. The reaction mixture was purified by preparative HPLC using a Waters ‘β Basic Hypersil’ reversed-phase column (5 microns silica, 30 mm diameter, 250 mm length) and decreasingly polar mixtures of water (containing 0.2% ammonium carbonate) and acetonitrile as eluent. There was thus obtained the title compound (0.17 g); ¹H NMR: (DMSOd₆) 1.15 (t, 3H), 2.6 (q, 2H), 3.9 (s, 2H), 4.1 (s, 3H), 6.3 (br s, 1H), 6.6 (d, 1H), 7.55 (d, 1H), 7.65 (s, 1H), 7.8 (m, 1H), 8.55 (s, 1H), 8.75 (d, 1H), 8.8 (s, 1H).

The 2-[5-(6-cyano-7-methoxyquinolin-4-yloxy)pyridin-2-yl]acetic acid used as a starting material was prepared as follows:—

A mixture of 4-chloro-6-cyano-7-methoxyquinoline (1.5 g; International Patent Application WO 02/12226, Example 1 thereof, which concerns analogous procedures to those described for the starting material in Example 1 of International Patent Application WO 98/13350 but where methanol is used instead of 2-methoxyethanol), tert-butyl (5-hydroxypyridin-2-yl)acetate (1.58 g), caesium carbonate (3.36 g) and DMA (40 ml) was stirred and heated to 85° C. for 2 hours. The resultant mixture was filtered and the filtrate was partitioned between ethyl acetate and water. The organic solution was washed with brine, dried over magnesium sulphate and evaporated. The residue was purified by column chromatography on silica using increasingly polar solvent mixtures of methylene chloride and methanol (from 100:0 to 1:4) as eluent. There was thus obtained tert-butyl 2-[5-(6-cyano-7-methoxyquinolin-4-yloxy)pyridin-2-yl]acetate as a solid (1.22 g); ¹H NMR: (DMSOd₆) 1.4 (s, 9H), 3.85 (s, 2H), 4.1 (s, 3H), 6.6 (d, 1H), 7.5 (d, 1H), 7.65 (s, 1H), 7.8 (dd, 1H), 8.55 (d, 1H), 8.8 (d, 1H), 8.85 (s, 1H).

A solution of the material so obtained in methylene chloride (20 ml) was cooled to 0° C. Water (0.5 ml) and trifluoroacetic acid (15 ml) were added and the reaction mixture was stirred at ambient temperature for 4 hours. The resultant mixture was evaporated and the residual trifluoroacetic acid was removed by azeotropic distillation with toluene under vacuum. The residue was dissolved in methylene chloride, diisopropylethylamine (2 ml) was added and the mixture was stirred at ambient temperature for 30 minutes. The resultant precipitate was collected by filtration, washed with diethyl ether and dried under vacuum. There was thus obtained 2-[5-(6-cyano-7-methoxyquinolin-4-yloxy)pyridin-2-yl]acetic acid (0.83 g); ¹H NMR: (DMSOd₆) 4.1 (s, 3H), 6.6 (d, 1H), 7.55 (d, 1H), 7.65 (s, 1H), 7.8 (m, 1H), 8.55 (d, 1H), 8.75 (d, 1H), 8.8 (s, 1H). Mass Spectrum: M+H⁺ 336.

### EXAMPLE 4

### N-(1-tert-butyl-3,4-dimethylpyrazol-5-yl)-2-[5-(6,7-dimethoxyquinolin-4-yloxy)-3-methoxypyridin-2-yl]acetamide

Using an analogous procedure to that described in Example 1, except that DMA (3 ml) was used in place as DMF as the reaction solvent, 2-[5-(6,7-dimethoxyquinolin-4-yloxy)-3-methoxypyridin-2-yl]acetic acid (0.7 g) was reacted with 5-amino-1-tert-butyl-3,4-dimethylpyrazole (0.4 g) in the presence of 2-(7-azabenzotriazol-1-yl)-1,1,3,3-tetramethyluronium hexafluorophosphate (1.1 g) and diisopropylethylamine (1 ml). The resultant mixture was partitioned between ethyl acetate and water. The organic phase was dried over magnesium sulphate and evaporated. The product was purified by column chromatography on silica using a 19:1 mixture of methylene chloride and methanol as eluent. There was thus obtained the title compound as a solid (0.711 g); ¹H NMR: (DMSOd₆) 1.49 (s, 9H), 1.71 (s, 3H), 2.04 (s, 3H), 3.82 (s, 3H), 3.86 (s, 2H), 3.94 (s, 3H), 3.95 (s, 3H), 6.57 (d, 1H), 7.42 (s, 1H), 7.49 (d, 1H), 7.52 (s, 1H), 8.09 (s, 1H), 8.51 (d, 1H), 9.47 (s, 1H); Mass Spectrum: M+H⁺ 520.

The 5-amino-1-tert-butyl-3,4-dimethylpyrazole used as a starting material was prepared as follows:—

1-tert-Butylhydrazine hydrochloride (1.55 g) was added to a stirred solution of 2-methyl-3-oxobutanenitrile (1 g) in ethanol (10 ml) and the resultant mixture was heated to 85° C. for 5 hours. The mixture was concentrated by evaporation and the residue was purified by column chromatography on silica using a solvent gradient from methylene chloride to a 7:3 mixture of methylene chloride and ethyl acetate as eluent. There was thus obtained the required amine (0.87 g); ¹H NMR: (DMSOd₆) 1.47 (s, 9H), 1.72 (s, 3H), 1.9 (s, 3H), 4.32 (br s, 2H); Mass Spectrum: M+H⁺ 168.

### EXAMPLE 5

### N-(4,5-dimethylpyrazol-3-yl)-2-[5-(6,7-dimethoxyquinolin-4-yloxy)-3-methoxypyridin-2-yl]acetamide

A mixture of N-(1-tert-butyl-3,4-dimethylpyrazol-5-yl)-2-[5-(6,7-dimethoxyquinolin-4-yloxy)-3-methoxypyridin-2-yl]acetamide (0.684 g), anisole (0.429 ml) and trifluoroacetic acid (8 ml) was stirred and heated to 90° C. for 4 days. The reaction mixture was cooled to ambient temperature, neutralised by the addition of a saturated aqueous sodium bicarbonate solution and extracted with ethyl acetate. The organic phase seas dried over magnesium sulphate and evaporated. The residue was purified by column chromatography on silica using a solvent gradient from 19:1 to 4:1 of methylene chloride and methanol as eluent. There was thus obtained the title compound as a solid (0.361 g); ¹H NMR: (DMSOd₆) 1.77 (s, 3H), 2.1 (s, 3H), 3.82 (s, 2H), 3.83 (s, 3H), 3.94 (s, 3H), 3.96 (s, 3H), 6.59 (d, 1H), 7.42 (s, 1H), 7.48 (d, 1H), 7.53 (s, 1H), 8.08 (d, 1H), 8.52 (d, 1H), 9.63 (br s, 1H), 12.0 (br s, 1H); Mass Spectrum: M+H⁺ 464.

### EXAMPLE 6

Using analogous procedures to those described in Examples 4 and 5, the appropriate 2-pyridin-2-ylacetic acid was reacted with the appropriate tert-butyl-protected aminopyrazole to give the compounds described in Table II.

TABLE II

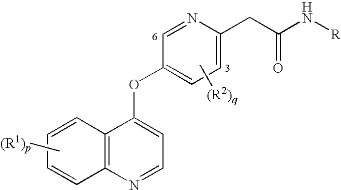

| No. & | | | |
|---|---|---|---|
| Note | (R¹)ₚ | (R²)_{q} | R |
| [1] | 6,7-dimethoxy | 3-methoxy | 4-ethyl-5-methylpyrazol-3-yl |
| [2] | 6,7-dimethoxy | 3-methoxy | 5-ethyl-4-methylpyrazol-3-yl |
| [3] | 5,7-dimethoxy | 3-methoxy | 4,5-dimethylpyrazol-3-yl |
| [4] | 7-ethoxy-6-methoxy | 3-methoxy | 4-ethylpyrazol-3-yl |
| [5] | 7-ethoxy-6-methoxy | 3-methoxy | 4,5-dimethylpyrazol-3-yl |
| [6] | 7-ethoxy-6-methoxy | 3-methoxy | 4-ethyl-5-methylpyrazol-3-yl |
| [7] | 7-ethoxy-6-methoxy | 3-methoxy | 5-ethyl-4-methylpyrazol-3-yl |
| [8] | 6-methoxy | 3-methoxy | 4,5-dimethylpyrazol-3-yl |
| [9] | 6-methoxy | 3-methoxy | 4-ethyl-5-methylpyrazol-3-yl |
| [10] | 6-methoxy | 3-methoxy | 5-ethyl-4-methylpyrazol-3-yl |
| [11] | 7-methoxy | 3-methoxy | 4,5-dimethylpyrazol-3-yl |
| [12] | 7-methoxy | 3-methoxy | 4-ethyl-5-methylpyrazol-3-yl |
| [13] | 7-methoxy | 3-methoxy | 5-ethyl-4-methylpyrazol-3-yl |
| [14] | 6-fluoro | 3-methoxy | 4-ethyl-5-methylpyrazol-3-yl |
| [15] | 6-fluoro | 3-methoxy | 5-ethyl-4-methylpyrazol-3-yl |
| [16] | 6,7-dimethoxy | 3-methoxy | 4,5-trimethylenepyrazol-3-yl |
| [17] | 7-ethoxy-6-methoxy | 3-methoxy | 4,5-trimethylenepyrazol-3-yl |
| [18] | 6-methoxy | 3-methoxy | 4,5-trimethylenepyrazol-3-yl |
| [19] | 7-methoxy | 3-methoxy | 4,5-trimethylenepyrazol-3-yl |
| [20] | 6-fluoro | 3-methoxy | 4,5-trimethylenepyrazol-3-yl |

Notes Characterising data for each product are shown below.
[1] ¹H NMR: (DMSOd₆) 0.99(t, 3 H), 2.12(s, 3 H), 2.26(q, 2 H), 3.82(m, 5 H), 3.94(s, 3 H), 3.95(s, 3 H), 6.58(d, 1 H), 7.42(s, 1 H), 7.48(s, 1 H), 7.53(s, 1 H), 8.07(d, 1 H), 8.51(d, 1 H), 9.52(br s, 1 H); Mass Spectrum: M + H⁺ 478.

The N-(1-tert-butyl-4-ethyl-3-methylpyrazol-5-yl)-2-[5-(6,7-dimethoxyquinolin-4-yloxy)-3-methoxypyridin-2-yl]acetamide used as a starting material was prepared as follows:—

Under an atmosphere of argon, a solution of a mixture of butyronitrile (20 ml) and methyl acetate (23.54 ml) in THF (40 ml) was added dropwise to a stirred suspension of potassium tert-butoxide (56.67 g) in THF that had been cooled to 15° C. The resultant mixture was stirred at ambient temperature for 16 hours. The solvent was evaporated and the residue was triturated under diethyl ether. The solid was isolated. Water (200 ml) was added and the mixture was acidified to pH4 by the addition of 12N aqueous hydrochloric acid. The aqueous mixture was extracted with methylene chloride and the organic extract was dried over magnesium sulphate and evaporated. There was thus obtained 2-ethyl-3-oxobutanenitrile (6.8 g); ¹H NMR: (CDCl₃) 1.11 (t, 3H), 1.93 (m, 2H), 2.39 (s, 3H), 3.37 (m, 1H).

1-tert-Butylhydrazine hydrochloride (5.11 g) was added to a stirred solution of 2-ethyl-3-oxobutanenitrile (3.4 g) in ethanol (35 ml) and the resultant mixture was heated to reflux for 12 hours. The mixture was evaporated. A saturated aqueous solution of sodium bicarbonate was added to the residue and the mixture was basified to pH9 by the addition of 2N aqueous sodium hydroxide. The mixture was extracted with ethyl acetate. The organic phase was washed with brine, dried over magnesium sulphate and evaporated. The residue was purified by column chromatography on silica using a solvent gradient from methylene chloride to a 3:2 mixture of methylene chloride and ethyl acetate as eluent. There was thus obtained 5-amino-1-tert-butyl-4-ethyl-3-methylpyrazole (2.95 g); ¹H NMR: (CDCl₃) 1.06 (t, 3H), 1.62 (s, 9H), 2.14 (s, 3H), 2.25 (q, 2H), 3.28 (br s, 2H).

Using an analogous procedure to that described in Example 4, 5-amino-1-tert-butyl-4-ethyl-3-methylpyrazole (0.1 g) was reacted with 2-[5-(6,7-dimethoxyquinolin-4-yloxy)-3-methoxypyridin-2-yl]acetic acid (0.2 g) to give the required starting material (0.143 g); ¹H NMR: (DMSOd₆) 0.98 (t, 3H), 1.49 (s, 9H), 2.06 (s, 3H), 2.17 (q, 2H), 3.82 (s, 3H), 3.85 (s, 2H), 3.94 (s, 3H), 3.96 (s, 3H), 6.57 (d, 1H), 7.42 (s, 1H), 7.49 (d, 1H), 7.52 (s, 1H), 8.07 (d, 1H), 8.51 (d, 1H), 9.47 (s, 1H); Mass Spectrum: M+H⁺ 534.

[2] ¹H NMR: (DMSOd₆) 1.13 (t, 3H), 1.77 (s, 3H), 2.5 (q, 2H), 3.83 (s, 3H), 3.94 (s, 2H), 3.95 (s, 3H), 6.57 (d, 1H), 7.42 (s, 1H), 7.48 (s, 1H), 7.53 (s, 1H), 8.07 (d, 1H), 8.51 (d, 1H), 9.59 (br s, 1H); Mass Spectrum: M+H⁺ 478.

The N-(1-tert-butyl-3-ethyl-4-methylpyrazol-5-yl)-2-[5-(6,7-dimethoxyquinolin-4-yloxy)-3-methoxypyridin-2-yl]acetamide used as a starting material was prepared as follows:—

Under an atmosphere of argon, n-butyllithium (2.5 M in hexane, 50 ml) was added slowly to a stirred solution of diisopropylamine (17.52 ml) in THF (100 ml) that had been cooled to −78° C. The resultant solution was maintained at −78° C. and propionitrile (8.92 ml) was added slowly. The mixture was stirred at −78° C. for 30 minutes. Methyl propionate (6 ml) was added dropwise and the mixture was stirred at −78° C. for 2 hours. The reaction mixture was allowed to warm to 0° C. and ice was added. The resultant solution was concentrated by evaporation and partitioned between diethyl ether and water. The aqueous solution was acidified to pH2 by the addition of 6N aqueous hydrochloric acid and extracted with diethyl ether. The resultant organic phase was dried over magnesium sulphate and evaporated. There was thus obtained 2-methyl-3-oxopentanenitrile (5.8 g); ¹H NMR: (CDCl₃) 1.35 (t, 3H), 1.51 (d, 3H), 2.69 (m, 1H), 2.82 (m, 1H), 3.48 (m, 1H).

Using an analogous procedure to that described in the portion of Note [1] immediately above that is concerned with the preparation of starting materials, 1-tert-butylhydrazine hydrochloride was reacted with 2-methyl-3-oxopentanenitrile (5.8 g). There was thus obtained 5-amino-1-tert-butyl-3-ethyl-4-methylpyrazole (6.64 g); ¹H NMR: (CDCl₃) 1.05 (t, 3H), 1.48 (s, 9H), 1.74 (s, 3H), 2.31 (q, 2H), 4.29 (br s, 2H); Mass Spectrum: M+H⁺ 182.

Using an analogous procedure to that described in Example 4, 5-amino-1-tert-butyl-3-ethyl-4-methylpyrazole (0.11 g) was reacted with 2-[5-(6,7-dimethoxyquinolin-4-yloxy)-3-methoxypyridin-2-yl]acetic acid (0.22 g) to give the required starting material (0.178 g); ¹H NMR: (DMSOd₆) 1.11 (t, 3H), 1.49 (s, 9H), 1.73 (s, 3H), 2.44 (q, 2H), 3.82 (s, 3H), 3.86 (s, 2H), 3.94 (s, 3H), 3.95 (s, 3H), 6.56 (d, 1H), 7.42 (s, 1H), 7.49 (d, 1H), 7.52 (s, 1H), 8.08 (d, 1H), 8.51 (d, 1H), 9.44 (br s, 1H); Mass Spectrum: M+H⁺ 534.

[3] ¹H NMR: (DMSOd₆) 1.75 (s, 3H), 2.1 (s, 3H), 3.77 (s, 2H), 3.8 (s, 3H), 3.83 (s, 3H), 3.91 (s, 3H), 6.6 (d, 1H), 6.67 (d, 1H), 7.02 (d, 1H), 7.31 (d, 1H), 7.86 (d, 1H), 8.58 (d, 1H), 9.58 (br s, 1H); Mass Spectrum: M+H⁺ 464.

The N-(1-tert-butyl-3,4-dimethylpyrazol-5-yl)-2-[5-(5,7-dimethoxyquinolin-4-yloxy)-3-methoxypyridin-2-yl]acetamide used as a starting material was prepared as follows:—

Using an analogous procedure to that described in Example 4, 5-amino-1-tert-butyl-3,4-dimethylpyrazole (0.1 g) was reacted with 2-[5-(5,7-dimethoxyquinolin-4-yloxy)-3-methoxypyridin-2-yl]acetic acid (0.185 g) to give the required starting material (0.185 g); ¹H NMR: (DMSOd₆) 1.48 (s, 9H), 1.7 (s, 3H), 2.03 (s, 3H), 3.8 (s, 2H), 3.82 (s, 3H), 3.91 (s, 3H), 6.6 (d, 1H), 6.67 (d, 1H), 7.03 (d, 1H), 7.32 (d, 1H), 7.85 (d, 1H), 8.59 (d, 1H), 9.45 (s, 1H); Mass Spectrum: M+H⁺ 520.

[4] ¹H NMR: (DMSOd₆) 1.01 (t, 3H), 1.36 (t, 3H), 2.23 (q, 2H), 3.76 (2s, 5H), 3.87 (s, 3H), 4.14 (q, 2H), 6.49 (d, 1H), 7.33 (s, 1H), 7.35 (m, 1H), 7.41 (d, 1H), 7.45 (s, 1H), 8.01 (d, 1H), 8.43 (d, 1H), 9.58 (br s, 1H); Mass Spectrum: M+H⁺ 478.

The N-(1-tert-butyl-4-ethylpyrazol-5-yl)-2-[5-(7-ethoxy-6-methoxyquinolin-4-yloxy)-3-methoxypyridin-2-yl]acetamide used as a starting material was prepared as follows:—

Under an atmosphere of argon, a solution of a mixture of butyronitrile (2.18 ml) and ethyl formate (2.11 ml) in THF (4 ml) was added dropwise to a stirred suspension of potassium tert-butoxide (6.18 g) in THF (40 ml) and the resultant mixture was stirred at ambient temperature for 16 hours. The solvent was evaporated and the residual solid was suspended in ether, isolated by filtration and washed with diethyl ether. The solid was dissolved in water and the solution was acidified to pH4 by the addition of 2N aqueous hydrochloric acid and extracted with methylene chloride. The organic extract was dried over magnesium sulphate. There was thus obtained 2-formylbutyronitrile (1 g) which was used without further purification.

1-tert-Butylhydrazine hydrate (1.72 g) and acetic acid (0.59 ml) were added in turn to a solution of 2-formylbutyronitrile (1 g) in ethanol (10 ml) and the resultant mixture was heated to reflux for 5 hours. The mixture was evaporated and a saturated aqueous sodium bicarbonate solution was added. The mixture was basified to pH9 with 2N aqueous sodium hydroxide and extracted with methylene chloride. The organic extract was dried over magnesium sulphate and evaporated. The residue was purified by column chromatography on silica using a solvent gradient from methylene chloride to a 1:1 mixture of methylene chloride and ethyl acetate as eluent. There was thus obtained 5-amino-1-tert-butyl-4-ethylpyrazole; ¹H NMR: (CDCl₃) 1.17 (t, 3H), 1.64 (s, 9H), 2.28 (q, 2H), 3.28 (br s, 2H), 7.15 (s, 1H); Mass Spectrum: M+H⁺ 168.

Using an analogous procedure to that described in Example 4, 5-amino-1-tert-butyl-4-ethylpyrazole (0.104 g) was reacted with 2-[5-(7-ethoxy-6-methoxyquinolin-4-yloxy)-3-methoxypyridin-2-yl]acetic acid (0.17 g) to give the required starting material (0.215 g); ¹H NMR: (DMSOd₆) 1.07 (t, 3H), 1.43 (t, 3H), 1.52 (s, 9H), 2.2 (q, 2H), 3.86 (s, 3H), 3.94 (s, 2H), 4.02 (s, 3H), 4.21 (q, 2H), 6.55 (d, 1H), 7.23 (s, 1H), 7.4 (s, 1H), 7.49 (d, 1H), 7.52 (s, 1H), 8.08 (d, 1H), 8.5 (d, 1H), 9.49 (br s, 1H); Mass Spectrum: M+H⁺ 534.

[5] ¹H NMR: (DMSOd₆) 1.43 (t, 3H), 1.76 (s, 3H), 2.1 (s, 3H), 3.83 (2s, 5H), 3.86 (s, 2H), 3.94 (s, 3H), 4.21 (q, 2H), 6.57 (d, 1H), 7.39 (s, 1H), 7.48 (d, 1H), 7.52 (s, 1H), 8.08 (d, 1H), 8.5 (d, 1H), 9.61 (br s, 1H); Mass Spectrum: M+H⁺ 478.

The N-(1-tert-butyl-3,4-dimethylpyrazol-5-yl)-2-[5-(7-ethoxy-6-methoxyquinolin-4-yloxy)-3-methoxypyridin-2-yl]acetamide used as a starting material was prepared as follows:—

Using an analogous procedure to that described in Example 4, 5-amino-1-tert-butyl-3,4-dimethylpyrazole (0.096 g) was reacted with 2-[5-(7-ethoxy-6-methoxyquinolin-4-yloxy)-3-methoxypyridin-2-yl]acetic acid (0.17 g) to give the required starting material (0.231 g); ¹H NMR: (DMSOd₆) 1.43 (t, 3H), 1.49 (s, 9H), 1.71 (s, 3H), 2.04 (s, 3H), 3.82 (s, 3H), 3.86 (s, 3H), 3.94 (s, 3H), 4.21 (q, 2H), 6.56 (d, 1H), 7.4 (s, 1H), 7.49 (d, 1H), 7.52 (s, 1H), 8.08 (d, 1H), 8.5 (d, 1H), 9.47 (s, 1H); Mass Spectrum: M+H⁺ 534.

[6] ¹H NMR: (DMSOd₆) 1.0 (t, 3H), 1.44 (t, 3H), 2.13 (s, 3H), 2.27 (q, 2H), 3.83 (2s, 5H), 4.22 (q, 2H), 6.58 (d, 1H), 7.41 (s, 1H), 7.49 (d, 1H), 7.53 (s, 1H), 8.08 (d, 1H), 8.51 (d, 1H), 9.57 (br s, 1H); Mass Spectrum: M+H⁺ 492.

The N-(1-tert-butyl-4-ethyl-3-methylpyrazol-5-yl)-2-[5-(7-ethoxy-6-methoxyquinolin-4-yloxy)-3-methoxypyridin-2-yl]acetamide used as a starting material was prepared as follows:—

Using an analogous procedure to that described in Example 4, 5-amino-1-tert-butyl-4-ethyl-3-methylpyrazole (0.112 g) was reacted with 2-[5-(7-ethoxy-6-methoxyquinolin-4-yloxy)-3-methoxypyridin-2-yl]acetic acid (0.17 g) to give the required starting material (0.209 g); ¹H NMR: (DMSOd₆) 0.99 (t, 3H), 1.44 (t, 3H), 1.5 (s, 9H), 2.07 (s, 3H), 2.18 (q, 2H), 3.83 (s, 3H), 3.86 (s, 2H), 3.95 (s, 3H), 4.22 (q, 2H), 6.57 (d, 1H), 7.41 (s, 1H), 7.5 (d, 1H), 7.53 (s, 1H), 8.08 (d, 1H), 8.51 (d, 1H), 9.47 (s, 1H); Mass Spectrum: M+H⁺ 548.

[7] ¹H NMR: (DMSOd₆) 1.14 (t, 3H), 1.45 (t, 3H), 1.79 (s, 3H), 2.51 (q, 2H), 3.52 (2s, 5H), 3.84 (s, 3H), 4.22 (q, 2H), 6.6 (d, 1H), 7.41 (s, 1H), 7.5 (d, 1H), 7.55 (s, 1H), 8.1 (d, 1H), 8.53 (d, 1H), 9.63 (br s, 1H); Mass Spectrum: M+H⁺ 492.

The N-(1-tert-butyl-3-ethyl-4-methylpyrazol-5-yl)-2-[5-(7-ethoxy-6-methoxyquinolin-4-yloxy)-3-methoxypyridin-2-yl]acetamide used as a starting material was prepared as follows:—

Using an analogous procedure to that described in Example 4, 5-amino-1-tert-butyl-3-ethyl-4-methylpyrazole (0.112 g) was reacted with 2-[5-(7-ethoxy-6-methoxyquinolin-4-yloxy)-3-methoxypyridin-2-yl]acetic acid (0.17 g) to give the required starting material (0.226 g); ¹H NMR: (DMSOd₆) 1.11 (t, 3H), 1.43 (t, 3H), 1.49 (s, 9H), 1.73 (s, 3H), 2.44 (q, 2H), 3.82 (s, 3H), 3.86 (s, 2H), 3.94 (s, 3H), 4.21 (q, 2H), 6.54 (d, 1H), 7.4 (s, 1H), 7.49 (d, 1H), 7.52 (s, 1H), 8.08 (d, 1H), 8.5 (d, 1H), 9.43 (s, 1H); Mass Spectrum: M+H⁺ 548.

[8] ¹H NMR: (DMSOd₆) 1.77 (s, 3H), 2.1 (s, 3H), 3.83 (2s, 5H), 3.94 (s, 3H), 6.7 (d, 1H), 7.49 (m, 2H), 7.59 (d, 1H), 7.97 (d, 1H), 8.1 (d, 1H), 8.58 (d, 1H), 9.67 (br s, 1H); Mass Spectrum: M+H⁺ 434.

The N-(1-tert-butyl-3,4-dimethylpyrazol-5-yl)-2-[5-(6-methoxyquinolin-4-yloxy)-3-methoxypyridin-2-yl]acetamide used as a starting material was prepared as follows:—

Using an analogous procedure to that described in Example 4, 5-amino-1-tert-butyl-3,4-dimethylpyrazole (0.14 g) was reacted with 2-[5-(6-methoxyquinolin-4-yloxy)-3-methoxypyridin-2-yl]acetic acid (0.22 g) to give the required starting material (0.258 g); ¹H NMR: (DMSOd₆) 1.49 (s, 9H), 1.72 (s, 3H), 2.04 (s, 3H), 3.83 (s, 3H), 3.87 (s, 2H), 3.93 (s, 3H), 6.69 (d, 1H), 7.48 (m, 1H), 7.52 (d, 1H), 7.58 (d, 1H), 7.97 (d, 1H), 8.1 (d, 1H), 8.58 (d, 1H), 9.48 (s, 1H); Mass Spectrum: M+H⁺490.

[9] ¹H NMR: (DMSOd₆) 1.0 (t, 3H), 2.13 (s, 3H), 2.27 (q, 2H), 3.84 (2s, 5H), 3.95 (s, 3H), 6.71 (d, 1H), 7.48 (m, 1H), 7.51 (d, 1H), 7.59 (d, 1H), 7.98 (d, 1H), 8.11 (d, 1H), 8.58 (d, 1H), 9.55 (br s, 1H), 12.02 (br s, 1H); Mass Spectrum: M+H⁺ 448.

The N-(1-tert-butyl-4-ethyl-3-methylpyrazol-5-yl)-2-[5-(6-methoxyquinolin-4-yloxy)-3-methoxypyridin-2-yl]acetamide used as a starting material was prepared as follows:—

Using an analogous procedure to that described in Example 4, 5-amino-1-tert-butyl-4-ethyl-3-methylpyrazole (0.152 g) was reacted with 2-[5-(6-methoxyquinolin-4-yloxy)-3-methoxypyridin-2-yl]acetic acid (0.22 g) to give the required starting material (0.282 g); ¹H NMR: (DMSOd₆) 0.98 (t, 3H), 1.49 (s, 9H), 2.06 (s, 3H), 2.17 (q, 2H), 3.83 (s, 3H), 3.86 (s, 2H), 3.93 (s, 3H), 6.69 (d, 1H), 7.49 (m, 1H), 7.51 (d, 1H), 7.58 (d, 1H), 7.97 (d, 1H), 8.1 (d, 1H), 8.58 (d, 1H), 9.47 (s, 1H); Mass Spectrum: M+H⁺ 504.

[10] ¹H NMR: (DMSOd₆) 1.13 (t, 3H), 1.78 (s, 3H), 2.5 (q, 2H), 3.83 (2s, 5H), 3.94 (s, 3H), 6.7 (d, 1H), 7.49 (m, 2H), 7.59 (d, 1H), 7.96 (d, 1H), 8.1 (d, 1H), 8.57 (d, 1H), 9.6 (br s, 1H); Mass Spectrum: M+H⁺ 448.

The N-(1-tert-butyl-3-ethyl-4-methylpyrazol-5-yl)-2-[5-(6-methoxyquinolin-4-yloxy)-3-methoxypyridin-2-yl]acetamide used as a starting material was prepared as follows:—

Using an analogous procedure to that described in Example 4, 5-amino-1-tert-butyl-3-ethyl-4-methylpyrazole (0.152 g) was reacted with 2-[5-(6-methoxyquinolin-4-yloxy)-3-methoxypyridin-2-yl]acetic acid (0.22 g) to give the required starting material (0.297 g); ¹H NMR: (DMSOd₆) 1.11 (t, 3H), 1.49 (s, 9H), 1.74 (s, 3H), 2.44 (q, 2H), 3.83 (s, 3H), 3.87 (s, 2H), 3.93 (s, 3H), 6.69 (d, 1H), 7.48 (m, 1H), 7.52 (d, 1H), 7.58 (d, 1H), 7.96 (d, 1H), 8.1 (d, 1H), 8.57 (d, 1H), 9.44 (s, 1H); Mass Spectrum: M+H⁺ 548.

[11] ¹H NMR: (DMSOd₆) 1.78 (s, 3H), 2.09 (s, 3H), 3.83 (s, 2H), 3.95 (s, 3H), 6.59 (d, 1H), 7.3 (m, 1H), 7.43 (m, 2H), 8.06 (s, 1H), 8.21 (d, 1H), 8.64 (d, 1H), 9.43 (br s, 1H), 11.88 (br s, 1H); Mass Spectrum: M+H⁺ 434.

The N-(1-tert-butyl-3,4-dimethylpyrazol-5-yl)-2-[5-(7-methoxyquinolin-4-yloxy)-3-methoxypyridin-2-yl]acetamide used as a starting material was prepared as follows:—

Under an atmosphere of argon, benzyl bromide (0.387 ml) was added to a stirred mixture of methyl 2-(5-hydroxy-3-methoxypyridin-2-yl)acetate (0.813 g), potassium carbonate (1.28 g) and DMF (9 ml). The resultant mixture was heated to 60° C. for 16 hours. The mixture was cooled to ambient temperature, diluted with water and extracted with ethyl acetate. The organic phase was washed with water, dried over magnesium sulphate and evaporated. The residue was purified by column chromatography on silica using a solvent gradient from methylene chloride to a 17:3 mixture of methylene chloride and ethyl acetate as eluent. There was thus obtained methyl 2-(5-benzyloxy-3-methoxypyridin-2-yl)acetate (0.754 g); ¹H NMR: (DMSOd₆) 3.59 (s, 3H), 3.68 (s, 2H), 3.79 (s, 3H), 5.2 (s, 2H), 7.14 (d, 1H), 7.35 (m, 1H), 7.41 (m, 2H), 7.47 (d, 2H), 7.86 (d, 1H); Mass Spectrum: M+H⁺ 288.

A mixture of the material so obtained, sodium hydroxide (0.314 g), water (1.5 ml) and methanol (8 ml) was stirred at ambient temperature for 2 days. The methanol was evaporated and the aqueous mixture was acidified to pH4.8 by the addition of 2N aqueous hydrochloric acid. The resultant precipitate was isolated, washed in turn with water and with diethyl ether and dried under vacuum. There was thus obtained 2-(5-benzyloxy-3-methoxypyridin-2-yl)acetic acid (0.624 g); ¹H NMR: (DMSOd₆) 3.59 (s, 2H), 3.79 (s, 3H), 5.19 (s, 2H), 7.12 (d, 1H), 7.35 (m, 1H), 7.41 (m, 2H), 7.47 (m, 2H), 7.85 (d, 1H); Mass Spectrum: M+H⁺ 274.

Diisopropylethylamine (1.19 ml), 5-amino-1-tert-butyl-3,4-dimethylpyrazole (0.455 g) and 2-(7-azabenzotriazol-1-yl)-1,1,3,3-tetramethyluronium hexafluorophosphate (1.12 g) were added in turn to a stirred mixture of 2-(5-benzyloxy-3-methoxypyridin-2-yl)acetic acid (0.62 g) and DMA (6 ml). The resultant mixture was stirred at ambient temperature for 16 hours. A saturated aqueous sodium bicarbonate solution was added and the mixture was extracted with ethyl acetate. The organic phase was washed with water, dried over magnesium sulphate and evaporated. The residue was purified by column chromatography on silica using a solvent gradient from methylene chloride to a 7:3 mixture of methylene chloride and ethyl acetate as eluent. There was thus obtained N-(1-tert-butyl-3,4-dimethylpyrazol-5-yl)-2-(5-benzyloxy-3-methoxypyridin-2-yl)acetamide (0.883 g); ¹H NMR: (DMSOd₆) 1.46 (s, 9H), 1.68 (s, 3H), 2.02 (s, 3H), 3.71 (s, 2H), 3.8 (s, 3H), 5.2 (s, 2H), 7.12 (d, 1H), 7.35 (m, 1H), 7.41 (m, 2H), 7.48 (m, 2H), 7.86 (d, 1H), 9.4 (s, 1H); Mass Spectrum: M+H⁺ 423.

A mixture of the material so obtained, ammonium formate (1.22 g), 10% palladium-on-carbon catalyst (0.15 g) and DMF (10 ml) was stirred and heated to 50° C. for 16 hours. The resultant mixture was cooled to ambient temperature and filtered. The filtrate was partitioned between water and ethyl acetate. The organic phase was washed with water, dried over magnesium sulphate and evaporated. The residue was purified by column chromatography on silica using a 19:1 mixture of methylene chloride and methanol as eluent. There was thus obtained N-(1-tert-butyl-3,4-dimethylpyrazol-5-yl)-2-(5-hydroxy-3-methoxypyridin-2-yl)acetamide (0.485 g); ¹H NMR: (DMSOd₆) 1.46 (s, 9H), 1.68 (s, 3H), 2.02 (s, 3H), 3.66 (s, 2H), 3.74 (s, 3H), 6.8 (d, 1H), 7.64 (d, 1H), 9.35 (s, 1H), 9.8 (br s, 1H); Mass Spectrum: M+H⁺ 333.

A mixture of a portion (0.189 g) of the material so obtained, 4-chloro-7-methoxyquinoline (0.11 g), 4-dimethylaminopyridine (0.208 g) and chlorobenzene (1.5 ml) was stirred and heated to 130° C. for 16 hours. The mixture was cooled to ambient temperature and ethyl acetate was added. The mixture was filtered and the filtrate was concentrated by evaporation. The residue was purified by column chromatography on silica using a solvent gradient comprising 97:3 to 93:7 mixtures of methylene chloride and methanol as eluent. There was thus obtained N-(1-tert-butyl-3,4-dimethylpyrazol-5-yl)-2-[5-(7-methoxyquinolin-4-yloxy)-3-methoxypyridin-2-yl]acetamide (0.173 g); ¹H NMR: (DMSOd₆) 1.49 (s, 9H), 1.71 (s, 3H), 2.04 (s, 3H), 3.82 (s, 3H), 3.86 (s, 2H), 3.95 (s, 3H), 6.56 (d, 1H), 7.31 (m, 1H), 7.43 (d, 1H), 7.51 (d, 1H), 8.08 (d, 1H), 8.22 (d, 1H), 8.64 (d, 1H), 9.48 (s, 1H); Mass Spectrum: M+H⁺ 490.

[12] ¹H NMR: (DMSOd₆+CF₃CO₂D) 1.08 (t, 3H), 2.3 (s, 3H), 2.52 (q, 2H), 3.87 (s, 3H), 4.0 (s, 2H), 4.06 (s, 3H), 7.02 (d, 1H), 7.64 (s, 1H), 7.66 (s, 1H), 7.77 (s, 1H), 8.29 (d, 1H), 8.53 (d, 1H), 9.07 (d, 1H); Mass Spectrum: M+H⁺ 448.

The N-(1-tert-butyl-4-ethyl-3-methylpyrazol-5-yl)-2-[5-(7-methoxyquinolin-4-yloxy)-3-methoxypyridin-2-yl]acetamide used as a starting material was prepared as follows:—

Using an analogous procedure to that described in Example 4, 5-amino-1-tert-butyl-4-ethyl-3-methylpyrazole (0.127 g) was reacted with 2-[5-(7-methoxyquinolin-4-yloxy)-3-methoxypyridin-2-yl]acetic acid (0.17 g) to give the required starting material (0.238 g); ¹H NMR: (DMSOd₆) 0.98 (t, 3H), 1.49 (s, 9H), 2.06 (s, 3H), 2.17 (q, 1H), 3.82 (s, 3H), 3.85 (s, 2H), 3.95 (s, 3H), 6.56 (d, 1H), 7.31 (m, 1H), 7.43 (d, 1H), 7.51 (d, 1H), 8.08 (d, 1H), 8.22 (d, 1H), 8.64 (d, 1H), 9.47 (s, 1H); Mass Spectrum: M+H⁺ 504.

[13] ¹H NMR: (DMSOd₆) 1.13 (t, 3H), 1.78 (s, 3H), 2.5 (q, 2H), 3.83 (m, 5H), 3.94 (s, 3H), 6.57 (d, 1H), 7.31 (d, 1H), 7.43 (d, 1H), 7.5 (s, 1H), 8.08 (d, 1H), 8.22 (d, 1H), 8.64 (d, 1H), 9.59 (br s, 1H); Mass Spectrum: M+H⁺ 448.

The N-(1-tert-butyl-3-ethyl-4-methylpyrazol-5-yl)-2-[5-(7-methoxyquinolin-4-yloxy)-3-methoxypyridin-2-yl]acetamide used as a starting material was prepared as follows:—

Using an analogous procedure to that described in Example 4, 5-amino-1-tert-butyl-3-ethyl-4-methylpyrazole (0.11 g) was reacted with 2-[5-(7-methoxyquinolin-4-yloxy)-3-methoxypyridin-2-yl]acetic acid (0.16 g) to give the required starting material (0.196 g); ¹H NMR: (DMSOd₆) 1.11 (t, 3H), 1.49 (s, 9H), 1.74 (s, 3H), 2.43 (q, 2H), 3.82 (s, 3H), 3.86 (s, 2H), 3.95 (s, 3H), 6.56 (d, 1H), 7.31 (m, 1H), 7.43 (d, 1H), 7.51 (d, 1H), 8.08 (d, 1H), 8.22 (d, 1H); 8.64 (d, 1H); 9.44 (s, 1H); Mass Spectrum: M+H⁺ 504.

[14] ¹H NMR: (DMSOd₆+CF₃CO₂D) 1.01 (t, 3H), 2.23 (s, 3H), 2.45 (q, 2H), 3.81 (s, 3H), 4.03 (s, 2H), 7.17 (d, 1H), 7.7 (s, 1H), 8.1 (m, 1H), 8.23 (s, 1H), 8.33 (m, 2H), 9.13 (d, 1H); Mass Spectrum: M+H⁺ 436.

The N-(1-tert-butyl-4-ethyl-3-methylpyrazol-5-yl)-2-[5-(6-fluoroquinolin-4-yloxy)-3-methoxypyridin-2-yl]acetamide used as a starting material was prepared as follows:—

Using an analogous procedure to that described in Example 4, 5-amino-1-tert-butyl-4-ethyl-3-methylpyrazole (0.125 g) was reacted with 2-[5-(6-fluoroquinolin-4-yloxy)-3-methoxypyridin-2-yl]acetic acid (0.175 g) to give the required starting material (0.164 g); ¹H NMR: (DMSOd₆) 0.99 (t, 3H), 1.5 (s, 9H), 2.07 (s, 3H), 2.18 (q, 2H), 3.83 (s, 3H), 3.87 (s, 2H), 6.78 (d, 1H), 7.55 (d, 1H), 7.79 (m, 1H), 8.02 (m, 1H), 8.12 (m, 2H), 8.73 (d, 1H), 9.48 (s, 1H); Mass Spectrum: M+H⁺ 492.

[15] ¹H NMR: (DMSOd₆+CF₃CO₂D) 1.17 (t, 3H), 2.02 (s, 3H), 2.67 (q, 2H), 3.86 (s, 3H), 4.08 (s, 2H), 7.21 (d, 1H), 7.73 (s, 1H), 8.14 (m, 1H), 8.29 (s, 1H), 8.35 (m, 1H), 8.4 (m, 1H), 9.2 (d, 1H); Mass Spectrum: M+H⁺ 436.

The N-(1-tert-butyl-3-ethyl-4-methylpyrazol-5-yl)-2-[5-(6-fluoroquinolin-4-yloxy)-3-methoxypyridin-2-yl]acetamide used as a starting material was prepared as follows:—

Using an analogous procedure to that described in Example 4,5-amino-1-tert-butyl-3-ethyl-4-methylpyrazole (0.107 g) was reacted with 2-[5-(6-fluoroquinolin-4-yloxy)-3-methoxypyridin-2-yl]acetic acid (0.15 g) to give the required starting material (0.16 g); ¹H NMR: (DMSOd₆) 1.11 (t, 3H), 1.49 (s, 9H), 1.74 (s, 3H), 2.44 (q, 2H), 3.83 (s, 3H), 3.87 (s, 2H), 6.77 (d, 1H), 7.55 (d, 1H), 7.77 (m, 1H), 8.0 (m, 1H), 8.13 (m, 2H), 8.72 (d, 1H), 9.44 (s, 1H); Mass Spectrum: M+H⁺ 492.

[16] ¹H NMR: (DMSOd₆+CF₃CO₂D) 2.48 (m, 2H), 2.67 (m, 2H), 2.82 (m, 2H), 3.87 (s, 3H), 4.01 (s, 2H), 4.06 (s, 3H), 4.08 (s, 3H), 7.03 (d, 1H), 7.65 (s, 1H), 7.75 (d, 1H), 7.79 (s, 1H), 8.29 (d, 1H), 8.93 (d, 1H); Mass Spectrum: M+H⁺ 476.

The N-(1-tert-butyl-3,4-trimethylenepyrazol-5-yl)-2-[5-(6,7-dimethoxyquinolin-4-yloxy)-3-methoxypyridin-2-yl]acetamide used as a starting material was prepared as follows:—

Under an atmosphere of nitrogen, 1-tert-butylhydrazine hydrochloride (1.5 g) was added to a stirred solution of 2-oxocyclopentane-1-carbonitrile (1.09 g) in ethanol (20 ml) and the resultant mixture was heated to reflux for 2 hours. The mixture was concentrated by evaporation. Water was added to the residue, followed by sufficient aqueous sodium bicarbonate solution until gas evolution ceased. The resultant mixture was extracted with diethyl ether, dried over magnesium sulphate and evaporated. There was thus obtained 5-amino-1-tert-butyl-3,4-trimethylenepyrazole (1.6 g); ¹H NMR: (CDCl₃) 1.64 (s, 9H), 2.35 (m, 1H), 2.48 (m, 2H), 2.67 (m, 2H), 3.47 (br s, 2H); Mass Spectrum: M+H⁺ 180.

Using an analogous procedure to that described in Example 4, 5-amino-1-tert-butyl-3,4-trimethylenepyrazole (0.107 g) was reacted with 2-[5-(6,7-dimethoxyquinolin-4-yloxy)-3-methoxypyridin-2-yl]acetic acid (0.17 g) to give the required starting material (0.158 g); ¹H NMR: (DMSOd₆) 1.52 (s, 9H), 2.25 (m, 2H), 2.39 (m, 2H), 2.5 (m, 2H), 3.82 (s, 3H), 3.83 (s, 2H), 3.94 (s, 3H), 3.95 (s, 3H), 6.56 (d, 1H), 7.42 (s, 1H), 7.49 (d, 1H), 7.52 (s, 1H), 8.08 (d, 1H), 8.51 (d, 1H), 9.48 (br s, 1H); Mass Spectrum: M+H⁺ 532.

[17] ¹H NMR: (DMSOd₆) 1.43 (t, 3H), 2.33 (m, 2H), 2.58 (m, 4H), 3.81 (2s, 5H), 3.94 (s, 3H), 4.21 (q, 2H), 6.56 (d, 1H), 7.4 (s, 1H), 7.47 (d, 1H), 7.52 (s, 1H), 8.07 (d, 1H), 8.5 (d, 1H), 10.18 (br s, 1H), 11.83 (br s, 1H); Mass Spectrum: M+H⁺ 490.

The N-(1-tert-butyl-3,4-trimethylenepyrazol-5-yl)-2-[5-(7-ethoxy-6-methoxyquinolin-4-yloxy)-3-methoxypyridin-2-yl]acetamide used as a starting material was prepared as follows:—

Using an analogous procedure to that described in Example 4, 5-amino-1-tert-butyl-3,4-trimethylenepyrazole (0.11 g) was reacted with 2-[5-(7-ethoxy-6-methoxyquinolin-4-yloxy)-3-methoxypyridin-2-yl]acetic acid (0.182 g) to give the required starting material (0.195 g); ¹H NMR: (DMSOd₆) 1.43 (t, 3H), 1.52 (s, 9H), 2.25 (m, 2H), 2.39 (m, 2H), 2.53 (m, 2H), 3.82 (s, 3H), 3.83 (s, 2H), 3.94 (s, 3H), 4.21 (q, 2H), 6.55 (d, 1H), 7.4 (s, 1H), 7.49 (d, 1H), 7.52 (s, 1H), 8.07 (d, 1H), 8.5 (d, 1H), 9.48 (s, 1H); Mass Spectrum: M+H⁺ 546.

[18] ¹H NMR: (DMSOd₆) 2.34 (m, 2H), 2.58 (m, 4H), 3.82 (2s, 5H), 3.94 (s, 3H), 6.7 (d, 1H), 7.49 (m, 2H), 7.58 (d, 1H), 7.97 (d, 1H), 8.09 (d, 1H), 8.57 (d, 1H); Mass Spectrum: M+H⁺ 446.

The N-(1-tert-butyl-3,4-trimethylenepyrazol-5-yl)-2-[5-(6-methoxyquinolin-4-yloxy)-3-methoxypyridin-2-yl]acetamide used as a starting material was prepared as follows:—

Using an analogous procedure to that described in Example 4, 5-amino-1-tert-butyl-3,4-trimethylenepyrazole (0.136 g) was reacted with 2-[5-(6-methoxyquinolin-4-yloxy)-3-methoxypyridin-2-yl]acetic acid (0.2 g) to give the required starting material (0.284 g); ¹H NMR: (DMSOd₆) 1.53 (s, 9H), 2.26 (m, 2H), 2.42 (m, 2H), 2.54 (m, 2H), 3.81 (s, 3H), 3.85 (s, 2H), 3.94 (s, 3H), 6.69 (d, 1H), 7.48 (m, 1H), 7.52 (d, 1H), 7.59 (d, 1H), 7.98 (d, 1H), 8.11 (d, 1H), 8.58 (d, 1H), 9.5 (s, 1H); Mass Spectrum: M+H⁺ 502.

[19] ¹H NMR: (DMSOd₆+CF₃CO₂D) 2.47 (m, 2H), 2.67 (m, 2H), 2.81 (m, 2H), 3.87 (s, 3H), 4.0 (s, 2H), 4.06 (s, 3H), 7.02 (d, 1H), 7.64 (s, 1H), 7.66 (s, 1H), 7.77 (s, 1H), 8.29 (s, 1H), 8.53 (s, 1H), 9.06 (s, 1H); Mass Spectrum: M+H⁺ 446.

The N-(1-tert-butyl-3,4-trimethylenepyrazol-5-yl)-2-[5-(7-methoxyquinolin-4-yloxy)-3-methoxypyridin-2-yl]acetamide used as a starting material was prepared as follows:—

Using an analogous procedure to that described in Example 4, 5-amino-1-tert-butyl-3,4-timethylenepyrazole (0.125 g) was reacted with 2-[5-(7-methoxyquinolin-4-yloxy)-3-methoxypyridin-2-yl]acetic acid (0.17 g) to give the required starting material (0.232 g); ¹H NMR: (DMSOd₆) 1.52 (s, 9H), 2.25 (m, 2H), 2.4 (m, 2H), 2.54 (m, 2H), 3.82 (s, 3H), 3.83 (s, 2H), 3.94 (s, 3H), 6.56 (d, 1H), 7.32 (m, 1H), 7.43 (s, 1H), 7.51 (s, 1H), 8.08 (s, 1H), 8.21 (d, 1H), 8.64 (d, 1H), 9.49 (br s, 1H); Mass Spectrum: M+H⁺ 502.

[20] ¹H NMR: (DMSOd₆) 2.36 (m, 2H), 2.57 (m, 4H), 3.82 (2s, 5H), 6.77 (d, 1H), 7.53 (d, 1H), 7.77 (m, 1H), 8.0 (m, 1H), 8.13 (m, 2H), 8.72 (m, 1H), 10.18 (br s, 1H); Mass Spectrum: M+H⁺ 434.

The N-(1-tert-butyl-3,4-trimethylenepyrazol-5-yl)-2-[5-(6-fluoroquinolin-4-yloxy)-3-methoxypyridin-2-yl]acetamide used as a starting material was prepared as follows:—

Using an analogous procedure to that described in Example 4, 5-amino-1-tert-butyl-3,4-trimethylenepyrazole (0.11 g) was reacted with 2-[5-(6-fluoroquinolin-4-yloxy)-3-methoxypyridin-2-yl]acetic acid (0.155 g) to give the required starting material (0.175 g); ¹H NMR: (DMSOd₆) 1.52 (s, 9H), 2.25 (m, 2H), 2.39 (m, 2H), 2.53 (m, 2H), 3.82 (s, 3H), 3.84 (s, 2H), 6.77 (d, 1H), 7.54 (d, 1H), 7.78 (m, 1H), 8.0 (m, 1H), 8.12 (d, 1H), 8.14 (m, 1H), 8.72 (m, 1H), 9.49 (br s, 1H); Mass Spectrum: M+H⁺ 490.

### EXAMPLE 7

### N-(5-isopropylpyrazol-3-yl)-2-[5-(6-fluoroquinolin-4-yloxy)-3-methoxypyridin-2-yl]acetamide

1-(3-Dimethylaminopropyl)-3-ethylcarbodiimide hydrochloride (0.437 g) and 2-hydroxypyridine N-oxide (0.253 g) were added in turn to a stirred mixture of 2-[5-(6-fluoroquinolin-4-yloxy)-3-methoxypyridin-2-yl]acetic acid (0.25 g), 3-amino-5-isopropylpyrazole (0.162 g), diisopropylethylamine (0.4 ml) and NMP (1.7 ml). The resultant mixture was stirred at ambient temperature for 7 hours and subsequently heated to 80° C. for 6 hours. The mixture was evaporated and the residue was purified by preparative HPLC using a Waters ‘Xterra’ reversed-phase column (5 microns silica, 30 mm diameter, 150 mm length) using decreasingly polar mixtures of water (containing 0.2% ammonium carbonate) and acetonitrile as eluent. There was thus obtained the title compound (0.047 g); ¹H NMR: (DMSOd₆) 1.19 (d, 6H), 3.81 (s, 3H), 3.84 (s, 2H), 6.27 (s, 1H), 6.75 (d, 1H), 7.53 (d, 1H), 7.77 (m, 1H), 8.01 (m, 1H), 8.13 (m, 2H), 8.72 (d, 1H), 10.42 (br s, 1H), 12.0 (br s, 1H); Mass Spectrum: M+H⁺ 436.

The 3-amino-5-isopropylpyrazole used as a starting material was prepared as follows:—

Acetonitrile (1.17 ml) was added dropwise to a stirred solution of n-butyllithium (1.6M in hexane, 14.06 ml) that had been cooled to −78° C. and the mixture was stirred at that temperature for 1 hour. Ethyl isobutyrate (1.5 ml) was added dropwise and the reaction medium was allowed to warm to −45° C. and stirred at that temperature for 2 hours. The resultant mixture was acidifed to pH2 by the addition of 2N aqueous hydrochloric acid and concentrated by evaporation. The residue was extracted with methylene chloride and the organic extract was dried over magnesium sulphate and evaporated. There was thus obtained 4-methyl-3-oxopentanenitrile (1.22 g); ¹H NMR: (CDCl₃) 1.18 (d, 6H), 2.82 (m, 1H), 3.52 (s, 2H).

A mixture of a portion (0.6 g) of the material so obtained, hydrazine hydrate (0.288 ml) and ethanol (45 ml) was heated at 70° C. for 12 hours. The solvent was evaporated and the residue was purified by column chromatography on silica using a 19:1 mixture of methylene chloride and methanol as eluent. There was thus obtained the required starting material (0.574 g); ¹H NMR: (DMSOd₆) 1.13 (d, 6H), 2.76 (m, 1H), 4.31 (br s, 2H), 5.17 (br s, 1H), 11.05 (br s, 1H); Mass Spectrum: M+H⁺ 126.

### EXAMPLE 8

### N-(5-cyclopropylpyrazol-3-yl)-2-[5-(6-fluoroquinolin-4-yloxy)-3-methoxypyridin-2-yl]acetamide

Using an analogous procedure to that described in Example 7, 2-[5-(6-fluoroquinolin-4-yloxy)-3-methoxypyridin-2-yl]acetic acid was reacted with 3-amino-5-cyclopropylpyrazole. There was thus obtained the title compound in 18% yield; ¹H NMR: (DMSOd₆) 0.64 (m, 2H), 0.9 (m, 2H), 1.85 (m, 1H), 3.81 (s, 3H), 3.83 (s, 2H), 6.13 (br s, 1H), 6.75 (d, 1H), 7.52 (d, 1H), 7.77 (m, 1H), 8.01 (m, 1H), 8.03 (s, 1H), 8.13 (m, 1H), 8.72 (d, 1H), 10.41 (br s, 1H); Mass Spectrum: M+H⁺ 434.

The 3-amino-5-cyclopropylpyrazole used as a starting material was prepared as follows:—

Using analogous procedures to those described in the portion of Example 7 hereinbefore that is concerned with the preparation of starting materials, ethyl cyclopropane-1-carboxylate was reacted with acetonitrile to give cyclopropyl cyanomethyl ketone which in turn was reacted with hydrazine hydrate to give the required starting material.

### EXAMPLE 9

### N-(5-isopropylpyrazol-3-yl)-2-[3-methoxy-5-(6-methoxyquinolin-4-yloxy)pyridin-2-yl]acetamide

Using an analogous procedure to that described in Example 7, except that the reaction mixture was heated to 70° C. for 3 hours, 2-[3-methoxy-5-(6-methoxyquinolin-4-yloxy)pyridin-2-yl]acetic acid was reacted with 3-amino-5-isopropylpyrazole. There was thus obtained the title compound in 26% yield; ¹H NMR: (DMSOd₆) 1.19 (d, 6H), 2.89 (m, 1H), 3.82 (s, 3H), 3.83 (s, 2H), 3.94 (s, 3H), 6.27 (br s, 1H), 6.66 (d, 1H), 7.48 (m, 1H), 7.5 (d, 1H), 7.59 (d, 1H), 7.97 (d, 1H), 8.1 (d, 1H), 8.57 (d, 1H), 10.41 (br s, 1H); Mass Spectrum: M+H⁺ 448.

### EXAMPLE 10

### N-(5-cyclopropylpyrazol-3-yl)-2-[3-methoxy-5-(6-methoxyquinolin-4-yloxy)pyridin-2-yl]acetamide

Using an analogous procedure to that described in Example 7, except that the reaction mixture was heated to 70° C. for 3 hours, 2-[3-methoxy-5-(6-methoxyquinolin-4-yloxy)pyridin-2-yl]acetic acid was reacted with 3-amino-5-cyclopropylpyrazole. There was thus obtained the title compound in 31% yield; ¹H NMR: (DMSOd₆) 0.64 (m, 2H), 0.89 (m, 2H), 1.85 (m, 1H), 3.81 (s, 3H), 3.82 (s, 3H), 3.94 (s, 3H), 6.14 (br s, 1H), 6.66 (d, 1H), 7.48 (m, 1H), 7.5 (d, 1H), 7.58 (d, 1H), 7.97 (d, 1H), 8.09 (d, 1H), 8.57 (d, 1H), 10.39 (br s, 1H); Mass Spectrum: M+H⁺ 446.

### EXAMPLE 11

### N-methyl-N-(5-methylthiazol-2-yl)-2-[5-(6,7-dimethoxyquinolin-4-yloxy)-3-methoxypyridin-2-yl]acetamide

Using an analogous procedure to that described in Example 1, except that DMA was used in place of DMF as the reaction solvent and the reaction mixture was heated to 50° C. for 5 hours, 2-[5-(6,7-dimethoxyquinolin-4-yloxy)-3-methoxypyridin-2-yl]acetic acid was reacted with 5-methyl-2-methylaminothiazole. The reaction product was purified by column chromatography on silica using a 19:1 mixture of methylene chloride and methanol as eluent. There was thus obtained the title compound in 46% yield; ¹H NMR: (DMSOd₆) 3.24 (d, 3H), 3.65 (s, 3H), 3.83 (s, 3H), 3.94 (s, 3H), 3.96 (s, 3H), 4.22 (s, 2H), 6.6 (d, 1H), 7.21 (q, 1H), 7.41 (s, 1H), 7.53 (s, 1H), 7.54 (d, 1H), 8.09 (d, 1H), 8.53 (d, 1H); Mass Spectrum: M+H⁺ 481.

The 5-methyl-2-methylaminothiazole used as a starting material was prepared as follows:—

Pyridine (0.107 ml) was added to a stirred suspension of 2-amino-5-methylthiazole (0.5 g) in acetic anhydride (0.944 ml). The resultant mixture was heated to 100° C. in a microwave oven for 10 minutes. The mixture was cooled to ambient temperature and diethyl ether was added. The precipitate was isolated and dried. There was thus obtained 2-acetamido-5-methylthiazole (0.634 g); ¹H NMR: (CDCl₃) 2.3 (s, 3H), 2.41 (s, 3H), 7.06 (br s, 1H); Mass Spectrum: M+H⁺ 157.

Under an atmosphere of argon, a 1M solution of lithium hexamethyldisilazane in THF (4.24 ml) was added dropwise to a stirred solution of 2-acetamido-5-methylthiazole (0.63 g) in THF (30 ml) that had been cooled to 0° C. After 10 minutes, the mixture was cooled to −30° C. and a solution of dimethyl sulphate (0.4 ml) in THF (4 ml) was added. The resultant mixture was stirred at −30° C. for 1 hour and at ambient temperature for 4 hours. The mixture was evaporated and the residue was purified by column chromatography on silica using a solvent gradient of 9:1 to 3:7 of methylene chloride and ethyl acetate as eluent. There was thus obtained 2-(N-methylacetamido)-5-methylthiazole (0.35 g); ¹H NMR: (CDCl₃) 2.38 (2s, 6H), 3.67 (s, 3H), 7.13 (s, 1H); Mass Spectrum: M+H⁺ 171.

A mixture of 2-(N-methylacetamido)-5-methylthiazole (0.35 g), sodium hydroxide (0.15 g) and methanol (10 ml) was stirred at ambient temperature for 16 hours. The mixture was evaporated. Water (5 ml) and methylene chloride (5 ml) were added and the basicity of the mixture was reduced by the addition of 2N aqueous hydrochloric acid (2 ml). A saturated solution of aqueous sodium bicarbonate was added to bring the pH to 8. The resultant aqueous phase was extracted with methylene chloride. The organic extract was dried over magnesium sulphate and evaporated. There was thus obtained 5-methyl-2-methylaminothiazole (0.26 g); ¹H NMR: (DMSOd₆) 2.19 (s, 3H), 2.75 (s, 3H), 6.67 (s, 1H), 7.22 (s, 1H); Mass Spectrum: M+H⁺ 129.

1: A quinoline derivative of the Formula I 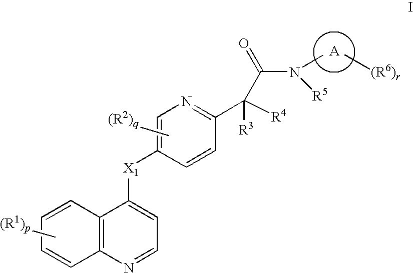
wherein
X¹ is O or N(R⁷) where R⁷ is hydrogen or (1-8C)alkyl;
p is 0, 1, 2 or 3;
each R¹ group, which may be the same or different, is selected from halogeno, trifluoromethyl, cyano, hydroxy, mercapto, amino, carboxy, (1-6C)alkoxycarbonyl, carbamoyl, (1-8C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulphinyl, (1-6C)alkylsulphonyl, (1-6C)alkylamino, di-[(1-6C)alkyl]amino, N-(1-6C)alkylcarbamoyl, N,N-di-[(1-6C)alkyl]carbamoyl, N-(1-6C)alkylsulphamoyl, N,N-di-[(1-6C)alkyl]sulphamoyl, (2-6C)alkanoyl, (2-6C)alkanoylamino and N-(1-6C)alkyl-(2-6C)alkanoylamino,
or from a group of the formula:
Q¹-X²-
wherein X² is selected from O, S, SO, SO₂, N(R⁸), CO, CON(R⁸), N(R⁸)CO, OC(R⁸)₂ and N(R⁸)C(R⁸)₂, wherein each R⁸ is hydrogen or (1-8C)alkyl, and Q¹ is aryl, aryl-(1-6C)alkyl, (3-8C)cycloalkyl, (3-8C)cycloalkyl-(1-6C)alkyl, (3-8C)cycloalkenyl, (3-8C)cycloalkenyl-(1-6C)alkyl, heteroaryl, heteroaryl-(1-6C)alkyl, heterocyclyl or heterocyclyl-(1-6C)alkyl,
and wherein any aryl, (3-8C)cycloalkyl, (3-8C)cycloalkenyl, heteroaryl or heterocyclyl group within a R¹ substituent optionally bears 1, 2 or 3 substituents, which may be the same or different, selected from halogeno, trifluoromethyl, cyano, nitro, hydroxy, amino, carboxy, carbamoyl, ureido, (1-8C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, (2-6C)alkanoyl, (2-6C)alkanoyloxy, N-(1-6C)alkylcarbamoyl, N,N-di-[(1-6C)alkyl]carbamoyl, (2-6C)alkanoylamino, N-(1-6C)alkyl-(2-6C)alkanoyl amino, N-(1-6C)alkylureido, N′-(1-6C)alkylureido, N′,N′-di-[(1-6C)alkyl]ureido, N,N′-di-[(1-6C)alkyl]ureido, N,N′,N′-tri-[(1-6C)alkyl]ureido, N-(1-6C)alkylsulphamoyl, N,N-di-[(1-6C)alkyl]sulphamoyl, (1-6C)alkanesulphonylamino and N-(1-6C)alkyl-(1-6C)alkanesulphonylamino,
or from a group of the formula:
-X³-R⁹
wherein X³ is a direct bond or is selected from O and N(R¹⁰), wherein R¹⁰ is hydrogen or (1-8C)alkyl, and R⁹ is halogeno-(1-6C)alkyl, hydroxy-(1-6C)alkyl, mercapto-(1-6C)alkyl, (1-6C)alkoxy-(1-6C)alkyl, (1-6C)alkylthio-(1-6C)alkyl, (1-6C)alkylsulphinyl-(1-6C)alkyl, (1-6C)alkylsulphonyl-(1-6C)alkyl, cyano-(1-6C)alkyl, amino-(1-6C)alkyl, (1-6C)alkylamino-(1-6C)alkyl, di-[(1-6C)alkyl]amino-(1-6C)alkyl, (2-6C)alkanoylamino-(1-6C)alkyl, N-(1-6C)alkyl-(2-6C)alkanoylamino-(1-6C)alkyl, (1-6C)alkoxycarbonylamino-(1-6C)alkyl, ureido-(1-6C)alkyl, N-(1-6C)alkylureido-(1-6C)alkyl, N′-(1-6C)alkylureido-(1-6C)alkyl, N′,N′-di-[(1-6C)alkyl]ureido-(1-6C)alkyl, N,N′-di-[(1-6C)alkyl]ureido-(1-6C)alkyl or N,N′,N′-tri-[(1-6C)alkyl]ureido-(1-6C)alkyl,
or from a group of the formula:
-X⁴-Q²
wherein X⁴ is a direct bond or is selected from O, CO and N(R¹¹), wherein R¹¹ is hydrogen or (1-8C)alkyl, and Q² is aryl, aryl-(1-6C)alkyl, heteroaryl, heteroaryl-(1-6C)alkyl, heterocyclyl or heterocyclyl-(1-6C)alkyl which optionally bears 1 or 2 substituents, which may be the same or different, selected from halogeno, hydroxy, (1-8C)alkyl and (1-6C)alkoxy,
and wherein any aryl, heteroaryl or heterocyclyl group within a substituent on R¹ optionally bears a (1-3C)alkylenedioxy group,
and wherein any heterocyclyl group within a R¹ substituent optionally bears 1 or 2 oxo or thioxo substituents,
and wherein any CH, CH₂ or CH₃ group within a R¹ substituent optionally bears on each said CH, CH₂ or CH₃ group one or more halogeno or (1-8C)alkyl substituents and/or a substituent selected from hydroxy, mercapto, amino, cyano, carboxy, carbamoyl, ureido, (1-6C)alkoxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylureido, N′-(1-6C)alkylureido, N′,N′-di-[(1-6C)alkyl]ureido, N,N′-di-[(1-6C)alkyl]ureido, N,N′,N′-tri-[(1-6C)alkyl]ureido, N-(1-6C)alkylsulphamoyl, N,N-di-[(1-6C)alkyl]sulphamoyl, (1-6C)alkanesulphonylamino and N-(1-6C)alkyl-(1-6C)alkanesulphonylamino,
and wherein adjacent carbon atoms in any (2-6C)alkylene chain within a R¹ substituent are optionally separated by the insertion into the chain of a group selected from O, S, SO, SO₂, N(R¹²), CO, CH(OR¹²), CON(R¹²), N(R¹²)CO, N(R¹²)CON(R¹²), SO₂N(R¹²), N(R¹²)SO₂, CH═CH and C≡C wherein R¹² is hydrogen or (1-8C)alkyl, or, when the inserted group is N(R¹²), R¹² may also be (2-6C)alkanoyl;
q is 0, 1 or 2;
each R² group, which may be the same or different, is selected from halogeno, trifluoromethyl, cyano, carboxy, hydroxy, amino, carbamoyl, (1-8C)alkyl, (2-8C)alkenyl, (2-8C)alkynyl, (1-6C)alkoxy, (1-6C)alkylamino, di-[(1-6C)alkyl]amino, N-(1-6C)alkylcarbamoyl, N,N-di-[(1-6C)alkyl]carbamoyl, halogeno-(1-6C)alkyl, hydroxy-(1-6C)alkyl, (1-6C)alkoxy-(1-6C)alkyl, cyano-(1-6C)alkyl, carboxy-(1-6C)alkyl, (1-6C)alkoxycarbonyl-(1-6C)alkyl, amino-(1-6C)alkyl, (1-6C)alkylamino-(1-6C)alkyl, di-[(1-6C)alkyl]amino-(1-6C)alkyl, carbamoyl-(1-6C)alkyl, N-(1-6C)alkylcarbamoyl-(1-6C)alkyl, N,N-di-[(1-6C)alkyl]carbamoyl-(1-6C)alkyl, (2-6C)alkanoylamino-(1-6C)alkyl and N-(1-6C)alkyl-(2-6C)alkanoylamino-(1-6C)alkyl;
R³ is hydrogen, (1-8C)alkyl, (2-8C)alkenyl or (2-8C)alkynyl;
R⁴ is hydrogen, (1-8C)alkyl, (2-8C)alkenyl, (2-8C)alkynyl, halogeno-(1-6C)alkyl, hydroxy-(1-6C)alkyl, (1-6C)alkoxy-(1-6C)alkyl, cyano-(1-6C)alkyl, carboxy-(1-6C)alkyl, amino-(1-6C)alkyl, (1-6C)alkylamino-(1-6C)alkyl, di-[(1-6C)alkyl]amino-(1-6C)alkyl, carbamoyl-(1-6C)alkyl, N-(1-6C)alkylcarbamoyl-(1-6C)alkyl, N,N-di-[(1-6C)alkyl]carbamoyl-(1-6C)alkyl, (1-6C)alkoxycarbonyl-(1-6C)alkyl, (2-6C)alkanoylamino-(1-6C)alkyl or N-(1-6C)alkyl-(2-6C)alkanoylamino-(1-6C)alkyl;
or R³ and R⁴ together with the carbon atom to which they are attached form a (3-8C)cycloalkyl group;
R⁵ is hydrogen, (1-8C)alkyl, (2-8C)alkenyl or (2-8C)alkynyl or a group of the formula:
-X⁵-R¹³
wherein X⁵ is a direct bond or is selected from O and N(R¹⁴), wherein R¹⁴ is hydrogen or (1-8C)alkyl, and R¹³ is halogeno-(1-6C)alkyl, hydroxy-(1-6C)alkyl, (1-6C)alkoxy-(1-6C)alkyl or cyano-(1-6C)alkyl;
Ring A is a 6-membered monocyclic or a 10-membered bicyclic aryl ring or a 5- or 6-membered monocyclic or a 9- or 10-membered bicyclic heteroaryl ring with up to three ring heteroatoms selected from oxygen, nitrogen and sulphur;
r is 0, 1, 2 or 3; and
each R⁶ group, which may be the same or different, is selected from halogeno, trifluoromethyl, cyano, hydroxy, mercapto, amino, carboxy, carbamoyl, sulphamoyl, ureido, (1-8C)alkyl, (2-8C)alkenyl, (2-8C)alkynyl, (1-6C)alkoxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, (2-6C)alkanoyl, (2-6C)alkanoyloxy, N-(1-6C)alkylcarbamoyl, N,N-di-[(1-6C)alkyl]carbamoyl, (2-6C)alkanoylamino, N-(1-6C)alkyl-(2-6C)alkanoylamino, N′-(1-6C)alkylureido, N′,N′-di-[(1-6C)alkyl]ureido, N-(1-6C)alkylsulphamoyl, N,N-di-[(1-6C)alkyl]sulphamoyl, (1-6C)alkanesulphonylamino and N-(1-6C)alkyl-(1-6C)alkanesulphonylamino,
or from a group of the formula:
-X⁶-R¹⁵
wherein X⁶ is a direct bond or is selected from O and N(R¹⁶), wherein R¹⁶ is hydrogen or (1-8C)alkyl, and R¹⁵ is halogeno-(1-6C)alkyl, hydroxy-(1-6C)alkyl, mercapto-(1-6C)alkyl, (1-6C)alkoxy-(1-6C)alkyl, (1-6C)alkylthio-(1-6C)alkyl, (1-6C)alkylsulphinyl-(1-6C)alkyl, (1-6C)alkylsulphonyl-(1-6C)alkyl, cyano-(1-6C)alkyl, amino-(1-6C)alkyl, (1-6C)alkylamino-(1-6C)alkyl, di-[(1-6C)alkyl]amino-(1-6C)alkyl, (2-6C)alkanoylamino-(1-6C)alkyl, N-(1-6C)alkyl-(2-6C)alkanoylamino-(1-6C)alkyl, carboxy-(1-6C)alkyl, (1-6C)alkoxycarbonyl-(1-6C)alkyl, carbamoyl-(1-6C)alkyl, N-(1-6C)alkylcarbamoyl-(1-6C)alkyl, N,N-di-[(1-6C)alkyl]carbamoyl-(1-6C)alkyl, sulphamoyl-(1-6C)alkyl, N-(1-6C)alkylsulphamoyl-(1-6C)alkyl, N,N-di-[(1-6C)alkyl]sulphamoyl-(1-6C)alkyl, ureido-(1-6C)alkyl, N-(1-6C)alkylureido-(1-6C)alkyl, N′-(1-6C)alkylureido-(1-6C)alkyl, N′,N′-di-[(1-6C)alkyl]ureido-(1-6C)alkyl, N,N′-di-[(1-6C)alkyl]ureido-(1-6C)alkyl, N,N′,N′-tri-[(1-6C)alkyl]ureido-(1-6C)alkyl, (1-6C)alkanesulphonylamino-(1-6C)alkyl or N-(1-6C)alkyl-(1-6C)alkanesulphonylamino-(1-6C)alkyl,
or from a group of the formula:
-X⁷-Q³
wherein X⁷ is a direct bond or is selected from O, S, SO, SO₂, N(R¹⁷), CO, CH(OR¹⁷), CON(R¹⁷), N(R¹⁷)CO, N(R¹⁷)CON(R¹⁷), SO₂N(R¹⁷), N(R¹⁷)SO₂, C(R¹⁷)₂O, C(R¹⁷)₂S and C(R¹⁷)₂N(R¹⁷), wherein each R¹⁷ is hydrogen or (1-8C)alkyl, and Q³ is aryl, aryl-(1-6C)alkyl, (3-8C)cycloalkyl, (3-8C)cycloalkyl-(1-6C)alkyl, (3-8C)cycloalkenyl, (3-8C)cycloalkenyl-(1-6C)alkyl, heteroaryl, heteroaryl-(1-6C)alkyl, heterocyclyl or heterocyclyl-(1-6C)alkyl,
or two R⁶ groups together form a bivalent group that spans adjacent ring positions on Ring A selected from OC(R¹⁸)₂O, OC(R¹⁸)₂C(R¹⁸)₂O, OC(R¹⁸)₂C(R¹⁸)₂, C(R¹⁸)₂OC(R¹⁸)₂, C(R¹⁸)₂C(R¹⁸)₂C(R¹⁸)₂, C(R¹⁸)₂C(R¹⁸)₂C(R¹⁸)₂C(R¹⁸)₂, OC(R¹⁸)₂N(R¹⁹), N(R¹⁹)C(R¹⁸)₂N(R¹⁹), N(R¹⁹)C(R¹⁸)₂C(R¹⁸)₂, N(R¹⁹)C(R¹⁸)₂C(R¹⁸)₂C(R¹⁸)₂, O C(R¹⁸)₂C(R¹⁸)₂N(R¹⁹), C(R¹⁸)₂N(R¹⁹)C(R¹⁸)₂, CO.N(R¹⁸)C(R¹⁸)₂, N(R¹⁸)CO.C(R¹⁸)₂, N(R¹⁹)C(R¹⁸)₂CO, CO.N(R¹⁸)CO, N(R¹⁹)N(R¹⁸)CO, N(R¹⁸)CO.N(R¹⁸), O.CO.N(R¹⁸), O.CO.C(R¹⁸)₂ and CO.OC(R¹⁸)₂ wherein each R¹⁸ is hydrogen, (1-8C)alkyl, (2-8C)alkenyl or (2-8C)alkynyl, and wherein R¹⁹ is hydrogen, (1-8C)alkyl, (2-8C)alkenyl, (2-8C)alkynyl or (2-6C)alkanoyl,
and wherein any aryl, (3-8C)cycloalkyl, (3-8C)cycloalkenyl, heteroaryl or heterocyclyl group within an R⁶ group optionally bears 1, 2 or 3 substituents, which may be the same or different, selected from halogeno, trifluoromethyl, cyano, nitro, hydroxy, amino, carboxy, carbamoyl, ureido, (1-8C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, (2-6C)alkanoyl, (2-6C)alkanoyloxy, N-(1-6C)alkylcarbamoyl, N,N-di-[(1-6C)alkyl]carbamoyl, (2-6C)alkanoylamino, N-(1-6C)alkyl-(2-6C)alkanoylamino, N′-(1-6C)alkylureido, N′,N′-di-[(1-6C)alkyl]ureido, N-(1-6C)alkylureido, N,N′-di-[(1-6C)alkyl]ureido, N,N′,N′-tri-[(1-6C)alkyl]ureido, N-(1-6C)alkylsulphamoyl, N,N-di-[(1-6C)alkyl]sulphamoyl, (1-6C)alkanesulphonylamino and N-(1-6C)alkyl-(1-6C)alkanesulphonylamino,
or from a group of the formula:
X⁸-R²⁰
wherein X⁸ is a direct bond or is selected from O and N(R²¹), wherein R²¹ is hydrogen or (1-8C)alkyl, and R²⁰ is halogeno-(1-6C)alkyl, hydroxy-(1-6C)alkyl, mercapto-(1-6C)alkyl, (1-6C)alkoxy-(1-6C)alkyl, (1-6C)alkylthio-(1-6C)alkyl, (1-6C)alkylsulphinyl-(1-6C)alkyl, (1-6C)alkylsulphonyl-(1-6C)alkyl, cyano-(1-6C)alkyl, amino-(1-6C)alkyl, (1-6C)alkylamino-(1-6C)alkyl, di-[(1-6C)alkyl]amino-(1-6C)alkyl, (2-6C)alkanoylamino-(1-6C)alkyl or N-(1-6C)alkyl-(2-6C)alkanoylamino-(1-6C)alkyl,
or from a group of the formula:
-X⁹-Q⁴
wherein X⁹ is a direct bond or is selected from O, CO and N(R²²), wherein R²² is hydrogen or (1-8C)alkyl, and Q⁴ is aryl, aryl-(1-6C)alkyl, heteroaryl, heteroaryl-(1-6C)alkyl, heterocyclyl or heterocyclyl-(1-6C)alkyl which optionally bears 1 or 2 substituents, which may be the same or different, selected from halogeno, hydroxy, (1-8C)alkyl and (1-6C)alkoxy,
and wherein any aryl, heteroaryl or heterocyclyl group within an R⁶ group optionally bears a (1-3C)alkylenedioxy group,
and wherein any heterocyclyl group within an R⁶ group optionally bears 1 or 2 oxo or thioxo substituents,
and wherein any CH, CH₂ or CH₃ group within an R⁶ group optionally bears on each said CH, CH₂ or CH₃ group one or more halogeno or (1-8C)alkyl substituents and/or a substituent selected from hydroxy, mercapto, amino, cyano, carboxy, carbamoyl, ureido, (2-8C)alkenyl, (2-8C)alkynyl, (1-6C)alkoxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N′-(1-6C)alkylureido, N′,N′-di-[(1-6C)alkyl]ureido, N-(1-6C)alkylureido, N,N′-di-[(1-6C)alkyl]ureido, N,N′,N′-tri-[(1-6C)alkyl]ureido, N-(1-6C)alkylsulphamoyl, N-(1-6C)alkylsulphamoyl, N,N-di-[(1-6C)alkyl]sulphamoyl, (1-6C)alkanesulphonylamino and N-(1-6C)alkyl-(1-6C)alkanesulphonylamino,
and wherein adjacent carbon atoms in any (2-6C)alkylene chain within an R⁶ group are optionally separated by the insertion into the chain of a group selected from O, S, SO, SO₂, N(R²³), N(R²³)CO, CON(R²³), N(R²³)CON(R²³), CO, CH(OR²³), N(R²³)SO₂, SO₂N(R²), CH═CH and C≡C wherein R²³ is hydrogen or (1-8C)alkyl, or, when the inserted group is N(R²³), R²³ may also be (2-6C)alkanoyl;
or a pharmaceutically-acceptable salt thereof.

2: The quinoline derivative of the Formula I according to claim 1 wherein:—
X¹ is O;
p is 2 and the R¹ groups are located at the 6- and 7-positions and the R¹ group at the 6-position is selected from cyano, hydroxy, methoxycarbonyl, ethoxycarbonyl, carbamoyl, methoxy, ethoxy, propoxy, N-methylcarbamoyl, N-ethylcarbamoyl, N,N-dimethylcarbamoyl, N,N-diethylcarbamoyl, pyrrolidin-1-ylcarbonyl, morpholinocarbonyl, piperidinocarbonyl and piperazin-1-ylcarbonyl, and the R¹ group at the 7-position is selected from methoxy, ethoxy, propoxy, 2-pyrrolidin-1-ylethoxy, 3-pyrrolidin-1-ylpropoxy, 4-pyrrolidin-1-ylbutoxy, pyrrolidin-3-yloxy, pyrrolidin-2-ylmethoxy, 2-pyrrolidin-2-ylethoxy, 3-pyrrolidin-2-ylpropoxy, 2-morpholinoethoxy, 3-morpholinopropoxy, 4-morpholinobutoxy, 2-(1,1-dioxotetrahydro-4H-1,4-thiazin-4-yl)ethoxy, 3-(1,1-dioxotetrahydro-4H-1,4-thiazin-4-yl)propoxy, 2-piperidinoethoxy, 3-piperidinopropoxy, 4-piperidinobutoxy, piperidin-3-yloxy, piperidin-4-yloxy, piperidin-3-ylmethoxy, 2-piperidin-3-ylethoxy, piperidin-4-ylmethoxy, 2-piperidin-4-ylethoxy, 2-homopiperidin-1-ylethoxy, 3-homopiperidin-1-ylpropoxy, 3-(1,2,3,6-tetrahydropyridin-1-yl)propoxy, 2-piperazin-1-ylethoxy, 3-piperazin-1-ylpropoxy, 2-homopiperazin-1-ylethoxy and 3-homopiperazin-1-ylpropoxy,
and wherein any heterocyclyl group within a substituent on R¹ optionally bears 1 or 2 substituents, which may be the same or different, selected from fluoro, chloro, trifluoromethyl, hydroxy, amino, methyl, ethyl, methoxy, methylenedioxy, ethylidendioxy and isopropylidenedioxy, and a pyrrolidin-2-yl, pyrrolidin-3-yl, piperidin-3-yl, piperidin-4-yl, piperazin-1-yl or homopiperazin-1-yl group within a R¹ substituent is optionally N-substituted with methyl, ethyl, propyl, allyl, 2-propynyl, methylsulphonyl, acetyl, propionyl, isobutyryl, 2-fluoroethyl, 2,2-difluoroethyl, 2,2,2-trifluoroethyl or cyanomethyl,
and wherein any heterocyclyl group within a substituent on R¹ optionally bears 1 or 2 oxo substituents,
and wherein any CH, CH₂ or CH₃ group within a R¹ substituent optionally bears on each said CH, CH₂ or CH₃ group one or more chloro groups or a substituent selected from hydroxy, amino, methoxy, methylsulphonyl, methylamino, dimethylamino, diisopropylamino, N-ethyl-N-methylamino and N-isopropyl-N-methylamino;
q is 0 or
q is 1 and the R² group which is located at the 2-position (relative to the C(R³)(R⁴) group) is selected from fluoro, chloro, trifluoromethyl, cyano, hydroxy, amino, methyl, methoxy, methylamino and dimethylamino;
each of R³ and R⁴ is hydrogen;
R⁵ is hydrogen, methyl or ethyl;
Ring A is a phenyl, pyridyl, pyrimidinyl, pyrazinyl or pyridazinyl ring; and
r is 0 or
r is 1 or 2 and one R⁶ group is located at the 3- or 4-position (relative to the CON(R⁵) group), and each R⁶ group, which may be the same or different, is selected from fluoro, chloro, trifluoromethyl, cyano, hydroxy, amino, methyl, methoxy, methylamino and dimethylamino, or
r is 1 or 2 and one R⁶ group is located at the 3- or 4-position (relative to the CON(R⁵) group) and is a group of the formula:
-X⁶-R¹⁵
wherein X⁶ is a direct bond or O and R¹⁵ is hydroxymethyl, 1-hydroxyethyl, 2-hydroxyethyl, 3-hydroxypropyl, methoxymethyl, 1-methoxyethyl, 2-methoxyethyl, 1-methoxy-1-methylethyl, 3-methoxypropyl, cyanomethyl, 1-cyanoethyl, 2-cyanoethyl, 3-cyanopropyl, aminomethyl, 1-aminoethyl, 2-aminoethyl, 3-aminopropyl, methylaminomethyl, 1-methylaminoethyl, 2-methylaminoethyl, 3-methylaminopropyl, ethylaminomethyl, 1-ethylaminoethyl, 2-ethylaminoethyl, 1-ethylamino-1-methylethyl, 3-ethylaminopropyl, isopropylaminomethyl, 1-isopropylaminoethyl, dimethylaminomethyl, 1-dimethylaminoethyl, 2-dimethylaminoethyl, 3-dimethylaminopropyl, phenyl, benzyl, cyclopropyl, cyclopentyl, cyclohexyl, thienyl, imidazolyl, thiazolyl, thiadiazolyl, pyrrolidinyl, morpholinyl, tetrahydro-1,4-thiazinyl, piperidinyl, homopiperidinyl, piperazinyl, homopiperazinyl, pyrrolidinylmethyl, 2-(pyrrolidinyl)ethyl, 3-(pyrrolidinyl)propyl, morpholinylmethyl, 2-(morpholinyl)ethyl, 3-(morpholinyl)propyl, piperidinylmethyl, 2-(piperidinyl)ethyl, 3-(piperidinyl)propyl, homopiperidinylmethyl, piperazinylmethyl, 2-(piperazinyl)ethyl, 3-(piperazinyl)propyl or homopiperazinylmethyl, provided that, when X⁶ is O, there are at least two carbon atoms between X⁶ and any heteroatom in the R¹⁵ group,
and wherein any aryl, (3-8C)cycloalkyl, heteroaryl or heterocyclyl group within the R⁶ group optionally bears a substituent selected from fluoro, chloro, trifluoromethyl, hydroxy, amino, methyl, methoxy, methylamino and dimethylamino and any such aryl, (3-8C)cycloalkyl, heteroaryl or heterocyclyl group within the R⁶ group optionally bears a further substituent selected from hydroxymethyl, cyanomethyl, aminomethyl, methylaminomethyl and dimethylaminomethyl,
and any second R⁶ group that is present is selected from fluoro, chloro, trifluoromethyl, cyano, hydroxy, amino, methyl, methoxy, methylamino and dimethylamino;
or a pharmaceutically-acceptable salt thereof.

3: The quinoline derivative of the Formula I according to claim 1 wherein:—
X¹ is O;
p is 2 and the R¹ groups, which may be the same or different, are located at the 6- and 7-positions and are selected from cyano, methoxy, ethoxy, propoxy, 2-hydroxyethoxy, 3-hydroxypropoxy, 2-methoxyethoxy, 3-methoxypropoxy, 2-methylsulphonylethoxy, 3-methylsulphonylpropoxy and 2-(2-methoxyethoxy)ethoxy;
q is 0 or
q is 1 and the R² group is fluoro, chloro, methyl or methoxy;
each of R³ and R⁴ is hydrogen;
R⁵ is hydrogen, methyl or ethyl;
Ring A is pyridyl, pyrimidinyl, pyrazinyl or pyridazinyl; and
r is 0, 1 or 2 and each R⁶ group that is present is selected from fluoro, chloro, trifluoromethyl, cyano, methyl, ethyl, propyl, isopropyl, tert-butyl, cyclopropyl, cyclobutyl, cyclopentyl, methoxy, ethoxy, methylamino, ethylamino, propylamino, isopropylamino, cyclopropylamino, 2-hydroxyethylamino, 2-methoxyethylamino, dimethylamino, N-cyclopropyl-N-methylamino, acetyl, hydroxymethyl, aminomethyl, methylaminomethyl, ethylaminomethyl, propylaminomethyl, isopropylaminomethyl, cyclopropylaminomethyl, dimethylaminomethyl, diethylaminomethyl, N-ethyl-N-methylaminomethyl, N-cyclopropyl-N-methylaminomethyl, pyrrolidin-1-yl, piperidino, morpholino, piperazin-1-yl, pyrrolidin-1-ylmethyl, morpholinomethyl, piperidinomethyl and piperazin-1-ylmethyl, and wherein any heterocyclyl group within the R⁶ group optionally bears a methyl or ethyl substituent;
or a pharmaceutically-acceptable salt thereof.

4: The quinoline derivative of the Formula I according to claim 1 wherein:—
Ring A is a 5-membered monocyclic heteroaryl ring with up to three ring heteroatoms selected from oxygen, nitrogen and sulphur; and
each of X, p, R¹,q, R², R³, R⁴, r and R⁶ has any of the meanings defined in claim 1;
or a pharmaceutically-acceptable salt thereof.

5: The quinoline derivative of the Formula I according to claim 1 wherein:—
X¹ is O;
p is 2 and the R¹ groups are located at the 6- and 7-positions and the R¹ group at the 6-position is selected from cyano, hydroxy, methoxycarbonyl, ethoxycarbonyl, carbamoyl, methoxy, ethoxy, propoxy, N-methylcarbamoyl, N-ethylcarbamoyl, N,N-dimethylcarbamoyl, N,N-diethylcarbamoyl, pyrrolidin-1-ylcarbonyl, morpholinocarbonyl, piperidinocarbonyl and piperazin-1-ylcarbonyl, and the R¹ group at the 7-position is selected from methoxy, ethoxy, propoxy, 2-pyrrolidin-1-ylethoxy, 3-pyrrolidin-1-ylpropoxy, 4-pyrrolidin-1-ylbutoxy, pyrrolidin-3-yloxy, pyrrolidin-2-ylmethoxy, 2-pyrrolidin-2-ylethoxy, 3-pyrrolidin-2-ylpropoxy, 2-morpholinoethoxy, 3-morpholinopropoxy, 4-morpholinobutoxy, 2-(1,1-dioxotetrahydro-4H-1,4-thiazin-4-yl)ethoxy, 3-(1,1-dioxotetrahydro-4H-1,4-thiazin-4-yl)propoxy, 2-piperidinoethoxy, 3-piperidinopropoxy, 4-piperidinobutoxy, piperidin-3-yloxy, piperidin-4-yloxy, piperidin-3-ylmethoxy, 2-piperidin-3-ylethoxy, piperidin-4-ylmethoxy, 2-piperidin-4-ylethoxy, 2-homopiperidin-1-ylethoxy, 3-homopiperidin-1-ylpropoxy, 3-(1,2,3,6-tetrahydropyridin-1-yl)propoxy, 2-piperazin-1-ylethoxy, 3-piperazin-1-ylpropoxy, 2-homopiperazin-1-ylethoxy and 3-homopiperazin-1-ylpropoxy,
and wherein any heterocyclyl group within a substituent on R¹ optionally bears 1 or 2 substituents, which may be the same or different, selected from fluoro, chloro, trifluoromethyl, hydroxy, amino, methyl, ethyl, methoxy, methylenedioxy, ethylidendioxy and isopropylidenedioxy, and a pyrrolidin-2-yl, pyrrolidin-3-yl, piperidin-3-yl, piperidin-4-yl, piperazin-1-yl or homopiperazin-1-yl group within a R¹ substituent is optionally N-substituted with methyl, ethyl, propyl, allyl, 2-propynyl, methylsulphonyl, acetyl, propionyl, isobutyryl, 2-fluoroethyl, 2,2-difluoroethyl, 2,2,2-trifluoroethyl or cyanomethyl,
and wherein any heterocyclyl group within a substituent on R¹ optionally bears 1 or 2 oxo substituents,
and wherein any CH, CH₂ or CH₃ group within a R¹ substituent optionally bears on each said CH, CH₂ or CH₃ group one or more chloro groups or a substituent selected from hydroxy, amino, methoxy, methylsulphonyl, methylamino, dimethylamino, diisopropylamino, N-ethyl-N-methylamino and N-isopropyl-N-methylamino;
q is 0 or
q is 1 and the R² group is selected from fluoro, chloro, trifluoromethyl, cyano, hydroxy, amino, methyl, methoxy, methylamino and dimethylamino;
each of R³ and R⁴ is hydrogen;
R⁵ is hydrogen, methyl or ethyl;
Ring A is a furyl, pyrrolyl, thienyl, oxazolyl, isoxazolyl, imidazolyl, pyrazolyl, thiazolyl, isothiazolyl, oxadiazolyl or thiadiazolyl ring; and
r is 0 or
r is 1 or 2 and one R⁶ group is located at the 3-position (relative to the CON(R⁵) group), and each R⁶ group, which may be the same or different, is selected from fluoro, chloro, trifluoromethyl, cyano, hydroxy, amino, methyl, ethyl, propyl, isopropyl, butyl, sec-butyl, isobutyl, tert-butyl, methoxy, ethoxy, methylamino, ethylamino, dimethylamino and diethylamino, or
r is 1 or 2 and one R⁶ group is located at the 3-position (relative to the CON(R⁵)group) and is a group of the formula:
-X⁶-R¹⁵
wherein X⁶ is a direct bond or O and R¹⁵ is hydroxymethyl, 1-hydroxyethyl, 2-hydroxyethyl, 3-hydroxypropyl, methoxymethyl, 1-methoxyethyl, 2-methoxyethyl, 1-methoxy-1-methylethyl, 3-methoxypropyl, cyanomethyl, 1-cyanoethyl, 2-cyanoethyl, 3-cyanopropyl, aminomethyl, 1-aminoethyl, 2-aminoethyl, 3-aminopropyl, methylaminomethyl, 1-methylaminoethyl, 2-methylaminoethyl, 3-methylaminopropyl, ethylaminomethyl, 1-ethylaminoethyl, 2-ethylaminoethyl, 1-ethylamino-1-methylethyl, 3-ethylaminopropyl, isopropylaminomethyl, 1-isopropylaminoethyl, dimethylaminomethyl, 1-dimethylaminoethyl, 2-dimethylaminoethyl, 3-dimethylaminopropyl, phenyl, benzyl, cyclopropyl, cyclopentyl, cyclohexyl, thienyl, imidazolyl, thiazolyl, thiadiazolyl, pyrrolidinyl, morpholinyl, tetrahydro-1,4-thiazinyl, piperidinyl, homopiperidinyl, piperazinyl, homopiperazinyl, pyrrolidinylmethyl, 2-(pyrrolidinyl)ethyl, 3-(pyrrolidinyl)propyl, morpholinylmethyl, 2-(morpholinyl)ethyl, 3-(morpholinyl)propyl, piperidinylmethyl, 2-(piperidinyl)ethyl, 3-(piperidinyl)propyl, homopiperidinylmethyl, piperazinylmethyl, 2-(piperazinyl)ethyl, 3-(piperazinyl)propyl or homopiperazinylmethyl, provided that, when X⁶ is O, there are at least two carbon atoms between X⁶ and any heteroatom in the R¹⁵ group,
and wherein any aryl, (3-8C)cycloalkyl, heteroaryl or heterocyclyl group within the R⁶ group optionally bears a substituent selected from fluoro, chloro, trifluoromethyl, hydroxy, amino, methyl, methoxy, methylamino and dimethylamino and any such aryl, (3-8C)cycloalkyl, heteroaryl or heterocyclyl group within the R⁶ group optionally bears a further substituent selected from hydroxymethyl, cyanomethyl, aminomethyl, methylaminomethyl and dimethylaminomethyl,
and any second R⁶ group that is present is selected from fluoro, chloro, trifluoromethyl, cyano, hydroxy, amino, methyl, methoxy, methylamino and dimethylamino;
or a pharmaceutically-acceptable salt thereof.

6: The quinoline derivative of the Formula I according to claim 1 wherein:—
X¹ is O;
p is 2 and
the first R¹ group is located at the 6-position and is selected from cyano, carbamoyl, methoxy, N-methylcarbamoyl and N,N-dimethylcarbamoyl, and
the second R¹ group is located at the 7-position and is selected from methoxy, ethoxy, propoxy, 2-hydroxyethoxy, 3-hydroxypropoxy, 2-methoxyethoxy, 3-methoxypropoxy, 2-methylsulphonylethoxy, 3-methylsulphonylpropoxy and 2-(2-methoxyethoxy)ethoxy;
q is 0 or
q is 1 and the R² group which is located at the 2-position (relative to the C(R³)(R⁴) group) is selected from fluoro, chloro, cyano, methyl and methoxy;
each of R³ and R⁴ is hydrogen;
R⁵ is hydrogen or methyl;
Ring A is selected from oxazolyl, isoxazolyl, imidazolyl, pyrazolyl, thiazolyl, isothiazolyl, oxadiazolyl and thiadiazolyl; and
r is 0, 1 or 2 and each R⁶ group that is present is selected from fluoro, chloro, trifluoromethyl, hydroxy, amino, methyl, ethyl, propyl, isopropyl, butyl, sec-butyl, isobutyl, tert-butyl, cyclopropyl, cyclobutyl, cyclopentyl, hydroxymethyl, 2-hydroxyethyl, methoxymethyl, 2-methoxyethyl, methylaminomethyl, ethylaminomethyl, isopropylaminomethyl, cyclopropylaminomethyl, dimethylaminomethyl, methoxy, ethoxy, methylamino, ethylamino, dimethylamino and diethylamino;
or a pharmaceutically-acceptable salt thereof.

7: The quinoline derivative of the Formula I according to claim 1 wherein:—
X¹ is O;
p is 2 and
the first R¹ group is located at the 6-position and is selected from cyano, carbamoyl, methoxy, N-methylcarbamoyl and N,N-dimethylcarbamoyl, and
the second R¹ group is located at the 7-position and is selected from methoxy, ethoxy, 2-hydroxyethoxy and 2-methoxyethoxy;
q is 0 or
q is 1 and the R² group which is located at the 2-position (relative to the C(R³)(R⁴) group) is selected from fluoro, chloro, cyano, methyl and methoxy;
each of R³ and R⁴ is hydrogen;
R⁵ is hydrogen or methyl;
Ring A is 2-oxazolyl, 3-isoxazolyl, 5-isoxazolyl, 2-imidazolyl, 3-pyrazolyl, 4-pyrazolyl, 2-thiazolyl, 3-isothiazolyl, 5-isothiazolyl, 1,2,4-oxadiazol-5-yl and 1,3,4-oxadiazol-5-yl; and
r is 1 or 2 and each R⁶ group that is present is selected from methyl, ethyl, propyl, isopropyl, tert-butyl, cyclopropyl, hydroxymethyl, 2-hydroxyethyl, methoxymethyl, 2-methoxyethyl, methylaminomethyl, ethylaminomethyl, isopropylaminomethyl, cyclopropylaminomethyl, dimethylaminomethyl, amino, methylamino, ethylamino, dimethylamino and diethylamino;
or a pharmaceutically-acceptable salt thereof.

8: The quinoline derivative of the Formula I according to claim 1 wherein:—
p is 0 or
p is 1 or 2 and the R¹ groups are located at the 6- and/or 7-positions and are selected from halogeno, trifluoromethyl, cyano, hydroxy, amino, carbamoyl, (1-6C)alkoxycarbonyl, (1-8C)alkyl, (2-8C)alkenyl, (2-8C)alkynyl, (1-6C)alkoxy, (2-6C)alkenyloxy, (2-6C)alkynyloxy, (1-6C)alkylamino, di-[(1-6C)alkyl]amino, N-(1-6C)alkylcarbamoyl and N,N-di-[(1-6C)alkyl]carbamoyl, and
q is 1 and the R² group is located at the 2-position (relative to the C(R³)(R⁴) group) and is selected from halogeno, trifluoromethyl, cyano, carbamoyl, hydroxy, amino, (1-8C)alkyl, (2-8C)alkenyl, (2-8C)alkynyl, (1-6C)alkoxy, (1-6C)alkylamino, di-[(1-6C)alkyl]amino, N-(1-6C)alkylcarbamoyl and N,N-di-[(1-6C)alkyl]carbamoyl;
and each of X¹, R³, R⁴, R⁵, Ring A, r and R⁶ has any of the meanings defined in claim 1.

9: The quinoline derivative of the Formula I according to claim 1 wherein:—
p is 0 or
p is 1 or 2 and the R¹ groups are located at the 6- and/or 7-positions and are selected from fluoro, chloro, cyano, carbamoyl, methoxycarbonyl, methoxy, ethoxy, N-methylcarbamoyl and N,N-dimethylcarbamoyl, and
q is 1 and the R² group which is located at the 2-position (relative to the C(R³)(R⁴)group) is selected from carbamoyl, methoxy, ethoxy, N-methylcarbamoyl and N,N-dimethylcarbamoyl;
and each of X¹, R³, R⁴, R⁵, Ring A, r and R⁶ has any of the meanings defined in claim 1.

10: The quinoline derivative of the Formula I according to claim 1 wherein:—
X¹ is O;
p is 0 or
p is 1 or 2 and the R¹ groups are located at the 6- and/or 7-positions and are selected from halogeno, trifluoromethyl, cyano, hydroxy, amino, carbamoyl, (1-6C)alkoxycarbonyl, (1-8C)alkyl, (2-8C)alkenyl, (2-8C)alkynyl, (1-6C)alkoxy, (2-6C)alkenyloxy, (2-6C)alkynyloxy, (1-6C)alkylamino, di-[(1-6C)alkyl]amino, N-(1-6C)alkylcarbamoyl and N,N-di-[(1-6C)alkyl]carbamoyl,
q is 1 and the R² group is located at the 2-position (relative to the C(R³)(R⁴) group) and is selected from halogeno, trifluoromethyl, cyano, carbamoyl, hydroxy, amino, (1-8C)alkyl, (2-8C)alkenyl, (2-8C)alkynyl, (1-6C)alkoxy, (1-6C)alkylamino, di-[(1-6C)alkyl]amino, N-(1-6C)alkylcarbamoyl and N,N-di-[(1-6C)alkyl]carbamoyl;
each of R³ and R⁴ is hydrogen;
R⁵ is hydrogen;
Ring A is a 5-membered monocyclic heteroaryl ring with up to three ring heteroatoms selected from oxygen, nitrogen and sulphur; and
r is 0, 1, 2 or 3 and each R⁶ group that is present, which may be the same or different, is selected from halogeno, trifluoromethyl, cyano, hydroxy, amino, (1-8C)alkyl, (2-8C)alkenyl, (2-8C)alkynyl, (1-6C)alkoxy, (1-6C)alkylamino, di-[(1-6C)alkyl]amino, (2-6C)alkanoylamino and N-(1-6C)alkyl-(2-6C)alkanoylamino;
or a pharmaceutically-acceptable salt thereof.

11: The quinoline derivative of the Formula I according to claim 1 wherein:—
X¹ is O;
p is 0 or
p is 1 or 2 and the R¹ groups are located at the 6- and or 7-positions and are selected from fluoro, chloro, trifluoromethyl, cyano, hydroxy, amino, carbamoyl, methoxycarbonyl, ethoxycarbonyl, methyl, ethyl, methoxy, ethoxy, methylamino, dimethylamino, N-methylcarbamoyl and N,N-dimethylcarbamoyl,
q is 1 and the R² group which is located at the 2-position (relative to the C(R³)(R⁴) group) is selected from fluoro, chloro, trifluoromethyl, cyano, carbamoyl, hydroxy, amino, methyl, ethyl, methoxy, ethoxy, methylamino, dimethylamino, N-methylcarbamoyl and N,N-dimethylcarbamoyl;
each of R³ and R⁴ is hydrogen;
R⁵ is hydrogen;
Ring A is a furyl, pyrrolyl, thienyl, oxazolyl, isoxazolyl, imidazolyl, pyrazolyl, thiazolyl, isothiazolyl, oxadiazolyl or thiadiazolyl ring; and
r is 1 or 2 and each R⁶ group, which may be the same or different, is selected from fluoro, chloro, trifluoromethyl, cyano, hydroxy, amino, methyl, ethyl, propyl, isopropyl, butyl, sec-butyl, isobutyl, tert-butyl, methoxy, ethoxy, methylamino, ethylamino, dimethylamino and diethylamino;
or a pharmaceutically-acceptable salt thereof.

12: The quinoline derivative of the Formula I according to claim 1 wherein:—
X¹ is O;
p is 0 or
p is 1 or 2 and the R¹ groups are located at the 6- and/or 7-positions and are selected from fluoro, cyano, carbamoyl, methoxycarbonyl, methoxy, ethoxy, N-methylcarbamoyl and N,N-dimethylcarbamoyl,
q is 1 and the R² group which is located at the 2-position (relative to the C(R³)(R⁴) group) is selected from methoxy and ethoxy;
each of R³ and R⁴ is hydrogen;
R⁵ is hydrogen;
Ring A is a furyl, pyrrolyl, thienyl, oxazolyl, isoxazolyl, imidazolyl, pyrazolyl, thiazolyl, isothiazolyl, oxadiazolyl or thiadiazolyl ring that bears one or two R⁶ groups and one R⁶ group is located at the 3-position (relative to the CON(R⁵) group); and
r is 1 or 2 and each R⁶ group, which may be the same or different, is selected from fluoro, chloro, trifluoromethyl, cyano, hydroxy, amino, methyl, ethyl, propyl, isopropyl, butyl, sec-butyl, isobutyl, tert-butyl, methoxy, ethoxy, methylamino, ethylamino, dimethylamino and diethylamino;
or a pharmaceutically-acceptable salt thereof.

13: The quinoline derivative of the Formula I according to claim 1 wherein
X¹ is O;
p is 0 or
p is 1 or 2 and the R¹ groups are located at the 6- and/or 7-positions and are selected from fluoro, cyano, carbamoyl, methoxycarbonyl, methoxy, ethoxy, N-methylcarbamoyl and N,N-dimethylcarbamoyl,
q is 1 and the R² group which is located at the 2-position (relative to the C(R³)(R⁴) group) is a methoxy group;
each of R³ and R⁴ is hydrogen;
R⁵ is hydrogen;
Ring A is 2-oxazolyl, 3-isoxazolyl, 3-pyrazolyl, 4-pyrazolyl and 2-thiazolyl; and
r is 1 or 2 and each R⁶ group that is present is selected from methyl, ethyl, propyl and isopropyl;
or a pharmaceutically-acceptable salt thereof.

14: The quinoline derivative of the Formula I according to claim 1 selected from:—
N-(1-ethyl-1H-pyrazol-4-yl)-2-[3-methoxy-5-(7-methoxyquinolin-4-yloxy)pyridin-2-yl]acetamide,
N-(1,3-dimethyl-1H-pyrazol-4-yl)-2-[3-methoxy-5-(7-methoxyquinolin-4-yloxy)pyridin-2-yl]acetamide,
N-(1-ethyl-1H-pyrazol-4-yl)-2-[5-(6-fluoroquinolin-4-yloxy)-3-methoxypyridin-2-yl]acetamide,
N-(4,5-dimethyl-1H-pyrazol-3-yl)-2-[5-(6,7-dimethoxyquinolin-4-yloxy)-3-methoxypyridin-2-yl]acetamide,
N-(4,5-dimethylisoxazol-3-yl)-2-[5-(6,7-dimethoxyquinolin-4-yloxy)-3-methoxypyridin-2-yl]acetamide,
N-(4,5-dimethylisoxazol-3-yl)-2-[3-methoxy-5-(7-methoxyquinolin-4-yloxy)pyridin-2-yl]acetamide,
N-(5-ethylisoxazol-3-yl)-2-[5-(6-fluoroquinolin-4-yloxy)-3-methoxypyridin-2-yl]acetamide and
N-(4,5-dimethylisoxazol-3-yl)-2-[5-(6-fluoroquinolin-4-yloxy)-3-methoxypyridin-2-yl]acetamide;
and pharmaceutically-acceptable salts thereof.

15: A process for the preparation of a quinoline derivative of the Formula I, or a pharmaceutically-acceptable salt thereof, according to claim 1 which comprises:—
(a) the reaction of a quinoline of the Formula II 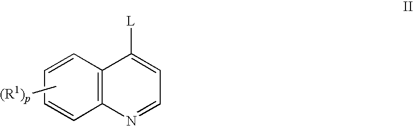
wherein L is a displaceable group and p and R¹ have any of the meanings defined in claim 1 except that any functional group is protected if necessary, with a 2-(2-pyridyl)acetamide of the Formula III 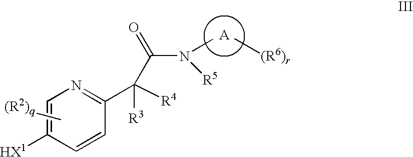
wherein X¹, q, R², R³, R⁴, R⁵, Ring A, r and R⁶ have any of the meanings defined in claim 1 except that any functional group is protected if necessary, whereafter any protecting group that is present is removed;
(b) the coupling of a quinoline of the Formula VII 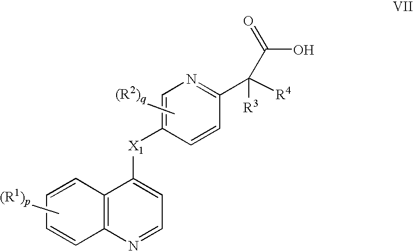
or a reactive derivative thereof, wherein p, R¹, X¹, q, R², R³ and R⁴ have any of the meanings defined in claim 1 except that any functional group is protected if necessary, with an amine of the Formula VI 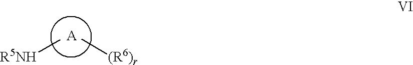
wherein R⁵, Ring A, r and R⁶ have any of the meanings defined in claim 1 except that any functional group is protected if necessary, whereafter any protecting group that is present is removed;
(c) for the production of those compounds of the Formula I wherein at least one R¹ group is a group of the formula
Q¹-X²-
wherein Q¹ is an aryl-(1-6C)alkyl, (3-7C)cycloalkyl-(1-6C)alkyl, (3-7C)cycloalkenyl-(1-6C)alkyl, heteroaryl-(1-6C)alkyl or heterocyclyl-(1-6C)alkyl group or an optionally substituted alkyl group and X² is an oxygen atom, the coupling of a quinoline of the Formula VIII 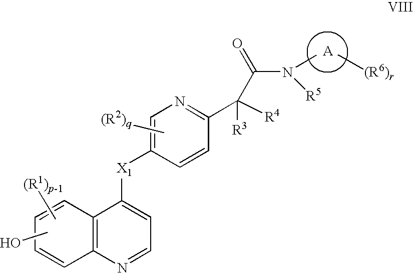
wherein each of p, R¹, X¹, q, R², R³, R⁴, R⁵, Ring A, r and R⁶ has any of the meanings defined in claim 1 except that any functional group is protected if necessary, with an appropriate alcohol wherein any functional group is protected if necessary, whereafter any protecting group that is present is removed;
(d) for the production of those compounds of the Formula I wherein a R⁶ group is a group of the formula -X⁶-R¹⁵ wherein X⁶ has any of the meanings defined in claim 1 and R¹⁵ is an amino-substituted (1-6C)alkyl group, the reaction of a compound of the Formula I wherein a R⁶ group is a group of the formula -X⁶-R¹⁵ wherein R¹⁵ is a halogeno-substituted (1-6C)alkyl group with an appropriate amine or with a nitrogen-containing heterocyclyl compound;
(e) for the production of those compounds of the Formula I wherein a R⁶ group is a group of the formula -X⁶-R¹⁵ wherein X⁶ has any of the meanings defined in claim 1 and R¹⁵ is an amino-substituted (1-6C)alkyl group, the reductive amination of a compound of the Formula I wherein a R⁶ group is a group of the formula -X⁶-R¹⁵ wherein R¹ is a formyl or (2-6C)alkanoyl group;
(f) for the production of those compounds of the Formula I wherein R⁵ is a (1-8C)alkyl group, the alkylation of a compound of the Formula I wherein R⁵ is hydrogen with a suitable alkylating agent; or
(g) for the production of those compounds of the Formula I wherein R¹ is a carboxy group, the cleavage of a compound of the Formula I wherein R¹ is a (1-6C)alkoxycarbonyl group;
and optionally forming a pharmaceutically-acceptable salt of a quinoline derivative of the Formula I by reaction of said quinoline derivative with a suitable acid.

16: A pharmaceutical composition which comprises a quinoline derivative of the Formula I, or a pharmaceutically-acceptable salt thereof, according to claim 1 in association with a pharmaceutically-acceptable diluent or carrier.

17-19. (canceled)

20: A method for treating a cell proliferative disorder in a warm-blooded animal in need of such treatment which comprises administering to said animal an effective amount of a quinoline derivative of the Formula I according to claim 1, or a pharmaceutically-acceptable salt thereof.

21: The method of claim 20 wherein said cell proliferative disorder is a solid tumour disease.
